(19) United States
(12) Patent Application Publication
(10) Pub. No.: US 2012/0233834 A1
(43) Pub. Date: Sep. 20, 2012

(54) SYSTEMS, DEVICES AND METHODS FOR ASSEMBLING AUTOMATIC INJECTION DEVICES AND SUB-ASSEMBLIES THEREOF

(75) Inventors: William P. Szechinski, Chicago, IL (US); Kenneth E. Howard, Kenosha, WI (US)

(73) Assignee: ABBOTT BIOTECHNOLOGY LTD., Hamilton (BM)

(21) Appl. No.: 13/424,194

(22) Filed: Mar. 19, 2012

Related U.S. Application Data
(60) Provisional application No. 61/454,097, filed on Mar. 18, 2011.

(51) Int. Cl. B23Q 17/00

(52) U.S. Cl. 29/407.01; 29/705

(57) ABSTRACT

Exemplary embodiments provide automated assembly systems, devices and methods for assembling components for use in forming an automatic injection device. Exemplary assembly systems monitor, in real time, the frictional forces experienced as a plurality of components are assembled. The detected forces are used in providing real-time feedback to automatically control the assembly process and to determine whether the components are assembled properly.

### RELATED APPLICATIONS

This application is a non-provisional of and claims the benefit of priority to U.S. Provisional Patent Application No. 61/454,097 titled “Systems, Devices and Methods for Assembling Automatic Injection Devices,” filed Mar. 18, 2011, the entire contents of which are hereby expressly incorporated herein by reference in their entirety.

### BACKGROUND

Automatic injection devices offer an alternative to manually-operated syringes for delivering therapeutic agents into patients' bodies and allow patients to self-administer injections. Automatic injection devices have been used to deliver medications under emergency conditions, for example, to administer epinephrine to counteract the effects of a severe allergic reaction. Automatic injection devices have also been described for use in administering anti-arrhythmic medications and selective thrombolytic agents during a heart attack (See, e.g., U.S. Pat. Nos. 3,910,260; 4,004,577; 4,689,042; 4,755,169; and 4,795,433). Various types of automatic injection devices are also described in, for example, U.S. Pat. Nos. 3,941,130; 4,261,358; 5,085,642; 5,092,843; 5,102,393; 5,267,963; 6,149,626; 6,270,479; and 6,371,939; and International Patent Publication No. WO/2008/005315.

Conventional automatic injection devices often include a housing that houses a syringe containing a therapeutic agent. A syringe actuation component (for example, a plunger) may be provided to compress and eject the therapeutic agent through an injection needle during an injection. A firing mechanism sub-assembly may be provided to cause the syringe to move forwardly within the housing so that the injection needle projects from the housing for an injection. The firing mechanism sub-assembly may also actuate the syringe actuation component so that the therapeutic agent is ejected through the injection needle. A syringe housing sub-assembly may be provided to facilitate movement of the syringe within the housing during an injection and to protect the injection needle after an injection is performed.

Conventional automatic injection devices often experience failure due to suboptimal assembly of the firing mechanism sub-assembly, the syringe housing sub-assembly or the overall assembly of the syringe into the housing of the automatic injection device. Certain conventional techniques of assembling automatic injection devices and sub-assemblies thereof rely on cam-driven mechanisms that drive certain components of the assembly relative to certain other components over fixed predetermined distances. However, the optimal distance over which the components need to be driven during assembly may vary depending on one or more factors that include, but are not limited to, variability in the materials forming the components, process variations in forming the components, the sizes and shapes of the components, and the like. Since a cam-driven mechanism often uses fixed distances for assembling components, the conventional techniques are unable to adapt the assembly process based on factors that vary depending on the specific components used.

As a result, many conventional assembly techniques fail to properly, consistently and reliably assemble the firing mechanism sub-assembly, the syringe housing sub-assembly or the overall assembly of the syringe into the housing of the automatic injection device. Unsuccessful or improper assembly adversely affects the proper operation and functioning of the assembled device. For example, in a conventional automatic injection device in which the syringe is inserted too far into the housing of the device during assembly, the injection needle may be inserted too far into a patient's body in use. Conversely, in a conventional automatic injection device in which the syringe is not inserted far enough into the housing of the device during assembly, the injection needle may not be inserted to a sufficient depth into a patient's body in use. As such, improper assembly is highly undesirable in automatic injection devices and their constituent sub-assemblies.

### SUMMARY

Exemplary embodiments provide assembly systems, devices and methods for assembling an automatic injection device or components of an automatic injection device, or both. Exemplary assembly systems monitor, in real time, the frictional forces experienced as a plurality of components are assembled. The detected forces are used in providing real-time feedback to automatically control the assembly process and to determine whether the components are being assembled properly.

In accordance with one exemplary embodiment, a method is provided for assembling a sub-assembly of components for use in forming an automatic injection device. The method includes cooperatively coupling a first component of the automatic injection device to a second component of the automatic injection device. The method also includes detecting one or more forces exerted against the cooperative coupling of the first component to the second component, and generating a trigger instruction upon verifying that one or more of the detected forces satisfy or do not satisfy one or more predefined force values. The method further includes automatically controlling the cooperative coupling of the first component to the second component in response to the trigger instruction.

In accordance with another exemplary embodiment, a system is provided for assembling a sub-assembly of components for use in forming an automatic injection device. The system includes an assembly station for cooperatively coupling a first component of the automatic injection device to a second component of the automatic injection device. The system also includes a force detection mechanism configured to detect one or more forces exerted against the cooperative coupling of the first component to the second component. The system further includes a controller programmed to automatically generate a trigger instruction upon verifying that the one or more of the detected forces satisfy or do not satisfy one or more predefined force values. The assembly station is configured to automatically control the cooperative coupling of the first component to the second component in response to the trigger instruction.

In accordance with another exemplary embodiment, a method is provided for assembling an automatic injection device. The method includes inserting a syringe assembly of the automatic injection device into a housing assembly of the automatic injection device, the syringe assembly comprising a first outer diameter greater than a second outer diameter, an inner surface of the housing assembly having a friction point. The method also includes detecting one or more forces generated as the first outer diameter and the second outer diameter of the syringe assembly are inserted past the friction point in the housing assembly, and generating a trigger instruction upon matching one or more of the detected forces to one or more predefined forces values. The method further includes controlling the insertion of the syringe assembly into the housing assembly in response to the trigger instruction.

In accordance with another exemplary embodiment, a system is provided for assembling an automatic injection device. The system includes an insertion mechanism for inserting a syringe assembly into a housing assembly of the automatic injection device, an outer surface of the syringe assembly comprising a first feature, an inner surface of the housing assembly having a friction point. The system also includes a force detection mechanism for detecting one or more forces generated as the first feature on the syringe assembly passes by the friction point in the housing assembly, and for generating a trigger instruction upon matching one or more of the detected forces to one or more predefined force values. An aspect of the insertion mechanism is specified or changed in response to the trigger instruction.

In accordance with another exemplary embodiment, a system is provided for assembling an automatic injection device. The system includes a mechanical member for inserting a syringe assembly into a housing assembly of the automatic injection device, an outer surface of the syringe assembly comprising a first feature, an inner surface of the housing assembly having a friction point. The system includes a motion generator for driving the mechanical member. The system also includes a force detection mechanism for detecting one or more forces generated as the first feature on the syringe assembly passes by the friction point in the housing assembly, and for generating a trigger instruction upon matching one or more of the detected forces to one or more corresponding predefined force values. An aspect of the operation of the motion generator is specified or changed in response to the trigger instruction.

### BRIEF DESCRIPTION OF THE DRAWINGS

The foregoing and other objects, aspects, features and advantages of exemplary embodiments will be more fully understood from the following description when read together with the accompanying drawings, in which:

FIG. 1 illustrates a perspective view of an exemplary automatic injection device in which caps that cover proximal and distal ends of the housing are removed from the housing.

FIG. 2 illustrates a perspective view of the exemplary automatic injection device of FIG. 1 in which the housing is capped using proximal and distal caps.

FIG. 3 (prior art) illustrates a cross-sectional schematic view of an exemplary automatic injection device before use.

FIG. 4 (prior art) illustrates a cross-sectional schematic view of the exemplary automatic injection device of FIG. 3 during a subsequent stage of operation.

FIG. 5 illustrates a perspective view of an exemplary automatic injection device including a syringe housing sub-assembly and a firing mechanism sub-assembly.

FIG. 6 illustrates an exploded perspective view of the firing mechanism sub-assembly of the exemplary automatic injection device of FIG. 5.

FIG. 7 illustrates a perspective view of a syringe actuation component of the exemplary firing mechanism sub-assembly of FIG. 6.

FIG. 8 illustrates an exploded perspective view of the syringe housing sub-assembly of the exemplary automatic injection device of FIG. 5.

FIG. 9 illustrates a perspective view of a syringe carrier of the exemplary syringe housing sub-assembly of FIG. 8.

FIGS. 10A and 10B illustrate cross-sectional views of an exemplary assembled automatic injection device offset by 90° angles from each other, in which the syringe housing sub-assembly and the firing mechanism sub-assembly are coupled together.

FIG. 11 illustrates a cross-sectional view of an exemplary assembled automatic injection device.

FIG. 12 illustrates a cross-sectional view of an exemplary automatic injection device housing an exemplary syringe.

FIG. 13A illustrates an exemplary perspective view of an assembly system that may be used to assemble the exemplary syringe housing sub-assembly.

FIG. 13B illustrates an exemplary perspective view of another assembly system that may be used to assemble the exemplary syringe housing sub-assembly.

FIGS. 14A and 14B are flowcharts illustrating an exemplary method for assembling a syringe housing sub-assembly for use in an automatic injection device.

FIG. 15 illustrates an exemplary force profile of the forces experienced at the press head during assembly of the syringe housing sub-assembly.

FIG. 16 illustrates another exemplary force profile of the forces experienced at the press head during assembly of the syringe housing sub-assembly.

FIG. 17 illustrates an exemplary force profile showing forces experienced at the press head during deployment of a shroud.

FIG. 18 illustrates another exemplary force profile showing forces experienced at the press head during deployment of a shroud.

FIGS. 19A and 19B illustrate an exemplary perspective view of an assembly system that may be used to assemble an exemplary firing mechanism sub-assembly.

FIGS. 20A and 20B illustrate an exemplary perspective view of another assembly system that may be used to assemble an exemplary firing mechanism sub-assembly.

FIGS. 21A and 21B are flowcharts illustrating an exemplary method for assembling a firing mechanism sub-assembly for use in an automatic injection device.

FIG. 22 illustrates an exemplary force profile of the forces experienced at the press head during assembly of the firing mechanism sub-assembly.

FIG. 23 illustrates another exemplary force profile of the forces experienced at the press head during assembly of the firing mechanism sub-assembly.

FIG. 24 illustrates a graph of exemplary force detections performed during assembly of the firing mechanism sub-assembly.

FIG. 25A is a schematic view of a first assembly state during assembly of the firing mechanism sub-assembly, in which the trigger anchoring portion of the syringe actuation component impinges upon and resists the inner cylindrical tube within the firing body.

FIG. 25B is a schematic view of the first assembly state of FIG. 25A rotated by about 90 degrees from the view of FIG. 25A.

FIG. 26A is a schematic view of a second assembly state during assembly a firing mechanism sub-assembly, in which the tabbed feet of the trigger anchoring portion passes through the distal end of the inner cylindrical tube of the firing body.

FIG. 26B is a schematic view of the second assembly state of FIG. 26A rotated by about 90 degrees from the view of FIG. 26A.

FIG. 27 illustrates a perspective view of an exemplary rigid needle shield and a characteristic force profile graph associated with the insertion of the rigid needle shield in which the distal end of the rigid needle shield is disposed exactly or approximately at a local friction point in the proximal cap.

FIG. 28 illustrates a perspective view of an exemplary rigid needle shield and a characteristic force profile graph associated with the insertion of the rigid needle shield in which the distal end of the rigid needle shield is disposed beyond a local friction point in the proximal cap toward the proximal end of the proximal cap.

FIG. 29 is a block diagram illustrating an exemplary insertion system that may be used in exemplary embodiments to insert a syringe into an automatic injection device.

FIG. 30A is a side view of an exemplary insertion system.

FIG. 30B is a perspective view of the exemplary insertion system of FIG. 30A.

FIG. 31 is a flowchart illustrating an exemplary method for inserting a syringe into the housing of an automatic injection device.

FIG. 32 illustrates a graph showing a characteristic force profile generated during the insertion of an exemplary rigid needle shield into the housing of an automatic injection device.

FIG. 33 illustrates a user interface associated with a motion generator driving the syringe into the housing of the automatic injection device associated with FIG. 32.

FIG. 34 illustrates a graph showing a characteristic force profile generated during the insertion of an exemplary rigid needle shield into the housing of an automatic injection device.

FIG. 35 illustrates a user interface associated with a motion generator driving the syringe into the housing of the automatic injection device associated with FIG. 34.

FIG. 36 is a flowchart illustrating another exemplary method for inserting a syringe into the housing of an automatic injection device.

FIG. 37 illustrates a graph showing a characteristic force profile generated during the insertion of an exemplary rigid needle shield into the housing of an automatic injection device.

FIG. 38 illustrates a user interface associated with a motion generator driving the syringe into the housing of the automatic injection device associated with FIG. 37.

FIG. 39 illustrates an empty graph for showing a characteristic force profile generated during the insertion of an exemplary rigid needle shield into the housing of an automatic injection device.

FIG. 40 illustrates a graph showing a characteristic force profile generated during the insertion of an exemplary rigid needle shield into the housing of an automatic injection device.

FIG. 41 illustrates a graph showing a characteristic force profile generated during the insertion of an exemplary rigid needle shield into the housing of an automatic injection device.

FIG. 42 illustrates a graph showing a characteristic force profile generated during the insertion of an exemplary rigid needle shield into the housing of an automatic injection device.

FIG. 43 illustrates a histogram of the density of syringes having different distances from the rigid needle shield to end of the proximal cap.

FIG. 44 illustrates a histogram of the density of syringes having different distances from the rigid needle shield to end of the proximal cap.

FIG. 45 illustrates a histogram of the density of syringes having different distances from the rigid needle shield to end of the proximal cap.

FIG. 46 illustrates a histogram of the density of syringes having different distances from the rigid needle shield to end of the proximal cap.

FIG. 47 illustrates a histogram of the density of syringes having different distances from the rigid needle shield to end of the proximal cap.

FIG. 48 illustrates different exemplary orientations of a rigid needle shield inside a proximal cap.

FIG. 49 illustrates a graph showing force profiles for different rigid needle shield orientations for syringes of a first type, i.e., Type 1.

FIG. 50 illustrates a graph showing force profiles for different rigid needle shield orientations for glass pre-fillable syringes of a second type, i.e., Type 2.

FIG. 51 illustrates a graph showing force profiles for different rigid needle shield orientations for glass pre-fillable syringes of a third type, i.e., Type 3.

FIG. 52 illustrates a histogram of the density of Type 1 syringes having different forces of insertion at different rigid needle shield orientations.

FIG. 53 illustrates a histogram of the density of Type 2 syringes having different forces of insertion at different rigid needle shield orientations.

FIG. 54 illustrates a histogram of the density of Type 3 syringes having different forces of insertion at different rigid needle shield orientations.

FIG. 55 illustrates a one-way ANOVA analysis of the effect of different rigid needle shield orientations on insertion forces for Type 1 syringes.

FIG. 56 illustrates a one-way ANOVA analysis of the effect of different rigid needle shield orientations on insertion forces for Type 2 syringes.

FIG. 57 illustrates a one-way ANOVA analysis of the effect of different rigid needle shield orientations on insertion forces for Type 3 syringes.

FIG. 58 illustrates a histogram of the density of different syringes requiring different proximal cap removal forces.

FIG. 59 illustrates exemplary elongation rings for elongating syringes for testing.

FIG. 60 illustrates an exemplary rubber grommet for elongating syringes for testing.

FIG. 61 illustrates an exemplary steel ring for elongating syringes for testing.

FIG. 62 illustrates a user interface associated with a motion generator driving the syringe into the housing of the automatic injection device.

FIG. 63 illustrates a graph showing a characteristic force profile generated during the insertion of an exemplary rigid needle shield into the housing of an automatic injection device.

FIG. 64 illustrates an exemplary rigid needle shield and a graph showing a characteristic force profile generated during the insertion of the exemplary rigid needle shield into the housing of an automatic injection device.

FIG. 65 illustrates a histogram of the density of different syringes having different distances between the rigid needle shield and the end of the proximal cap.

FIG. 66 illustrates a graph showing a characteristic force profile generated during the insertion of an exemplary rigid needle shield into the housing of an automatic injection device.

FIG. 67 illustrates a graph showing a characteristic force profile generated during the insertion of an exemplary rigid needle shield into the housing of an automatic injection device.

FIG. 68 illustrates a user interface associated with a motion generator driving the syringe into the housing of the automatic injection device.

FIG. 69 illustrates a graph showing a characteristic force profile generated during the insertion of an exemplary rigid needle shield into the housing of an automatic injection device.

FIG. 70 illustrates a histogram of the density of different syringes having different distances between the rigid needle shield and the end of the proximal cap.

FIG. 71 illustrates a user interface associated with a motion generator driving the syringe into the housing of the automatic injection device.

FIG. 72 illustrates an empty graph for plotting a characteristic force profile generated during the insertion of an exemplary rigid needle shield into the housing of an automatic injection device.

FIG. 73 illustrates a graph showing a characteristic force profile generated during the insertion of an exemplary rigid needle shield into the housing of an automatic injection device.

FIG. 74 illustrates a histogram of the density of different syringes having different distances between the rigid needle shield and the end of the proximal cap.

FIG. 75 illustrates a graph showing a characteristic force profile generated during the insertion of an exemplary rigid needle shield into the housing of an automatic injection device.

FIG. 76 illustrates a graph showing a characteristic force profile generated during the insertion of an exemplary rigid needle shield into the housing of an automatic injection device.

FIG. 77 illustrates a block diagram of an exemplary computing device that may be used in an exemplary assembly system.

### DETAILED DESCRIPTION

Exemplary embodiments address the shortcomings of conventional techniques for assembling automatic injection devices by providing assembly systems, devices and methods that monitor the forces experienced during the assembly process to adapt the assembly process to the particular components being assembled. This ensures proper, consistent and reliable assembly of the components of a syringe housing sub-assembly, a firing mechanism sub-assembly or an overall automatic injection device, regardless of material and process variations in the components assembled.

In an exemplary automated process of assembling a syringe housing sub-assembly, a syringe carrier may be held in place by an assembly system with a distal portion of a proximal housing component positioned over the syringe carrier. A biasing mechanism and a stepped shroud may be positioned within a proximal portion of the proximal housing component. During the assembly process, the stepped shroud may be inserted into the proximal housing component so that the biasing mechanism is compressed between the syringe carrier and the shroud. The assembly process may automatically detect and monitor the forces experienced against the compression of the biasing mechanism. The detected forces may be used in a feedback mechanism to automatically control or alter one or more aspects of the assembly process. This force feedback mechanism allows the assembly system to automatically and reliably determine the end point of the insertion of the shroud.

That is, in exemplary embodiments, the shroud is not inserted over a fixed predetermined distance in order to assemble the syringe housing sub-assembly. Rather, the exemplary assembly process is automatically controlled based on one or more forces that are detected during the process and that may be used as feedback to accelerate, decelerate, start and/or stop the insertion of the shroud for assembly with the syringe carrier. This allows the exemplary assembly process to automatically accommodate for variability in the components of the syringe housing sub-assembly, and to thereby achieve reliable assembly of any set of components. In contrast, conventional assembly processes using mechanical cams insert one or more components over a fixed predetermined distance to assemble them with one or more other components. The use of a fixed predetermined insertion process, without the benefit of feedback from force measurements, prevents the conventional processes from accommodating for variability in the components, and may result in improper assembly of the syringe housing sub-assemblies.

In an exemplary automated process of assembling a firing mechanism sub-assembly, a firing button may be positioned between a distal cap and a firing body. A distal portion of a biasing mechanism may be positioned within the hollow barrel portion of the firing body. A syringe actuation component may be positioned at the proximal end of the firing body. During the automated assembly process, the syringe actuation component may be inserted into the hollow barrel portion of the firing body by an assembly system, causing compression of the biasing mechanism.

The automated assembly process may automatically detect and monitor the force experienced against the compression of the biasing mechanism. The detected forces may be used in an automated feedback mechanism to control or alter one or more aspects of the assembly process. This force feedback mechanism allows the assembly system to automatically and reliably determine the end point of the insertion of the syringe actuation component into the firing body. That is, in exemplary embodiments, a syringe actuation component is not inserted over a fixed predetermined distance in order to assemble the firing mechanism sub-assembly. Rather, the exemplary assembly process is automatically controlled based on one or more forces that are detected during the process and that may be used as feedback to accelerate, decelerate, start and/or stop the insertion of the syringe actuation component into the firing body. This allows the exemplary assembly process to accommodate for variability in the components of the syringe housing sub-assembly, and to thereby achieve reliable assembly of any set of components. In contrast, conventional assembly processes using mechanical cams insert one or more components over a fixed predetermined distance to assemble them with one or more other components. The use of a fixed predetermined insertion process, without the benefit of feedback from force measurements, prevents the conventional processes from accommodating for variability in the components, and may result in improper assembly of the syringe housing sub-assemblies.

In an exemplary automated process of assembling an automatic injection device, a syringe assembly may be assembled with a housing assembly in a controlled automated manner. The housing assembly may include a housing of the device fitted with a proximal cap for covering an injection needle. The syringe assembly may include a syringe housing sub-assembly coupled to a syringe and a firing mechanism sub-assembly. The proximal end of the syringe may be coupled to an injection needle that is covered by a rigid needle shield and, optionally, a soft needle shield. During assembly, the syringe assembly is moved toward the housing assembly and/or the housing assembly is moved toward the syringe assembly, such that the rigid needle shield is inserted to an appropriate insertion depth into the proximal cap. Exemplary embodiments may detect one or more forces values and/or the force profile generated during the assembly process in order to consistently and reliably insert the syringe assembly a desired distance into the housing assembly. In one example, one or more force values or characteristic force features may be detected and used to determine, in real time, when the assembly is completed or is near completion.

Automatic injection devices provided in accordance with exemplary embodiments may be used for administering any type of substance into a patient's body including, but not limited to, liquid therapeutic agents, e.g., adalimumab (HUMIRA®), golimumab, etc.

### I. DEFINITIONS OF EXEMPLARY TERMS

Certain terms are defined in this section to facilitate understanding of exemplary embodiments.

The terms “automatic injection device,” “autoinjector” and “autoinjector pen” refer to a device that enables a patient to self-administer a dose of a substance, such as a liquid medication, wherein the automatic injection device differs from a standard syringe by the inclusion of a firing mechanism sub-assembly for automatically delivering the substance into the patient's body by injection when the firing mechanism sub-assembly is engaged. In an exemplary embodiment, the automatic injection device may be wearable on the patient's body.

The automatic injection device, e.g., autoinjector pen, of exemplary embodiments may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody, antibody portion, or other TNFαinhibitor may vary according to factors such as the disease state, age, sex, and weight of the patient, and the ability of the antibody, antibody portion, or other TNFα inhibitor to elicit a desired response in the patient. A therapeutically effective amount is also one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in patients prior to or at an earlier stage of disease, the prophylactically effective amount will be less than the therapeutically effective amount.

The term “substance” refers to any type of drug, biologically active agent, biological substance, chemical substance or biochemical substance that is capable of being administered in a therapeutically effective amount to a patient employing exemplary automatic injection devices. Exemplary substances include, but are not limited to, agents in a liquid state. Such agents may include, but are not limited to, adalimumab (HUMIRA®) and proteins that are in a liquid solution, e.g., fusion proteins and enzymes. Examples of proteins in solution include, but are not limited to, Pulmozyme (Dornase alfa), Regranex (Becaplermin), Activase (Alteplase), Aldurazyme (Laronidase), Amevive (Alefacept), Aranesp (Darbepoetin alfa), Becaplermin Concentrate, Betaseron (Interferon beta-1b), BOTOX (Botulinum Toxin Type A), Elitek (Rasburicase), Elspar (Asparaginase), Epogen (Epoetin alfa), Enbrel (Etanercept), Fabrazyme (Agalsidase beta), Infergen (Interferon alfacon-1), Intron A (Interferon alfa-2a), Kineret (Anakinra), MYOBLOC (Botulinum Toxin Type B), Neulasta (Pegfilgrastim), Neumega (Oprelvekin), Neupogen (Filgrastim), Ontak (Denileukin diftitox), PEGASYS (Peginterferon alfa-2a), Proleukin (Aldesleukin), Pulmozyme (Dornase alfa), Rebif (Interferon beta-1a), Regranex (Becaplermin), Retavase (Reteplase), Roferon-A (Interferon alfa-2), TNKase (Tenecteplase), and Xigris (Drotrecogin alfa), Arcalyst (Rilonacept), NPlate (Romiplostim), Mircera (methoxypolyethylene glycol-epoetin beta), Cinryze (C1 esterase inhibitor), Elaprase (idursulfase), Myozyme (alglucosidase alfa), Orencia (abatacept), Naglazyme (galsulfase), Kepivance (palifermin) and Actimmune (interferon gamma-1b).

A protein in solution may also be an immunoglobulin or antigen-binding fragment thereof, such as an antibody or antigen-binding portion thereof. Examples of antibodies that may be used in an exemplary automatic injection device include, but are not limited to, chimeric antibodies, non-human antibodies, human antibodies, humanized antibodies, and domain antibodies (dAbs). In an exemplary embodiment, the immunoglobulin or antigen-binding fragment thereof, is an anti-TNFα and/or an anti-IL-12 antibody (e.g., it may be a dual variable domain immunoglobulin (DVD) Ig™). Other examples of immunoglobulins or antigen-binding fragments thereof that may be used in the methods and compositions of exemplary embodiments include, but are not limited to, 1D4.7 (anti-IL-12/IL-23 antibody; Abbott Laboratories); 2.5(E)mg1 (anti-IL-18; Abbott Laboratories); 13C5.5 (anti-IL-13 antibody; Abbott Laboratories); J695 (anti-IL-12; Abbott Laboratories); Afelimomab (Fab 2 anti-TNF; Abbott Laboratories); HUMIRA (adalimumab) Abbott Laboratories); Campath (Alemtuzumab); CEA-Scan Arcitumomab (fab fragment); Erbitux (Cetuximab); Herceptin (Trastuzumab); Myoscint (Imciromab Pentetate); ProstaScint (Capromab Pendetide); Remicade (Infliximab); ReoPro (Abciximab); Rituxan (Rituximab); Simulect (Basiliximab); Synagis (Palivizumab); Verluma (Nofetumomab); Xolair (Omalizumab); Zenapax (Daclizumab); Zevalin (Ibritumomab Tiuxetan); Orthoclone OKT3 (Muromonab-CD3); Panorex (Edrecolomab); Mylotarg (Gemtuzumab ozogamicin); golimumab (Centocor); Cimzia (Certolizumab pegol); Soliris (Eculizumab); CNTO 1275 (ustekinumab); Vectibix (panitumumab); Bexxar (tositumomab and I¹³¹ tositumomab); and Avastin (bevacizumab).

Additional examples of immunoglobulins, or antigen-binding fragments thereof, that may be used in the methods and compositions of exemplary embodiments include, but are not limited to, proteins comprising one or more of the following: the D2E7 light chain variable region (SEQ ID NO: 1), the D2E7 heavy chain variable region (SEQ ID NO: 2), the D2E7 light chain variable region CDR3 (SEQ ID NO: 3), the D2E7 heavy chain variable region CDR3 (SEQ ID NO:4), the D2E& light chain variable region CDR2 (SEQ ID NO: 5), the D2E7 heavy chain variable region CDR2 (SEQ ID NO: 6), the D2E7 light chain variable reion CDR1 (SEQ ID NO: 7), the D2E7 heavy chain variable region CDR1 (SEQ ID NO: 8), the 2SD4 light chain variable region (SEQ ID NO: 9), the 2SD4 heavy chain variable region (SEQ ID NO: 10), the 2SD4 light chain variable CDR3 (SEQ ID NO: 11), the EP B12 light chain variable CDR3 (SEQ ID NO: 12), the VL10E4 light chain variable CDR3 (SEQ ID NO: 13), theVL100A9 light chain variable CDR3 (SEQ ID NO: 14), the VLL100D2 light chain variable CDR3 (SEQ ID NO: 15), the VLLOF4 light chain variable CDR3 (SEQ ID NO: 16), theLOE5 light chain variable CDR3 (SEQ ID NO: 17), the VLLOG7 light chain variable CDR3 (SEQ ID NO: 18), the VLLOG9 light chain variable CDR3 (SEQ ID NO: 19), the VLLOH1 light chain variable CDR3 (SEQ ID NO: 20), the VLLOH10 light chain variable CDR3 (SEQ ID NO: 21), the VL1B7 light chain variable CDR3 (SEQ ID NO: 22), the VL1C1 light chain variable CDR3 (SEQ ID NO: 23), the VL0.1F4 light chain variable CDR3 (SEQ ID NO: 24), the VL0.1H8 light chain variable CDR3 (SEQ ID NO: 25), the LOE7.A light chain variable CDR3 (SEQ ID NO: 26), the 2SD4 heavy chain variable region CDR (SEQ ID NO: 27), theVH1B 11 heavy chain variable region CDR (SEQ ID NO: 28), the VH1D8 heavy chain variable region CDR (SEQ ID NO: 29), the VH1A11 heavy chain variable region CDR (SEQ ID NO: 30), the VH1B 12 heavy chain variable region CDR (SEQ ID NO: 31), the VH1E4 heavy chain variable region CDR (SEQ ID NO: 32), the VH1F6 heavy chain variable region CDR (SEQ ID NO: 33), the 3C-H2 heavy chain variable region CDR (SEQ ID NO: 34), and the VH1-D2.N heavy chain variable region CDR (SEQ ID NO: 35).

The term “human TNFα” (abbreviated herein as hTNFα, or simply hTNF) refers to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. (1984) Nature 312:724-729; Davis, J. M., et al. (1987) Biochem. 26:1322-1326; and Jones, E. Y., et al. (1989) Nature 338:225-228. The term human TNFα is intended to include recombinant human TNFα (rhTNFα), which can be prepared by standard recombinant expression methods or purchased commercially (R & D Systems, Catalog No. 210-TA, Minneapolis, Minn.). TNFα is also referred to as TNF.

The term “TNFα inhibitor” refers to an agent that interferes with TNFα activity. The term also includes each of the anti-TNFα human antibodies (used interchangeably herein with TNFα antibodies) and antibody portions described herein as well as those described in U.S. Pat. Nos. 6,090,382; 6,258,562; 6,509,015; 7,223,394; and 6,509,015. In one embodiment, the TNFα inhibitor used in the invention is an anti-TNFα antibody, or a fragment thereof, including infliximab (Remicade®, Johnson and Johnson; described in U.S. Pat. No. 5,656,272); CDP571 (a humanized monoclonal anti-TNF-alpha IgG4 antibody); CDP 870 (a humanized monoclonal anti-TNF-alpha antibody fragment); an anti-TNF dAb (Peptech); CNTO 148 (golimumab; Centocor, see WO 02/12502 and U.S. Pat. No. 7,521,206 and U.S. Pat. No. 7,250,165); and adalimumab (HUMIRA® Abbott Laboratories, a human anti-TNF mAb, described in U.S. Pat. No. 6,090,382 as D2E7). Additional TNF antibodies that may be used in the invention are described in U.S. Pat. Nos. 6,593,458; 6,498,237; 6,451,983; and 6,448,380. In another embodiment, the TNFα inhibitor is a TNF fusion protein, e.g., etanercept (Enbrel®, Amgen; described in WO 91/03553 and WO 09/406,476). In another embodiment, the TNFα inhibitor is a recombinant TNF binding protein (r-TBP-I) (Serono).

In one embodiment, the term “TNFα inhibitor” excludes infliximab. In one embodiment, the term “TNFα inhibitor” excludes adalimumab. In another embodiment, the term “TNFαinhibitor” excludes adalimumab and infliximab.

In one embodiment, the term “TNFα inhibitor” excludes etanercept, and, optionally, adalimumab, infliximab, and adalimumab and infliximab.

In one embodiment, the term “TNFα antibody” excludes infliximab. In one embodiment, the term “TNFα antibody” excludes adalimumab. In another embodiment, the term “TNFαantibody” excludes adalimumab and infliximab.

The term “treatment” refers to therapeutic treatment, as well as prophylactic or suppressive measures, for the treatment of a disorder, such as a disorder in which TNFα is detrimental, e.g., rheumatoid arthritis.

The term “patient” refers to any type of animal, human or non-human, that may be injected a substance using exemplary automatic injection devices.

The terms “pre-filled syringe/device” and “pre-fillable syringe/device” encompass a syringe/device that is filled with a substance immediately prior to administration of the substance to a patient and a syringe/device that is filled with a substance and stored in this pre-filled form for a period of time before administration of the substance to a patient.

The term “firing mechanism” refers to a mechanism that, when engaged by a firing engagement mechanism, automatically delivers a substance contained in an automatic injection device into a patient's body. A firing engagement mechanism may be any type of mechanism that engages and triggers the firing mechanism including, but not limited to, a firing button that may be pushed by a patient to trigger the firing mechanism.

The term “distal” refers to a portion, end or component of an exemplary automatic injection device that is farthest from an injection site on a patient's body when the device is held against the patient for an injection or for mimicking an injection.

The term “proximal” refers to a portion, end or component of an exemplary automatic injection device that is closest to an injection site on a patient's body when the device is held against the patient for an injection or for mimicking an injection.

The term “friction point” refers to a location or region in or on a first component of an automatic injection device that resists with frictional force the entry of one or more structural features of a second component past the friction point. In an exemplary embodiment, the friction point may include a local constriction that locally decreases the diameter of a hollow internal bore of the first component. In an exemplary embodiment, the friction point may include a protrusion that extends inwardly from the inner wall of the first component into a bore or cavity of the first component. The protrusion may extend radially continuously along the entire circumference of the inner wall, or may extend radially in two or more non-contiguous sections along the circumference of the inner wall. In an exemplary embodiment, the friction point may include one or more ornamental components, e.g., raised letterings, etc., provided on the inner wall of the first component.

The term “force profile” refers to a graph, trace or plot of force values detected during an assembly process.

The term “trigger condition” refers to one or more force features in a force profile that, when measured or detected, is used to set or change in one or more aspects of an assembly process. An exemplary “trigger condition” may include the satisfaction of a “trigger force,” a set of “trigger forces,” or both a “trigger force” and a “trigger hysteresis.”

The term “trigger force” refers to a force value that, when measured or detected in the force profile, satisfies at least a part of a trigger condition.

The term “trigger hysteresis” or “differential trigger force” refers, directly or indirectly, to an earlier force value that, when measured or detected in a force profile before measurement or detection of the trigger force value, satisfies a part of the trigger condition. The earlier force may be higher or lower than the trigger force. In an exemplary embodiment, the trigger hysteresis directly refers to the earlier force. In another exemplary embodiment, the trigger hysteresis refers to a force difference between an earlier force and the trigger force.

If the earlier force is higher than the trigger force, in an exemplary embodiment, the trigger condition may be satisfied if the measured or detected forces fall from the earlier force to the trigger force without intermediate rises. If the earlier force is lower than the trigger force in an exemplary embodiment, the trigger condition may be satisfied if the measured or detected forces rise from the earlier force to the trigger force without immediate falls.

The term “trigger” refers to an output or instruction generated in exemplary embodiments in response to the satisfaction of a trigger condition. In an exemplary embodiment, the trigger may generally indicate that the trigger condition has been satisfied. In some exemplary embodiments, the trigger may contain or result in the generation of one or more instructions for controlling a motion generator that drives the syringe insertion process, for example, starting, stopping, accelerating, decelerating the motion generator, and the like.

### II. EXEMPLARY AUTOMATIC INJECTION DEVICES

Exemplary embodiments will be described below with reference to certain illustrative embodiments. While exemplary embodiments are described with respect to using an automatic injection device to provide an injection of a dose of a liquid substance, one of ordinary skill in the art will recognize that exemplary embodiments are not limited to the illustrative embodiments and that exemplary automatic injection devices may be used to inject any suitable substance into a patient. In addition, components of exemplary automatic injection devices and methods of assembling and using exemplary automatic injection devices are not limited to the illustrative embodiments described below.

A syringe of an exemplary automatic injections device may contain a dose of a TNFαinhibitor. In an exemplary embodiment, the TNFα inhibitor may be a human TNFα antibody or antigen-biding portion thereof. In an exemplary embodiment, the human TNFα antibody or antigen-binding portion thereof may be adalimumab or golimumab.

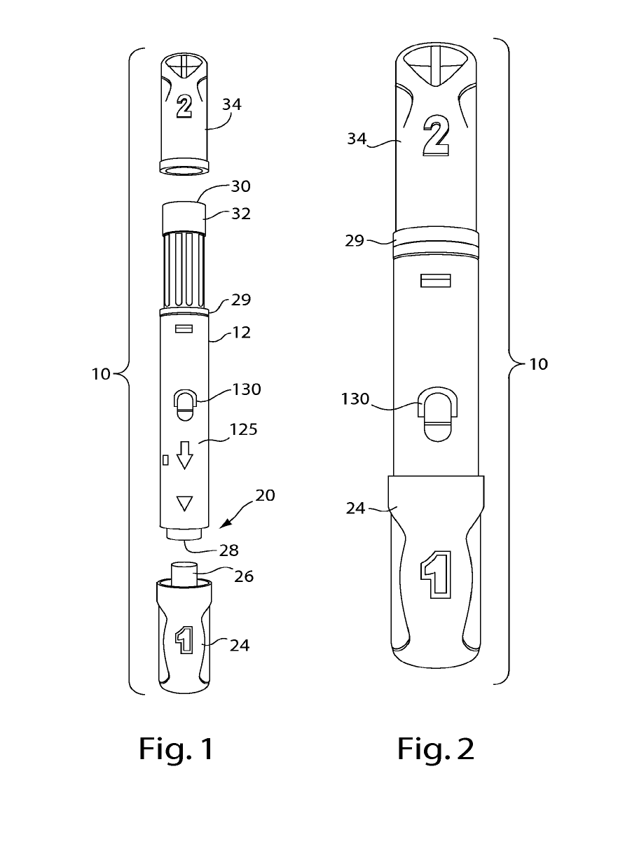

FIGS. 1 and 2 illustrate an exemplary automatic injection device 10 suitable for injecting a dose of a substance, such as a liquid drug, into a patient. FIG. 1 illustrates a perspective view of the exemplary automatic injection device 10 in which caps that cover proximal and distal ends of the housing are removed. FIG. 2 illustrates a perspective view of the exemplary automatic injection device 10 of FIG. 1 in which the proximal and distal ends of the housing are capped using proximal and distal caps.

Referring to FIG. 1, the automatic injection device 10 includes a housing 12 for housing a container, such as a syringe, containing a dose of a substance to be injected into a patient's body. The housing 12 has a tubular configuration, although one of ordinary skill in the art will recognize that the housing 12 may have any size, shape and configuration capable of housing a syringe or other container. While exemplary embodiments will be described with respect to a syringe mounted in the housing 12, one of ordinary skill in the art will recognize that the automatic injection device 10 may employ any other suitable container for storing and dispensing a substance, for example, a cartridge.

The exemplary syringe is preferably slidably mounted in the housing 12, as described in detail below. When the device 10 is in an inactivated position, the syringe is sheathed and retracted within the housing 12. When the device 10 is actuated, a needle coupled to a proximal end of the syringe projects from a proximal end 20 of the housing 12 to allow ejection of the substance from the syringe into the patient's body. As shown, the proximal end 20 of the housing 12 includes an opening 28 through which the needle of the syringe projects when the device 10 is actuated.

Referring still to FIG. 1, a distal end 30 of the housing 12 includes a firing engagement mechanism, e.g., a firing button 32, configured to actuate a firing mechanism. The housing 12 also houses the firing mechanism, e.g., one or more actuators, configured to drive the syringe from a sheathed or retracted position within the housing 12 (in which the needle does not project from the housing 12) to a projecting position (in which the needle projects from the housing 12). The firing mechanism is configured to subsequently expel the substance from the syringe through the needle into the patient's body.

The exemplary automatic injection device 10 may include a removable proximal cap 24 (or needle cap) for covering the proximal end 20 of the housing 12 to prevent exposure of the needle prior to an injection. In the illustrative embodiment, the proximal cap 24 may include a boss 26 for locking and/or joining the proximal cap 24 to the housing 12 until the patient is ready to activate the device 10. Alternatively, the proximal cap 24 may include a threaded screw portion, and the internal surface of the housing 12 at opening 28 may include a screw thread. Any suitable mating mechanism may be used in accordance with the teachings of exemplary embodiments.

The exemplary automatic injection device 10 may include a removable distal cap 34 configured to cover the firing button 32 to prevent exposure and accidental engagement of the firing button 32 prior to an injection. A step 29 may be formed at the distal end of the housing 12 to accommodate the distal cap 34. In an exemplary embodiment, the distal cap 34 may be coupled to the firing button 32 in a snap-fit. In another exemplary embodiment, the distal cap 34 may include a boss for locking and/or joining the distal cap 34 to the firing button 32 of the device 10 until the patient is ready to activate the device 10. In another exemplary embodiment, the distal cap 34 may include a threaded screw portion, and a surface of the firing button 32 may include a screw thread. Any suitable mating mechanism may be used in accordance with the teachings of exemplary embodiments.

The housing 12 and caps 24, 34 may include graphics, symbols and/or numbers to facilitate use of the automatic injection device 10. For example, the housing 12 may include an arrow 125 on an outer surface pointing towards the proximal end 20 of the device 10 to indicate how the device 10 should be held relative to the patient (i.e., with the proximal end 20 placed on the injection site). In addition, the proximal cap 24 is labeled with a “1” to indicate that a patient should remove the proximal cap 24 of the device first, and the distal cap is labeled with a “2” to indicate that the distal cap 34 should be removed after the proximal cap 24 is removed in preparation for an injection. One of ordinary skill in the art will recognize that the automatic injection device 10 may have any suitable graphics, symbols and/or numbers to facilitate patient instruction, or the automatic injection device 10 may omit such graphics, symbols and/or numbers.

The housing 12 may include a display window 130 to allow a patient to view the contents of the syringe housed within the housing 12. The window 130 may be an opening in the sidewall of the housing 12, or may include a transparent material or layer provided in the housing 12 to allow viewing of the interior of the device 10.

The housing 12 may be formed of any suitable surgical material including, but not limited to, plastic and other known materials.

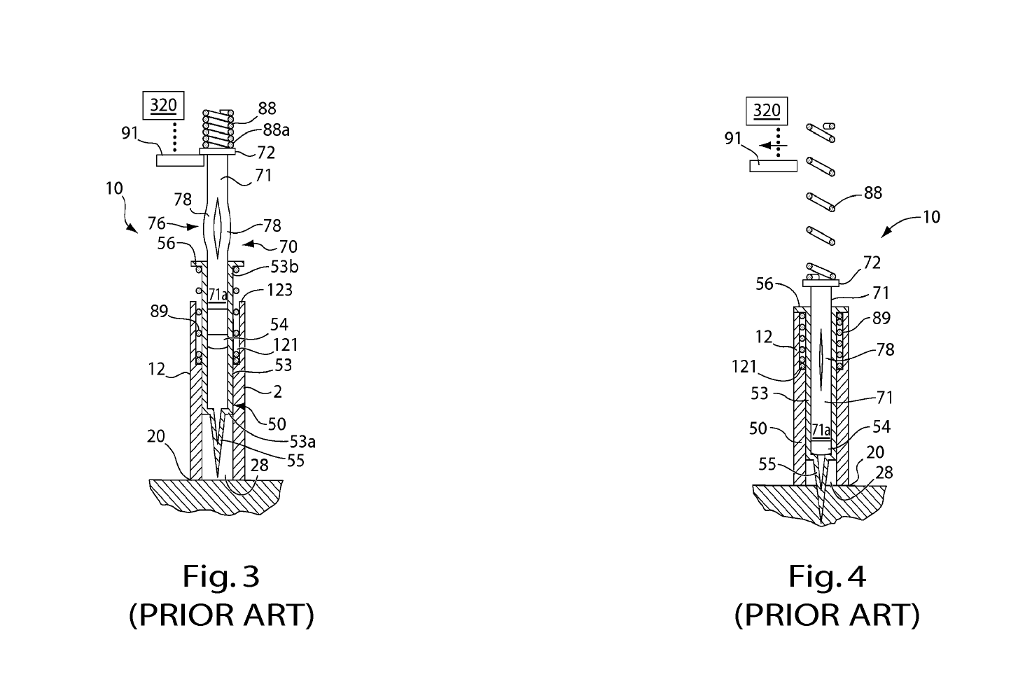

FIGS. 3 and 4 (prior art) are cross-sectional schematic views of the components of an exemplary automatic injection device 10. FIG. 3 (prior art) illustrates a cross-sectional schematic view of the exemplary automatic injection device 10 prior to use. FIG. 4 (prior art) illustrates a cross-sectional schematic view of the exemplary automatic injection device 10 of FIG. 3 during a post-injection stage of operation.

As illustrated in FIGS. 3 and 4, a syringe 50 or other suitable container for a substance is disposed within the interior of the housing 12 of the device 10. An exemplary syringe 50 may include a hollow barrel portion 53 for holding a dose of a liquid substance to be injected into a patient's body. An exemplary barrel portion 53 is substantially cylindrical in shape, although one of ordinary skill in the art will recognize that the barrel portion 53 may have any suitable shape or configuration. A seal, illustrated as a bung 54, seals the dose within the barrel portion 53. The syringe 50 may also include a hollow needle 55 connected to and in fluid communication with the barrel portion 53, through which the dose can be ejected by applying pressure to the bung 54. The hollow needle 55 extends from a proximal end 53a of the barrel portion 53. A distal end 53b of the barrel portion 53 includes a flange 56, or other suitable mechanism, for abutting a stop 123 in the housing 12 to limit the movement of the syringe 50 within the housing 12, as described below. One of ordinary skill in the art will recognize that exemplary embodiments are not limited to the illustrative syringe 50 and that any suitable container for containing a dose of a substance to be injected may be used in accordance with the teachings of exemplary embodiments.

The automatic injection device 10 shown in FIGS. 3 and 4 may include a syringe actuation component 70, illustrated as a plunger, for selectively injecting the dose contained in the syringe 50 into a patient's body. The exemplary plunger 70 may include a rod portion 71 having a first end 71a connected to and/or in fluid communication with the bung 54 for selectively applying pressure to the bung 54 to expel the dose through the needle 55. The plunger 70 may include a flanged second end 72. In an exemplary embodiment, the plunger 70 may include more or fewer components than those illustrated in FIGS. 3 and 4. In an exemplary embodiment, the device 10 may include more or fewer actuators than those illustrated in FIGS. 3 and 4.

The plunger 70 may be biased forward towards the proximal end 20 of the device 10 by a first biasing mechanism, illustrated as a coil spring 88, disposed about or above the flanged second end 72 of the plunger 70. A proximal end 88a of the coiled spring 88 may abut the flanged second end 72 of the plunger 70 to selectively apply pressure to the plunger 70 and to move the plunger 70 toward the injection site on the patient's body. Alternatively, the plunger 70 may extend through the center of the spring 88.

As illustrated in FIG. 3, prior to use of the device 10, the coil spring 88 (or another suitable mechanism) may be compressed between the plunger 70 and the housing 12, thus storing energy. A trigger 91, which may be activated by any suitable actuation means such as the firing button 32, may retain the plunger 70 and the first biasing mechanism 88 in a retracted, latched position before the firing button 32 is activated. The trigger 91 may latch the flanged second end 72 of the plunger 70. When the firing button 32 or other actuation means is activated, the trigger 91 may release the flanged second end 72 of the plunger 70, allowing the coil spring 88 to propel the plunger 70 towards the first end of the device 10.

A second biasing mechanism, illustrated as an exemplary coil spring 89, may hold the syringe 50 in a retracted position within the housing 12 prior to use, as shown in FIG. 3. In the retracted position, the needle 55 may be preferably sheathed entirely within the housing 12. The exemplary syringe coil spring 89 may be disposed about the distal portion of the barrel portion 53 and may be seated in a shelf 121 formed within the housing 12. The distal end of the coil spring 89 may abut the flanged distal end 56 of the syringe 50. The spring force of the second biasing mechanism 89 may push the flanged distal end 56 of the syringe 50 away from the proximal end 20 of the housing 12, thereby holding the syringe 50 in the retracted position until activated. Other components of the device 10 may also be used to position the syringe 50 relative to the housing 12.

The first biasing mechanism 88 and the second biasing mechanism 89 may have any suitable configuration and tension suitable for use in biasing certain components of the device. For example, the first biasing mechanism 88 may have any suitable size, shape, energy and properties suitable for driving the plunger 70 and the syringe 50 forward when released or actuated. The second biasing mechanism 89 may have any suitable size, shape, energy and properties suitable for retracting the syringe 50 prior to actuation of the first biasing mechanism 88. Other suitable means for facilitating movement of the plunger 70 and/or syringe 50 may also be used.

Referring still to the illustrative embodiment of FIGS. 3 and 4, the plunger 70 may include a rod portion 71 and an exemplary radially compressible expanded portion 76 at the center of the plunger 70 between proximal and distal solid portions of the rod portion 71. In an exemplary embodiment, the expanded portion 76 may be aligned along the central axis of the rod portion 71. In an illustrative embodiment, the rod 71 may be split and expanded to form a pair of projecting elbows 78 that encircle a longitudinal slit or void and that define the radially compressible expanded portion 76. The projecting elbows 78 may be pre-formed as part of the molded plunger 70 or, alternatively, may be attached to the plunger 70 separately. The projecting elbows 78 may be compressible so that they can be moved radially inwardly to cause that portion of the rod 71 to adopt a circumference similar to the rest of the rod 71. The compressible expanded portion 76 facilitates movement of the syringe 50.

When an activation means 320 activates the trigger 91 to release the plunger 70, the spring force of the coil spring 88 propels the plunger 70 forward. The activation means 320 may have any suitable size, shape, configuration and location suitable for releasing the plunger 70 or otherwise activating the device 10. For example, the activation means 320 may include a firing button 32 formed at a distal end 30 of the housing 12, and/or may include another suitable device, such as a latch, twist-activated switch and other devices known in the art. While the illustrative activation means 320 is located towards a distal end 30 of the device 10, one of ordinary skill in the art will recognize that the activation means 320 may be positioned at any suitable location on the device 10.

During a first operational stage, the plunger 70 pushes the syringe 50 forward such that the tip of the needle 55 projects from the proximal end 20 of the housing 12. The initial biasing force provided by the first coil spring 88 is sufficient to overcome the biasing force of the second coil spring 89 to allow movement of the syringe 50 against the backward biasing force of the second coil spring 89. In the first operational stage, the expanded region 76 of the plunger 70, formed by the projecting elbows 78, may rest against the flanged distal end 56 of the barrel portion 53, or may initially partially enter the barrel portion 53 and, in turn, at least temporarily halt due to stiction forces. This prevents the plunger 70 from traveling within the syringe barrel portion 53. In this manner, by stiction or abutment of the flanged distal end 56, all biasing force from the first coil spring 88 is applied to move the syringe 50 forward towards the proximal end 20 of the device 10.

The forward motion of the syringe 50 towards the proximal end 20 of the device 10 may continue against the biasing force of the coil spring 89 until the flanged distal end 56 of the barrel portion 53 abuts the stop 123 in the housing 12, thereby forming a stopping mechanism 56, 123. One of ordinary skill in the art will recognize that other stopping mechanisms may be employed and that exemplary embodiments are not limited to the illustrative stopping mechanism.

The first operational stage may propel the tip of the needle 55 through the opening 28 at the proximal end 20 of the device 10, so that the needle 55 may pierce the patient's skin. During this stage, the syringe barrel portion 53 may preferably remain sealed without expelling the substance through the needle 55. The interference caused by the stopping mechanism 56, 123 may maintain the needle 55 in a selected position extending from the proximal open end 28 of the device 10 during subsequent steps. Until the stopping mechanism 56, 123 stops the movement of the syringe 50, the compressible expanded portion 76 of the plunger 70 may prevent movement of the plunger 70 relative to the barrel portion 53. The stopping mechanism 56, 123 may be positioned at any suitable location relative to the open proximal end 20 to allow the syringe 50 to penetrate the skin by any suitable depth suitable for an injection.

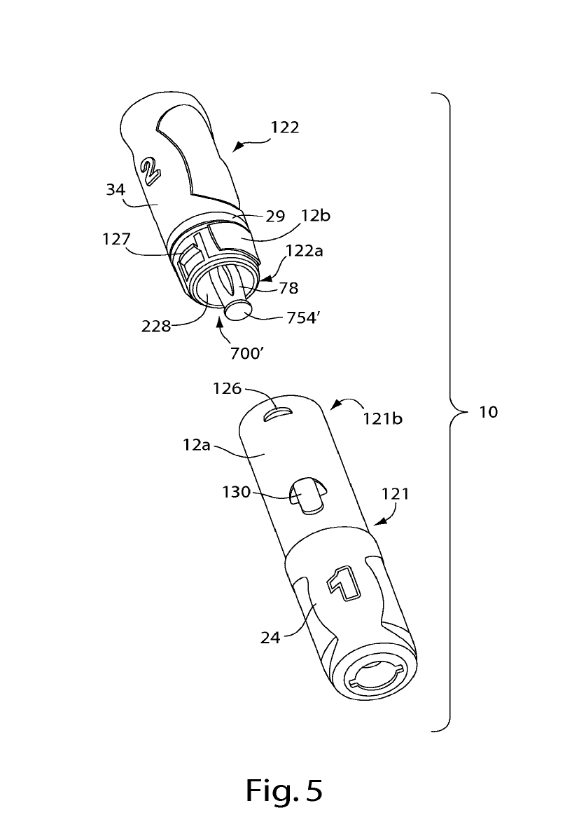

The second operational stage commences after the stop 123 of the housing 12 catches the flanged portion 56, stopping farther movement of the barrel portion 53. During this stage, the continued biasing force of the coil spring 88 may continue to push the plunger 70 relative to the housing 12, as shown in FIG. 5. The biasing force may cause the elbows 78 of the plunger 70 to compress radially inward and slide into the interior of the barrel portion 53. While the interference between components 123 and 56 may retain the barrel portion 53 in a selected position (with the needle 55 exposed) and with the elbows 78 in a collapsed stage, the coil spring 88 may push the plunger 70 within the barrel portion 53. After the plunger 70 overcomes the necessary force to allow the elbows 78 to compress and extend into the barrel portion 53, the plunger 70 may apply pressure to the bung 54, causing ejection of the substance contained in the syringe 50 through the projecting needle 55. Because the needle 55 was made to penetrate the patient's skin in the first operational stage, the substance contained in the barrel portion 53 of the syringe 50 is injected directly into a portion of the patient's body.

FIG. 5 illustrates a perspective view of an exemplary automatic injection device 10 including an exemplary syringe housing sub-assembly 121 and an exemplary firing mechanism sub-assembly 122. In an exemplary embodiment, the automatic injection device 10 may include two interlocking components: a syringe housing sub-assembly 121 containing the proximal components of the device 10 (e.g., proximal housing component 12a, syringe barrel 53, coil spring 89, needle 55 and other proximal components, etc.), and a firing mechanism sub-assembly 122 containing the distal components of the device 10 (e.g., firing body 12b, syringe actuation component 700′ having a pressurizer 754′ extending out of an opening 228 at the proximal end 122a of the firing mechanism sub-assembly 122, etc.). The syringe housing sub-assembly 121 and the firing mechanism sub-assembly 122 may be coupled through any suitable means. In an exemplary embodiment, a proximal end 122a of the firing mechanism sub-assembly 122 may be sized and configured to be inserted into a distal end 121b of the syringe housing sub-assembly 121. In addition, one or more tabs 127 at the proximal end 122a of the firing mechanism sub-assembly 122 may snap-fit into corresponding openings 126 at the distal end 121b of the syringe housing assembly 122 to ensure alignment and coupling of the two assemblies 121, 122 and the components housed therein.

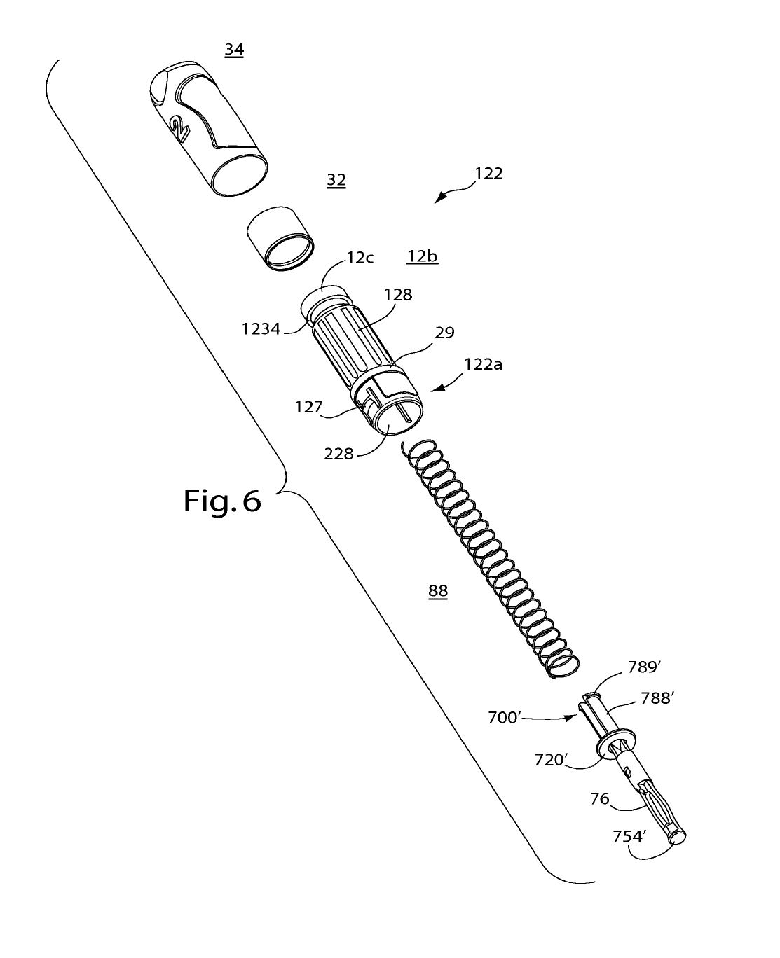

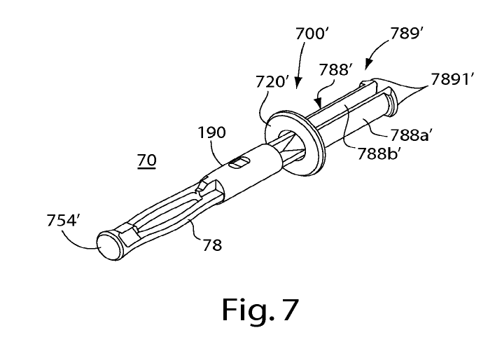

FIG. 6 illustrates an exploded perspective view of the firing mechanism assembly 122 of the exemplary automatic injection device of FIG. 5. FIG. 7 illustrates a perspective view of an exemplary syringe actuation component 700′. The firing mechanism sub-assembly 122 may include the firing body 12b (also called the distal housing component) having a hollow internal bore for housing the biasing mechanism 88 and a distal portion of the syringe actuation component 700′. The firing body 12b may include an opening 228 at the proximal end 122a to allow entry of the biasing mechanism 88 and the syringe actuation component 700′ during assembly of the firing mechanism sub-assembly 122. The firing body 12b may have one or more ridges or grooves on its outer surface 128 to identify it and to facilitate gripping of the device 10. The firing body 12b may include one or more tabs 127 at or near the proximal end 122a of the firing mechanism sub-assembly 122 configured to snap-fit into corresponding openings 126 on the distal end 121b of the syringe housing assembly 122. The firing body 12b may also include a narrowed distal wall 1234 for supporting the distal end of the spring 88. The firing body 12b may also include a distal anchoring cap 12c over which the anchoring portion 789′ of the syringe actuation component 700′ may be supported.

The firing mechanism sub-assembly 122 may also include a syringe actuator, illustrated as a syringe actuation component 700′, which extends from the proximal end 122a of the firing body 12b for driving the syringe 50 forward within the housing 12 in a first operational stage, and for actuating the bung 54 to expel the contents of the syringe 50 in a second operational stage. The proximal end of the syringe actuation component 700′ may include be configured as a pressurizer 754′ for engaging and driving the bung 54. Distal to the pressurizer 754′, a pair of elbows 76 may be provided with a central longitudinal slit or void. The elbows 76 may be aligned along a central axis of the syringe actuation component 700′ and may extend between the pressurizer 754′ and a solid rod portion 70 of the syringe actuation component 700′. The syringe actuation component 700′ may include an indicator 190 at the solid rod portion 70 distal to the elbows 78. During operation of the device 10 and after completion of an injection, the indicator 190 is configured to align with the window 130 on the housing 12 to indicate at least partial completion of the injection. The indicator 190 preferably has a distinctive color or design to represent completion of an injection.

The illustrative syringe actuation component 700′ further includes a retaining flange 720′ for holding the actuating coil spring 88 in a compressed position until actuation. The retaining flange 720′ is sized, dimensioned and formed of a material that preferably allows the syringe actuation component 700′ to slidably and easily move within the housing 12 when the device 10 is actuated. Extending distally from the retaining flange 720′, the syringe actuation component 700′ forms a base 788′, for the actuating coil spring 88. The base 788′ terminates in a trigger anchoring portion 789′. The illustrative base 788′ may comprise flexible arms 788a′, 788b′ around which the spring 88 coils. The trigger anchoring portion 789′ may comprise tabbed feet 7891′ extending from the base 788′ and configured to selectively engage the anchoring cap 12c of the firing body 12b. The firing button 32 coupled to the distal end of the firing body 12b is configured to hold the trigger anchoring portion 789′ retracted until activation. When activated, the firing button 32 releases the trigger anchoring portion 789′, allowing the coil spring 88 to propel the syringe actuation component 700′ towards the proximal end 20 of the device 10.

In a retracted, anchored position shown FIGS. 6 and 7, the trigger anchoring portion 789′ interacts with the housing 12, which holds the tabbed feet 7891′ in a latched position against the biasing force of the coil spring 88, to maintain the syringe actuation component 700′ in a retracted position. In this position, the flange 720′ retracts the spring 88 against the distal wall 1234 of the firing body 12b. An opening in the anchoring cap 12c allows the firing button 32 access to the anchoring portion 789′ of the syringe actuation component 700′. In the retracted position, the pressurizer 754′ of the syringe actuation component 700′ extends out of an opening 228 at the proximal end 122a of the firing body 12b.

When the firing body 12b couples to a corresponding syringe actuation mechanism 121, the pressurizer 754′ extends into the barrel portion of a syringe housed therein. The pressurizer 754′ may be integral with, the same as, connected to, or otherwise in communication with the bung 54 of a syringe 50 housed in the device 10 and may have any suitable size, shape and configuration suitable for applying pressure to the bung 54. In one embodiment, the pressurizer 754′ has a cross-section corresponding to the shape of the barrel portion 53 of a corresponding syringe 50 so as to substantially seal the barrel portion 53, and the pressurizer 754′ is configured to slidably move within the barrel portion 53 to apply pressure to the bung 54 and actuate the syringe 50.

In the illustrative embodiment of FIGS. 6 and 7, the syringe actuation component 700′ constitutes a single, integrated mechanism for anchoring a corresponding syringe 50, spring 88 and other components, actuating and moving the syringe 50 to a protracted position, and separately expelling the contents of the syringe 50.

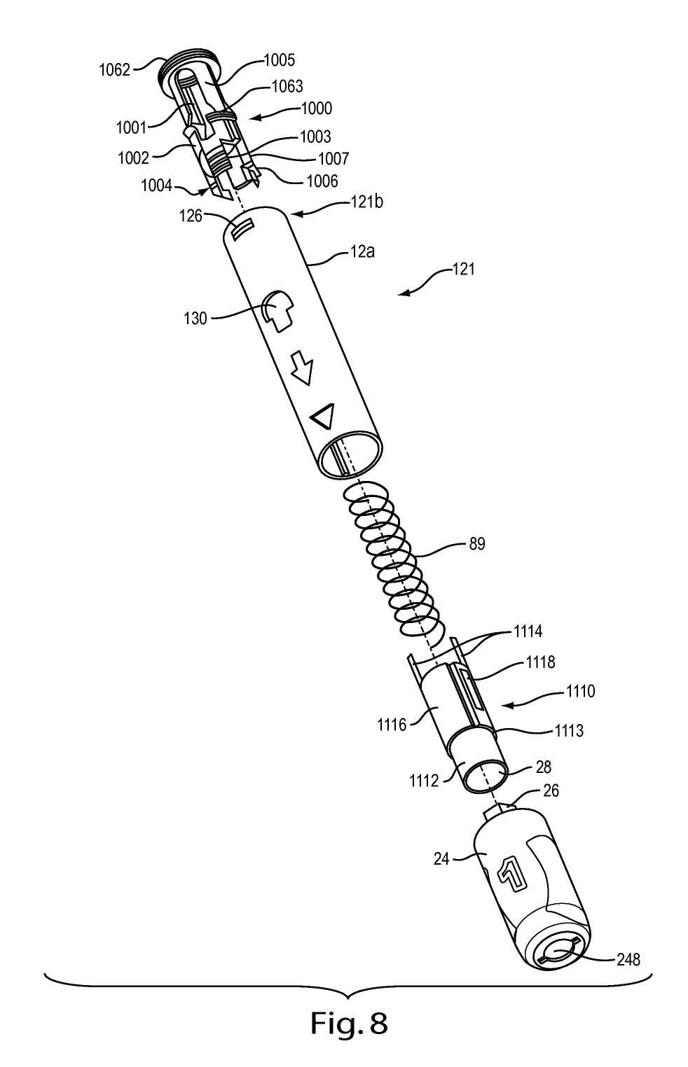

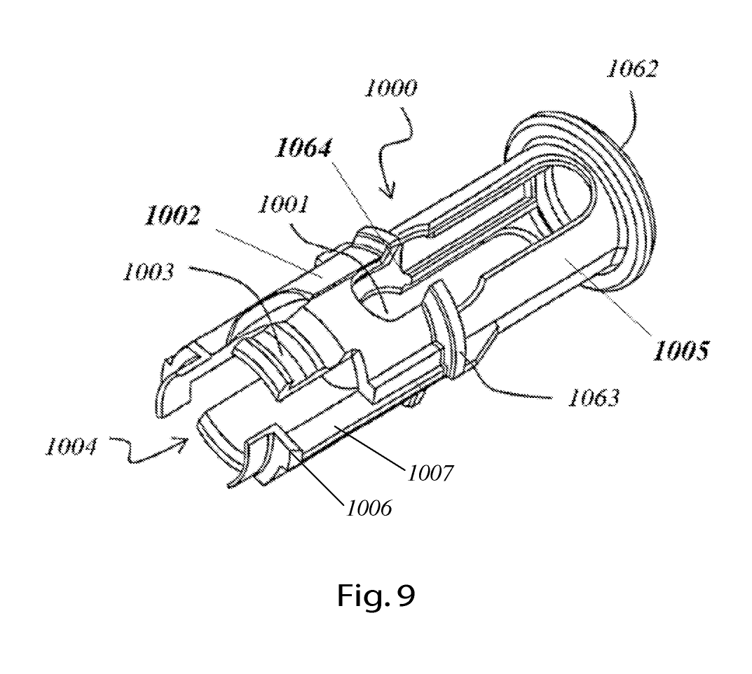

FIG. 8 is an exploded perspective view of an exemplary syringe housing sub-assembly 121 which is configured to couple to and interact with the firing mechanism sub-assembly 122 of FIG. 7. The components of the syringe housing sub-assembly 121 are cooperatively configured to house a syringe 50 containing a substance to be injected and to facilitate operation of the device 10 in the two different operational stages as described above. The syringe housing sub-assembly 121 includes a syringe carrier 1000 configured to movably hold a syringe. FIG. 9 illustrates a perspective view of an exemplary syringe carrier 1000. The syringe housing sub-assembly 121 also includes a shroud 1110 configured to protectively cover a needle 55 before, during or after use in an injection. The syringe carrier 1000 and the shroud 1110 may be coupled together with a second biasing member 89 positioned therebetween. The syringe carrier 1000, the shroud 1110 and the biasing member 89 may be placed within the hollow bore of a proximal housing component 12a whose proximal end may be covered by the proximal cap 24.

The proximal housing component 12a is a portion of the syringe housing 12 that provides a hollow structural member for accommodating the second biasing mechanism 89, the syringe carrier 1000 and the shroud 1110 of the syringe housing sub-assembly 121. The proximal housing component 12a may be a tubular member having a tubular side wall, i.e., may have a substantially cylindrical shape with a substantially circular cross-section. The proximal housing component 12a may extend from a proximal end to a distal end along the longitudinal axis of the automatic injection device. The proximal housing component 12a may be coupled to the firing body 12b at or near the distal end, and may be coupled to the proximal cap 24 at or near the proximal end. The proximal housing component 12a may include one or more windows 130 formed or provided in its side wall to allow a user to view the contents of the syringe 50 disposed inside the proximal housing component 12a.

The shroud 1110 is a structural member that, when deployed, provides a protective covering for the needle before, during and/or after the use of the needle in an injection. The components of the syringe housing sub-assembly 121 are cooperatively configured to hold the shroud 1110 in a retracted position relative to the proximal housing component 12a during an injection and to automatically deploy the shroud 1110 relative to the proximal housing component 12a during or after an injection. In an exemplary embodiment, the shroud 1110 may be positioned at or may form the proximal end 20 of the housing 12. The shroud 1110 may include a main tubular body portion 1116 having a tubular side wall, i.e., may have a substantially cylindrical shape with a substantially circular cross-section. The main tubular body portion 1116 may extend from a proximal end to a distal end along the longitudinal axis of the automatic injection device.

The main tubular body portion 1116 may include one or more slots 1118 extending longitudinally along the body portion. In an exemplary embodiment, the slot 1118 may provide a longitudinal track for the movement of a raised rail edge or tabbed foot 1006 of the syringe carrier 1000 as the syringe carrier 1000 and/or the shroud 1110 move relative to each other. When the shroud 1110 moves toward the syringe carrier 1000 during retraction of the shroud, the tabbed foot 1006 of the syringe carrier 1000 may travel toward the proximal end of the device along the slot 1118. Conversely, when the shroud 1110 moves away from the syringe carrier 1000 during deployment of the shroud, the tabbed foot 1006 of the syringe carrier 1000 may travel toward the distal end of the device along the slot 1118.

The distal end of the main tubular body portion 1116 may be configured as a rim and may be coupled to one or more distal arms 1114 that are spaced apart from each other. In an exemplary embodiment, two spaced-apart distal arms 1114 are coupled to the distal end of the main tubular body portion 1116. The distal arms 1114 may take any suitable shape including, but not limited to, a substantially cylindrical shape with a circular cross-section, a substantially extended box shape with a rectangular or square cross-section, etc. In an exemplary embodiment, the distal arms 1114 may extend substantially parallel to each other and to the longitudinal axis of the device. In another exemplary embodiment, the distal arms 1114 may extend at an angle to the longitudinal axis of the device such that they diverge from each other relative to attachment points on the shroud 1110.

The proximal end of the main tubular body portion 1116 may be coupled to a proximal tubular portion 1112. In an exemplary embodiment, the proximal tubular portion 1112 may cover part or all of the needle 55 after an injection. In an exemplary embodiment, the main tubular portion 1116 may cover part or all of the needle 55 after an injection. The proximal tubular portion 1112 of the shroud 1110 may be a tubular member having a tubular side wall, i.e., may have a substantially cylindrical shape with a substantially circular cross-section. The proximal tubular portion 1112 may extend from a proximal end to a distal end along the longitudinal axis of the automatic injection device. The proximal end of the proximal tubular portion 1112 may have a proximal opening 28. The proximal opening 28 may allow the syringe needle 55 to project outwardly and to penetrate an injection site during operation of the device 10. The distal end of the proximal tubular portion 1112 may be coupled to or may extend from the proximal end of the main tubular body portion 1116 of the shroud 1110.

In an exemplary embodiment, the proximal tubular portion 1112 of the shroud 1110 may have a cross-sectional diameter smaller than the cross-sectional diameter of the main tubular body portion 1116. In this exemplary embodiment, a stepped portion 1113 may be formed at the coupling between the distal end of the proximal tubular portion 1112 and the proximal end of the main tubular body portion 1116. The stepped portion 1113 may form a forward stop for the biasing member 89 that is disposed at least partly inside the shroud 1110. The stepped portion 1113 may confine the biasing member 89 and prevent farther forward movement of the biasing member 89 towards the proximal end of the device 10.

The syringe carrier 1000 is a structural member that envelopes the distal half of a syringe 50 used in the device 10. The syringe carrier 1000 may be configured to hold and guide the syringe 50 within the housing 12 to allow the syringe 50 to move forward to an injecting position. The syringe 50 may rest in the syringe carrier 1000 and both may be contained within the proximal housing component 12a. During operation of the device 10, the syringe 50 and the syringe carrier 1000 move proximally forward within the proximal housing component 12a.

In an exemplary embodiment, the syringe carrier 1000 is stationary within the proximal housing component 12a and the syringe 50 selectively and controllably slides within and relative to the syringe carrier 1000. In another exemplary embodiment, the syringe carrier 1000 is slidably disposed within the proximal housing component 12a and selectively carries the syringe 50 within the housing 12. The syringe carrier 1000 may have any suitable configuration, shape and size suitable for carrying or guiding the syringe 50 within the proximal housing component 12a. The syringe carrier 1000 is also configured to cooperate with the shroud 1110 in order to automatically deploy the shroud 1110 during and/or after an injection.

The syringe carrier 1000 may include a proximal tubular portion 1002 that is substantially tubular and has a tubular side wall, i.e., has a substantially cylindrical shape with a substantially circular cross-section. The side wall of the proximal tubular portion 1002 may optionally include one or more raised structures, e.g., a longitudinally-extending rail 1007. The rail 1007 may include a tabbed foot 1006. When the syringe carrier 1000 is assembled with the shroud 1110, the tabbed foot 1006 may fit within the slot 1118 of the shroud 1110, such that the two components cooperatively form a locking mechanism for the syringe carrier 1000 and the shroud 1110. In the assembled configuration, the tabbed foot 1006 may travel longitudinally within the slot 1118 but is restricted from disengaging from the slot 1118. That is, a forward movement of the tabbed foot 1006 of the carrier 1000 may be stopped by the proximal end of the slot 1118 of the shroud 1100. At the same time, the rail 1007 fits along internal longitudinal grooves provided in the main tubular body portion 1116 of the shroud 1110, and moves longitudinally along the tracks provided by the grooves. In an exemplary embodiment, the grooves may be provided near the distal end of the shroud 1110 and may extend for an exemplary length of about 2 mm.

The proximal end of the proximal tubular portion 1002 may be coupled to or may extend into a proximal anchor portion 1003. The proximal anchor portion 1003 may have an exemplary outer diameter of about 12.60 mm in an exemplary embodiment. The proximal anchor portion 1003 of the syringe carrier 1000 may limit the movement of the syringe 50 in a distal, rearward direction. In an exemplary embodiment, the proximal end of the proximal anchor portion 1003 may include a syringe carrier coupler 1004 that extends in the proximal direction past the proximal anchor portion 1003 to facilitate coupling of the syringe carrier 1000 with the distal end of the biasing member 89 and the distal end of the shroud 1110.

The distal end of the proximal tubular portion 1002 may be coupled to a proximal portion of a distal tubular portion 1005 that is substantially tubular and has a tubular side wall, i.e., has a substantially cylindrical shape with a substantially circular cross-section. The distal end of the distal tubular portion 1005 may be coupled to or may extend to form a flanged distal end 1062 that may serve as a damper for the syringe 50. The flanged distal end 1062 may extend radially from the distal tubular portion 1005, and may have a larger cross-sectional diameter than the distal tubular portion 1005.

The side wall of the distal tubular portion 1005 may include one or more windows 1001 that allow a user to view the contents of the syringe 50 disposed inside the housing 12. In some exemplary embodiments, the windows 1001 may extend into the proximal tubular portion 1002. In other exemplary embodiments, the windows 1001 may be restricted to either the proximal tubular portion 1002 or the distal tubular portion 1005.

In an exemplary embodiment, the cross-sectional diameter of the distal tubular portion 1005 may be smaller than the cross-sectional diameter of the proximal tubular portion 1002. In this embodiment, there may be stepped portion 1064 formed at the coupling between the distal end of the proximal tubular portion 1002 and the proximal end of the distal tubular portion 1005. The stepped portion 1064 may form a substantially perpendicular surface between the planes of the tubular portions or may form an inclined surface at an angle relative to the planes of the tubular portions.

The region between the proximal 1002 and the distal 1005 tubular portions may include an intermediate flange 1063 that extends radially from the tubular portions. The intermediate flange 1063 may be a radially continuous structure or a radially discontinuous structure, and may have a larger cross-sectional diameter than the tubular portions. The intermediate flange 1063 may be configured to engage with an interior stop or flange 256 of the proximal housing component 12a to limit the movement of the syringe 50 in the proximal, forward direction. Upon actuation of the syringe carrier 1000 the syringe carrier 1000 moves toward the proximal end of the device until the intermediate flange 1063 of the syringe carrier 1000 abuts against the interior stop or flange 256 of the proximal housing component 12a. This limits farther movement of the syringe carrier 1000 and the syringe 50 in the proximal, forward direction.

In order to expose the needle for an injection, the shroud 1110 is retracted in the distal, backward direction against the biasing force of the biasing member 89. When the syringe needle is in use during an injection, the shroud 1110 may be pushed to or held in a retracted position toward the distal end of the device. During retraction, as the shroud 1110 moves relative to the syringe carrier 1000, the tabbed foot 1006 of the rail 1007 of the syringe carrier 1000 moves in a relative manner longitudinally toward the proximal end of the device along the slot 1118 of the shroud 1110. At the same time, the rail 1007 of the syringe carrier 1000 moves in a relative manner longitudinally along the inner grooves in the shroud 1110. The shroud retraction process is complete and further movement of the shroud 1110 is stopped when the tabbed foot 1006 reaches the proximal end of the slot 1118. Since the tabbed foot 1006 is fit into the slot 1118 in a locking manner, the tabbed foot 1006 does not disengage from the slot 1118 and prevents farther backward or distal motion of the shroud 1110.

In the retracted position of the shroud 1110, the distal rim or end of the main tubular body portion 1116 may abut the proximal side of the stop or flange 256 provided on the inner surface of the proximal housing component 12a. In an exemplary embodiment, in the retracted position, the distal arms 1114 may extend in the distal direction beyond the intermediate flange 1063 of the syringe carrier 1000.

In order to cover the needle before and/or after an injection, the shroud 1110 is deployed in the proximal, forward direction along the biasing force of the biasing member 89. In the deployed position, the shroud 1110 protectively covers the syringe needle during or after use and prevents accidental needle stick injuries. During deployment, as the shroud 1110 moves relative to the syringe carrier 1000, the tabbed foot 1006 of the rail 1007 of the syringe carrier 1000 moves in a relative manner longitudinally toward the distal end of the device along the slot 1118 of the shroud 1110. At the same time, the rail 1007 of the syringe carrier 1000 moves in a relative manner longitudinally along the inner grooves in the shroud 1110. The shroud deployment process is complete and further movement of the shroud 1110 is stopped when the tabbed foot 1006 reaches the distal end of the slot 1118. Since the tabbed foot 1006 is fit into the slot 1118 in a locking manner, the tabbed foot 1006 does not disengage from the slot 1118 and prevents farther proximal or forward motion of the shroud 1110.

After the shroud 1110 has deployed, the distal arms 1114 ensure that the shroud 1110 is not retracted again due to a backward force applied to the shroud in the distal direction. In exemplary embodiments, the distal arms 1114 of exemplary shrouds 1110 may resist shroud retraction against a maximum force known as the “override force.” In an exemplary embodiment, during deployment, the shroud 1110 twistingly moves within the proximal housing component 12a of the device such that the distal end of the distal arms 1114 of the shroud 1110 rest against the interior stop or flange 256 of the housing. The interior stop or flange 256 thus prevents farther distal or backward movement of the shroud 1110 after the shroud has been deployed. This locking mechanism ensures that the syringe needle is protectively covered after the device has been used, and prevents accidental needle stick injuries caused by accidental retraction of the shroud. Exemplary override forces may range from about 80 N to about 200 N, although override forces are not limited to this exemplary range.

As illustrated in FIG. 8, the biasing member 89 extends between the proximal end of the syringe carrier coupler 1004 of the syringe carrier 1000 and the stepped portion 1113 of the shroud 1110. In an exemplary embodiment, the biasing member 89 may hold the syringe 50 in a retracted position within the housing 12 prior to use. In another exemplary embodiment, the syringe carrier 1000 holding the syringe 50 may be locked to the interior flange 256 in the housing. This interaction may hold the syringe 50 in a retracted position within the housing before use. With the aid of the boss 26 of the proximal cap 24, this interaction is able to lock the syringe carrier 1000 and the syringe 50 in place during shipping, shock, dropping, vibration, and the like.

In the retracted position, the needle 55 may be preferably sheathed entirely within the housing 12. The exemplary syringe coil spring 89 may be disposed about the proximal portion of the barrel portion 53 of the syringe 50 and may be seated in a shelf formed within the housing interior 12. The top end of the coil spring 89 may abut the flanged distal end 56 of the syringe 50. The spring force of the second biasing mechanism 89 may push the flanged distal end 56 of the syringe 50 away from the proximal end 20 of the housing 12, thereby holding the syringe 50 in the retracted position until activated. Other components of the device 10 may also position the syringe 50 relative to the housing 12.

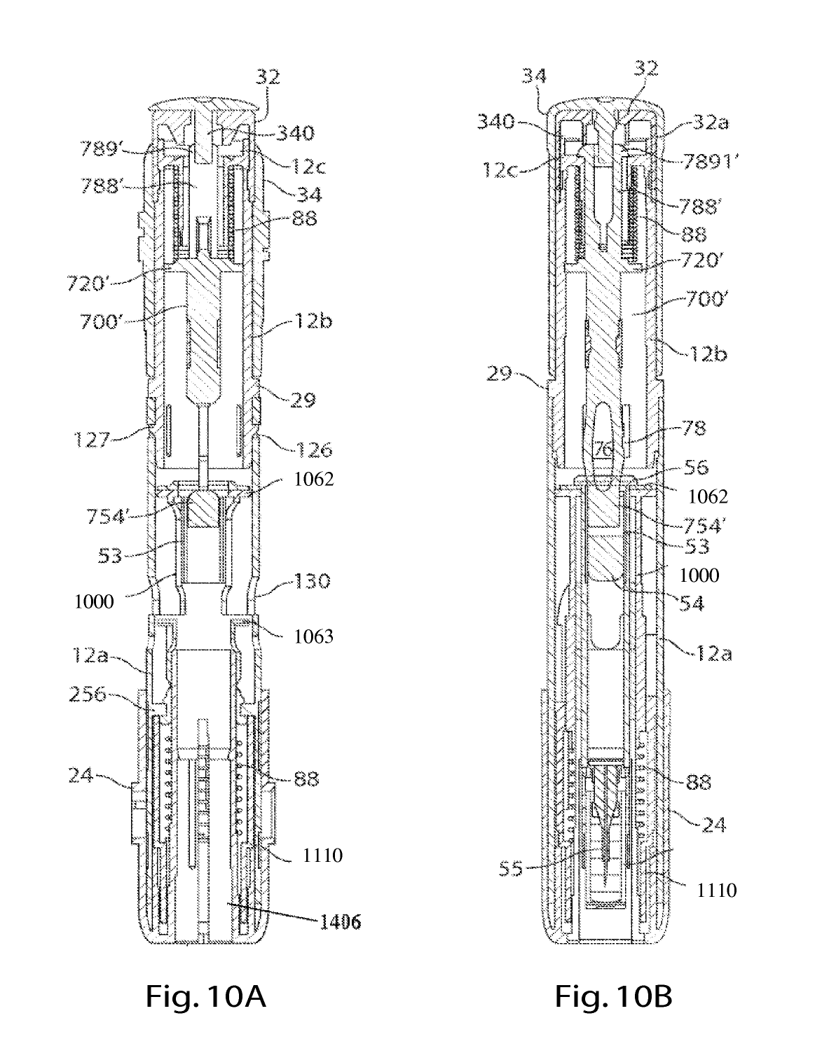

FIGS. 10A and 10B are cross-sectional views at 90° offset angles from each other, illustrating an assembled automatic injection device, wherein the syringe housing sub-assembly 121 and the firing mechanism sub-assembly 122 of FIG. 5 are coupled together, such that the pressurizer 754′ of the syringe actuation component 700′ extends into the barrel portion 53 of a syringe 50 housed in the syringe housing sub-assembly 121 and is in communication with a bung 54 of the syringe 50. Referring again to FIGS. 7 and 10B, the syringe actuation component 700′ includes, at its proximal end 700a′, a pressurizing end 754′ for applying pressure to the bung 54, a plunger rod portion 70 with a compressible expanded portion 76 (illustrated as the plunger elbows 78), as well as other components, such as components for anchoring the coil spring 88 to the syringe actuation component 700′, as described below. The compressible expanded portion 76 facilitates movement of a corresponding syringe 50 into a projecting position and expulsion of the contents of the syringe 50. Alternatively, the syringe actuation component 700′ may comprise multiple actuators for moving and/or promoting actuation of the syringe 50.

As shown in FIG. 10B, the trigger anchoring portion 789′ of the syringe actuation component 700′ is anchored at the distal end of the housing 12 by the firing button 32. When a patient activates the firing button 32, driving arms 32a connected to the firing button 32 compress the tabbed feet 7891′ of the trigger anchoring portion 789′ inwards, thereby decreasing the distance (plunger arm width) between the tabbed feet of the plunger arms 788a′, 788b′. This releases the syringe actuation mechanism 700′ and the spring 88.

In an exemplary embodiment, during a first operational stage, the plunger 70 advances under the spring force of the spring 88 and enters the bore of the syringe 50. The elbows 78 of the plunger 70 may compress radially inwardly, at least partly, as the plunger 70 enters the bore of the syringe 50. In an exemplary embodiment, the radially inward compression of the elbows 78 may cause the plunger 70 to elongate or lengthen along the longitudinal axis. In an exemplary embodiment, the pressurizing end 754′ of the plunger 70 may initially be spaced from the bung 54, and the plunger 70 may move toward the bung 54 during the first operational stage until the pressurizing end 754′ of the plunger 70 comes into initial contact with the bung 54.

During a second operational stage, the pressurizing end 754′ of the plunger 70 pushes against the bung 54. In this stage, the elbows 78 of the plunger 70 exert frictional forces against the inner wall of the syringe, which impedes the forward movement of the pressurizing end 754′ against the bung 54. Furthermore, the incompressible nature of the dose of the liquid therapeutic substance in the syringe acts against the forward movement of the pressurizing end 754′ against the bung 54. As a result, the combination of the frictional forces exerted by the elbows 78 and the resistance force of the liquid inside the syringe 50 impedes farther movement of the pressurizing end 754′ against the bung 54. When the combination of these forces exceeds the forces holding the syringe carrier 1000 in place, the syringe 50 and the syringe carrier 1000 are caused to move forward toward the proximal end of the device under the force of the spring 88. During the forward movement of the syringe, the initial biasing force provided by the first coil spring 88 is sufficient to overcome the biasing force of the second coil spring 89 to allow movement of the syringe 50 against the backward biasing force of the second coil spring 89. The forward movement of the syringe 50 causes the tip of the needle 55 to project from the proximal end 20 of the housing 12.

In this exemplary embodiment, during a third operational stage, when the syringe carrier 1000 is fully extended in the housing of the device, the plunger 70 moves farther into the bore of the syringe 50. In an exemplary embodiment, the radially inward compression of the elbows 78 may cause the plunger 70 to elongate or lengthen along the longitudinal axis. As the plunger 70 moves into the syringe 50, the pressurizing end 754′ of the plunger 70 pushes the bung 54 into the syringe 50 and causes the contents of the syringe 50 to be ejected from the syringe through the needle 55.

In another exemplary embodiment, after the spring 88 is released, the plunger 70 may advance under the spring force of the spring 88 and enter the bore of the syringe 50, and the elbows 78 of the plunger 70 may compress radially inwardly, at least partly, as the plunger enters the bore of the syringe 50. In an exemplary embodiment, the radially inward compression of the elbows 78 may cause the plunger 70 to elongate or lengthen along the longitudinal axis.

The pressurizing end 754′ of the plunger 70 may initially be spaced from the bung 54 in an exemplary embodiment, and the plunger 70 may move toward the bung 54 until the pressurizing end 754′ of the plunger 70 comes into initial contact with the bung 54. The pressurizing end 754′ of the plunger 70 may subsequently push against the bung 54. The elbows 78 of the plunger 70 may exert frictional forces against the inner wall of the syringe, which impedes the forward movement of the pressurizing end 754′ against the bung 54. Furthermore, the incompressible nature of the dose of the liquid therapeutic substance in the syringe acts against the forward movement of the pressurizing end 754′ against the bung 54. As a result, the combination of the frictional forces exerted by the elbows 78 and the resistance force of the liquid inside the syringe 50 may impede farther movement of the pressurizing end 754′ against the bung 54.

When the combination of these forces exceeds the forces holding the syringe carrier 1000 in place, the syringe 50 and the syringe carrier 1000 are caused to move forward toward the proximal end of the device under the force of the spring 88. During the forward movement of the syringe, the initial biasing force provided by the first coil spring 88 is sufficient to overcome the biasing force of the second coil spring 89 to allow movement of the syringe 50 against the backward biasing force of the second coil spring 89. The forward movement of the syringe 50 causes the tip of the needle 55 to project from the proximal end 20 of the housing 12. In this exemplary embodiment, when the syringe carrier 1000 is fully extended in the housing of the device, the elbows 78 of the plunger 70 may compress radially inwardly to a greater extent and the plunger 70 may move farther into the bore of the syringe 50. In an exemplary embodiment, the radially inward compression of the elbows 78 may cause the plunger 70 to elongate or lengthen along the longitudinal axis. As the plunger 70 moves into the syringe 50, the pressurizing end 754′ of the plunger 70 may push the bung 54 into the syringe 50 and cause the contents of the syringe 50 to be ejected from the syringe through the needle 55.

In another exemplary embodiment, prior to operation, the compressible expanded portion 76, illustrated as elbows 78, of the syringe actuation component 700′ rests above the flanged distal end 56 of the syringe 50 to allow the compressible expanded portion 76, when pushed by a released coil spring 88, to apply pressure to the syringe barrel portion 53, thereby moving the syringe 50 forward within the housing 12 when actuated. In this exemplary embodiment, in the first operational stage, the expanded region 76 of the plunger 70, formed by the projecting elbows 78, rests against the flanged distal end 56 of the barrel portion 53. This prevents the plunger 70 from traveling within the syringe barrel portion 53.

In this manner, all biasing force from the first coil spring 88 is applied to move the syringe 50 and the syringe carrier 1000 forward towards the proximal end 20 of the device 10. The forward motion of the syringe 50 and the syringe carrier 1000 towards the proximal end 20 of the device 10 may continue against the biasing force of the coil spring 88 until the flanged distal end 56 of the barrel portion 53 abuts a stopping mechanism, such as a stop 256 on the proximal housing component 12a shown in FIG. 10B. One of ordinary skill in the art will recognize that alternate stopping mechanisms may be employed and that exemplary embodiments are not limited to the illustrative stopping mechanism.

The first operational stage may propel the tip of the needle 55 through the opening 28 at the proximal end 20 of the device 10, so that the needle 55 may pierce the patient's skin. During this stage, the syringe barrel portion 53 may preferably remain sealed without expelling the substance through the needle 55. The interference caused by the stopping mechanism may maintain the needle 55 in a selected position extending from the proximal open end 28 of the device 10 during subsequent steps. Until the stopping mechanism stops the movement of the syringe 50, the compressible expanded portion 76 of the plunger 70 may prevent movement of the plunger 70 relative to the barrel portion 53. The stopping mechanism may be positioned at any suitable location relative to the open proximal end 20 to allow the syringe 50 to penetrate the skin by any suitable depth suitable for an injection.

In this exemplary embodiment, the second operational stage commences after the stopping mechanism of the housing 12 catches the flanged portion 56, stopping further movement of the barrel portion 53. During this stage, the continued biasing force of the spring 88 continues to move the syringe actuation component 700′ forward, causing the compressible expanded portion 76 to compress radially inwardly and move into the barrel portion 53 of the syringe 50. In an exemplary embodiment, the radially inward compression of the elbows 78 may cause the plunger 70 to elongate along the longitudinal axis. The forward motion of the syringe actuation component 700′ within the barrel portion 53 causes the pressurizer 754′ to apply pressure to the bung 54, causing expulsion of the syringe contents into an injection site. Because the needle 55 was made to penetrate the patient's skin in the first operational stage, the substance contained in the barrel portion 53 of the syringe 50 is injected directly into a portion of the patient's body.

As also shown in FIGS. 10A and 10B, the distal cap 34 may include a stabilizing protrusion 340 that extends through the firing button 32 and between the tabbed feet 7891′ of the syringe actuation component 700′ to stabilize the components of the device prior to activation.

In the exemplary embodiment shown in FIG. 10A, a removable rigid needle shield 1406 is coupled to the proximal end of the syringe 50 for protectively covering the syringe needle 55. The rigid needle shield 1406 covers and protects a soft needle shield which keeps the syringe needle 55 sterile before use. Together, the rigid needle shield 1406 and the soft needle shield are meant to prevent accidental needle stick injuries that could be caused by an exposed needle. In an exemplary embodiment, the rigid needle shield 1406 is a hollow tubular member with a substantially cylindrical wall having an inner bore with a substantially circular cross-section. The outer cross-sectional diameter of the cylindrical wall may be substantially constant over the length of the rigid needle shield 1406 or may vary over the length of the rigid needle shield 1406. An exemplary rigid needle shield 1406 may be formed of one or more rigid materials including, but not limited to, polypropylene.

In an exemplary embodiment, a removable soft needle shield (not shown) is provided within the bore of the rigid needle shield 1406 to provide a sealing layer between the syringe needle 55 and the rigid needle shield 1406. An exemplary soft needle shield may be formed of one or more resilient materials including, but not limited to, rubber.

In the syringe needle assembly shown in FIGS. 10A and 10B, the syringe needle 55 is covered by the soft needle shield and the rigid needle shield 1406. The rigid needle shield 1406 is, in turn, covered by the proximal removable cap 24 of the automatic injection device. The proximal removable cap 24 is provided in the automatic injection device for covering the proximal end of the housing of the automatic injection device to prevent exposure of the needle prior to an injection.

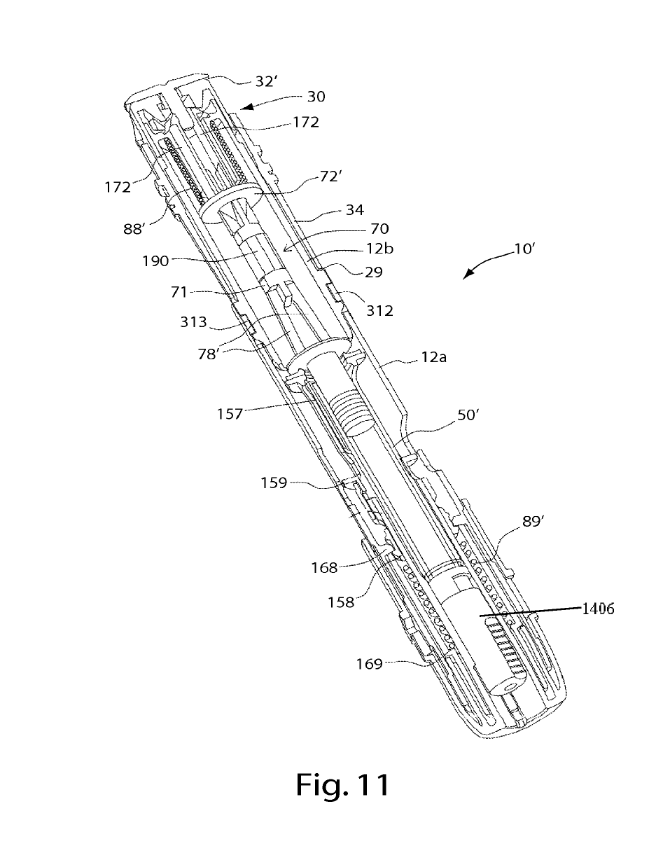

FIG. 11 is a cross-sectional view of an assembled automatic injection device 10′. The illustrative embodiment of the automatic injection device 10′ includes two mating proximal and distal housing components 12a, 12b. The proximal and distal housing components 12a, 12b mate to form a complete housing. As shown, a proximal housing component 12a, forming a proximal end of the housing, receives a proximal end of the distal housing components 12b.

A removable rigid needle shield 1406 is coupled to the proximal end of the syringe 50′ for protectively covering the syringe needle (not shown).

A cooperating projection 312 and groove 313, or a plurality of cooperating projections 312 and grooves 313, facilitate mating of the proximal and distal housing components 12a, 12b in the illustrative embodiment. Other suitable mating mechanisms may alternatively be employed. A shelf 29 formed on an outer surface of the distal housing component 12b to form a stop for the removable distal cap 34.

As shown, the firing button 32′ may be a cap covering the distal end of the distal housing component 12b. The illustrative firing button 32′ slides relative to the distal housing component 12b to actuate a syringe actuator, such as the plunger 70. The illustrative firing button 32′ releasably retains flexible anchoring arms 172 of the plunger 70′. When depressed, the firing button 32′ releases the flexible anchoring arms 172 to allow a first biasing mechanism, illustrated as spring 88′ to propel the plunger 70′ towards the proximal end of the device 10′.

In the embodiment of FIG. 11, the plunger 70′ further includes a flange 72′ located between the compressible expanded portion 78′ and the distal end of the plunger rod 71′. A first biasing mechanism 88′ is seated between an interior distal end of the housing and the flange 72′ to bias the plunger 70 towards the proximal end of the housing. When the firing button 34′ releases the anchoring arms 172, the coil spring 88′, or other suitable biasing mechanism propels the plunger 70′ towards the proximal end 20 of the device 10.

The plunger 70′ further includes an indicator 190 formed at an intermediate portion of the plunger rod 71 between the flange 72′ and the compressible expanded portion, illustrated as flexible elbows 78′. The indicator 190 may indicate to the patient of the device 10′ when the dose from the syringe 50 has been fully or substantially fully ejected. In the illustrative embodiment, the indicator 190 is formed on a portion of the plunger rod 71′ between the compressible expanded central portion 76 and the flange 72′. As the plunger rod 71 moves during operation, the indicator 190 advances towards and aligns with window 130 in the housing as the dose empties from the syringe. The indicator 190, which is preferably a different color or pattern from the substance being injected, fills the window 130 entirely to indicate that the dosage has been ejected. Any suitable indicator may be used.

The syringe 50′ of FIG. 11 may include protrusions or other suitable component to facilitate controlled movement of the syringe within the housing 12′. For example, with reference to FIG. 11, the syringe 50′ includes a sleeve 157 forming a proximal protrusion 158 for abutting a proximal side of a first protrusion 168 formed on an inner surface of the housing 12′ for limited movement of the syringe 50′ in the distal direction within the housing 12′. The sleeve 157 may also form a flange 159 that may abut the distal side of the first protrusion 168 to limit movement of the syringe 50′ in the proximal direction during an injection.

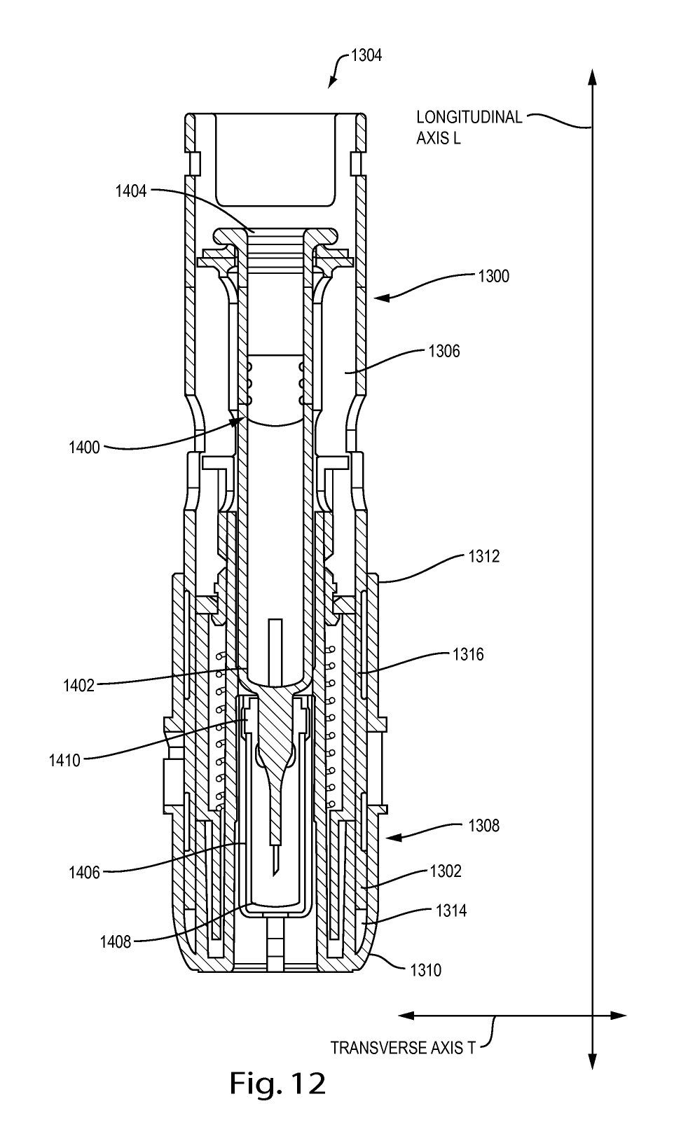

In the embodiment of FIG. 12, the second biasing mechanism, illustrated as coil spring 89′ is disposed about a proximal portion of the syringe 50′. A shelf 169 formed at a proximal inner surface of the housing 12′ receives a proximal end of the coil spring 89′. The proximal protrusion 158 of the syringe sleeve 157, or another suitably disposed mechanism, receives the distal end of the coil spring 89′. As described above, the second biasing mechanism 89′ biases the syringe 50′ in a retracted position within the housing 12′ until activation of the device 10.

FIG. 12 illustrates a cross-sectional view taken along the longitudinal axis L of the housing 1300 of an automatic injection device housing an exemplary syringe 1400. The housing 1300 of the automatic injection device extends substantially along the longitudinal axis L between a proximal end 1302 and a distal end 1304. The housing 1300 includes a hollow internal bore 1306 for accommodating the syringe 1400 and other related components, e.g., the syringe needle, a soft needle shield covering the syringe needle, a rigid needle shield 1406 covering the syringe needle and the soft needle shield, etc.

The proximal end 1302 of the housing 1300 includes or is fitted with a removable proximal cap 1308. The proximal cap 1308 extends substantially along the longitudinal axis L between a proximal end 1310 and a distal end 1312. The proximal cap 1308 includes a hollow internal bore 1314 for accommodating part or the entire length of a rigid needle shield 1406. In an exemplary embodiment, the hollow internal bore 1314 of the proximal cap 1308 may also accommodate a proximal portion of the syringe body 1400.

The syringe 1400 extends substantially along the longitudinal axis L between a proximal end 1402 and distal end 1404. The proximal end 1402 of the syringe 1400 is coupled to a syringe needle that may be covered by the removable rigid needle shield 1406. In some exemplary embodiments, the syringe needle may be covered by a removable soft needle shield that is, in turn, covered by the rigid needle shield 1406. The rigid needle shield 1406 extends substantially along the longitudinal axis L between a closed proximal end 1408 and an open distal end 1410 that abuts the proximal end 1402 of the syringe 1400. Exemplary lengths of rigid needle shields 1406 range from about 5 mm to about 30 mm, but are not limited to this range. In exemplary embodiments, the syringe 1400 may be housed within the housing 1300 of the automatic injection device such that the rigid needle shield 1406 is disposed partly or entirely within the proximal cap 1308.

In an exemplary embodiment, the internal bore 1314 of the proximal cap 1308 includes a friction point 1316, e.g., a local constriction or protrusion, that creates an area of increased frictional resistance against the insertion of the rigid needle shield 1406 into the bore 1314 of the proximal cap 1308. In an exemplary embodiment, the friction point 1316 may be located nearer the distal end 1312 of the proximal cap 1308 than the proximal end 1310 of the proximal cap 1308. In an exemplary embodiment, the friction point 1316 may be located substantially equidistant from the proximal end 1310 and the distal end 1312 of the proximal cap 1308.

During an exemplary assembly process, the syringe 1400 fitted, at its proximal end 1402, with the rigid needle shield 1406 is inserted into the housing 1300 of the automatic injection device such that the proximal ends of the syringe and the rigid needle shield move toward the proximal end 1302 of the housing 1300.

During an exemplary assembly process, the syringe 1400 fitted, at the proximal end, with the rigid needle shield 1406 is inserted into the housing 1300 fitted, at the proximal end, with the proximal cap 1308. Force profiles used in exemplary embodiments constitute a profile of the resistance forces exerted by the friction point 1316 against the entry of one or more different structural or ornamental features on the syringe 1400 and/or the rigid needle shield 1406 past the friction point 1316 as the syringe 1400 fitted with the rigid needle shield 1406 is inserted into the proximal cap 1308 during the syringe insertion process. In exemplary embodiments, the proximal cap of an exemplary automatic injection device may include a friction point at a different location than that shown in FIG. 12. In exemplary embodiments, the proximal cap of an exemplary automatic injection device may include two or more friction points located at a single location or located at different locations on the inner wall of the rigid needle shield 1406. In some exemplary embodiments, the friction point 1316 may be located on the inner wall of the housing 1300 itself.

In an exemplary embodiment, the rigid needle shield 1406 has a characteristic outer cross-sectional diameter over its length. In an exemplary embodiment, the outer surface of the rigid needle shield 1406 may include one or more friction points, e.g., one or more local increases in the outer cross-sectional diameter of the rigid needle shield 1406. In an exemplary embodiment, the local increases in diameter may be due to one or more structural or ornamental features that project from the outer surface of the rigid needle shield 1406. During the syringe insertion process, the portions of the rigid needle shield 1406 having increased diameters may result in characteristic force peaks in the force profile as the entry of those portions past the friction point 1316 in the proximal cap 1308 is resisted by higher frictional forces than the entry of portions of the rigid needle shield 1406 that have smaller diameters.

In an exemplary embodiment, the outer surface of the rigid needle shield 1406 may include one or more local decreases in the outer cross-sectional diameter. In an exemplary embodiment, the local decreases in diameter may be due to one or more structural dents or depressions provided on the outer surface of the rigid needle shield 1406. During the syringe insertion process, the portions of the rigid needle shield 1406 with the decreased diameters may result in characteristic force troughs in the force profile as the entry of those portions past the friction point 1316 in the proximal cap 1308 is resisted by lower frictional forces than the entry of portions of the rigid needle shield 1406 that have greater diameters.

### III. EXEMPLARY ASSEMBLY OF A SYRINGE HOUSING SUB-ASSEMBLY

In an exemplary automated method of assembling an automatic injection device, assembly of the syringe housing sub-assembly 121 illustrated in FIG. 8 may be performed separately from assembly of the firing mechanism subassembly 122 illustrated in FIG. 6. The assembled syringe housing sub-assembly 121 may then be assembled with the assembled firing mechanism sub-assembly 122 to form the automatic injection device.

An exemplary automated assembly process of assembling the syringe housing sub-assembly 121 may be performed in a fast and efficient manner. Exemplary time periods over which a syringe housing sub-assembly 121 may be assembled may range from about 1 second to about 30 seconds, but are not limited to this exemplary range.

In an exemplary automated method of assembling the syringe housing sub-assembly 121, the syringe carrier 1000 may be held in place by an assembly system with a distal portion of the proximal housing component 12a positioned over the syringe carrier 1000. The biasing mechanism 89 and the stepped shroud 1110 may be positioned within a proximal portion of the proximal housing component 12a. During the assembly process, the stepped shroud 1110 may be inserted into the proximal housing component 12a and the distal arms 1114 of the stepped shroud 1110 may be forced inward in order to couple the stepped shroud 1110 to the syringe carrier 1000. Insertion of the stepped shroud 1110 toward the syringe carrier 1000 within the proximal housing component 12a may cause the tabbed foot 1006 of the syringe carrier 1000 to fit within the slot 1118 of the shroud 1110, such that the two components cooperatively form a locking mechanism for the syringe carrier 1000 and the shroud 1110. In the assembled configuration, the tabbed foot 1006 may travel longitudinally within the slot 1118 but is restricted from disengaging from the slot 1118. This is because forward movement of the tabbed foot 1006 of the carrier 1000 may be stopped at the proximal end of the slot 1118 of the shroud 1100. At the same time, the rail 1007 fits along internal longitudinal grooves provided in the main tubular body portion 1116 of the shroud 1110, and moves longitudinally along the tracks provided by the grooves.

The assembly process may automatically detect and monitor the frictional forces exerted against the insertion of the shroud 1110 into the syringe carrier 1000. The detected forces may be used in a feedback mechanism to control or alter one or more aspects of the assembly process. This force feedback mechanism allows the assembly system to automatically and reliably determine the end point of the insertion of the shroud 1110 for assembly with the syringe carrier 1000. That is, the shroud 1110 is not inserted over a fixed predetermined distance in order to assemble the syringe housing sub-assembly 121. Rather, the assembly process is controlled based on one or more forces detected during the process and that may be used as feedback to accelerate, decelerate, start and/or stop the insertion of the shroud 1110 for assembly with the syringe carrier 1000. This allows the exemplary assembly process to accommodate for variability in the components of the syringe housing sub-assembly, and to thereby achieve reliable assembly of any set of components. In contrast, conventional assembly processes using mechanical cams insert one or more components over a fixed predetermined distance to assemble them with one or more other components. The use of a fixed predetermined insertion distance, without the benefit of feedback from force measurements, prevents the conventional processes from accommodating for variability in the components, and may result in improper assembly of the syringe housing sub-assemblies.

In one example, when one or more detected force values are determined to be substantially equal to one or more predefined force values, the exemplary assembly system may determine that the shroud 1110 is fully inserted and assembled with the syringe carrier 1000, and may terminate the assembly process. Alternatively or additionally, when one or more detected force values are determined to be substantially equal to one or more predefined force values, the assembly system may determine that the shroud 1110 is approaching full insertion toward the syringe carrier 1000, and may decelerate the assembly process.

In another example, when a portion of the detected force profile is determined to substantially match a predefined force profile, the assembly system may determine that the shroud 1110 is fully inserted and assembled with the syringe carrier 1000, and may terminate the assembly process. Alternatively or additionally, when a portion of the detected force profile is determined to substantially match a predefined force profile, the assembly system may determine that the shroud 1110 is approaching full insertion toward the syringe carrier 1000, and may decelerate the assembly process. A force profile may be generated in exemplary embodiments by detecting and plotting force values against incremental distances over which the shroud 1110 is inserted during the assembly process.

In an exemplary embodiment, the forces values may be detected at one or more load cells used in the assembly process, for example, load cells manufactured by the Kistler Group. In an exemplary embodiment, the detected forces may be displayed on one or more visual display interfaces, for example, CoMo View® interfaces manufactured by the Kistler Group.

In an exemplary embodiment, the compression of the biasing mechanism 89 may be monitored during the assembly process.

In an exemplary embodiment, the assembly system may monitor the functionality and deployment of the shroud 1110 during or after the shroud 1110 is fully assembled with the syringe carrier 1000. The assembly system may monitor the force required to deploy the shroud 1110 over a percentage of its fully deployed distance in order to determine whether the shroud 1110 will be successfully and reliably deployed during operation of the automatic injection device. The shroud 1110 is only partially deployed during this testing phase to prevent complete and irreversible lockout of the shroud 1110. The percentage of the fully deployed distance monitored may range from about 50% to about 98% in some exemplary embodiments. The percentage of the fully deployed distance monitored is about 95% in one exemplary embodiment.

If the force required to deploy the shroud 1110 is at or below one or more predefined force values, exemplary embodiments may determine that the shroud 1110 will deploy reliably, and may reposition the shroud 1110 toward the syringe carrier 1000 to return the shroud to its non-deployed state. On the other hand, if the force required to deploy the shroud 1110 is above one or more predefined force values, exemplary embodiments may determine that the shroud 1110 is incorrectly assembled or is defective. In this case, in one example, the shroud 1110 may be discarded.

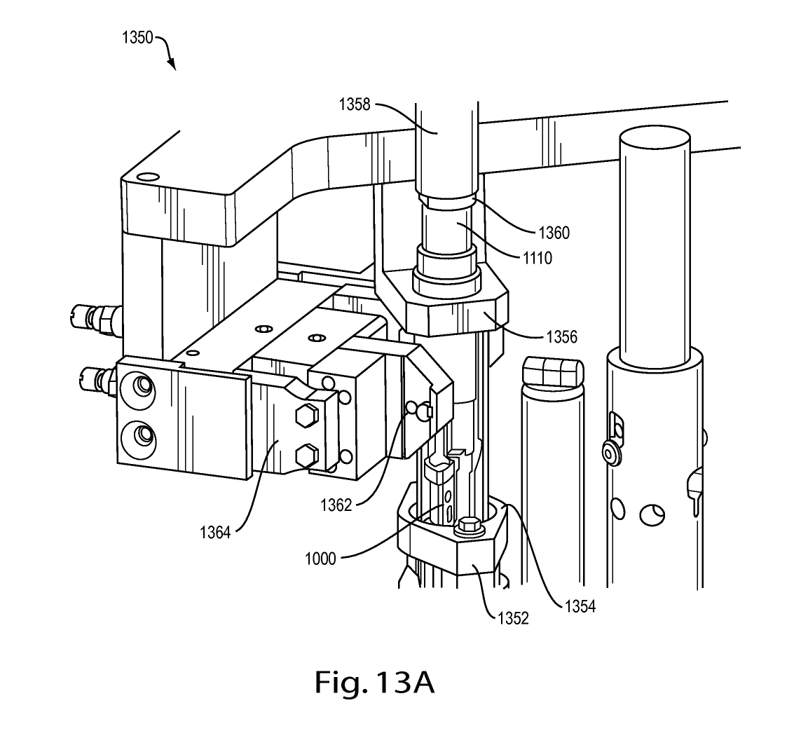

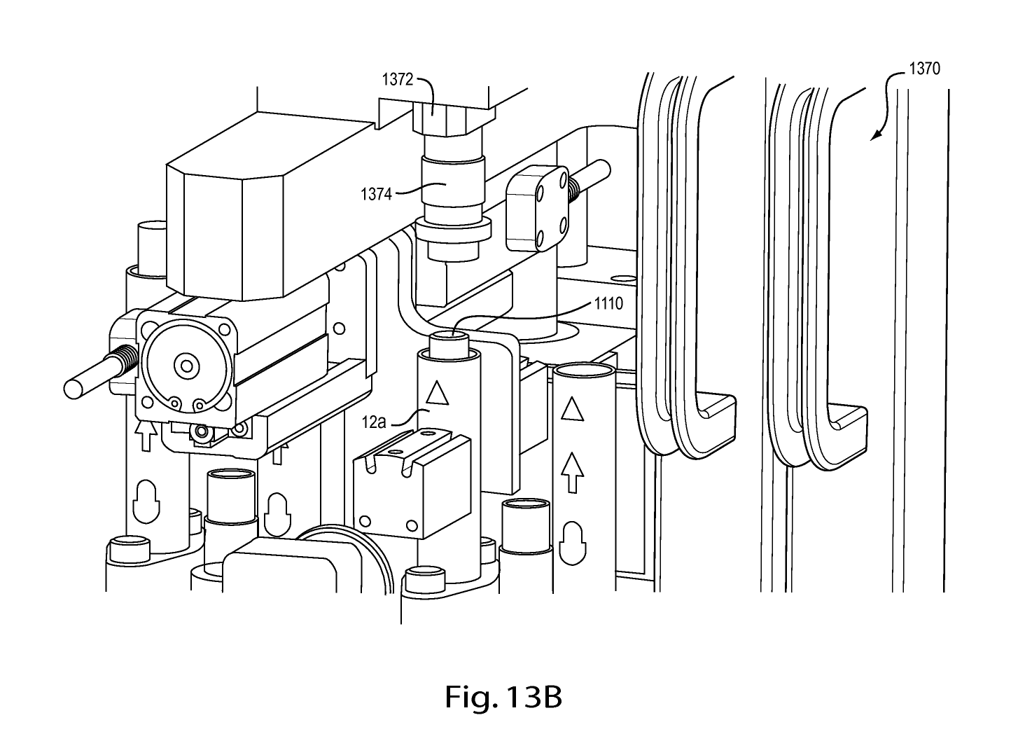

FIG. 13A illustrates an exemplary perspective view of an assembly system 1350 that may be used to assemble the exemplary syringe housing sub-assembly 121. In FIG. 13A, the assembly system 1350 is in a pre-assembly state in which the components of the syringe housing sub-assembly 121 are ready for assembly but have not been assembled yet. An exemplary assembly system 1350 may be an assembly system produced by sortimat, an affiliate of ATS Automation.

The assembly system 1350 may include an assembly pallet 1352 for supporting and holding one or more components of the syringe housing sub-assembly 121 in a vertical orientation during the assembly process. In an exemplary embodiment, the assembly pallet 1352 may be configured as a substantially cylindrical component with a central recessed portion 1354 for accommodating and supporting the components. In an exemplary embodiment, the assembly pallet 1352 may support the bottom portions of the proximal housing component 12a (pictured transparently in FIG. 13A) and the syringe carrier 1000 positioned within the bore of the proximal housing component 12a. In an exemplary embodiment, the shroud 1110 may be positioned within the bore of the proximal housing component 12a above the syringe carrier 1000, and the biasing mechanism 89 may be arranged between the syringe carrier 1000 and the shroud 1110.

The assembly system 1350 may include a gripping mechanism 1356 for supporting the side portion of one or more components of the syringe housing sub-assembly 121 so that the orientations are held in a vertical orientation during the assembly process. In an exemplary embodiment, the gripping mechanism 1356 may be configured as a solid mechanism oriented horizontally and including a central bore for accommodating one or more components so that the sidewall of the central bore supports the side portions of the components. In an exemplary embodiment, the gripping mechanism 1356 may support the side portions of the shroud 1110.

The assembly system 1350 may include a mechanical member 1358 with a terminal end configured as a press head 1360. The press head 1360 may configured to contact and press downward on the proximal end of the shroud 1110 to couple the shroud 1110 with the syringe carrier 1000 within the proximal housing component 12a. The press head 1360 may include or be associated with one or more force and/or pressure sensors, e.g., one or more piezoelectric load cells, for detecting and monitoring forces and/or pressures experienced during the assembly process. In an exemplary embodiment, the piezoelectric sensor includes a quartz crystal and two steel rings that generate an electrical charge when subjected to mechanical force or stress. The charge generated by the sensor may be directly proportional to the mechanical force applied to the sensor. In an exemplary embodiment, the force detected by the force sensor may be the frictional force with which the insertion of the shroud 1110 toward the syringe carrier 1000 is resisted during the assembly process. An exemplary force sensor may include, but is not limited to, a direct piezoelectric load cell manufactured by the Kistler Group.

The assembly system 1350 may include one or more motion generators (not pictured) that provide a motion for moving the mechanical member 1358. An exemplary motion generator may include, but is not limited to, a servomotor that drives the mechanical member 1358 in an upward or downward direction along the vertical axis. In an exemplary embodiment, the motion generator may be couplable to the mechanical member 1358 via a drive system (not pictured). In some embodiments, the drive system may be configured as a worm drive. The drive system may be coupled to the mechanical member 1358 via a flange or other coupler. In an exemplary embodiment, the drive system allows macro incremental movements of the press head 1360 of the mechanical member 1358 along the vertical axis V on the order of about 1 mm. In an exemplary embodiment, the drive system allows micro incremental movements of the press head 1360 of the mechanical member 1358 along the vertical axis V on the order of about 0.1 mm.

In an exemplary embodiment, the assembly system 1350 may include one or more shroud arm assembly mechanisms 1362 for contacting the sides of the distal arms 1114 of the shroud 1110 and for pushing the arms 1114 horizontally inward to be accommodated within the hollow bore of the proximal housing component 12a. In an exemplary embodiment, the shroud arm assembly mechanism 1362 may be configured as one or more pins that are spaced around the shroud 1110 and that extend substantially horizontally toward the arms 1114 of the shroud 1110. When actuated during the assembly process, the shroud arm assembly mechanism 1362 may move inward toward the arms 1114, make contact with the arms 1114 and push the arms 1114 inward so that the arms 1114 may be accommodated within the bore of the proximal housing component 12a. The assembly system 1350 may also include an actuator 1364 for driving the shroud arm assembly mechanism 1362.

FIG. 13B illustrates an exemplary perspective view of another assembly system 1370 that may be used to assemble the exemplary syringe housing sub-assembly 121. In FIG. 13B, the assembly system 1370 is in a post-assembly state in which the components of the syringe housing sub-assembly 121 have been assembled. This is illustrated by the hollow bore of the proximal housing component 12a having assembled therein the syringe carrier 1000, the biasing mechanism 89, and the shroud 1110. Similar to the assembly system 1350 of FIG. 13A, the assembly system 1370 may include a motion generator 1372 configured to drive a mechanical member 1374 with a terminal end configured as a press head, and one or more other suitable components. Components common between FIGS. 13A and 13B are described with reference to FIG. 13A.

Although the exemplary assembly systems 1350 and 1370 are described as inserting the shroud 1110 toward the syringe carrier 1000, the same or a different assembly system may be used to hold the shroud 1110 in place while the syringe carrier 1000 is inserted toward the shroud 1110.

The assembly systems 1350 and 1370 of FIGS. 13A and 13B, respectively, may include a motion control computing device for controlling one or more control parameters for the motion generator. The motion control computing device may be provided integrally with the motion generator or separately from the motion generator. Exemplary control parameters of the motion generator controllable using the motion control computing device include, but are not limited to, starting/stopping of the motion generator, distance traveled, distance left to travel, speed, acceleration, deceleration, different phases of motion of the motion generator, etc. One or more control parameters may be set or altered by the motion control computing device based on one or more control factors including, but not limited to, a trigger instruction or signal generated when a particular force feature is detected in the force profile (e.g., the motion generator may be stopped when the trigger instruction or signal is received), after the crossing of a predefined distance over which the shroud 1110 is inserted into the proximal housing component 12a (e.g., the insertion speed of the shroud 1110 may be reduced after the shroud 1110 is inserted over a predefined distance into the proximal housing component 12a), the lapse of a predefined period of time (e.g., the insertion speed of the shroud 1110 may be reduced after a predefined period of time has elapsed), etc.

In an exemplary embodiment, the assembly process may be divided into one or more phases with each phase having an associated set of control parameters, and the control parameters may be set and/or changed automatically by the motion control computing device based on the particular phase of the assembly process at a given time.

In an exemplary embodiment, the motion control computing device may be pre-programmed to control the motion generator in a desired manner during the assembly insertion process. The pre-programming of the motion control computing device may be overridden or altered by a user before or during the assembly process. In another exemplary embodiment, the motion control computing device may not be pre-programmed, and a user may use the motion control computing device to enter and control a programming of the motion generator before or during the assembly process.

The assembly systems 1350 and 1370 may include one or more trigger generation computing devices connectable to the force/pressure sensor for measuring the forces and/or pressures exerted during the assembly process and for measuring the displacement of the press head during the assembly process based on an output from the force/pressure sensor. The trigger generation computing device may perform one or more functions including, but not limited to, measuring in real-time the forces detected by the force/pressure sensor, measuring in real-time the pressures detected by the force/pressure sensor, determining in real-time whether one or more trigger conditions are satisfied by the force profile, generating a trigger instruction or signal when one or more trigger conditions are met, sending the trigger instruction or signal to the motion control computing device to control the motion generator, and the like.

In an exemplary embodiment, the trigger generation computing device may be pre-programmed to recognize one or more characteristic force features in the force profile that, when generated, satisfy a trigger condition. The pre-programming of the trigger generation computing device may be overridden or altered by a user before or during the assembly process. In another exemplary embodiment, the trigger generation computing device may not be pre-programmed, and a user may set one or more characteristic force features that, when generated, satisfy a trigger condition before or during the assembly process.

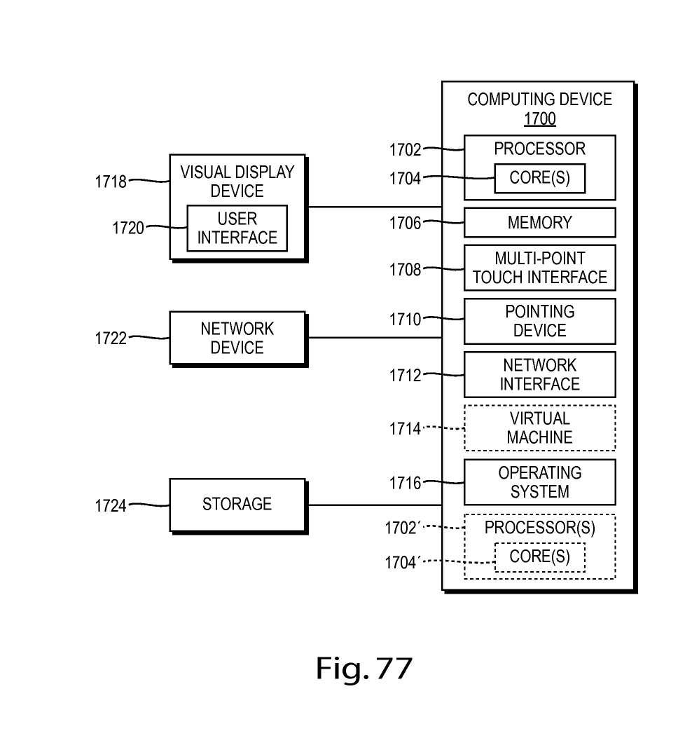

FIG. 77 illustrates a block diagram of an exemplary computing device that may be used in exemplary embodiments as the motion control computing device and/or the trigger generation computing device. The exemplary computing device is described below in connection with FIG. 77.

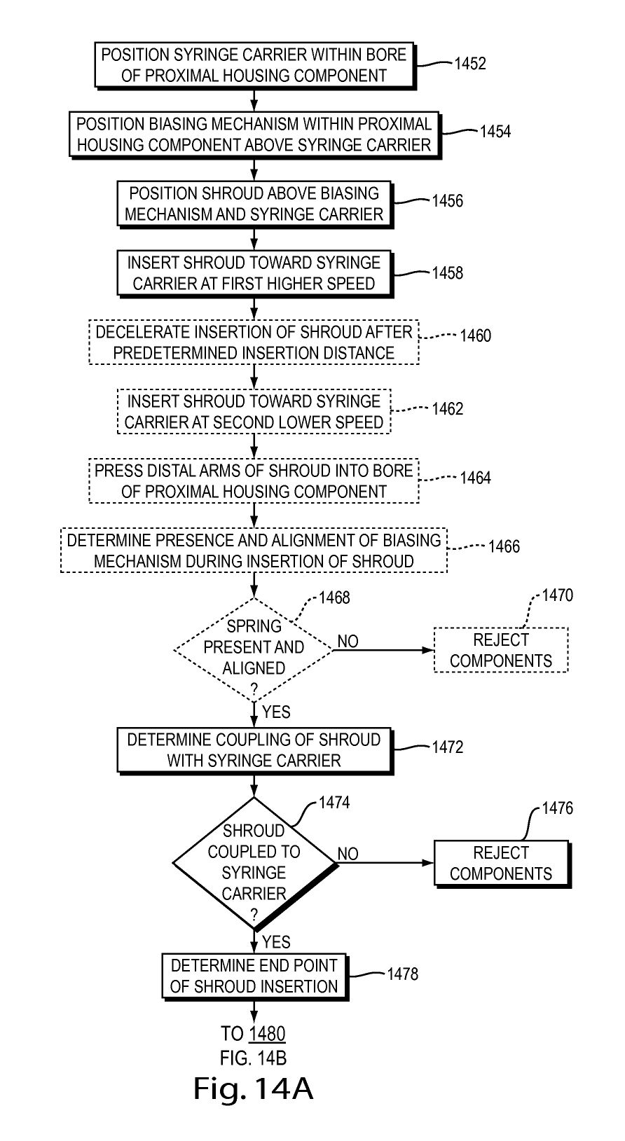

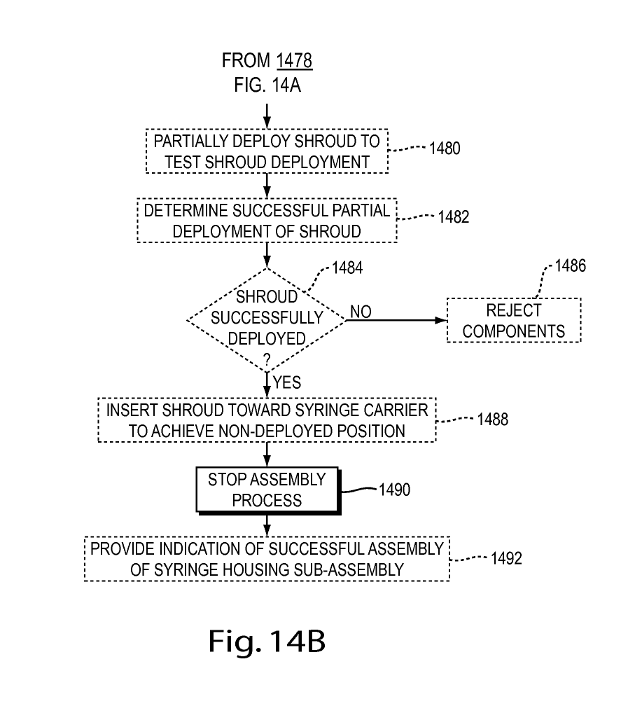

FIGS. 14A and 14B are flowcharts illustrating an exemplary method for assembling a syringe housing sub-assembly for use in an automatic injection device. Forces experienced at the press head may be detected and monitored during the assembly method.

In step 1452, the syringe carrier 1000 may be positioned within the hollow bore of the proximal housing component 12a. In step 1454, the biasing mechanism 89 may be positioned within the bore of the proximal housing component 12a above the syringe carrier 1000. In step 1456, the shroud 1110 may be positioned above the biasing mechanism 89 and the syringe carrier 1000 such that the biasing mechanism 89 is accommodated between the syringe carrier 1000 and the shroud 1110.

In step 1458, the press head may insert the shroud 1110, at a first higher speed, within the bore of the proximal housing component 12a toward the syringe carrier 1000. In step 1460, after the shroud 1110 has been inserted over a predetermined distance, it may be determined that the assembly process is approaching completion and the movement of the press head may be decelerated. In step 1462, the press head may insert the shroud 1110, at a second lower speed, within the bore of the proximal housing component 12a toward the syringe carrier 1000.

In step 1464, the distal arms 1114 of the shroud 1110 may be pressed radially inward to fit within the bore of the proximal housing component 12a.

In step 1466, exemplary embodiments may determine whether the biasing mechanism 89 is present and correctly aligned with the side walls of the shroud 1110. In one example, absence of the biasing mechanism 89 may cause the forces experienced by the press head to fall below one or more predetermined thresholds. In another example, if the biasing mechanism 89 is incorrectly assembled with the sidewalls of the shroud 1110, the press head may experience higher forces than one or more predetermined thresholds. Exemplary embodiments may determine that the biasing mechanism 89 is absent if the forces experienced during compression of the biasing mechanism 89 are lower than one or more predetermined thresholds. Exemplary embodiments may also determine that the biasing mechanism 89 is present but incorrectly assembled if the forces experienced during compression of the biasing mechanism 89 are higher than one or more predetermined thresholds. If exemplary embodiments determine in step 1468 that the biasing mechanism 89 is absent or incorrectly assembled, the assembly process may be terminated and the components discarded in step 1470. Otherwise, the method may progress to step 1472.

In step 1472, exemplary embodiments may determine whether the shroud 1110 is correctly coupled to the syringe carrier 1000. Exemplary embodiments may determine that the shroud 1110 is correctly coupled to the syringe carrier 1000 if one or more forces experienced by the press head indicate a decreasing force profile within a predetermined insertion distance range, indicating that the tabbed foot 1006 of the syringe carrier 1000 has been snapped into place within the slot 1118 of the shroud 1110, such that the two components cooperatively form a locking mechanism for the syringe carrier 1000 and the shroud 1110. If exemplary embodiments determine in step 1474 that the shroud 1110 is incorrectly coupled to the syringe carrier 1000, the assembly process may be terminated and the components discarded in step 1476. Otherwise, the method may progress to step 1478.

In step 1478, exemplary embodiments may determine an end point of the shroud insertion, i.e., that the shroud 1110 has been inserted to an appropriate position relative to the syringe carrier 1000.

Exemplary embodiments may then test deployment of the shroud 1110. In step 1480, exemplary embodiments may instruct the press head to stop movement toward the syringe carrier 1000, the assembly system to cause partial deployment of the shroud 1110, and the press head to move away from the syringe carrier 1000. Deployment of the shroud 1110 may be tested by partially deploying the shroud 1110 so that the shroud 1110 moves away from the syringe carrier 1000 while the tabbed foot 1006 of the syringe carrier 1000 is still coupled to the slot 1118 of the shroud 1110. During deployment of the shroud 1110, the slidability of the tabbed foot 1006 back and forth within the slot 1118 allows the shroud 1110 and the syringe carrier 1000 to move relative to each other but to still be coupled.

In step 1482, exemplary embodiments may determine whether the shroud 1110 was successfully partially deployed. Exemplary embodiments may determine that the shroud 1110 was unsuccessfully deployed if one or more forces experienced at the press head are lower than a predetermined threshold, indicating that the press head has lost contact with the shroud 1110 as the press head moves away from the syringe carrier 1000. If exemplary embodiments determine in step 1484 that the shroud 1110 has not deployed, the assembly process may be terminated and the components discarded in step 1486. Otherwise, the method may progress to step 1488.

In step 1488, exemplary embodiments may instruct the press head to again move toward the syringe carrier 1000 to insert the deployed shroud toward the syringe carrier 1000 to its non-deployed position. During step 1488, the slidability of the tabbed foot 1006 back and forth within the slot 1118 allows the shroud 1110 and the syringe carrier 1000 to be remained coupled.

In step 1490, upon return of the shroud 1110 to its non-deployed state, the assembly process may be complete. Exemplary embodiments may instruct the press head to stop movement toward the syringe carrier 1000 and to return to its original position. In step 1492, the assembly system may provide a visual and/or auditory indication that the syringe housing sub-assembly 121 has been successfully and correctly assembled. The syringe housing sub-assembly 121 may subsequently be used to form an automatic injection device.

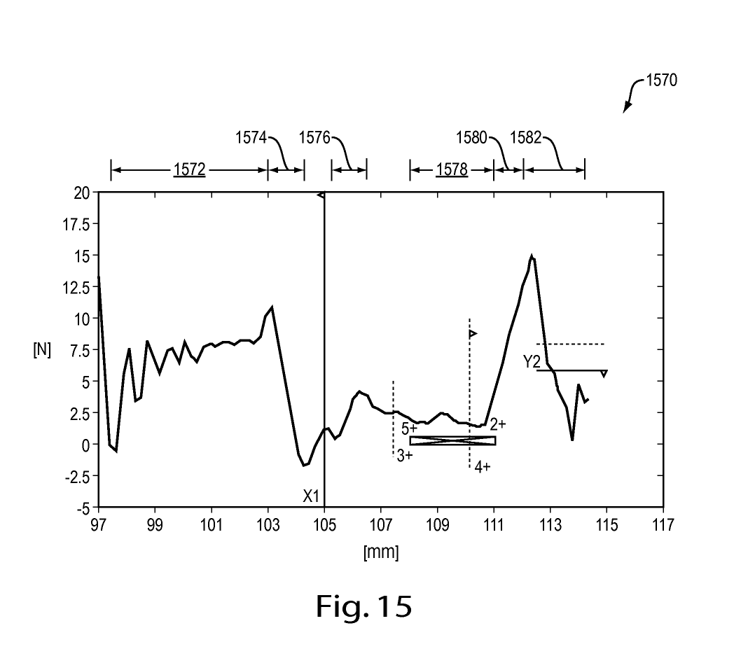

FIG. 15 illustrates an exemplary force profile 1570 of the forces experienced at the press head during assembly of the syringe housing sub-assembly 121. The y-axis of the force profile denotes the frictional forces (in N) detected by an exemplary force sensor at the press head. The x-axis of the force profile denotes the distance (in mm) moved by the press head toward the syringe carrier (indicated on the profile as moving from left to right) and/or away from the syringe carrier (indicated on the profile as moving from right to left).

A first portion 1572 of the force profile shows gradually increasing forces experienced when the biasing mechanism 89 is compressed by the movement of the shroud 1110 toward the syringe carrier 1000. Exemplary forces in the first portion 1572 may range from about 0 N to about 11 N over an exemplary insertion distance of about 97 mm to about 103 mm in some exemplary embodiments.

The movement of the press head toward the syringe carrier may be decelerated after the press head has been inserted over a predetermined distance, for example, at 103 mm. Exemplary embodiments may detect that the predetermined distance has been traveled and may instruct the press head to decelerate. A second portion 1574 of the force profile shows a rapid decrease in the forces experienced when the speed of the press head is reduced. Exemplary forces in the second portion 1574 may drop from about 11 N to about −2 N over an exemplary insertion distance of about 103 mm to about 104 mm in some exemplary embodiments.

A third portion 1576 of the force profile shows an increase in the forces experienced when the distal arms 1114 of the shroud 1110 are forced radially inward to be accommodated within the bore of the proximal housing component 12a. Exemplary forces in the third portion 1576 may range from about 2.5 N to about 5 N over an insertion distance of about 106 mm to about 107 mm in some exemplary embodiments.

A fourth portion 1578 of the force profile shows substantially stable forces (i.e., forces not undergoing rapid increases or decreases) as the shroud 1110 is moved farther toward the syringe carrier 1000. In the fourth portion 1578, exemplary embodiments may determine whether the biasing mechanism 89 is present and correctly aligned within the side walls of the shroud 1110. In an exemplary embodiment, if the forces experienced by the press head over an insertion distance of about 108 mm to about 111 mm fall within a range of about 0 N to about 0.6 N, the motion control computing device may determine that the biasing mechanism 89 is absent in the assembly, because presence and compression of the biasing mechanism 89 would result in higher forces in the fourth portion 1578 of the force profile. If exemplary embodiments determine that the forces do fall within this proscribed range, the press head may be instructed to return to its original position, the assembly process may be terminated, and the components may be discarded.

A fifth portion 1580 of the force profile shows a rapid increase in the forces experienced as the tabbed feet 1006 of the syringe carrier 1000 impinge upon and resist the distal end of the shroud 1110. Exemplary forces in the fifth portion 1580 may increase from about 2 N to about 15 N over an insertion distance of about 110 mm to about 112 mm in some exemplary embodiments.

A sixth portion 1582 of the force profile shows a rapid decrease in the forces experienced as the tabbed feet 1006 of the syringe carrier 1000 snap into place within the slot 1118 of the shroud 1110. The slidable positioning of the tabbed feet 1006 in the slot 1118 allows relative movement between the shroud 1110 and the syringe carrier 1000, which reduces the frictional forces exerted against the farther movement of the press head toward the syringe carrier 1000. Exemplary forces in the sixth portion 1582 may decrease from about 15 N to about 0 N over an insertion distance of about 112 mm to about 114 mm in some exemplary embodiments.

In the sixth portion 1582, exemplary embodiments may determine whether one or more force values detected during the assembly process match a trigger condition that indicates that the end point of the insertion of the shroud 1110 has been reached or is close to being reached. In an exemplary embodiment, the trigger condition may be set to be one or more force values that appear in the sixth portion 1582 of the force profile, for example, about 6 N. The trigger hysteresis may be set to be a small force value, for example, about 2 N. The x-axis range within which the trigger force and the trigger hysteresis are detected or measured may be set to be between about 112 mm and about 115 mm. The approach is indicated to be “from above,” which indicates that the trigger condition is satisfied if the force falls from about 8 N to about 6 N within an x-axis range of between 112 mm and about 115 mm. If the trigger condition is satisfied, exemplary embodiments may instruct the press head to return to its original position as the assembly process has been completed.

One of ordinary skill in the art will recognize that exemplary force profile 1570 may have fewer or additional features corresponding to interactions among the components. One of ordinary skill in the art will recognize that the force and insertion distance values used in the exemplary method of FIG. 1570 are exemplary, and that any suitable force and insertion distance values may be used to determine when the assembly process should be stopped and to determine whether the components are assembled correctly.

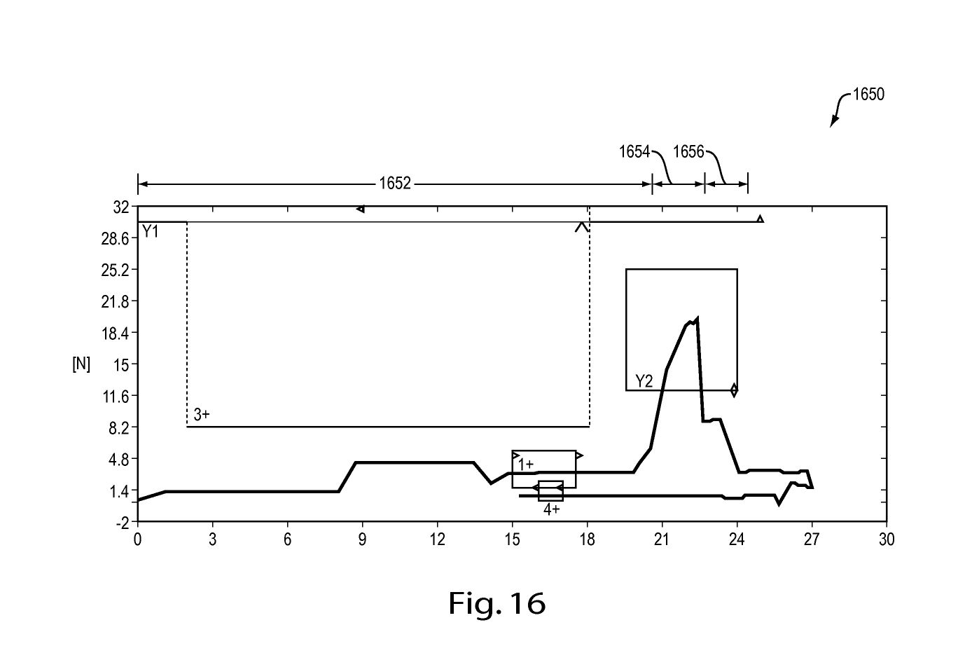

FIG. 16 illustrates another exemplary force profile 1650 of the forces experienced at the press head during assembly of the syringe housing sub-assembly 121. The y-axis of the force profile denotes the frictional forces (in N) detected by an exemplary force sensor at the press head. The x-axis of the force profile denotes the distance (in mm) moved by the press head toward the syringe carrier (indicated on the profile as moving from left to right) and/or away from the syringe carrier (indicated on the profile as moving from right to left). The force profile 1650 of FIG. 16 was generated by a different assembly system than the force profile 1550 of FIG. 15.

A first portion 1652 of the force profile shows gradually increasing forces experienced when the biasing mechanism 89 is compressed by the movement of the shroud 1110 toward the syringe carrier 1000. Exemplary forces in the first portion 1652 may range from about 0 N to about 3.5 N over an exemplary insertion distance of about 0 mm to about 20 mm in some exemplary embodiments.

In the first portion 1652 of the force profile, exemplary embodiments may determine whether the biasing mechanism 89 is present and correctly aligned within the side walls of the shroud 1110. In an exemplary embodiment, if the detected forces exceed 8 N over an insertion distance of about 2 mm to about 18 mm, exemplary embodiments may determine that the biasing mechanism 89 is incorrectly assembled. In this case, the press head may be instructed to return to its original position, the assembly process may be terminated, and the components may be discarded. In an exemplary embodiment, if the detected forces fall below a range of about 1.5 N to about 5.5 N within an insertion distance of about 15 mm and about 17.5 mm, exemplary embodiments may determine that the biasing mechanism 89 is absent. In this case, the press head may be instructed to return to its original position, the assembly process may be terminated, and the components may be discarded.

A second portion 1654 of the force profile shows a rapid increase in the forces experienced as the tabbed feet 1006 of the syringe carrier 1000 impinge upon and resist the distal portion of the shroud 1110. Exemplary forces in the second portion 1654 may increase from about 4 N to about 20 N over an insertion distance of about 20 mm to about 22.5 mm in some exemplary embodiments.

A third portion 1656 of the force profile shows a rapid decrease in the forces experienced as the tabbed feet 1006 of the syringe carrier 1000 snap into place within the slot 1118 of the shroud 1110. The slidable positioning of the tabbed feet 1006 in the slot 1118 allows relative movement between the shroud 1110 and the syringe carrier 1000, which reduces the frictional forces exerted against the farther movement of the press head toward the syringe carrier 1000. Exemplary forces in the third portion 1656 may decrease from about 20 N to about 0 N over an insertion distance of about 22.5 mm to about 27 mm in some exemplary embodiments.

Exemplary embodiments may determine whether one or more force values detected during the second and third portions 1654, 1656 of the force profile match one or more trigger conditions indicating that the shroud 1110 and the syringe carrier 1000 have been correctly assembled. In an exemplary embodiment, if the detected forces rise higher than and drop below about 12 N over an insertion distance of about 19.5 mm to about 24 mm (while never exceeding about 25 N), exemplary embodiments may determine that this corresponds to a peak in the force which corresponds to proper coupling of the tabbed feet 1006 of the syringe carrier 1000 and the slot 1118 of the shroud 1110. If the detected forces do not fall within the above range, this may indicate that the components have not been correctly assembled. In this case, the press head may be instructed to return to its original position, the assembly process may be terminated, and the components may be discarded.

In the second and third portions 1654, 1656 of the force profile, exemplary embodiments may determine whether one or more force values detected during the assembly process match a trigger condition that indicates that the end point of the insertion of the shroud has been reached. In an exemplary embodiment, the trigger condition may be set to be one or more force values that appear on the third portion 1656 of the force profile, for example, about 12 N. The x-axis range within which the trigger force is detected or measured may be set to be between about 20 mm and about 24 mm. The approach is indicated to be “from above,” which indicates that the trigger condition is satisfied if the force is about 12 N and has a decreasing trend within an x-axis range of between 20 mm and about 24 mm. If the trigger condition is satisfied, the press head may be instructed to return to its original position and the assembly process is completed.

In an exemplary embodiment, if, at any time during the assembly process, the detected forces exceed a maximum threshold of about 30 N, the assembly process may be terminated and the components may be discarded.

One of ordinary skill in the art will recognize that exemplary force profile 1650 may have fewer or additional features corresponding to interactions between the components. One of ordinary skill in the art will recognize that the force and insertion range values used in the exemplary method of FIG. 16 are exemplary, and that any suitable force and insertion range values may be used to determine when the assembly process should be stopped and to determine whether the components are assembled correctly.

In an exemplary embodiment, if the trigger condition is satisfied in the third portion 1656 of the force profile, the deployment of the shroud 1110 may be tested by causing the shroud 1110 to deploy partially, for example, about 95% of its full deployment distance. The shroud 1110 is only partially deployed during this testing phase to prevent complete and irreversible lockout of the shroud 1110. The assembly system may cause partial deployment of the shroud 1110 by causing the press head to press down the entire syringe housing sub-assembly 121 into the assembly pallet. This causes the syringe carrier 1000 to advance toward the shroud 1112, thereby compressing the biasing mechanism 89. The proximal housing component 12a is held in place and the press head is lifted away from the syringe carrier 1000, while monitoring the forces experienced by the force sensor. This causes the shroud 1110 to be deployed.

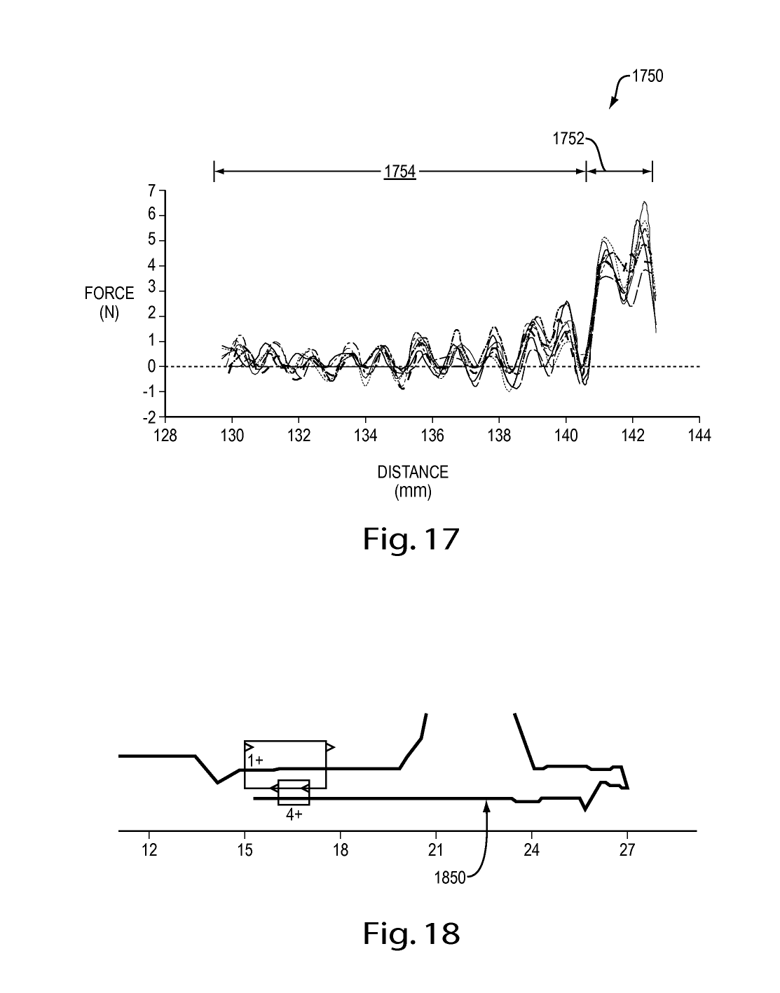

FIG. 17 illustrates an exemplary force profile 1750 showing forces experienced at the press head during the deployment of the shroud 1110. The y-axis of the force profile denotes the frictional forces (in N) detected by an exemplary force sensor at the press head. The x-axis of the force profile denotes the distance (in mm) moved by the press head away from the syringe carrier (indicated on the profile as moving from right to left). The oscillation seen in the force profile 1750 is a result of the high speed of travel of the press head.

A first portion 1752 of the force profile shows high levels of force since the biasing mechanism 89 is almost fully compressed in the initial stage of shroud deployment, which gives rise to high frictional forces exerted against the press head. In an exemplary embodiment, the forces at the first portion 1752 may range from about 4 N to about 6 N over an insertion distance of about 143 mm to about 140 mm in some exemplary embodiments. A second portion 1754 of the force profile shows lower levels of force as the biasing mechanism 89 decompresses with the deployment of the shroud 1110. In an exemplary embodiment, the mean value of the forces at the second portion 1754 may range from about 0.5 N to about 1.5 N.

FIG. 18 illustrates another exemplary force profile 1850 showing forces experienced at the press head during the deployment of the shroud 1110. The y-axis of the force profile denotes the frictional forces (in N) detected by an exemplary force sensor at the press head. The x-axis of the force profile denotes the distance (in mm) moved by the press head away from the syringe carrier (indicated on the profile as moving from right to left).

Exemplary embodiments may determine that the shroud 1110 has successfully deployed if the detected forces fall between about 0.1 N to about 2 N over an insertion distance of about 17 mm to about 16 mm in some exemplary embodiments, as the force profile runs from the right to the left. If the detected forces are below 0.1 N, this may indicate that contact between the shroud 1110 and the press head has been lost, indicating that the shroud has failed to deploy. On the other hand, if the forces are above 2 N, this may indicate that there may be an anomaly in the biasing mechanism 89.

If the detected forces do not satisfy the above exemplary range, the syringe housing sub-assembly may be discarded as the shroud 1110 has failed to deploy. On the other hand, if the detected forces satisfy the range, exemplary embodiments may determine that the shroud 1110 will deploy reliably, and may reposition the shroud 1110 toward the syringe carrier 1000 using the press head to return the shroud 1110 to its non-deployed state. The syringe housing sub-assembly 121 is then ready for assembly with other components to form an automatic injection device.

Although an exemplary assembly of a syringe housing sub-assembly is described with reference to inserting the shroud 1110 toward the syringe carrier 1000, one of ordinary skill in the art will appreciate that exemplary embodiments may also be used to insert the syringe carrier 1000 toward the shroud 1110, and/or to insert the syringe carrier 100 and the shroud 1110 toward each other in order to assemble the syringe housing sub-assembly.

### IV. EXEMPLARY ASSEMBLY OF A FIRING MECHANISM SUB-ASSEMBLY

In an exemplary automated method of assembling an automatic injection device, assembly of the firing mechanism subassembly 122 illustrated in FIG. 6 may be performed separately from assembly of the syringe housing sub-assembly 121 illustrated in FIG. 8. The assembled firing mechanism subassembly 122 may then be assembled with the assembled syringe housing sub-assembly 121 to form the automatic injection device. Exemplary time periods over which a firing mechanism sub-assembly 122 may be assembled may range from about 1 second to about 30 seconds, but are not limited to this exemplary range.

In an exemplary automated method of assembling the firing mechanism sub-assembly 122, the firing button 32 may be positioned between the distal cap 34 and the firing body 12b. A distal portion of the biasing mechanism 88 may be positioned within the hollow barrel portion of the firing body 12b, and the syringe actuation component 700′ may be positioned at the proximal end of the firing body 12b. During the assembly process, the syringe actuation component 700′ may be inserted into the hollow barrel portion of the firing body 12b by an automatic assembly system. Insertion of the syringe actuation component 700′ into the firing body 12b may cause the arms 788′ of the syringe actuation component 700′ to be accommodated within the biasing mechanism 88 positioned inside the firing body 12b. The flange 720′ of the syringe actuation component 700′ may provide a stop mechanism for the biasing mechanism 88 so that insertion of the syringe actuation component 700′ causes compression of the biasing mechanism 88 into the barrel of the firing body 12b. In an exemplary embodiment, the firing button 32 may be assembled between the distal cap 34 and the firing body 12b (for example, by pressing the distal cap 34 toward the firing body 12b and snapping clicking it into place) before the syringe actuation component 700′ is inserted into the firing body 12b. In another exemplary embodiment, the firing button 32 may be assembled between the distal cap 34 and the firing body 12b (for example, by pressing the distal cap 34 toward the firing body 12b and snapping or clicking it into place) after the syringe actuation component 700′ is inserted into the firing body 12b.

The automated assembly process may automatically detect and monitor the forces experienced as a result of pressing the syringe actuation component 700′ into the firing body 12b. The detected forces may be used in a feedback mechanism to control or alter one or more aspects of the assembly process. This force feedback mechanism allows the assembly station to automatically and reliably determine the completion of the insertion of the syringe actuation component 700′ into the firing body 12b, and to determine whether the sub-assembly has been correctly assembled. That is, syringe actuation component 700′ is not inserted over a fixed predetermined distance in order to assemble the firing mechanism sub-assembly 122. Rather, the exemplary assembly process is automatically controlled based on one or more forces that are detected during the process and that may be used as feedback to accelerate, decelerate, start and/or stop the insertion of the syringe actuation component 700′ into the firing body 12b. This allows the exemplary assembly process to accommodate for variability in the components of the firing mechanism sub-assembly 122, and to thereby achieve reliable assembly of any set of components. In contrast, conventional assembly processes using mechanical cams insert one or more components over a fixed predetermined distance to assemble them with one or more other components. The use of fixed predetermined insertion distances, without the benefit of feedback from force measurements, prevents the conventional processes from accommodating for variability in the components, and may result in improper assembly of the firing mechanism sub-assemblies.

In one example, when one or more detected force values are determined to be substantially equal to one or more predefined force values, the assembly system may determine that the syringe actuation component 700′ is fully inserted into the firing body 12b, and may terminate the insertion process. Alternatively or additionally, when one or more detected force values are determined to be substantially equal to one or more predefined force values, the assembly system may determine that the syringe actuation component 700′ is approaching full insertion into the firing body 12b, and may decelerate the insertion process.

In another example, when a portion of the detected force profile is determined to substantially match a predefined force profile, the assembly system may determine that the syringe actuation component 700′ is fully inserted into the firing body 12b, and may terminate the insertion process. Alternatively or additionally, when a portion of the detected force profile is determined to substantially match a predefined force profile, the assembly system may determine that the syringe actuation component 700′ is approaching full insertion into the firing body 12b, and may decelerate the insertion process. A force profile may be generated in exemplary embodiments by detecting and plotting force values against incremental distances over which the syringe actuation component 700′ is made to travel during the assembly process.

In an exemplary embodiment, the forces may be detected at one or more load cells, for example, load cells manufactured by the Kistler Group. In an exemplary embodiment, the detected forces may be displayed on one or more visual display interfaces, for example, CoMo View® interfaces manufactured by the Kistler Group.

In an exemplary embodiment, the compression of the biasing mechanism 88 may be monitored during the assembly process.

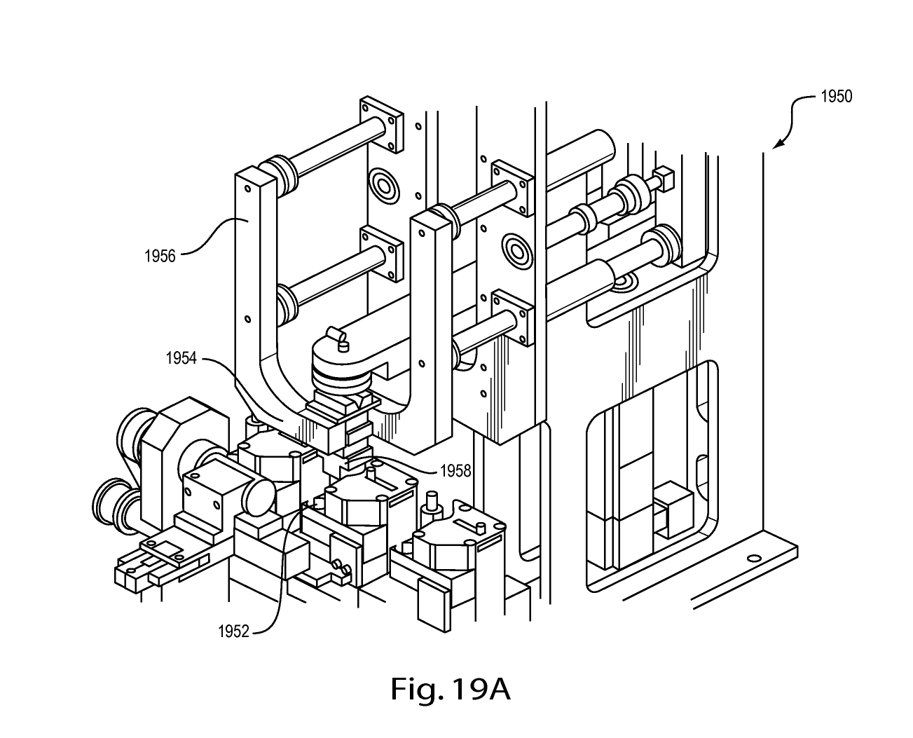

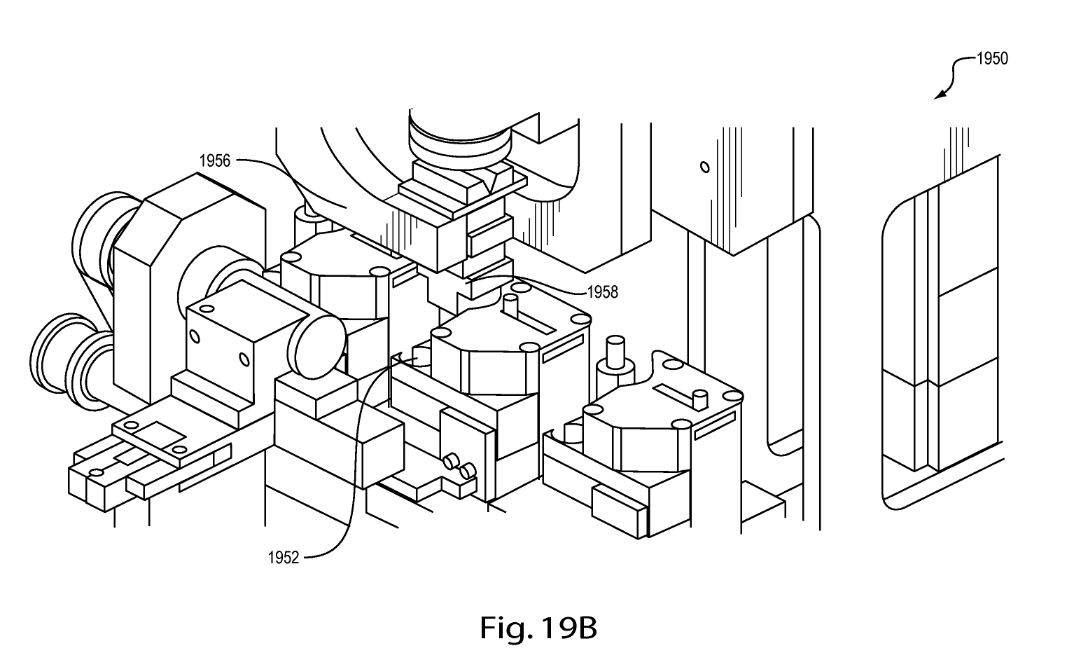

FIGS. 19A and 19B illustrate an exemplary perspective view of an assembly system 1950 that may be used to assemble an exemplary firing mechanism sub-assembly 122. FIG. 19B is a close-up view of the exemplary assembly system 1950 of FIG. 19A. An exemplary assembly system 1950 may be an assembly system produced by sortimat, an affiliate of ATS Automation.

The assembly system 1950 may include an assembly pallet 1952 for supporting and holding one or more components of the firing mechanism sub-assembly 122 in a vertical orientation during the assembly process. In an exemplary embodiment, the assembly pallet 1952 may be configured as a substantially cylindrical component with a central recessed portion for accommodating and supporting the bottom portions of one or more components. In an exemplary embodiment, the assembly pallet 1952 may support the distal portion of the firing body 12b.

The assembly system 1950 may include a gripping mechanism 1954 for supporting the side portion of one or more components of the firing mechanism sub-assembly 122 so that the components are held in a vertical orientation during the assembly process. In an exemplary embodiment, the gripping mechanism 1954 may be configured as a solid mechanism oriented horizontally and including a central bore for accommodating the components. In an exemplary embodiment, the gripping mechanism 1954 may support the side portions of the biasing mechanism 88 so that the biasing mechanism 88 is aligned in a vertical orientation during the assembly process. This minimizes wobbling of the biasing mechanism 88 during the assembly process and ensures proper alignment of the biasing mechanism 88 with the firing body 12b.

The assembly system 1950 may include a mechanical member 1956 with a terminal end configured as a press head 1958. The press head 1958 may be configured to contact and press downward on the proximal end of the syringe actuation component 700′ to couple the syringe actuation component 700′ with the firing body 12b. The press head 1958 may include or be associated with one or more force and/or pressure sensors, e.g., one or more piezoelectric load cells, for detecting and monitoring forces and/or pressures experienced during the assembly process. In an exemplary embodiment, the piezoelectric sensor includes a quartz crystal and two steel rings that generate an electrical charge when subjected to mechanical force or stress. The charge generated by the sensor may be directly proportional to the mechanical force applied to the sensor. In an exemplary embodiment, the force detected by the force sensor may be the frictional force with which the insertion of the syringe actuation component 700′ toward the firing body 12b is resisted during the assembly process. An exemplary force sensor may include, but is not limited to, a direct piezoelectric load cell manufactured by the Kistler Group.

The assembly system 1950 may include one or more motion generators (not pictured) that provide a motion for moving the mechanical member 1956. An exemplary motion generator may include, but is not limited to, a servomotor that drives the mechanical member 1956 in an upward or downward direction along the vertical axis. In an exemplary embodiment, the motion generator may be couplable to the mechanical member 1956 via a drive system (not pictured). In some embodiments, the drive system may be configured as a worm drive. The drive system may be coupled to the mechanical member 1956 via a flange or other coupler. In an exemplary embodiment, the drive system allows macro incremental movements of the press head 1958 along the vertical axis V on the order of about 1 mm. In an exemplary embodiment, the drive system allows micro incremental movements of the press head 1958 along the vertical axis V on the order of about 0.1 mm.

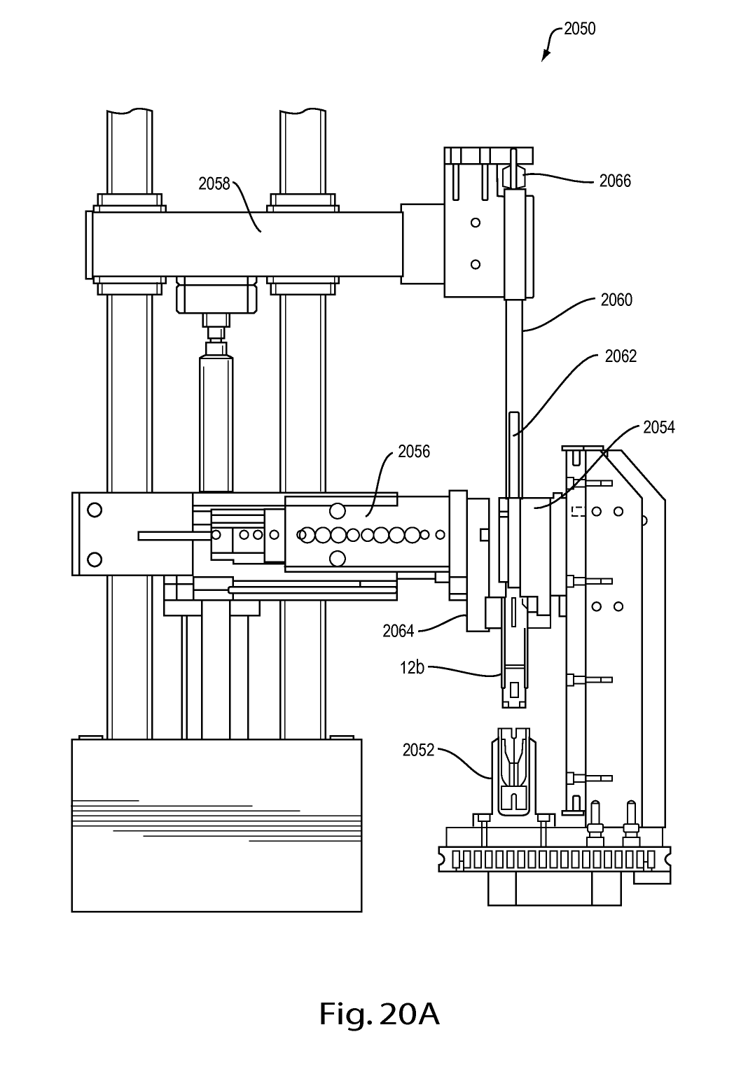

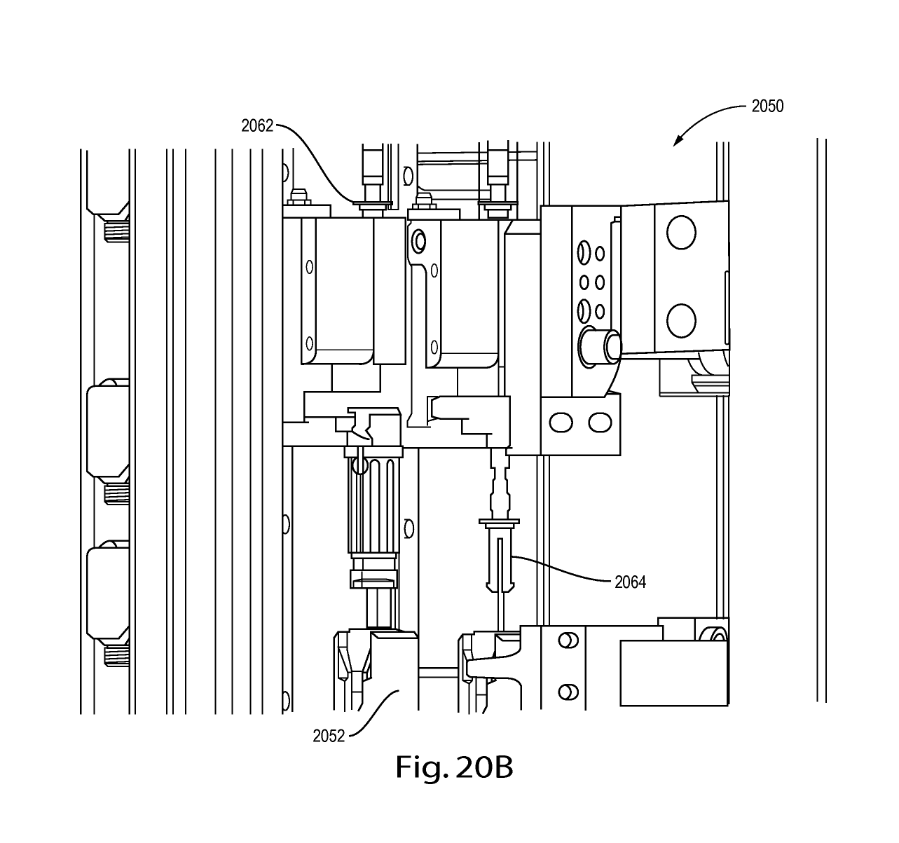

FIGS. 20A and 20B illustrate an exemplary perspective view of another assembly system 2050 that may be used to assemble an exemplary firing mechanism sub-assembly 122.

FIG. 20A is a side view and FIG. 20B is a front view of the exemplary assembly system 2050. An exemplary assembly system 2050 may be an assembly system produced by sortimat, an affiliate of ATS Automation.

The assembly system 2050 may include a cap holder 2052 configured for holding the distal cap 34 and the firing button 32 in a vertical orientation. The assembly system 2050 may include an assembly pallet 2054 aligned above the cap holder 2052 and configured for supporting the distal portion of the firing body 12b. The assembly system 2050 may include a first mechanical member 2056 coupled to the assembly pallet 2054 that is configured to slide the assembly pallet 2054 toward the cap holder 2052 in order to couple the firing body 12b to the firing button 32 and the distal cap 34.

The assembly system 2050 may include a second mechanical member 2058 with a terminal end configured as a press head 2060. The press head 2060 may be configured to contact and press downward on the proximal end of the syringe actuation component 700′ to couple the syringe actuation component 700′ with the firing body 12b. FIGS. 20A and 20B illustrate an initial position 2062 of the syringe actuation component 700′ before the start of the assembly process and a final position 2064 of the syringe actuation component 700′ after the assembly process in which the syringe actuation component 700′ is coupled to the firing body 12b.

The press head 2060 may include or be associated with one or more force and/or pressure sensors 2066, e.g., one or more piezoelectric load cells, for detecting and monitoring forces and/or pressures experienced during the assembly process. In an exemplary embodiment, the piezoelectric sensor includes a quartz crystal and two steel rings that generate an electrical charge when subjected to mechanical force or stress. The charge generated by the sensor may be directly proportional to the mechanical force applied to the sensor. In an exemplary embodiment, the force detected by the force sensor 2066 may be the frictional force with which the insertion of the syringe actuation component 700′ toward the firing body 12b is resisted during the assembly process. An exemplary force sensor 2066 may include, but is not limited to, a direct piezoelectric load cell manufactured by the Kistler Group.

The assembly system 2050 may include one or more motion generators (not pictured) that provide a motion for drive the first and second mechanical members 2056, 2058. An exemplary motion generator may include, but is not limited to, a servomotor that drives the mechanical members in an upward or downward direction along the vertical axis. In an exemplary embodiment, the motion generator may be couplable to the mechanical members via a drive system (not pictured). In some embodiments, the drive system may be configured as a worm drive. The drive system may be coupled to the mechanical members via a flange or other coupler. In an exemplary embodiment, the drive system allows macro incremental movements of the mechanical members along the vertical axis V on the order of about 1 mm. In an exemplary embodiment, the drive system allows micro incremental movements of the mechanical members along the vertical axis V on the order of about 0.1 mm.

Although the exemplary assembly system 2050 is described as inserting the syringe actuation component 700′ toward the firing body 12b, the same or a different assembly system may be used to hold the syringe actuation component 700′ in place while the firing body 12b is inserted toward the syringe actuation component 700′.

The assembly system 2050 may include a motion control computing device for controlling one or more control parameters for the motion generator. The motion control computing device may be provided integrally with the motion generator or separately from the motion generator. Exemplary control parameters of the motion generator controllable using the motion control computing device include, but are not limited to, starting/stopping of the motion generator, distance traveled, distance left to travel, speed, acceleration, deceleration, different phases of motion of the motion generator, etc. One or more control parameters may be set or altered by the motion control computing device based on one or more control factors including, but not limited to, a trigger instruction or signal generated when a particular force feature is detected in the force profile (e.g., the motion generator may be stopped when the trigger instruction or signal is received), the crossing of a predefined distance over which the syringe actuation component 700′ is inserted into the firing body 12b (e.g., the insertion speed of the syringe actuation component 700′ may be reduced after it is inserted a predefined distance into the firing body 12b), the lapse of a predefined period of time (e.g., the insertion speed of the syringe actuation component 700′ may be reduced after a predefined period of time has elapsed), etc.

In an exemplary embodiment, the assembly process may be divided into one or more phases with each phase having an associated set of control parameters, and the control parameters may be set and/or changed automatically by the motion control computing device based on the particular phase of the assembly process at a given time.

In an exemplary embodiment, the motion control computing device may be pre-programmed to control the motion generator in a desired manner during the assembly insertion process. The pre-programming of the motion control computing device may be overridden or altered by a user before or during the assembly process. In another exemplary embodiment, the motion control computing device may not be pre-programmed, and a user may use the motion control computing device to enter and control a programming of the motion generator before or during the assembly process.

The assembly system 2050 may include one or more trigger generation computing devices connectable to the force/pressure sensor 2066 for measuring the forces and/or pressures exerted during the assembly process and for measuring the displacement of the press head 2060 during the assembly process based on an output from the force/pressure sensor 2066. The trigger generation computing device may perform one or more functions including, but not limited to, measuring in real-time the forces detected by the force/pressure sensor 2066, measuring in real-time the pressures detected by the force/pressure sensor 2066, detecting in real-time that one or more trigger conditions are satisfied by the force profile, generating a trigger instruction or signal when one or more trigger conditions are met, sending the trigger instruction or signal to the motion control computing device to control the motion generator, and the like.

In an exemplary embodiment, the trigger generation computing device may be pre-programmed to recognize one or more characteristic force features in the force profile that, when generated, satisfy a trigger condition. The pre-programming of the trigger generation computing device may be overridden or altered by a user before or during the assembly process. In another exemplary embodiment, the trigger generation computing device may not be pre-programmed, and a user may set one or more characteristic force features that, when generated, satisfy a trigger condition before or during the assembly process.

FIG. 77 illustrates a block diagram of an exemplary computing device that may be used in exemplary embodiments as the motion control computing device and/or the trigger generation computing device. The exemplary computing device is described below in connection with FIG. 77.

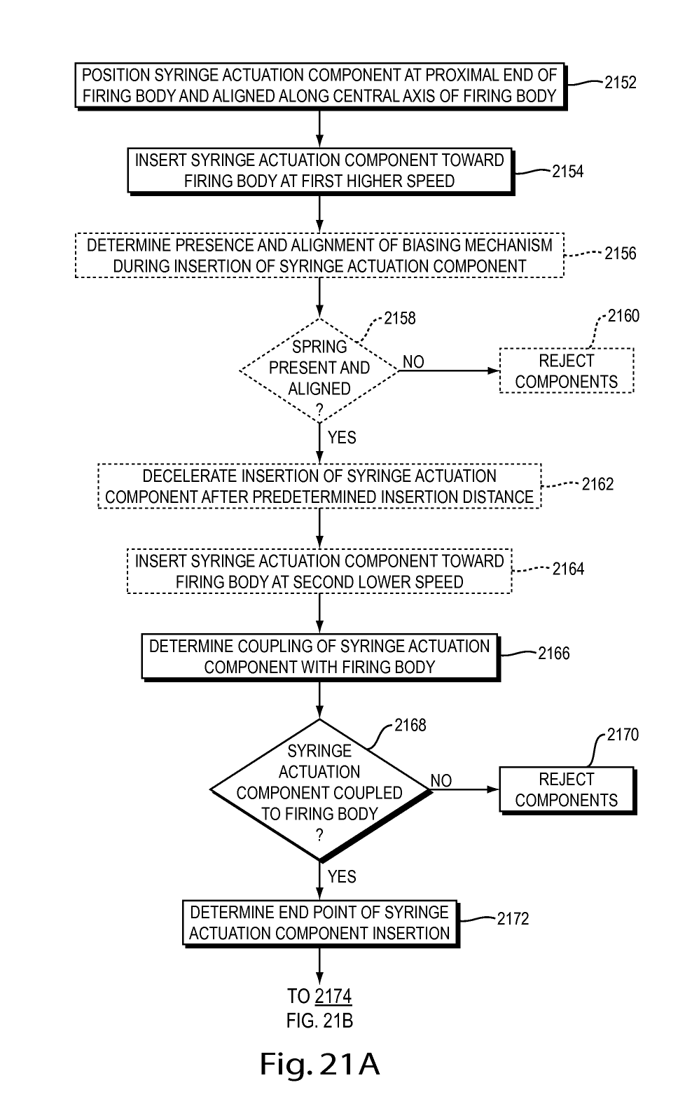

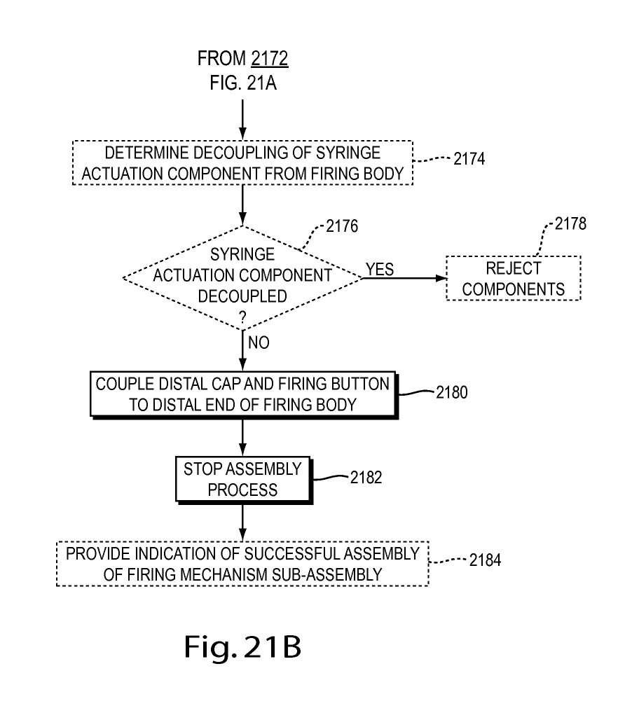

FIGS. 21A and 21B are flowcharts illustrating an exemplary method for assembling a firing mechanism sub-assembly 122 for use in an automatic injection device. Forces experienced at the press head may be detected and monitored during the assembly method.

In step 2152, the syringe actuation component 700′ may be positioned at the proximal end of the firing body 12b so that the central axis of the syringe actuation component 700′ is aligned along the central axis of the firing body 12b. In step 2154, the press head may insert the syringe actuation component 700′, at a first higher speed, toward the firing body 12b.

In step 2156, exemplary embodiments may determine whether the biasing mechanism 88 is present and correctly aligned between the firing body 12b and the syringe actuation component 700′. In one example, absence of the biasing mechanism 88 may cause the forces experienced at the press head to fall below one or more predetermined thresholds. In another example, if the biasing mechanism 88 is incorrectly assembled so that the mechanism 88 is squeezed outside the distal arms 788′ of the syringe actuation component 700′, the press head may experience higher forces than one or more predetermined thresholds. Exemplary embodiments may determine that the biasing mechanism 88 is absent if the forces experienced during compression of the biasing mechanism 88 are below one or more predetermined thresholds. Exemplary embodiments may determine that the biasing mechanism 88 is incorrectly assembled if the forces experienced during compression of the biasing mechanism 88 are above one or more predetermined thresholds. If it is determined in step 2158 that the biasing mechanism 88 is absent or incorrectly assembled, the assembly process may be terminated and the components discarded in step 2160. Otherwise, the method may progress to step 2162.

In step 2162, after the syringe actuation component 700′ has been inserted over a predetermined distance, it may be determined that the assembly process is approaching completion and the movement of the press head may be decelerated. In step 2164, the press head may insert the syringe actuation component 700′, at a second lower speed, within the bore of the firing body 12b.

In step 2166, exemplary embodiments may determine whether the syringe actuation component 700′ is properly and reliably coupled to the firing body 12b. Exemplary embodiments may determine that the syringe actuation component 700′ is correctly coupled to the firing body 12b if one or more forces experienced at the press head indicate a decreasing force profile within a predetermined insertion distance, indicating that the trigger anchoring portion 789′ of the syringe actuation component 700′ has snapped into place over the anchoring cap 12c of the firing body 12b. If exemplary embodiments determine in step 2168 that the syringe actuation component 700′ is improperly coupled to the firing body 12b, the assembly process may be terminated and the components discarded in step 2170. Otherwise, the method may progress to step 2172.

In step 2172, exemplary embodiments may determine the end point of the insertion of the syringe actuation component 700′ into the firing body, i.e., the point at which farther insertion may be stopped. When this end point is reached, the press head may be instructed to stop movement of the syringe actuation component 700′ toward the firing body 12b and to move away from the sub-assembly.

In step 2174, as the press head moves away from the sub-assembly, exemplary embodiments may determine whether the syringe actuation component 700′ becomes decoupled from the firing body 12b, which indicates failed assembly of the firing mechanism sub-assembly. If the syringe actuation component 700′ is securely coupled to the firing body 12b, the forces experienced at the press head are lower because there is no component pressing against the press head as the press head returns to its original position. In this case, exemplary embodiments may determine that the syringe actuation component 700′ is securely coupled to the firing body 12b if the detected forces fall within an acceptable low range. On the other hand, if the syringe actuation component 700′ becomes decoupled from the firing body 12b, the forces experienced at the press head are higher because, as the syringe actuation component 700′ moves away from the firing body 12b (under action of the biasing mechanism 88), it presses against the press head as the press head returns to its original position. In this case, in step 2176, exemplary embodiments may determine that the syringe actuation component 700′ has decoupled from the firing body 12b if the detected forces are higher than an acceptable low range. In the case of a decoupling, exemplary embodiments may discard the components of the sub-assembly in step 2178.

In step 2180, the firing button 32 may be positioned between the distal cap 34 and the firing body 12b. The distal cap 34 may be pressed toward the firing body 12b to couple the distal cap 34 and the firing button 32 to the firing body 12b in one motion.

In step 2182, exemplary embodiments may end the assembly process. In step 2184, the assembly system may provide a visual and/or auditory indication that the firing mechanism sub-assembly 122 has been successfully and correctly assembled. The firing mechanism sub-assembly 122 may subsequently be used to form an automatic injection device.

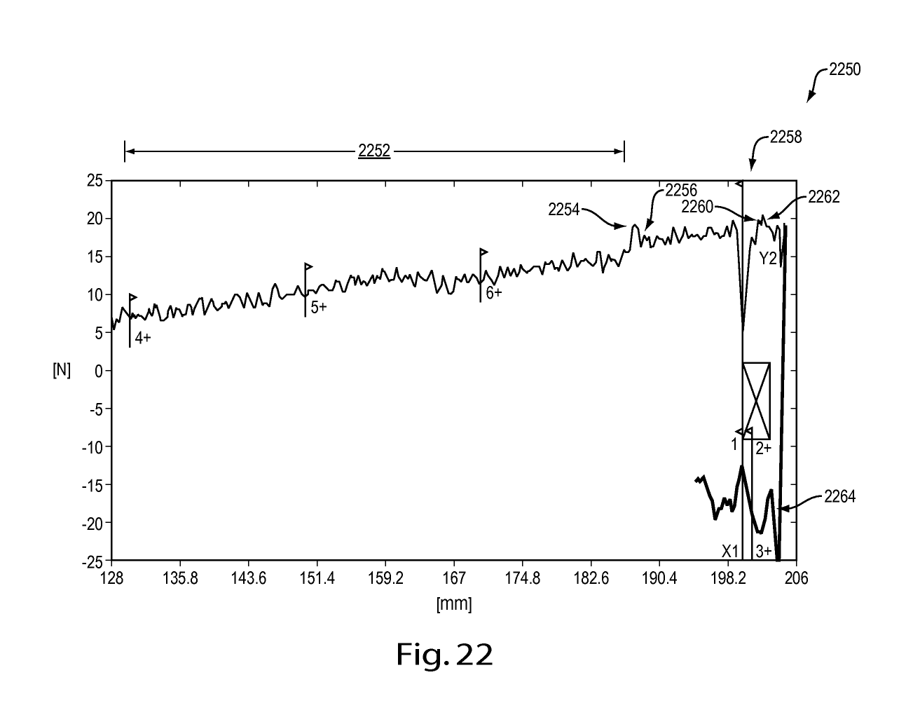

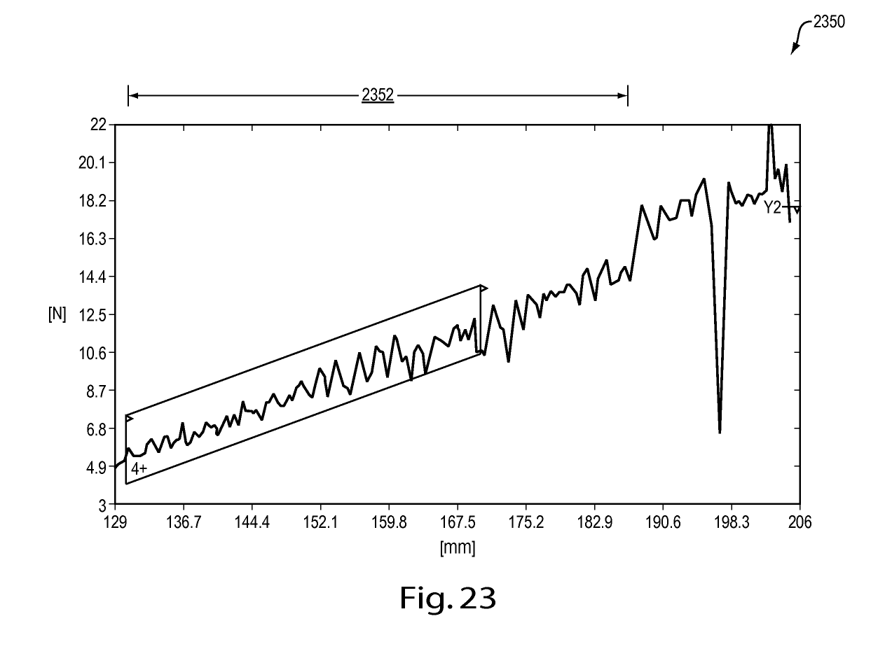

FIGS. 22 and 23 illustrate exemplary force profiles 2250, 2350 of the forces experienced at the press head during assembly of the firing mechanism sub-assembly 122. The y-axis of the force profile denotes the frictional forces (in N) detected by an exemplary force sensor at the press head. The x-axis of the force profile denotes the distance (in mm) moved by the press head toward the firing body 12b (indicated on the profile as moving from left to right) and/or away from the firing body 12b (indicated on the profile as moving from right to left).

A first portion 2252 of the force profile shows gradually increasing forces experienced when the biasing mechanism 88 is compressed by the movement of the syringe actuation component 700′ toward the firing body 12b. Exemplary forces in the first portion 2252 may range from about 4.8 N to about 14.5 N over an insertion distance of about 129 mm to about 198 mm in some exemplary embodiments. The oscillations or random spikes at the first portion 2252 of the force profile are a result of the buckling and compression of the biasing mechanism 88.

In the first portion 2252 of the force profile, exemplary embodiments may determine whether the syringe actuation component 700′ is properly and reliably coupled to the firing body 12b. FIGS. 22 and 23 illustrate two exemplary methods of making this determination. In the exemplary method of FIG. 22, if the forces experienced at the press head fall within predefined force ranges at one or more discrete points during insertion of the syringe actuation component 700′, exemplary embodiments may determine that the syringe actuation component 700′ is correctly and reliably coupled to the firing body 12b. A first determination made at an insertion distance of about 130 mm may be used to determine that the spring is present and correctly aligned if the forces at that distance range from about 3 N and about 9 N as the force trace travels from left to right. A second determination made at an insertion distance of about 150 mm may be used to determine that the spring is present and correctly aligned if the forces at that distance range from about 6 N and about 12 N as the force trace travels from left to right. A third determination made at an insertion distance of about 170 mm may be used to determine that the spring is present and correctly aligned if the forces at that distance range from about 9 N and about 15 N as the force trace travels from left to right. If the forces do not fall within the acceptable ranges at any of the first, second and third determinations, the sub-assembly is considered a reject, the press head is instructed to return to its original position, the assembly process is terminated, and the components of the assembly may be discarded. The first, second and third determinations are spaced out along the first portion 2252 of the force profile to best capture the progressive compression of the biasing mechanism 88. One of ordinary skill in the art will recognize that the first, second and third determinations may be performed at any suitable points in the force profile, and that more or fewer determinations may be made.

In the exemplary method of FIG. 23, if the forces in the first portion 2352 of the profile 2350 fall within an acceptable rectangular range, exemplary embodiments may determine that the syringe actuation component 700′ is correctly and reliably coupled to the firing body 12b. The left-hand side of the rectangle extends between about 130 mm, 4 N and about 130 mm, 7 N in which the force trace travels from left to right; the right-hand side of the rectangle extends between about 170 mm, 10.5 N and about 170 mm, 14 N in which the force trace travels from left to right; the top side of the rectangle extends between about 130 mm, 7 N and about 170 mm, 14 N; and the bottom side of the rectangle extends between about 130 mm, 4 N and about 170 mm, 10.5 N. If the forces experienced at the press head are lower than the rectangular range, this may indicate absence of the biasing mechanism 88 because presence and compression of the biasing mechanism 88 would result in higher forces at the first portion 2352 of the force profile. On the other hand, if the forces experienced at the press head are higher than the rectangular range, this may indicate that the biasing mechanism 88 is incorrectly aligned with respect to the syringe actuation component 700′ and the firing body 12b. In either case, the press head may be instructed to return to its original position, the assembly process may be terminated, and the components of the assembly may be discarded.

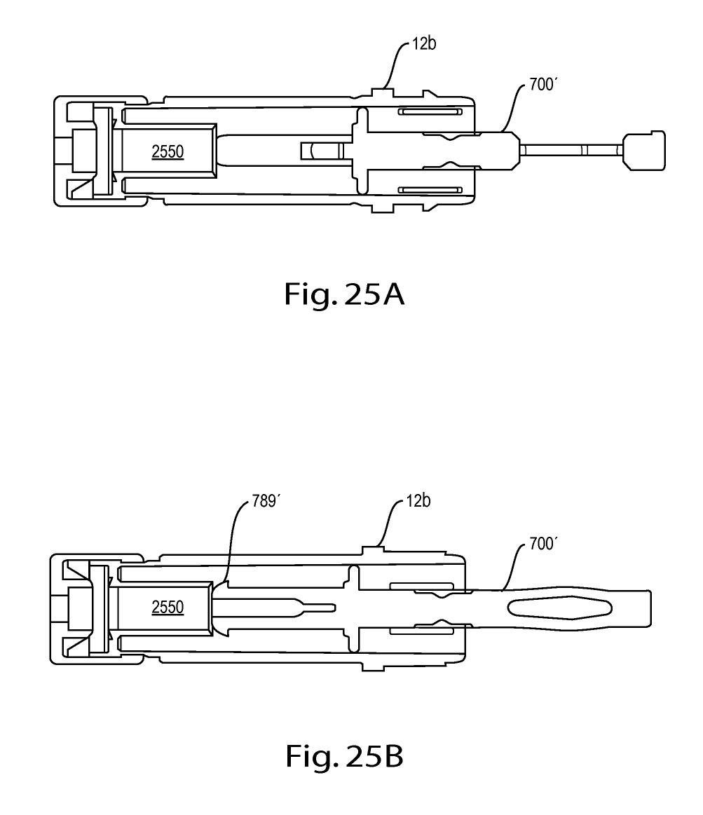

Referring to FIG. 22, a second portion 2254 of the force profile shows a small but rapid increase in the forces experienced at the press head caused when the trigger anchoring portion 789′ of the syringe actuation component 700′ impinges upon and resists a narrowed or necked region within the hollow bore of the firing button 12b. Exemplary forces in the second portion 2254 of the force profile may increase from about 14.4 N to about 18.2 N over an insertion distance of about 186 mm to about 188 mm in some exemplary embodiments. As illustrated in FIGS. 25A and 25B, an exemplary embodiment, the narrowed or necked portion may be formed by an inner cylindrical tube 2550 formed within the hollow bore of the firing body 12b. The inner cylindrical tube 2550 may have a narrower inner diameter than the inner diameter of the firing body 12b. FIG. 25A is a schematic view of a first assembly state during assembly of the firing mechanism sub-assembly 122, in which the trigger anchoring portion 789′ of the syringe actuation component 700′ impinges upon and resists the inner cylindrical tube 2550 within the firing body 12b. FIG. 25B is a schematic view of the first assembly state of FIG. 25A rotated by about 90 degrees from the view of FIG. 25A.

Still referring to FIG. 22, a third portion 2256 of the force profile shows a small but rapid decrease in the force experienced at the press head caused when the tabbed feet 7891′ of the trigger anchoring portion 789′ are squeezed inwardly to fit within the inner cylindrical tube 2550 of the firing body 12b. Exemplary forces in the third portion 2256 of the force profile may decrease from about 18.2 N to about 16.3 N over an insertion distance of about 188 mm to about 190 mm in some exemplary embodiments.

The movement of the press head toward the syringe carrier may be decelerated after the press head has been inserted over a predetermined distance, for example, about 195 mm. Exemplary embodiments may detect that the press head has traveled the predetermined distance and, in response, may instruct the press head to decelerate. A fourth portion 2258 of the force profile shows a rapid decrease in the forces experienced when the speed of the press head is reduced upon the press head traveling a predetermined distance. Exemplary forces in the fourth portion 2258 may drop from about 19 N to about 6.8 N over an exemplary insertion range of about 195 mm to about 196 mm in some exemplary embodiments.

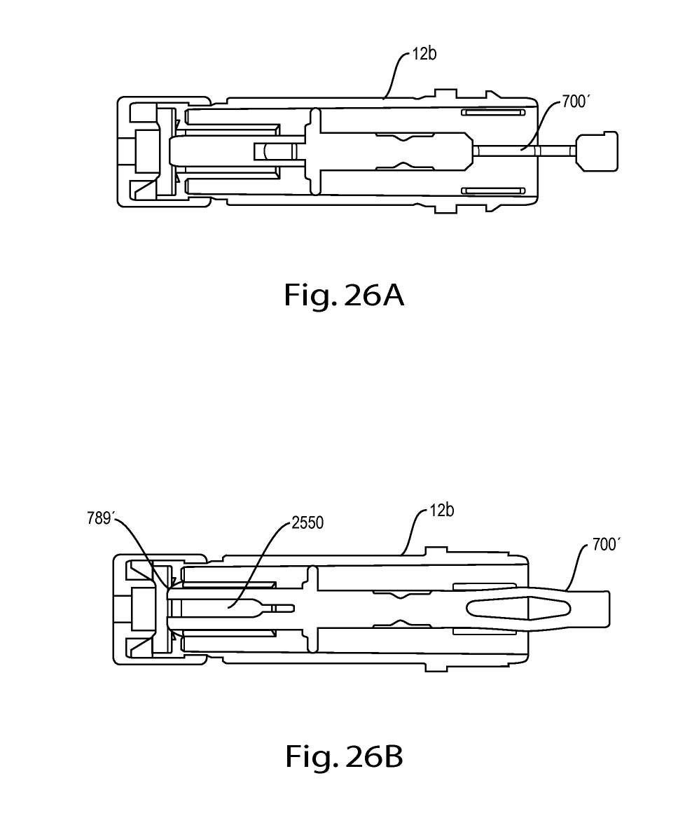

A fifth portion 2260 of the force profile shows a rapid increase in the force experienced at the press head caused when the tabbed feet 7891′ of the trigger anchoring portion 789′ are squeezed at the distal end of the inner cylindrical tube 2550 of the firing body 12b. Exemplary forces in the fifth portion 2260 may range from about 18.5 N to about 22 N over an insertion distance of about 202 mm to about 204 mm in some exemplary embodiments. FIG. 26A is a schematic view of a second assembly state during assembly a firing mechanism sub-assembly 122, in which the tabbed feet 7891′ of the trigger anchoring portion 789′ passes through the distal end of the inner cylindrical tube 2550 of the firing body 12b. FIG. 26B is a schematic view of the second assembly state of FIG. 26A rotated by about 90 degrees from the view of FIG. 26A.

A sixth portion 2262 of the force profile shows a rapid decrease in the forces experienced at the press head caused when the tabbed feet 7891′ of the trigger anchoring portion 789′ snap over the distal end of the inner cylindrical tube 2550 and rest on top of the distal end of the firing body 12d. Exemplary forces in the sixth portion 2262 may range from about 22 N to about 17 N over an insertion distance of about 204 mm to about 205 mm in some exemplary embodiments.

Exemplary embodiments may determine whether one or more force values detected during the assembly process match a trigger force condition, indicating that the end point of the insertion of the syringe actuation component 700′ has been reached. The trigger condition may be set to be one or more force values that appear on the sixth portion of the force profile. In the exemplary embodiment illustrated in FIG. 22, the trigger force may be set to about 15 N to about 17 N, and the x-axis range over which the trigger force is detected or measured may be set to be between about 204 mm and about 205 mm. In the exemplary embodiment illustrated in FIG. 23, the trigger force may be set to about 18 N, and the x-axis range over which the trigger force is detected or measured may be set to be between about 204 mm and about 206 mm. The approach is indicated to be “from above,” which indicates that the trigger condition is satisfied if the force is at about 18 N within an x-axis range of between 204 mm and about 206 mm. If the trigger condition is satisfied in the force profiles of FIGS. 22 and 23, exemplary embodiments may instruct the press head to return to its original position as the assembly process has been completed.

Exemplary embodiments may continue to detect forces experienced at the press head as the press head returns to its original position in order to test whether the syringe actuation component 700′ is decoupled from the firing body 12b. A seventh portion 2264 of the force profile running from the right to the left of the x-axis shows the forces experienced during the return of the press head. Exemplary embodiments may determine that the syringe actuation component 700′ has become decoupled from the firing body 12b if the detected forces fall between about −9 N to about 1 N over an insertion distance range of about 200 mm to about 203 mm in some exemplary embodiments, as the force profile runs from the right to the left. In one example, forces may be detected over an insertion distance range of about 200 mm to about 201 mm in some exemplary embodiments, as the force profile runs from the right to the left. The detected forces may then be compared to determine if they fall within the proscribed range of between about −9 N to about 1 N over an insertion distance range of about 200 mm to about 203 mm.

If the detected forces in the seventh portion 2264 of the force profile fall within the above-mentioned proscribed range, this indicates that the syringe actuation component 700′ has decoupled from the firing body 12d and is pressing against the press head to give rise to higher-than-normal forces. In this case, the components of the firing mechanism sub-assembly 122 may be discarded. Otherwise, if the detected forces in the seventh portion 2264 of the force profile are lower than the proscribed range, this indicates that the syringe actuation component 700′ is reliably secured to the firing body 12b. The firing mechanism sub-assembly 122 is then ready for assembly with other components to form an automatic injection device.

One of ordinary skill in the art will recognize that exemplary force profiles 2250 and 2350 may have fewer or additional features corresponding to interactions among the components. One of ordinary skill in the art will recognize that the force and insertion range values used in the exemplary method of FIGS. 22 and 23 are exemplary, and that any suitable force and insertion range values may be used to determine when the assembly process should be stopped and to determine whether the components are assembled correctly.

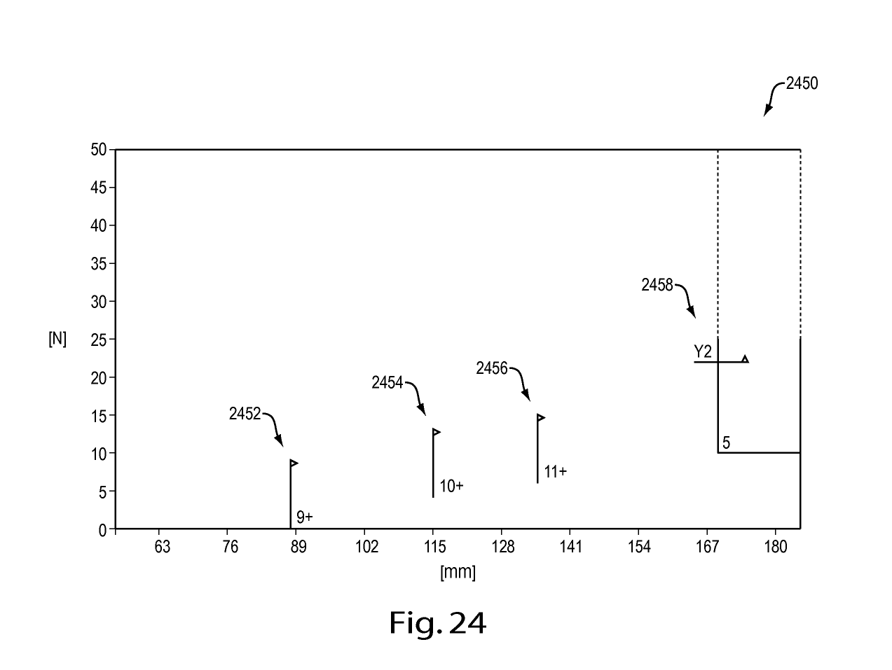

FIG. 24 illustrates a graph of exemplary force detections performed during assembly of the firing mechanism sub-assembly 122. The y-axis of the force profile denotes the frictional forces (in N) detected by an exemplary force sensor at the press head. The x-axis of the force profile denotes the distance (in mm) moved by the press head toward the firing body 12b (indicated on the profile as moving from left to right) and/or away from the firing body 12b (indicated on the profile as moving from right to left).

In the exemplary method of FIG. 24, if the forces experienced by the press head fall an exemplary range at one or more discrete points during insertion of the syringe actuation component 700′, exemplary embodiments may determine that the syringe actuation component 700′ is correctly and reliably coupled to the firing body 12b. A first determination 2452 made at an insertion distance of about 88 mm may be used to determine that the spring is present and correctly aligned if the forces at that distance range from about 0 N and about 9 N as the force trace travels from left to right. A second determination 2454 made at an insertion distance of about 115 mm may be used to determine that the spring is present and correctly aligned if the forces at that distance range from about 4 N and about 13 N as the force trace travels from left to right. A third determination 2456 made at an insertion distance of about 132 mm may be used to determine that the spring is present and correctly aligned if the forces at that distance range from about 7 N and about 15 N as the force trace travels from left to right.

If the forces do not fall within the acceptable ranges at any of the first, second and third determinations, the sub-assembly is considered a reject, the press head is instructed to return to its original position, the assembly process is terminated, and the components of the assembly may be discarded. The first, second and third determinations are spaced out to best capture the progressive compression of the biasing mechanism 88. One of ordinary skill in the art will recognize that the first, second and third determinations may be performed at any suitable points in the force profile, and that more or fewer determinations may be made.

Exemplary embodiments may determine whether the syringe actuation component 700′ has been coupled to the firing body 12b by determining whether the detected forces match a specified range of about 10 N to about 26 N over an insertion distance range of about 169 mm to about 185 mm in some exemplary embodiments. If the detected forces falls within the specific range, this indicates that the firing mechanism sub-assembly 122 has been correctly assembled. Otherwise, it is determined that the firing mechanism sub-assembly 122 is incorrectly assembled, and the components of the sub-assembly are discarded.

Exemplary embodiments may determine whether one or more force values detected during the assembly process match a trigger condition 2458, indicating that the end point of the insertion of the syringe actuation component 700′ has been reached. In the exemplary embodiment illustrated in FIG. 24, the trigger force may be set to about 22 N, and the x-axis range over which the trigger force is detected or measured may be set to be between about 165 mm and about 178 mm. The approach is indicated to be “from below,” which indicates that the trigger condition is satisfied if the force is at about 18 N within an x-axis range of between 165 mm and about 178 mm. If the trigger condition is satisfied in the force profiles of FIG. 24, exemplary embodiments may instruct the press head to return to its original position as the assembly process has been completed.

Although an exemplary assembly of a firing mechanism sub-assembly is described with reference to inserting the syringe actuation component 700′ toward the firing body 12b, one of ordinary skill in the art will appreciate that exemplary embodiments may also be used to insert the firing body 12b toward the syringe actuation component 700′, and/or to insert the syringe actuation component 700′ and the firing body 12b toward each other in order to assemble the firing mechanism sub-assembly.

### V. EXEMPLARY ASSEMBLY OF A SYRINGE INTO A HOUSING OF AN AUTOMATIC INJECTION DEVICE

In an exemplary method of assembling an automatic injection device, a syringe assembly may be assembled with a housing assembly in a controlled automated manner. The housing assembly may include a housing of the device fitted with a proximal cap for covering an injection needle. The syringe assembly may include a syringe housing sub-assembly coupled to a syringe and a firing mechanism sub-assembly. The proximal end of the syringe may be coupled to an injection needle that is covered by a rigid needle shield and, optionally, a soft needle shield. During assembly, the syringe assembly is moved toward the housing assembly and/or the housing assembly is moved toward the syringe assembly, such that the rigid needle shield is inserted to an appropriate insertion depth into the proximal cap.

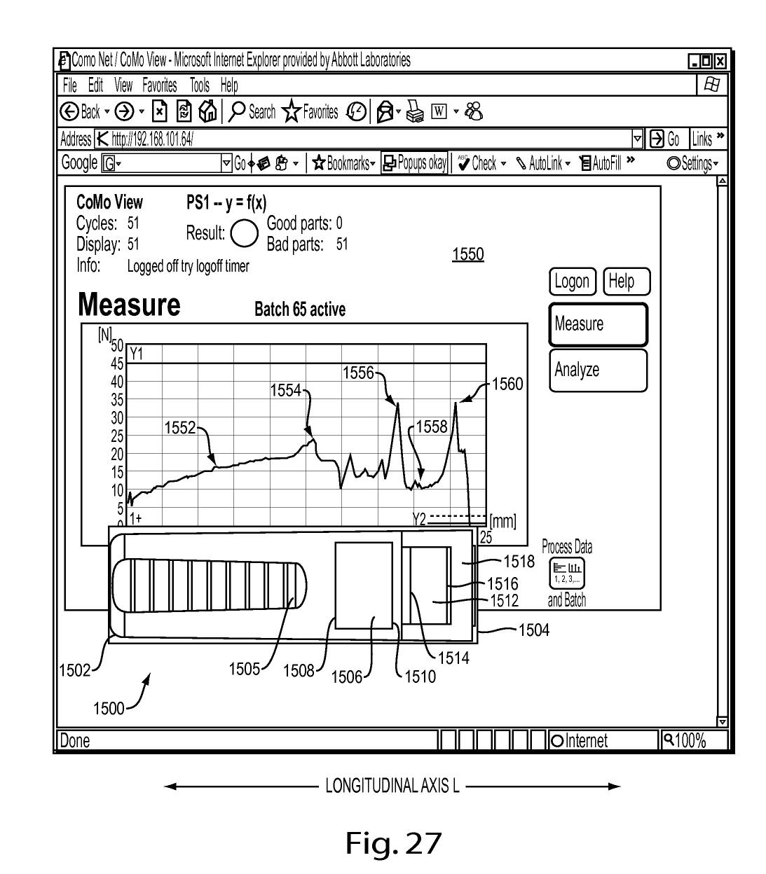

FIG. 27 illustrates a perspective view of an exemplary rigid needle shield 1500 and a characteristic force profile graph 1550 associated with the insertion of the rigid needle shield 1500 into a proximal cap, in which the distal end 1504 of the rigid needle shield 1500 is disposed exactly or approximately at a local friction point in the needle. The characteristic force profile graph 1550 is associated with the insertion of the rigid needle shield 1500 into the proximal cap. In graph 1550, the y-axis indicates the forces exerted by the friction point during syringe insertion (in N) and the x-axis indicates the displacement of the proximal end 1502 of the rigid needle shield 1500 past the friction point in the proximal cap. The force profile graph 1550 traces the frictional forces exerted by the friction point in the proximal cap against the different structural or ornamental features on the outer surface of the rigid needle shield 1500 as the rigid needle shield 1500 is inserted past the friction point toward the proximal end of the proximal cap.

Exemplary rigid needle shields usable in exemplary embodiments are not limited to the rigid needle shield 1500 illustrated in FIG. 27, and may be configured in other suitable sizes, shapes and configurations. For example, other exemplary rigid needle shields may include more or fewer structural or ornamental features than those illustrated in FIG. 27. One of ordinary skill in the art will appreciate that the characteristic force profile will vary based on the particular size, shape and configuration of the associated rigid needle shield and the particular size, shape and configuration of the associated proximal cap.

The outer surface of the rigid needle shield 1500 may include a feature 1505 that projects from the outer surface. The length of the rigid needle shield 1500 extending between the proximal end 1502 of the rigid needle shield 1500 and the proximal end 1508 of the feature 1505 may be associated with relatively low but rising forces 1552 in the force profile 1550 that are generated as the length of the rigid needle shield passes by the friction point in the proximal cap. The feature 1505 may be associated with a “first characteristic peak” 1554 in the force profile 1550 that is generated as the feature 1505 passes by the friction point in the proximal cap. One of ordinary skill in the art will recognize that one or more intermediate peaks may appear on the force profile between the start of the force profile and the first characteristic peak 1554.

The outer surface of the rigid needle shield 1500 may include a feature 1506 that projects from the outer surface, e.g., a logo of the manufacturer of the rigid needle shield 1500 (“BD Logo” shown in FIG. 27). The feature 1506 may extend substantially along the longitudinal axis L between a proximal end 1508 and a distal end 1510. In an exemplary embodiment, the feature 1506 may have a length of about 6 mm, and may extend along the longitudinal axis L from about 12 mm from the proximal end 1502 of the rigid needle shield 1500 to about 18 mm from the proximal end 1502 of the rigid needle shield 1500. A ridge at or near the distal end 1510 of the feature 1506 may be associated with a “second characteristic peak” 1556 in the force profile 1550 that is generated as the ridge passes by the friction point in the proximal cap. One of ordinary skill in the art will recognize that one or more intermediate peaks may appear on the force profile between the first characteristic peak 1554 and the second characteristic peak 1556.

In an exemplary embodiment, when the syringe is near the desired insertion depth in the housing of the automatic injection device, the ridge at or near the distal end 1510 of the feature 1506 passes by the friction point in the proximal cap. In this exemplary embodiment, the appearance in the force profile 1550 of the second characteristic peak 1556, within an x-axis range that corresponds to the location of the distal end 1510 of the feature 1506, may indicate that the syringe insertion process is not complete but is near completion. The appearance of the second characteristic peak 1556 may be used to slow down, stop or otherwise control the motion generator driving the syringe into the housing of the automatic injection device.

In an exemplary embodiment, the detection of all or part of the second characteristic peak 1556 may be used to drive the motion generator so that the syringe is inserted a farther predetermined distance into the housing after detection of the second characteristic peak. In an exemplary embodiment, in the final desired configuration, the distal end 1504 of the rigid needle shield 1500 sits at or near the friction point in the proximal cap. In this embodiment, the farther predetermined distance may be set to be the distance between the ridge at or near the distal end 1510 of the feature 1506 and the distal end 1504 of the rigid needle shield 1500 in order to achieve the desired final configuration.

The outer surface of the rigid needle shield 1500 may include a feature 1512 that creates a depression in the outer surface, e.g., a window that securely mates the rigid needle shield 1500 to a soft rigid needle shield housed within the rigid needle shield (“window” shown in FIG. 27). The feature 1512 may extend substantially along the longitudinal axis L from a proximal end 1514 to a distal end 1516. In an exemplary embodiment, the feature 1512 may have a length of about 3 mm, and may extend along the longitudinal axis L from about 20 mm from the proximal end 1502 of the rigid needle shield 1500 to about 23 mm from the proximal end 1502 of the rigid needle shield 1500. The feature 1512 may be associated with a “first characteristic trough” 1558 in the force profile 1550 that is generated as the feature 1512 passes by the friction point in the proximal cap. One of ordinary skill in the art will recognize that one or more intermediate troughs or depressions may appear on the force profile between the start of the force profile and the first characteristic trough 1558.

In an exemplary embodiment, when the syringe is near the desired insertion depth, the feature 1512 passes by the friction point in the proximal cap. In this exemplary embodiment, the appearance in the force profile 1550 of the first characteristic trough 1558, within an x-axis range that corresponds to the location of the feature 1512, may indicate that the syringe insertion process is not complete but is near completion. The appearance of the first characteristic trough 1558 may be used to slow down, stop or otherwise control the motion generator driving the syringe into the housing of the automatic injection device.

In an exemplary embodiment, the detection of all or part of the first characteristic trough 1558 may be used to drive the motion generator so that the syringe is inserted a farther predetermined distance into the housing. In an exemplary embodiment, in the final desired configuration, the distal end 1504 of the rigid needle shield 1500 sits at the friction point in the proximal cap. In this exemplary embodiment, the farther predetermined distance may be set to be approximately the distance between the feature 1512 and the distal end 1504 of the rigid needle shield 1500 in order to achieve the desired final configuration.

The outer surface of the rigid needle shield 1500 may include a projecting portion 1518 at or adjacent to its distal end 1504. The distal end 1504 of the rigid needle shield 1500 may thus may be associated with a “third characteristic peak” 1560 in the force profile 1550 that is generated as the distal end 1504 passes by the friction point in the proximal cap. One of ordinary skill in the art will recognize that one or more intermediate peaks may appear on the force profile between the second characteristic peak 1556 and the third characteristic peak 1560.

In an exemplary embodiment, when the syringe is at or near the desired insertion depth, the distal end 1504 of the rigid needle shield 1500 passes by the friction point in the proximal cap. In this exemplary embodiment, the appearance in the force profile 1550 of the third characteristic peak 1560, within an x-axis range that corresponds to the location of the distal end 1504 of the rigid needle shield 1500, may indicate that the syringe insertion process is complete or close to completion. The appearance of the third characteristic peak 1560 may be used to slow down, stop or otherwise control the motion generator driving the syringe into the housing of the automatic injection device.

In an exemplary embodiment, in the final desired configuration, the distal end 1504 of the rigid needle shield 1500 sits at the friction point in the proximal cap. In this exemplary embodiment, the motion generator may be stopped immediately upon detection of all or part of the third characteristic peak 1560 since the third characteristic peak indicates that the final desired configuration has been achieved.

During the syringe insertion process, exemplary embodiments may use the detection or measurement of one or more characteristic force features in the force profile to determine the point at which the syringe insertion process is completed or near completion. Exemplary embodiments may therefore use detection of the one or more characteristics force features to control the syringe insertion process. Exemplary force features usable in exemplary embodiments include, but are not limited to, part or the entirety of the first characteristic peak, part or the entirety of the second characteristic peak, part or the entirety of the first characteristic trough, part or the entirety of the third characteristic peak, part or the entirety of the fourth characteristic peak, a combination of two or more of the above-mentioned force features, etc.

Exemplary embodiments may use any suitable technique to detect any of the characteristic force features in a force profile. In an exemplary embodiment, the forces generated during the syringe insertion process may be detected and analyzed to determine if they satisfy a trigger condition. The trigger condition may specify one or more predefined trigger force values and one or more predefined trigger hysteresis values. If the forces generated during the syringe insertion process satisfy the trigger condition, then a trigger instruction or signal may be generated to control an aspect of the syringe insertion process.

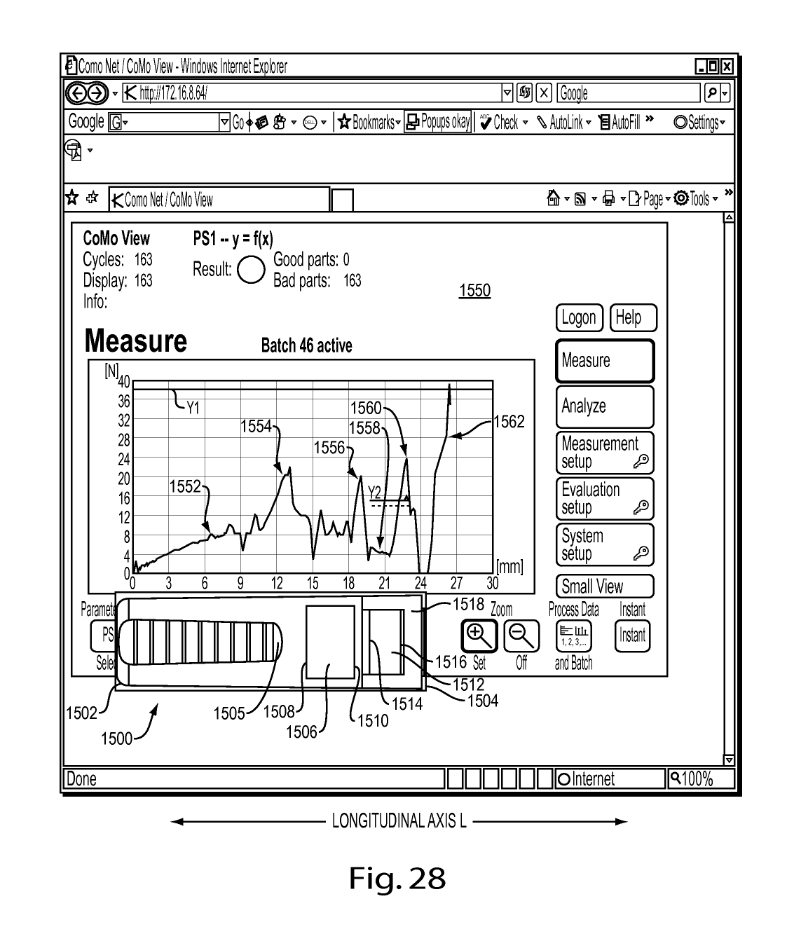

FIG. 28 illustrates a perspective view of an exemplary rigid needle shield 1500 and a characteristic force profile graph 1550 associated with the insertion of the rigid needle shield 1500 in which the distal end 1504 of the rigid needle shield 1500 is disposed beyond a local friction point in the proximal cap toward the proximal end of the proximal cap.

In the exemplary embodiment shown in FIG. 28, in the assembled configuration of the rigid needle shield in the proximal cap, the distal end 1504 of the rigid needle shield 1500 may have traveled beyond the friction point in the proximal cap toward the proximal end of the proximal cap. During the syringe insertion process, after the entire length of the rigid needle shield 1500 has passed the friction point in the proximal cap, the syringe body (not shown) begins to pass by the friction point in the proximal cap. The syringe body may be associated with a “fourth characteristic peak” 1562 in the force profile 1550 that is generated as the syringe body begins to pass by the friction point in the proximal cap. One of ordinary skill in the art will recognize that one or more intermediate peaks may appear on the force profile between the third characteristic peak 1560 and the fourth characteristic peak 1562.

In an exemplary embodiment, insertion of the rigid needle shield beyond the friction point in the proximal cap toward the proximal end of the proximal cap (as shown in FIG. 28) is undesirable. In this case, detection of all or part of the fourth characteristic peak 1562 may be used to determine that the syringe insertion process has failed and that the syringe has been inserted too far into the housing. In an exemplary embodiment, upon detection of the failure, the syringe and housing assembly may be discarded. In another exemplary embodiment, upon detection of the failure, the insertion process may be restarted or adjusted to achieve the desired insertion depth of the syringe in the housing.

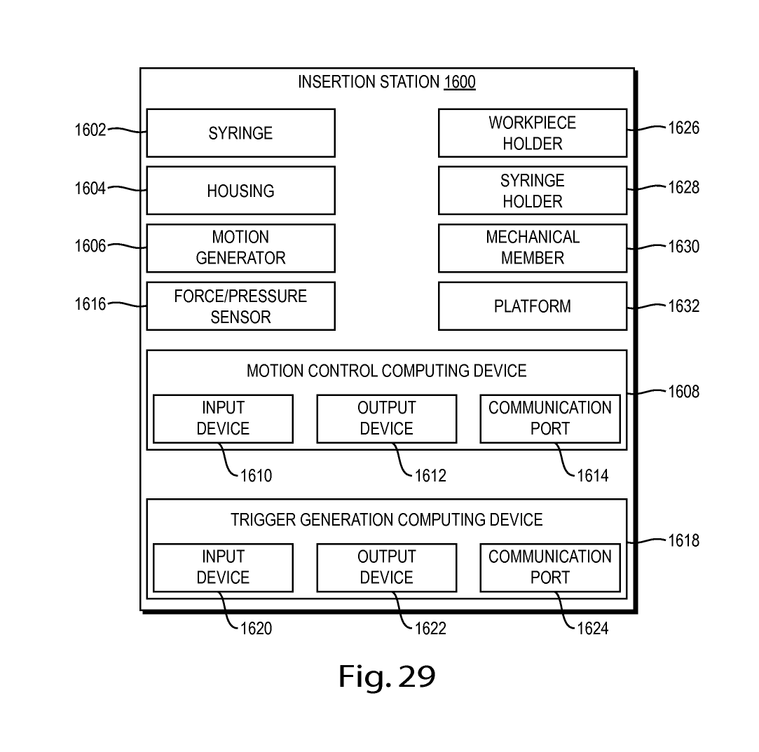

FIG. 29 is a block diagram illustrating an exemplary syringe insertion system 1600 that may be used in exemplary embodiments to assemble an automatic injection device by inserting a syringe 1602 into the housing 1604 of the automatic injection device. The insertion system 1600 includes one or more motion generators 1606 for driving the syringe 1602 toward the proximal end of the housing 1604 and/or in some embodiments for driving the housing 1604 toward the distal end of the syringe 1602 via the motion of one or more mechanical members 1630.

The insertion system 1600 may include a workpiece holder 1626 for releasably securing the housing 1604 and a syringe holder 1628 for releasably securing the syringe 1602 during the syringe insertion process. The insertion system 1600 may include a platform 1632 for supporting one or more components of the insertion system 1600, e.g., the workpiece holder 1626, the syringe holder 1628, the motion generator 1606, the mechanical members 1630, etc.

The insertion system 1600 may include one or more motion control computing devices 1608 for controlling one or more control parameters of the motion generator 1606. The motion control computing device 1608 may be provided integrally with the motion generator 1606 or separately from the motion generator 1606. Exemplary control parameters of the motion generator 1606 controllable using the motion control computing device 1608 include, but are not limited to, times of activation/deactivation of the motion generator, distance traveled, distance to travel, speed, acceleration, deceleration, different phases of motion of the motion generator, etc. One or more control parameters may be set or altered by the motion control computing device 1608 based on one or more control factors including, but not limited to, a trigger instruction or signal generated when a particular force feature is detected in the force profile (e.g., the motion generator may be stopped when the trigger instruction or signal is received), the crossing of a predefined distance over which the syringe is inserted into the housing (e.g., the insertion speed may be reduced after the syringe is inserted a predefined distance into the housing), the lapse of a predefined period of time (e.g., the insertion speed may be reduced after a predefined period of time has elapsed), etc.

In an exemplary embodiment, the syringe insertion process may be divided into one or more phases with each phase having an associated set of control parameters, and the control parameters may be set and/or changed automatically by the motion control computing device 1608 based on the particular phase of the insertion process at a given time.

In an exemplary embodiment, the motion control computing device 1700 may be pre-programmed to control the motion generator 1606 in a desired manner during the syringe insertion process. The pre-programming of the motion control computing device 1700 may be overridden or altered by a user before or during the syringe insertion process. In another exemplary embodiment, the motion control computing device 1608 may not be pre-programmed, and a user may use the motion control computing device 1608 to enter and control a programming of the motion generator 1606 before or during the syringe insertion process.

The motion control computing device 1608 may include one or more input devices 1610, e.g., a touch-screen display device, a keyboard, etc., to allow a user to enter or alter one or more control parameters for controlling the motion generator 1606. The motion control computing device 1608 may include one or more output devices 1612, e.g., a display device, a printer, etc., to output one or more control parameter values for the motion generator 1606 or any other information associated with the syringe insertion process. In an exemplary embodiment, the input device 1610 and the output device 1612 may be provided in one integral device so that a user may view and alter any parameters associated with the motion generator 1606 on the same device. In another exemplary embodiment, the input device 1610 and the output device 1612 may be provided as separate devices.

The motion control computing device 1608 may include one or more communication ports 1614, e.g., ports of a network device, for receiving instructions, data and/or trigger instructions or signals from other devices in the insertion system 1600. For example, the motion control computing device 1608 may use the communication port 1614 to receive a trigger instruction or signal generated by a trigger generation computing device 1618 based on the force profile of the syringe insertion process. An exemplary trigger instruction or signal may instruct the motion control computing device 1608 to control the motion generator 1606 in a particular manner including, but not limited to, starting, stopping, accelerating, decelerating, moving by a predetermined fixed distance, moving for a predetermined fixed time period, etc.

In an exemplary embodiment in which the motion control computing device 1608 is provided separately from the motion generator 1606, the communication port 1614 may be used to send instructions, data and/or trigger instructions or signals from the motion control computing device 1608 to the motion generator 1606 wirelessly or via a wire or cable.

In an exemplary embodiment, the motion control computing device 1608 may be programmed so that, in response to a trigger instruction or signal for changing an aspect of the motion of the motion generator 1606, the motion control computing device 1608 immediately implements the change to the motion of the motion generator 1606. For example, in response to a trigger instruction or signal to stop the motion of the motion generator 1606, the motion control computing device 1608 may automatically and immediately stop the motion of the motion generator 1608.

In another exemplary embodiment, the motion control computing device 1608 may be programmed so that, in response to a trigger instruction or signal for changing an aspect of the motion of the motion generator 1606, the motion control computing device 1608 implements the change to the motion of the motion generator 1606 after a predetermined fixed time delay or after the syringe has traveled a predetermined fixed distance after receipt of the trigger instruction or signal. For example, in response to a trigger instruction or signal to stop the motion of the motion generator 1606, the motion control computing device 1608 may stop the motion of the motion generator 1606 after the syringe has traveled a predetermined fixed distance or for a predetermined fixed time period after receipt of the trigger instruction or signal.

An exemplary motion control computing device 1608 may include, but is not limited to, a Rexroth IndraControl VCP25 computer system equipped with a touch screen available from Bosch Rexroth AG.

FIG. 77 illustrates a block diagram of an exemplary computing device that may be used in exemplary embodiments as the motion control computing device 1608 to control the motion generator 1606. The exemplary computing device is described below in connection with FIG. 77.

The insertion system 1600 may include one or more force and/or pressure sensors 1616 for detecting and monitoring in real-time the forces and/or pressures exerted by the friction point in the proximal cap during the syringe insertion process. In an exemplary embodiment, the force sensor 1616 includes one or more piezoelectric load cells that employ piezoelectric sensors for detecting and monitoring the force profile. In an exemplary embodiment, the piezoelectric sensor includes a quartz crystal and two steel rings that generate an electrical charge when subjected to mechanical force or stress. The charge generated by the sensor may be directly proportional to the mechanical force applied to the sensor. In an exemplary embodiment, the force detected by the force sensor 1616 may be the frictional force with which the friction point in the proximal cap of the automatic injection device resists the insertion of different structural features on the syringe and/or the rigid needle shield during the syringe insertion process. An exemplary force sensor 1616 may include, but is not limited to, a direct piezoelectric load cell manufactured by the Kistler Group.

The insertion system 1600 may include one or more trigger generation computing devices 1618 connectable to the force/pressure sensor 1616 for measuring the forces and/or pressures exerted during the syringe insertion process and for measuring the displacement of the syringe during the syringe insertion process based on an output from the force/pressure sensor 1616. The trigger generation computing device 1618 may perform one or more functions including, but not limited to, measuring in real-time the forces detected by the force/pressure sensor 1616, measuring in real-time the pressures detected by the force/pressure sensor 1616, detecting in real-time that one or more trigger conditions are satisfied by the force profile, generating a trigger instruction or signal when one or more trigger conditions are met, sending the trigger instruction or signal to the motion control computing device 1608 to control the motion generator 1606, etc.

In an exemplary embodiment, the trigger generation computing device 1618 may be pre-programmed to recognize one or more characteristic force features in the force profile that, when generated, satisfy a trigger condition. The pre-programming of the trigger generation computing device 1618 may be overridden or altered by a user before or during the syringe insertion process. In another exemplary embodiment, the trigger generation computing device 1618 may not be pre-programmed, and a user may set one or more characteristic force features that, when generated, satisfy a trigger condition before or during the syringe insertion process. The trigger generation computing device 1618 may include one or more input devices 1620, e.g., a touch-screen display device, a keyboard, etc., to allow a user to enter or alter the specifications for one or more trigger conditions.

The trigger generation computing device 1618 may include one or more communication ports 1624, e.g., one or more ports of a network device, for receiving instructions and/or data from the force sensor 1616. The trigger generation computing device 1618 may be connected to the force sensor 1616 over a wired or wireless network including, but not limited to, the TCP/IP protocol suite, Ethernet, and other networking formats and protocols. The trigger generation computing device 1618 may use the communication port 1624 to receive data and/or instructions encoded in electrical signals (e.g., voltage signals) from the force sensor 1616. The data and/or instructions received from the force sensor 1616 may be used by the trigger generation computing device 1618 to measure and monitor in real-time the associated force values and to trace the force profile of the syringe insertion process. The trigger generation computing device 1618 may monitor the force profile to detect one or more characteristic force features associated with a trigger condition. Upon satisfaction or detection of a trigger condition or upon satisfaction or detection of some other condition, the trigger generation computing device 1618 may generate a trigger instruction or signal. The trigger generation computing device 1618 may use the communication port 1624 to send the trigger instruction or signal to the motion control computing device 1608 to control an aspect of the motion of the motion generator 1606. The trigger instruction or signal may be used to accelerate, decelerate, start, stop or otherwise control the motion of the motion generator 1606 during the syringe insertion process in order to insert the syringe to a desired depth in the housing of the automatic injection device.

The trigger generation computing device 1618 may include one or more output devices 1622, e.g., a display device, a printer, etc., for outputting the specifications for one or more trigger conditions, the detection of a trigger condition, or any other information associated with the syringe insertion process. In an exemplary embodiment, the trigger generation computing device 1618 may output raw data associated with the syringe insertion process, e.g., the forces generated and associated insertion distances and times. In an exemplary embodiment, the trigger generation computing device 1618 may determine and output processed and formatted data associated with the syringe insertion process, e.g., a display of a force profile graph in real-time during the syringe insertion process, other visualizations of the syringe insertion process, and the like. The trigger generation computing device 1618 may output real-time data received from the force sensor 1616 during the syringe insertion process or non real-time data that is stored in a storage device.

The trigger generation computing device 1618 may use the output device 1622, upon completion of the syringe insertion process, to indicate whether the syringe insertion process was successful (i.e., the syringe was inserted to the desired depth into the housing) or whether the syringe insertion process was unsuccessful (i.e., the syringe was inserted too far or not far enough into the housing). The indication provided on the output device 1622 may also indicate other information including, but not limited to, the actual insertion depth of the syringe in the housing, the desired insertion depth, the difference between the desired and the actual insertion depths, the type of the syringe, the type of the rigid needle shield, the type of the automatic injection housing, etc. The indications may allow a user to determine if the assembled automatic injection device is suitable for use by a patient, e.g., when the syringe is inserted exactly or approximately to the desired insertion depth. The indications may also allow a user to determine if the assembled automatic injection device needs to be readjusted before use by a patient or if the assembled device is to be scrapped, e.g., when the syringe is inserted to an insertion depth substantially larger or substantially smaller than the desired insertion depth.

In an exemplary embodiment, the input device 1620 and the output device 1622 may be provided in one integral device so that a user may view and alter any parameters associated with a trigger condition on the same device. In another exemplary embodiment, the input device 1620 and the output device 1622 may be provided as separate devices.

An exemplary trigger generation computing device 1618 may include, but is not limited to, the ControlMonitor CoMo View® control monitor manufactured by the Kistler Group.

FIG. 77 illustrates a block diagram of an exemplary computing device that may be used in exemplary embodiments as the trigger generation computing device 1618. The exemplary computing device is described below in connection with FIG. 77.

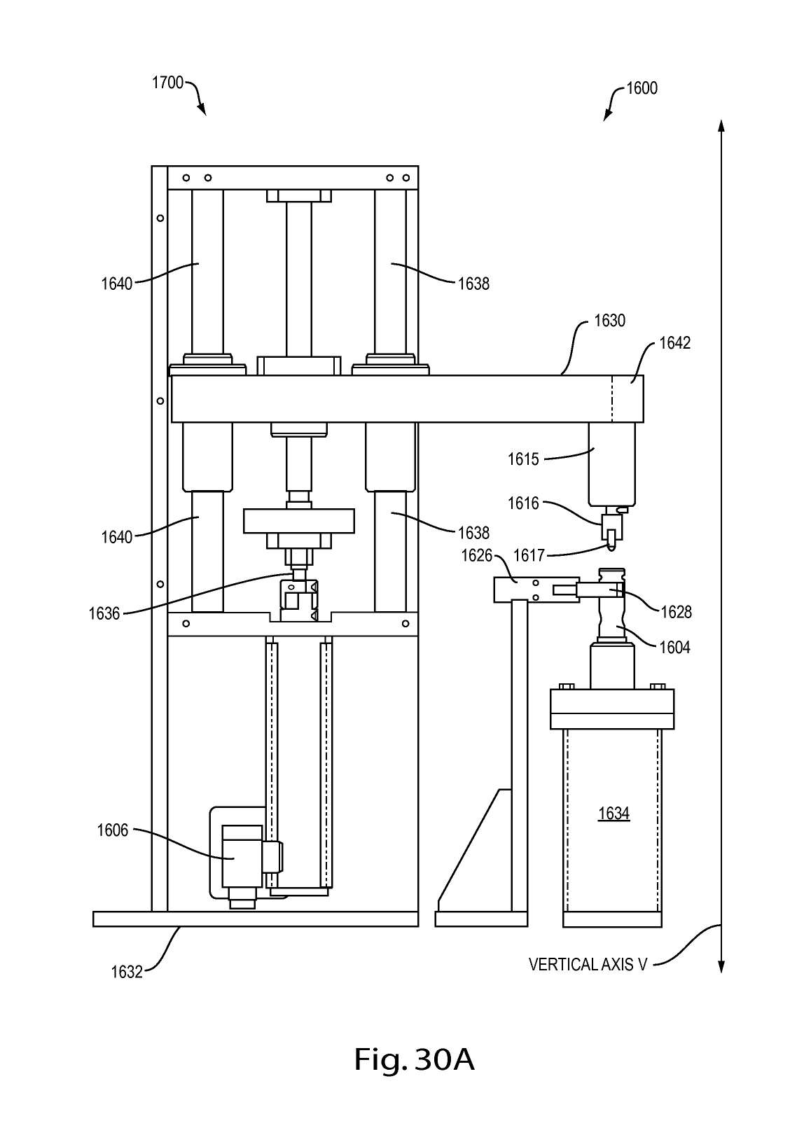

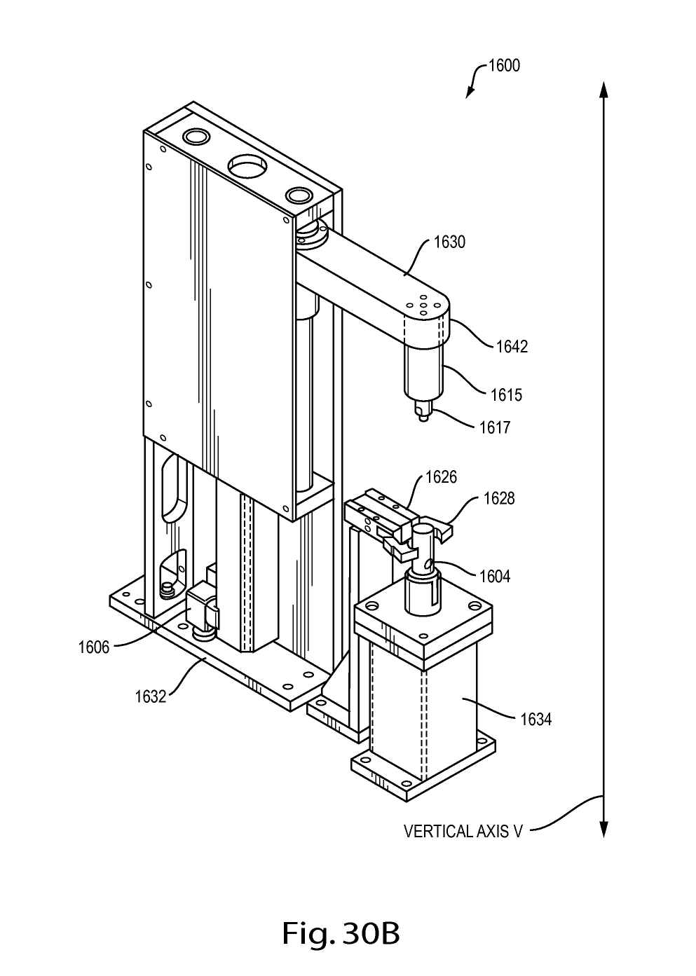

FIG. 30A is a schematic view of an exemplary insertion system 1600, and FIG. 30B is a perspective view of the exemplary insertion system 1600 of FIG. 30A. The insertion system 1600 may include one or more computing devices 1700 that may perform as a motion control computing device and/or as a trigger generation computing device.

The insertion system 1600 may include a housing platform 1634 for supporting a housing 1604 of an automatic injection device in a vertical orientation along the vertical axis V, and a workpiece holder 1626 for releasably securing the housing 1604 in the vertical orientation on the housing platform 1634 during insertion of a syringe into the housing. In an exemplary embodiment, the workpiece holder 1626 may include a releasable syringe holder 1628 for releasably securing a syringe in a vertical orientation along the vertical axis V during insertion of the syringe into the housing 1604 of the automatic injection device. The syringe holder 1628 may extend substantially perpendicularly relative to the vertical axis V of the insertion system 1600.

The insertion system 1600 may include one or more motion generators 1606 that provides a motion for performing the syringe insertion process. An exemplary motion generator 1606 may include, but is not limited to, a servomotor that drives the mechanical member 1630 in an upward or downward direction along the vertical axis V. The motion generator 1606 may be coupled to a horizontal mechanical member 1630 directly or through one or more other mechanical members. A terminal end 1642 of the mechanical member 1630 may be moved upward and/or downward along the vertical axis V by the motion of the motion generator 1606.

The motion generator 1606 may be couplable to the mechanical member 1630 via a drive system 1636. In some embodiments, the drive system 1636 may be configured as a worm drive. The drive system 1636 may be coupled to the mechanical member 1630 via a flange or other coupler. The drive system 1636 may include one or more vertical guide members 1638, 1640 to guide the horizontal member 1630 along the vertical axis V and to prevent movement of the horizontal member 1630 in a horizontal or tangential direction relative to the vertical axis V. In an exemplary embodiment, the drive system 1636 allows macro incremental movements of the terminal end 1642 of the mechanical member 1630 along the vertical axis V on the order of about 1 mm. In an exemplary embodiment, the drive system 1636 allows micro incremental movements of the terminal end 1642 of the mechanical member 1630 along the vertical axis V on the order of about 0.1 mm.

The terminal end 1642 of the mechanical member 1630 may be coupled to a distancing member 1615 that distances the terminal end 1642 from a press head 1617. The press head 1617 may be driven downward toward a syringe so that the syringe is inserted into the housing of an automatic injection device. A force and/or pressure sensor 1616, e.g., one or more piezoelectric load cells, may be provided for detecting and measuring the forces and/or pressures generated during the syringe insertion process. In the exemplary embodiment illustrated in FIG. 30A, the force sensor 1616 is provided between the distancing member 1615 and the press head 1617. One of ordinary skill in the art will recognize that the force sensor 1616 may be provided at any other suitable location.

In an exemplary embodiment, the terminal end 1642 of the mechanical member 1630 provided with the force sensor 1616 may initially be spaced from and disposed vertically above the distal end of the syringe. During an “approach stage” in the syringe insertion process, the mechanical member 1630 may be driven vertically downwardly along the vertical axis V toward the direction of the platform 1632 by the motion generator 1606 such that the force sensor 1616 initially comes into contact with the distal end of the syringe and subsequently drives the syringe vertically downwardly into the housing 1604 of the automatic injection device.

As mentioned above, in some exemplary embodiments, the terminal end 1642 of the mechanical member 1630 may be coupled to the workpiece holder 1626 and/or the syringe holder 1628 in order to drive the housing of the automatic injection device toward the syringe and/or the syringe toward the housing of the automatic injection device. In these exemplary embodiments, the force sensor 1616 may remain stationary, but may come into contact with the distal end of the syringe and/or the housing in order to record the forces and/or pressures exerted during the syringe insertion process as the housing of the automatic injection device is driven toward the syringe.

In other exemplary embodiments, the motion generator 1606 may drive both the force sensor 1616 and the workpiece holder 1626 to move toward each other in order to drive the syringe held by the syringe holder 1628 into the housing 1604 held by the workpiece holder 1626.

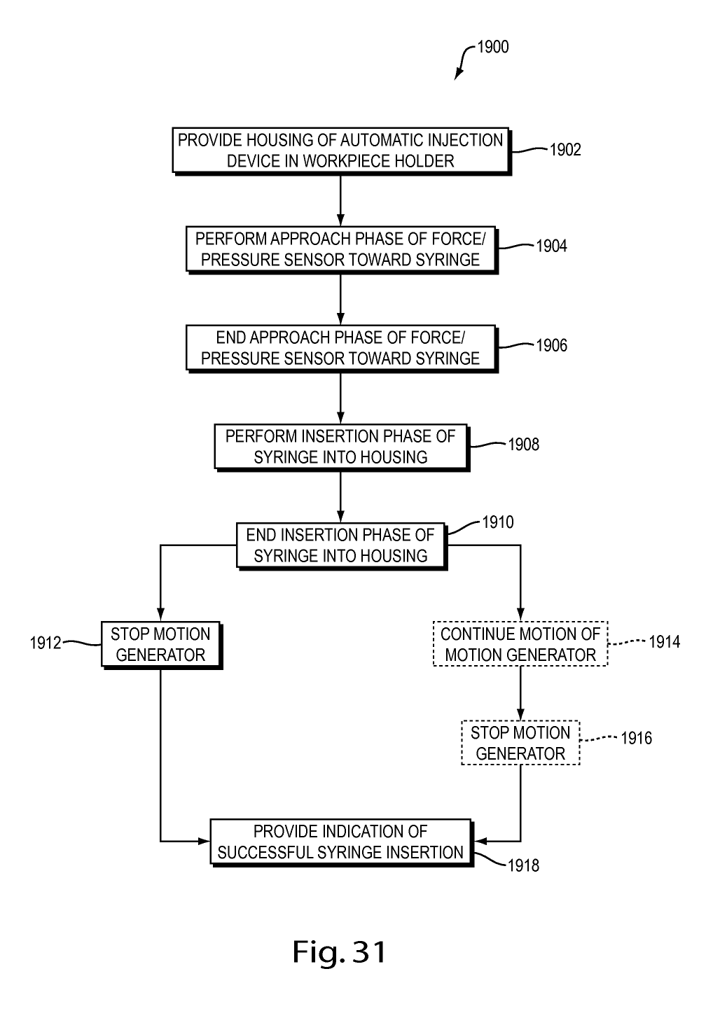

FIG. 31 is a flowchart illustrating an exemplary method 1900 for inserting a syringe into the housing of an automatic injection device. In step 1902, a syringe and a housing of an automatic injection device may be provided in an insertion system. In an exemplary embodiment, the housing may be provided in a workpiece holder in the insertion system. In exemplary embodiments, a mechanical member of the insertion system may be moved upward or downward to drive the syringe into the housing. A terminal end of the mechanical member may be provided with a force and/or pressure sensor. In an exemplary embodiment, the terminal end of the mechanical member may initially be spaced from the distal end of the syringe.

In step 1904, in an “approach phase” of the syringe insertion process, the mechanical member with the attached force sensor is moved toward the distal end of the syringe. In an exemplary embodiment, the approach speed of the mechanical member may be substantially constant. In another exemplary embodiment, the approach speed of the mechanical member may be variable during the approach phase. The exemplary approach speed may range from about 30,000 mm/min to about 35,000 mm/min, but is not limited to this exemplary range. In an exemplary embodiment, the exemplary approach speed may be about 33,000 mm/min. The exemplary acceleration or deceleration in the approach phase may range from about 5,000 mm/s² to about 10,000 mm/s², but is not limited to this exemplary range. In an exemplary embodiment, the exemplary acceleration/deceleration is about 7,000 mm/s².

In step 1906, the approach phase is halted when the force sensor makes contact with the distal end of the syringe. In an exemplary embodiment, the force sensor may detect the contact based on increased forces or an initial detection of a force. In this case, the force detected by the force sensor may be used to trigger the motion generator to end the approach phase of motion. In another exemplary embodiment, in which the force sensor is initially spaced by a predetermined distance from the distal end of the syringe, the motion generator may be triggered to end the approach phase of motion after the mechanical member travels a predetermined distance. In another exemplary embodiment, when the mechanical member has traveled for a time interval corresponding to the predetermined distance (in which the time duration equals the predetermined distance divided by the average speed of the approach phase), the motion generator may be triggered to end the approach phase of motion.

In step 1908, in an “insertion phase” of the syringe insertion process, the tip of the force sensor in conjunction with the motion generator drives the syringe into the housing of the automatic injection device. Some exemplary approach speeds of the mechanical member, and in turn the force sensor may range from about 3,500 mm/min to about 10,000 mm/min, but are not limited to this exemplary range. Other exemplary approach speeds may range from about 5,000 mm/min to about 7,500 mm/min, but are not limited to this exemplary range. An exemplary insertion speed may be lower than an exemplary approach speed in order to allow more precise stoppage of the motion generator when a trigger condition is detected during the insertion phase so that the syringe is stopped at a desired insertion depth. An exemplary acceleration/deceleration speed of the mechanical member and, in turn the force sensor, in the insertion phase ranges from about 75,000 mm/s² to about 85,000 mm/s², but is not limited to this exemplary range. An exemplary acceleration/deceleration is about 80,000 mm/s². An exemplary insertion acceleration/deceleration may be higher than an exemplary approach acceleration/deceleration in order to allow a fast or immediate stoppage of the motion generator when a trigger condition is detected during the insertion phase. In an exemplary embodiment, the mechanical member and, in turn, the force sensor smoothly decelerates from a higher speed in the approach phase to a lower speed in the insertion phase of motion without stopping.

In step 1910, the insertion phase is ended when a halt trigger condition is satisfied, e.g., when a predetermined trigger force and a predetermined trigger hysteresis constituting a halt trigger condition are detected during the syringe insertion process within a desired range of insertion depths of the syringe. Values of the trigger force, the trigger hysteresis, and the range of insertion depths are selected based on the characteristic force profile of the type of syringe and automatic injection device so that the detection of the trigger condition indicates that the syringe is near the desired insertion depth or exactly or approximately at the desired insertion depth.

In an exemplary embodiment, in step 1912, if the halt trigger condition is satisfied indicating that the syringe is exactly or approximately at the desired insertion depth, the motion generator may be triggered to immediately stop farther movement of the mechanical member. In this case, the syringe may stop moving immediately or may move a farther short distance, e.g., from about 0.1 to about 0.3 mm, due to a delay in the trigger instruction or signal reaching or affecting the motion generator.

In another exemplary embodiment, in step 1914, if the halt trigger condition is satisfied indicating that the syringe is near, but not at the desired insertion depth, the motion generator may continue moving the mechanical member for a farther predetermined distance, e.g., from about 1 mm to about 5 mm, after the trigger condition is satisfied. This allows the syringe to continue moving into the housing until it is approximately at the desired insertion depth. The predetermined distance may be determined based on the characteristic force profile of the type of syringe and housing used. For example, in a characteristic force profile, the trigger condition may be satisfied when the syringe is spaced by the predetermined distance from the desired insertion depth. In this case, after the trigger condition is satisfied, the motion generator may be operated to move the mechanical member the predetermined distance. In step 1916, after the syringe is moved to the desired insertion depth in the housing, the motion of the motion generator is stopped and the syringe insertion process is complete.

In step 1918, upon completion of the syringe insertion process, an indication may be provided to a user on an output device, e.g., a display device, on whether the syringe insertion process has been successful (i.e., the syringe was inserted to the desired insertion depth into the housing) or whether the syringe insertion process has been unsuccessful (i.e., the syringe was inserted too far or not far enough into the housing). The indication may also indicate other information including, but not limited to, the actual insertion depth of the syringe into the housing, the desired insertion depth, the difference between the desired and the actual insertion depths, the type of the syringe, the type of the rigid needle shield, the type of the automatic injection housing, etc. These indications may allow the user to determine if the assembled automatic injection device is suitable for use by a patient, e.g., when the syringe is inserted exactly or approximately to the desired insertion depth. These indications may also allow the user to determine if the assembled automatic injection device needs to be readjusted before use by a patient or if the assembled device is to be scrapped, e.g., when the syringe is inserted to an insertion depth greater or less than the desired insertion depth.

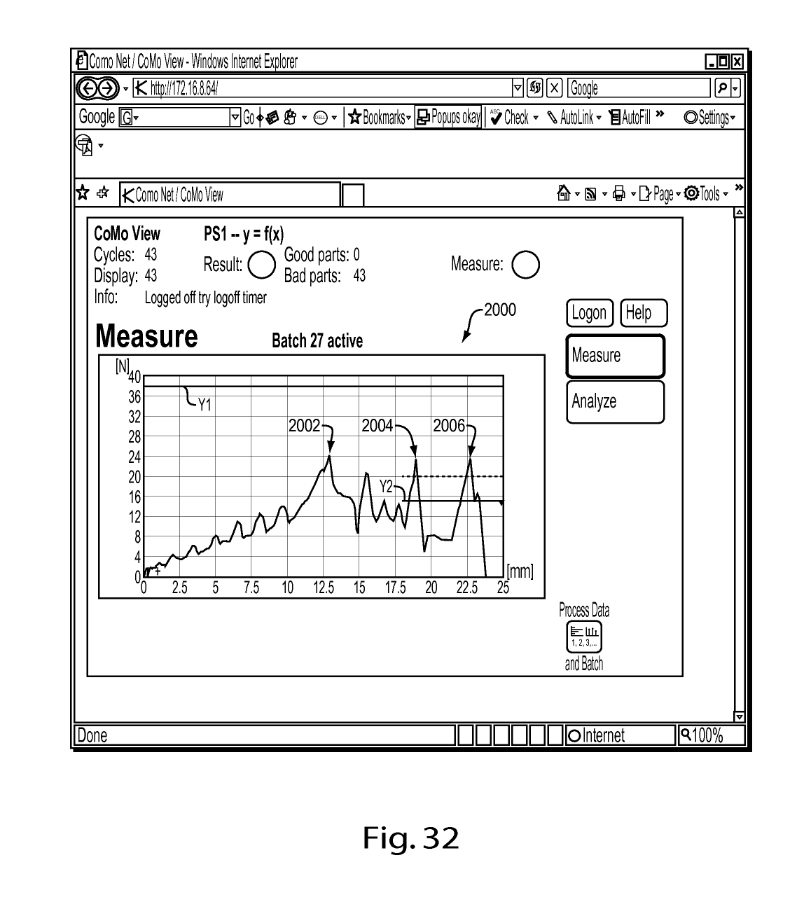

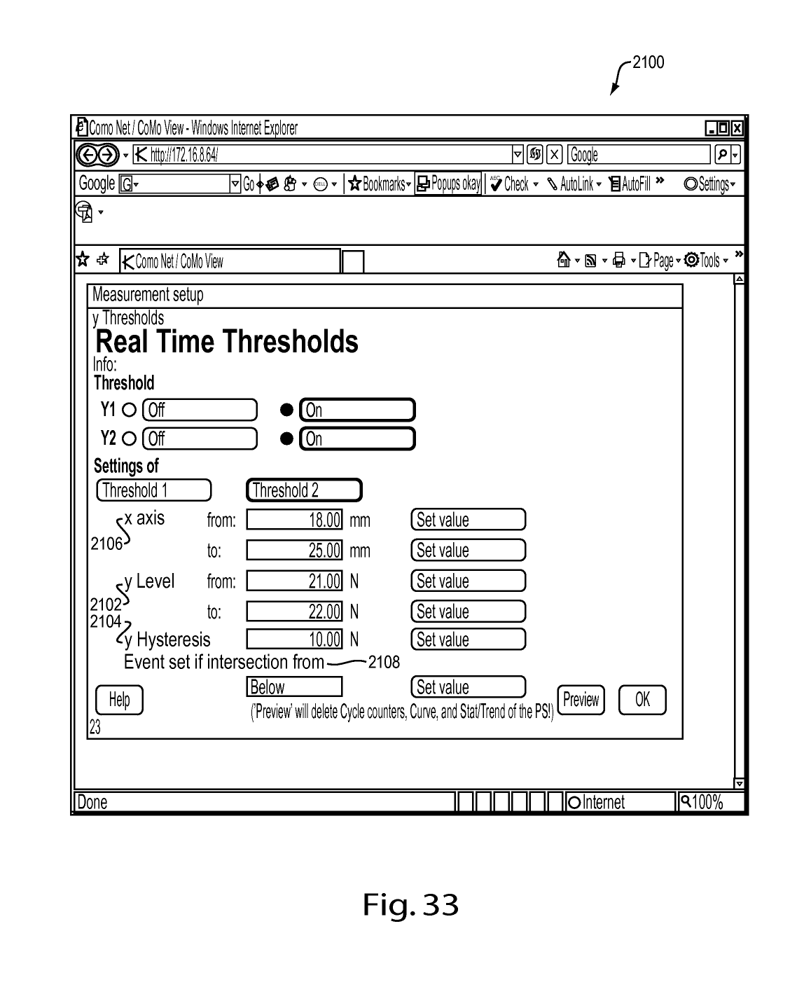

FIGS. 32 and 33 illustrate a syringe insertion method corresponding to exemplary method 1900 illustrated in FIG. 31, in which the trigger condition is selected so that detection of the trigger condition indicates that the syringe is exactly or approximately at the desired insertion depth or a particular distance away from the desired insertion depth.

FIG. 32 illustrates a user interface and a graph showing a characteristic force profile 2000 of a rigid needle shield having an exemplary length of about 25 mm that is generated during its insertion into the housing of an automatic injection device. The y-axis of the graph denotes the frictional forces (in N) detected by an exemplary force sensor as different structural or ornamental features on or in the rigid needle shield pass by a friction point in the proximal cap of the automatic injection device. The x-axis of the graph denotes the distance (in mm) that the proximal end of the rigid needle shield is inserted past the friction point toward the proximal end of the proximal cap.

A first characteristic peak 2002, e.g., about 24 N, occurs when a first feature on the rigid needle shield passes by the friction point in the proximal cap. The first characteristic peak occurs within an x-axis range of between about 10 mm and about 15 mm. A second characteristic peak 2004, e.g., about 23 N, occurs at a subsequent time when a second feature on the shield passes by the friction point in the proximal cap. The second characteristic peak occurs within an x-axis range of between about 18 mm and about 19 mm. A third characteristic peak 2006, e.g., about 24 N, occurs at a subsequent time when the distal end of the rigid needle shield passes by the friction point in the proximal cap. The third characteristic peak occurs within an x-axis range of between about 22 mm and about 25 mm.

FIG. 33 illustrates a user interface 2100 associated with a motion generator driving the syringe into the housing of the automatic injection device. The user interface 2100 displays and allows a user to enter the specification of a halt trigger condition. The specification of an exemplary halt trigger condition may specify values for the trigger force 2102, the trigger hysteresis 2104, and the x-axis range 2106 within which the trigger force and the trigger hysteresis are detected or measured. In this exemplary embodiment, the appearance of an upward sloping portion of a second characteristic peak 2004 within its characteristic x-axis range is used as the trigger condition to indicate that the syringe is exactly or approximately at the desired insertion depth or a particular distance away from the desired insertion depth.

In an exemplary embodiment, the trigger force is set to be a force value that appears on the upward slope of the second characteristic peak, e.g., about 21 N. The trigger hysteresis is set to be a lower force value, e.g., about 10 N. The x-axis range within which the trigger force and the trigger hysteresis are detected or measured is set to be between about 18 mm and about 25 mm. The approach 2108 is indicated to be “from below,” which indicates that the trigger condition is satisfied if the force rises from about 11 N to about 21 N within an x-axis range of between 18 mm and about 25 mm.

In the exemplary embodiment illustrated in FIGS. 32 and 33, during the insertion phase, the trigger condition is detected when the force rises from about 11 N (the trigger force value minus the trigger hysteresis value) to about 21 N (the trigger force value) within an x-axis range of between about 18 mm and about 25 mm. This force characteristic corresponds to a portion of the second characteristic peak 2004 associated with a second feature passing by the friction point in the proximal cap.

In an exemplary embodiment, in the desired assembled device, the second feature of the rigid needle shield sits at the friction point in the proximal cap. In this exemplary embodiment, the detection of all or a portion of the second characteristic peak 2004 as the trigger condition may indicate that the syringe is exactly or approximately at the desired insertion depth. In this exemplary embodiment, upon detection of the trigger condition, the motion generator may be immediately stopped and the syringe insertion process is complete. However, in an exemplary embodiment, due to a delay between the generation of a trigger instruction and stoppage of the motion generator, the syringe may continue to move farther into the housing for a short distance, e.g., about 0.1 to about 0.5 mm.

In another exemplary embodiment, in the desired assembled device, the second feature of the rigid needle shield sits farther inward from the friction point toward the proximal end of the proximal cap. In this exemplary embodiment, the detection of all or a portion of the second characteristic peak 2004 as the trigger condition may indicate that the syringe is a particular distance away from the desired insertion depth. In this exemplary embodiment, upon detection of the trigger condition, the motion generator may continue to move the syringe into the housing of the automatic injection device for a particular distance or a particular period of time (depending on the insertion speed). The distance may be the farther distance that the second feature of the rigid needle shield must travel past the friction point in the proximal cap to reach its final desired location. An exemplary distance may range from between about 1 mm to about 10 mm, but is not limited to this exemplary embodiment. The motion generator is subsequently stopped and the syringe insertion process is complete.

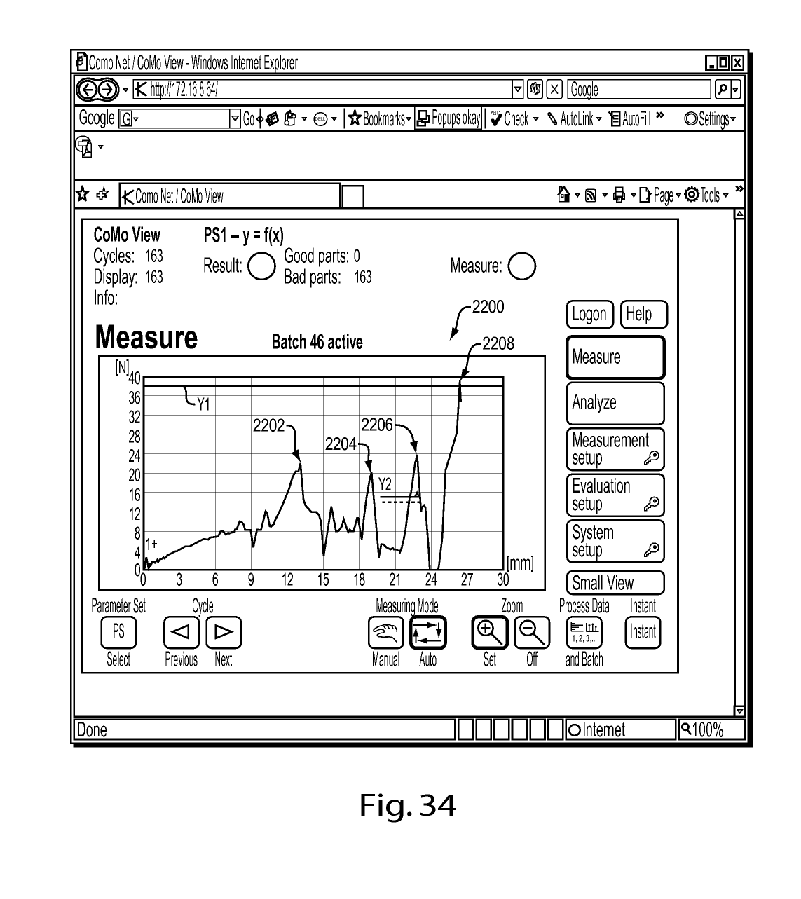

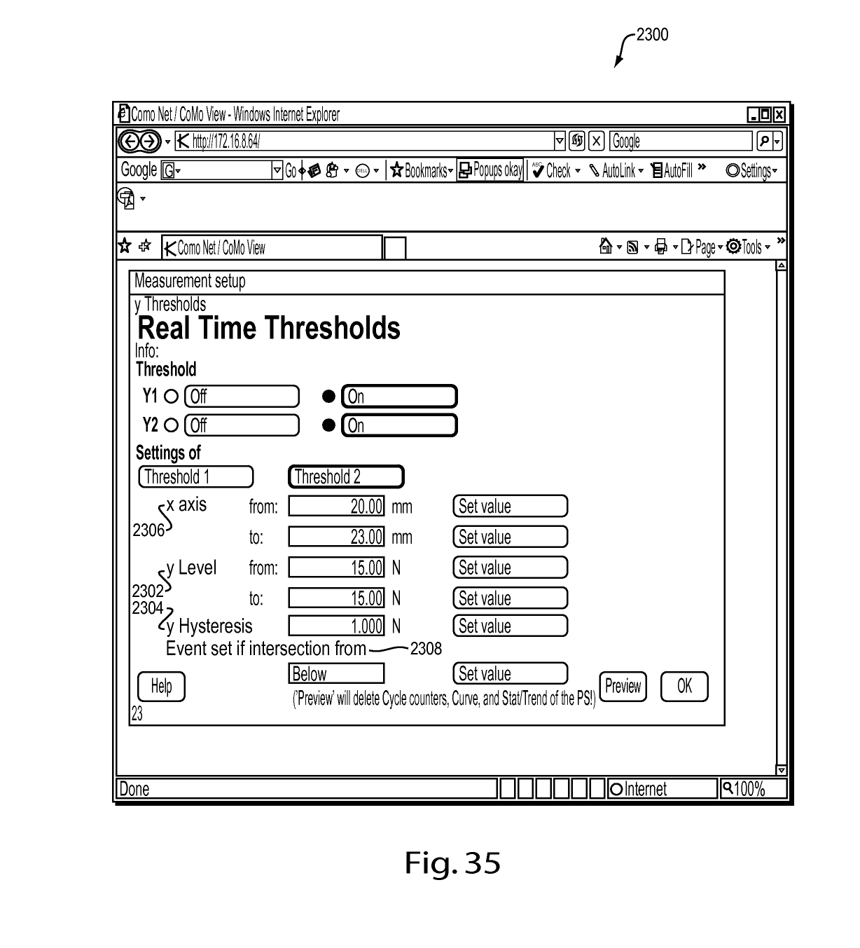

FIGS. 34 and 35 illustrate a syringe insertion method corresponding to exemplary method 1900 illustrated in FIG. 31, in which values of the trigger force, the trigger hysteresis, and the range of insertion depths are selected so that the detection of the trigger force and the trigger hysteresis indicates that the syringe is exactly or approximately at the desired insertion depth or a particular distance away from the desired insertion depth.

FIG. 34 illustrates a user interface and a graph showing a characteristic force profile 2200 of a rigid needle shield having an exemplary length of about 26 mm that is generated during its insertion into the housing of an automatic injection device. The y-axis of the graph denotes the forces (in N) detected by the force sensor as different structural features on the rigid needle shield pass by a friction point in the proximal cap. The x-axis of the graph denotes the distance (in mm) that the proximal end of the rigid needle shield is inserted past the friction point in the proximal cap.

A first characteristic peak 2202, e.g., about 23 N, occurs when a first feature on the rigid needle shield passes by the friction point in the proximal cap. The first characteristic peak occurs within an x-axis range of between about 12 mm and about 14 mm. A second characteristic peak 2204, e.g., about 20 N, occurs at a subsequent time when a second feature on the shield passes by the friction point in the proximal cap. The second characteristic peak occurs within an x-axis range of between about 18 mm and about 20 mm. A third characteristic peak 2206, e.g., about 24 N, occurs at a subsequent time when the distal end of the rigid needle shield passes by the friction point in the proximal cap. The third characteristic peak occurs within an x-axis range of between about 20 mm and about 23 mm. A fourth characteristic peak 2208, e.g., above about 30 N, occurs at a subsequent time when the proximal end of the syringe body begins to pass by the friction point in the proximal cap. The fourth characteristic peak occurs within an x-axis range of between about 25 mm and about 30 mm.

FIG. 35 illustrates a user interface 2300 associated with a motion generator driving the syringe into the housing of the automatic injection device. The user interface 2300 displays and may be used to enter a specification of a trigger condition. The specification of an exemplary trigger condition specifies values for the trigger force 2302, the trigger hysteresis 2304, and the x-axis range 2306 over which the trigger is detected. In this exemplary embodiment, the appearance of an upward sloping portion of the third characteristic peak within its characteristic x-axis range is used as the trigger condition to indicate that the syringe is exactly or approximately at the desired insertion depth or a particular distance away from the desired insertion depth.

In an exemplary embodiment, the trigger force is set to be a force value that appears on the upward slope of the third characteristic peak, e.g., about 15 N. The trigger hysteresis is set to be a lower force value, e.g., about 1 N. The x-axis range within which the trigger force and the trigger hysteresis are detected is set to be between about 20 mm and about 23 mm. The approach 2308 is indicated to be “from below,” which indicates that the trigger condition is satisfied if the force rises from about 14 N to about 15 N within a range on the x-axis of between 20 mm to about 23 mm.

In the exemplary embodiment illustrated in FIGS. 34 and 35, during the insertion phase, the trigger condition is detected when the force rises from about 14 N (the trigger force value minus the trigger hysteresis value) to about 15 N (the trigger force value) within an x-axis range of between about 20 mm and about 23 mm. The detection of the trigger condition corresponds to the distal end of the rigid needle shield passing by the friction point in the proximal cap.

In an exemplary embodiment, in the desired assembled device, the distal end of the rigid needle shield sits at the friction point in the proximal cap. In this exemplary embodiment, the detection of all or a portion of the third characteristic peak 2006 as the trigger condition may indicate that the syringe is exactly or approximately at the desired insertion depth. In this exemplary embodiment, upon detection of the trigger condition, the motion generator may be immediately stopped and the syringe insertion process is complete. However, in an exemplary embodiment, due to a delay between the generation of a trigger instruction and stoppage of the motion generator, the syringe may continue to move farther into the housing for a short distance, e.g., about 0.1 to about 0.5 mm.

In another exemplary embodiment, in the desired assembled device, the distal end of the rigid needle shield sits farther inward from the friction point toward the proximal end of the proximal cap. In this exemplary embodiment, the detection of all or a portion of the third characteristic peak 2006 as the trigger condition may indicate that the syringe is a particular distance away from the desired insertion depth. In this exemplary embodiment, upon detection of the trigger condition, the motion generator may continue to move the syringe into the housing of the automatic injection device for a particular distance or a particular period of time (depending on the insertion speed). The distance may be the farther distance that the distal end of the rigid needle shield must travel past the friction point in the proximal cap to reach its final desired location. An exemplary distance may range from between about 1 mm to about 10 mm, but is not limited to this exemplary embodiment. The motion generator is subsequently stopped and the syringe insertion process is complete.

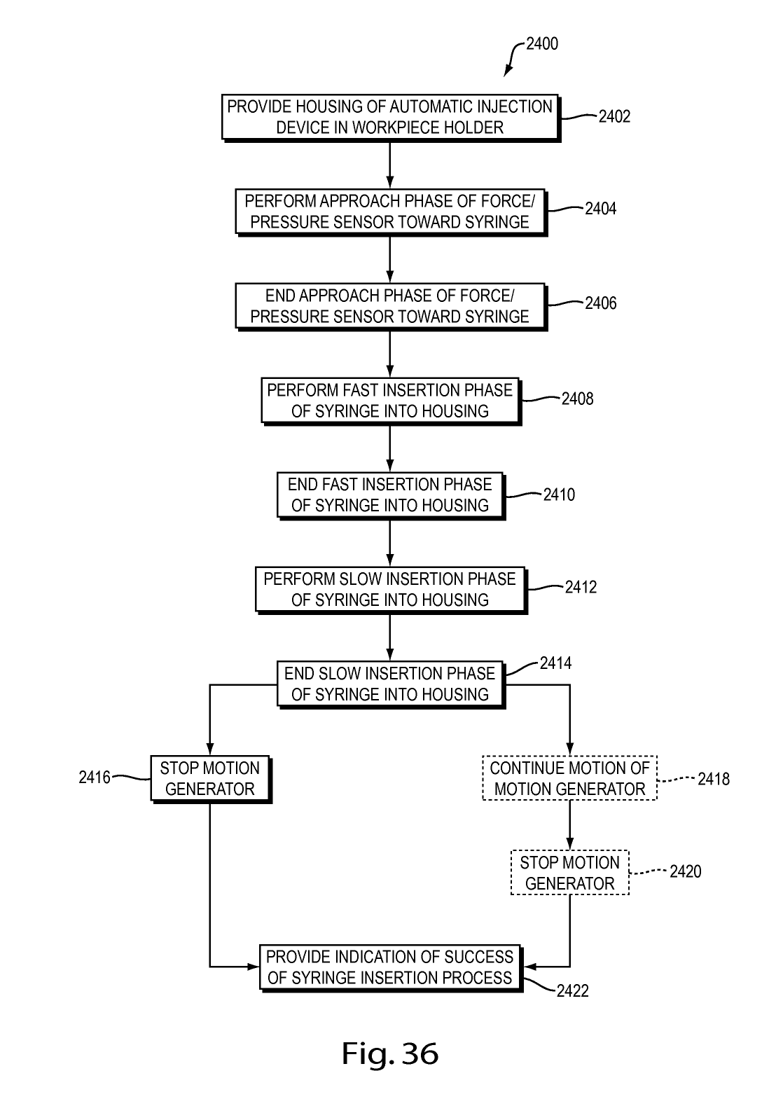

FIG. 36 is a flowchart illustrating an exemplary method 2400 for inserting a syringe into the housing of an automatic injection device. In step 2402, a syringe and a housing of an automatic injection device may be provided in an insertion system. In an exemplary embodiment, the housing may be provided in a workpiece holder in the insertion system. In exemplary embodiments, a terminal end of a mechanical member of the insertion system may be moved upward or downward to drive the syringe into the housing. The terminal end of the mechanical member may be provided with a force and/or pressure sensor. In an exemplary embodiment, the terminal end of the mechanical member provided with the force sensor may initially be spaced from the distal end of the syringe.

In step 2404, in an “approach phase” of the syringe insertion process, the mechanical member provided with the force sensor is moved toward the distal end of the syringe. In an exemplary embodiment, the approach speed may be substantially constant during the approach phase. In another exemplary embodiment, the approach speed may be variable during the approach phase. An exemplary approach speed of the motion generator ranges from about 30,000 mm/min to about 35,000 mm/min, but is not limited to this exemplary range. An exemplary approach speed is about 33,000 mm/min. An exemplary acceleration or deceleration of the motion generator in the approach phase ranges from about 5,000 mm/s² to about 10,000 mm/s², but is not limited to this exemplary range. An exemplary acceleration/deceleration is about 7,000 mm/s².

In step 2406, the approach phase is ended when the force sensor makes contact with the distal end of the syringe. In an exemplary embodiment, the force sensor may detect the contact based on increased forces or detection of a force. In this case, the force detected by the force sensor may be used to trigger the motion generator to end the approach phase of motion. In another exemplary embodiment, in which the mechanical member is initially spaced by a predetermined distance from the distal end of the syringe, the motion generator may be triggered to end the approach phase of motion after the mechanical member has traveled the predetermined distance. In another exemplary embodiment, when the mechanical member has traveled for a time interval corresponding to the predetermined distance (in which the duration of time equals the predetermined distance divided by the average speed of the approach phase), the motion generator may be triggered to end the approach phase of motion.

In exemplary method 2400, the “insertion phase” may be divided into an earlier “fast insertion phase” and a later “slow insertion phase,” so that the trigger condition is detected during the later slow insertion phase. This decreases the distance required to stop the motion generator by operating it at a slower speed during the slow insertion phase, thus resulting in a more precise stopping distance. This allows a lower trigger force to be used near the end of the third characteristic peak, in an exemplary embodiment, which indicates that the syringe insertion process is close to completion. The selection of this trigger force generated at the distal end of the syringe reduces variability in the insertion depth that might otherwise be caused by varying lengths of the rigid needle shield. For example, if the trigger condition was selected to be the second characteristic peak, this could potentially introduce variability in the insertion depth due to variability in the lengths of the rigid needle shields.

In step 2408, in an earlier fast insertion phase of the syringe insertion process, the force sensor coupled to the mechanical member drives the syringe into the housing of the automatic injection device. Exemplary fast insertion speeds of the motion generator may range from about 5,000 mm/min to about 10,000 mm/min, but are not limited to this exemplary range. An exemplary fast insertion speed is about 7,000 mm/min. An exemplary fast insertion speed may be lower than an exemplary approach speed in order to allow precise stoppage of the motion generator when the trigger condition is detected, which ensures that the syringe is stopped at a desired insertion depth. An exemplary acceleration/deceleration speed of the motion generator in the fast insertion phase ranges from about 10,000 mm/s² to about 50,000 mm/s², but is not limited to this exemplary range. An exemplary acceleration/deceleration is about 30,000 mm/s². An exemplary fast insertion acceleration/deceleration may be higher than an exemplary approach acceleration/deceleration in order to allow a fast or immediate stoppage of the motion generator when the trigger condition is detected. In an exemplary embodiment, the motion generator may smoothly decelerate from a higher speed in the approach phase to a lower speed in the earlier fast insertion phase of motion without stopping.

In step 2410, the fast insertion phase is ended after the motion generator has moved the syringe a particular distance within the housing. The distance may be selected so that it is shorter than the total distance the syringe must be moved within the housing to reach the desired insertion depth. The distance may range from between about 5 mm to about 20 mm, but is not limited to this exemplary range. In an exemplary embodiment, the distance is about 15 mm.

In step 2412, in a later slower insertion phase of the syringe insertion process, the force sensor coupled to the mechanical member drives the syringe into the housing of the automatic injection device. Exemplary slow insertion speeds of the motion generator may range from about 500 mm/min to about 1,500 mm/min, but are not limited to this exemplary range. An exemplary slow insertion speed is about 1,000 mm/min. An exemplary slow insertion speed may be lower than an exemplary fast insertion speed in order to allow precise stoppage of the motion generator when the trigger condition is detected, which ensures that the syringe is stopped at a desired insertion depth. An exemplary acceleration/deceleration speed of the motion generator in the slow insertion phase ranges from about 60,000 mm/s² to about 100,000 mm/s², but is not limited to this exemplary range. An exemplary acceleration/deceleration is about 80,000 mm/s². An exemplary slow insertion acceleration/deceleration may be higher than an exemplary fast insertion acceleration/deceleration in order to allow a fast or immediate stoppage of the motion generator when the trigger is detected. In an exemplary embodiment, the motion generator may smoothly decelerate from a higher speed in the fast insertion phase to a lower speed in the slower insertion phase of motion without stopping.

In step 2414, the slower insertion phase is ended when a predetermined trigger force and a predetermined trigger hysteresis constituting a trigger condition are detected or measure within a desired range of insertion depths of the syringe. Values of the trigger force, the trigger hysteresis, and the range of insertion depths may be selected so that the detection of the trigger condition indicates that the syringe is near the desired insertion depth or exactly or approximately at the desired insertion depth.

In an exemplary embodiment, in step 2416, if the trigger is generated when the syringe is exactly or approximately at the desired insertion depth, the motion generator may be triggered to immediately stop movement. In this case, the syringe may stop moving immediately or may move a farther short distance after satisfaction of the trigger condition, e.g., from about 0.1 to about 0.3 mm, due to a delay in a trigger instruction or signal reaching or affecting the motion generator.

In another exemplary embodiment, in step 2418, if the trigger is generated when the syringe is near but not at the desired insertion depth, the motion generator may continue moving the syringe into the housing for a farther predetermined distance, e.g., from about 1 mm to about 10 mm, even after the trigger condition is satisfied. This allows the syringe to continue moving until it is approximately at the desired insertion depth. The predetermined distance may be determined based on the characteristic force profile of the type of syringe and housing used. For example, in a characteristic force profile, the trigger may be generated when the syringe is spaced by the predetermined distance from the desired insertion depth. In this case, after the trigger condition is satisfied, the motion generator may be operated until the syringe has traveled the farther predetermined distance into the housing. In step 2420, the motion of the motion generator is stopped and the syringe insertion process is complete.

In step 2422, upon completion of the syringe insertion process, an indication may be provided on an output device, e.g., a display device, on whether the syringe insertion process has been successful (i.e., the syringe was inserted to the desired insertion depth into the housing) or whether the syringe insertion process has been unsuccessful (i.e., the syringe was inserted too far or not far enough into the housing). The indication may also indicate other information including, but not limited to, the actual insertion depth of the syringe into the housing, the desired insertion depth, the difference between the desired and the actual insertion depths, the type of the syringe, the type of the rigid needle shield, the type of the automatic injection housing, etc. These indications may allow a user to determine if the assembled automatic injection device is suitable for use by a patient, e.g., when the syringe is inserted exactly or approximately to the desired insertion depth. These indications may also allow a user to determine if the assembled automatic injection device needs to be readjusted before use by a patient or if the assembled device is to be scrapped, e.g., when the syringe is inserted to an insertion depth greater or less than the desired insertion depth.

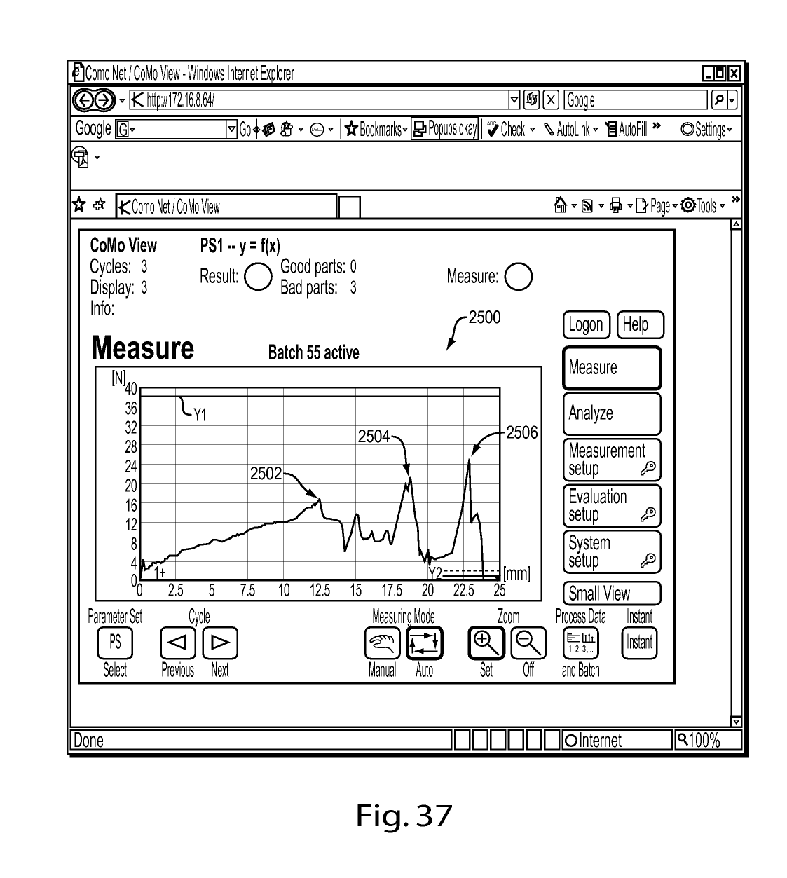

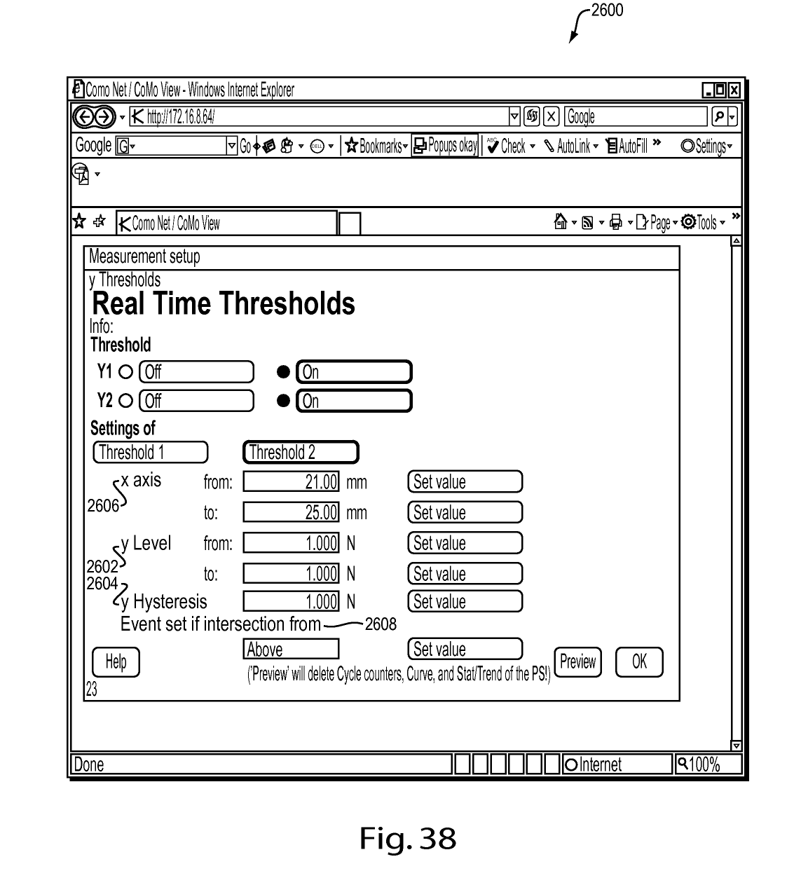

FIGS. 37 and 38 illustrate an exemplary syringe insertion force profile corresponding to exemplary method 2400 illustrated in FIG. 36, in which the trigger condition is selected so that the detection of the trigger condition indicates that the syringe is exactly or approximately at the desired insertion depth or a particular distance away from the desired insertion depth.

FIG. 37 illustrates a user interface and a graph showing a characteristic force profile 2500 of a rigid needle shield having an exemplary length of about 25 mm during its insertion into the housing of an automatic injection device. The y-axis of the graph denotes the forces (in N) detected by the force sensor as different structural features on the rigid needle shield pass by a friction point in the proximal cap. The x-axis of the graph denotes the distance (in mm) that the proximal end of the rigid needle shield is inserted past the friction point toward the proximal end of the proximal cap.

A first characteristic peak 2502, e.g., about 17 N, occurs when a first feature on the rigid needle shield passes by the friction point in the proximal cap. The first characteristic peak occurs within an x-axis range of between about 11 mm and about 14 mm. A second characteristic peak 2504, e.g., about 22 N, occurs at a subsequent time when a second feature on the rigid needle shield passes by the friction point in the proximal cap. The second characteristic peak occurs within an x-axis range of between about 18 mm and about 20 mm. A third characteristic peak 2506, e.g., about 26 N, occurs at a subsequent time when the distal end of the rigid needle shield passes by the friction point in the proximal cap. The third characteristic peak occurs within an x-axis range of between about 21 mm and about 25 mm.

FIG. 38 illustrates a user interface 2600 associated with a motion generator driving the syringe into the housing of the automatic injection device. The user interface 2600 displays and allows a user to enter the specification of a trigger condition. The specification of an exemplary trigger condition may specify values entered for the trigger force 2602, the trigger hysteresis 2604, and the x-axis range 2606 within which the trigger force and the trigger hysteresis are detected In this exemplary embodiment, the appearance of a later downward sloping portion of the third characteristic peak 2506 within its characteristic x-axis range is used as the trigger condition to indicate that the syringe is exactly or approximately at the desired insertion depth or a particular distance away from the desired insertion depth.

In an exemplary embodiment, the trigger force 2602 is set to be a lower force value that appears on the final downward slope of the third characteristic peak 2506, e.g., about 1 N, and the trigger hysteresis 2604 is set to be about 1 N. The x-axis range 2606 within which the trigger is detected is set to be between about 21 mm and about 25 mm. The approach 2608 is indicated to be “from above,” which indicates that the trigger condition is satisfied if the force falls from about 2 N to about 1 N within a range on the x-axis of between 21 mm to about 25 mm.

In the exemplary embodiment illustrated in FIGS. 37 and 38, during the slow insertion phase, the trigger condition is detected when the force falls from about 2 N (the trigger hysteresis value minus the trigger force value) to about 1 N (the trigger force value) within an x-axis range of between about 21 mm and about 25 mm. The detection of the trigger condition corresponds to the distal end of the rigid needle shield passing by the friction point in the proximal cap.

In an exemplary embodiment, in the desired assembled device, the distal end of the rigid needle shield sits at the friction point in the proximal cap. In this exemplary embodiment, the detection of all or a portion of the third characteristic peak 2506 as the trigger condition may indicate that the syringe is exactly or approximately at the desired insertion depth. In this exemplary embodiment, upon detection of the trigger condition, the motion generator may be immediately stopped and the syringe insertion process is complete. However, in an exemplary embodiment, due to a delay between the generation of a trigger instruction and stoppage of the motion generator, the syringe may continue to move farther into the housing for a short distance, e.g., about 0.1 to about 0.5 mm.

In another exemplary embodiment, in the desired assembled device, the distal end of the rigid needle shield sits farther inward from the friction point toward the proximal end of the proximal cap. In this exemplary embodiment, the detection of all or a portion of the third characteristic peak 2506 as the trigger condition may indicate that the syringe is a particular distance away from the desired insertion depth. In this exemplary embodiment, upon detection of the trigger condition, the motion generator may continue to move the syringe into the housing of the automatic injection device for a particular distance or a particular period of time (depending on the insertion speed). The distance may be the farther distance that the distal end of the rigid needle shield must travel past the constriction in the proximal cap to reach its final desired location. An exemplary distance may range from between about 1 mm to about 10 mm, but is not limited to this exemplary embodiment. The motion generator is subsequently stopped and the syringe insertion process is complete.

### VI. EXEMPLARY COMPUTING DEVICES

Exemplary embodiments may include a motion control computing device for controlling one or more control parameters for a motion generator during an assembly process. An exemplary motion control computing device may include one or more input devices, e.g., a touch-screen display device, a keyboard, etc., to allow a user to enter or alter one or more control parameters for controlling the motion generator. The motion control computing device may include one or more output devices, e.g., a display device, a printer, etc., to output one or more control parameter values for the motion generator or any other information associated with the assembly process. In an exemplary embodiment, the input device and the output device may be provided in one integral device so that a user may view and alter any parameters associated with the motion generator on the same device. In another exemplary embodiment, the input device and the output device may be provided as separate devices.

The motion control computing device may include one or more communication ports, e.g., ports of a network device, for receiving instructions, data and/or trigger instructions or signals from other devices in the assembly systems. For example, the motion control computing device may use the communication port to receive a trigger instruction or signal generated by a trigger generation computing device based on the force profile generated during the assembly process. An exemplary trigger instruction or signal may instruct the motion control computing device to control the motion generator in a particular manner including, but not limited to, starting, stopping, accelerating, decelerating, moving by a predetermined fixed distance, moving for a predetermined fixed time period, etc.

In an exemplary embodiment in which the motion control computing device is provided separately from the motion generator, the communication port may be used to send instructions, data and/or trigger instructions or signals from the motion control computing device to the motion generator wirelessly or via a wire or cable.

In an exemplary embodiment, the motion control computing device may be programmed so that, in response to a trigger instruction or signal for changing an aspect of the motion of the motion generator, the motion control computing device immediately implements the change to the motion of the motion generator. For example, in response to a trigger instruction or signal to stop the motion of the motion generator, the motion control computing device may automatically and immediately stop the motion of the motion generator. In another exemplary embodiment, the motion control computing device may be programmed so that, in response to a trigger instruction or signal for changing an aspect of the motion of the motion generator, the motion control computing device implements the change to the motion of the motion generator after a predetermined fixed time delay or after the press head has traveled a predetermined fixed distance after receipt of the trigger instruction or signal. For example, in response to a trigger instruction or signal to stop the motion of the motion generator, the motion control computing device may stop the motion of the motion generator after the press head has traveled a predetermined fixed distance or for a predetermined fixed time period after receipt of the trigger instruction or signal.

An exemplary motion control computing device may include, but is not limited to, a Rexroth IndraControl VCP25 computer system equipped with a touch screen available from Bosch Rexroth AG.

Exemplary embodiments may provide a trigger generation computing device for measuring the forces and/or pressures exerted during an assembly process and for measuring the displacement of a press head during the assembly process based on an output from a force/pressure sensor. The trigger generation computing device may include one or more input devices, e.g., a touch-screen display device, a keyboard, etc., to allow a user to enter or alter the specifications for one or more trigger conditions. The trigger generation computing device may include one or more communication ports, e.g., one or more ports of a network device, for receiving instructions and/or data from the force sensor. The trigger generation computing device may be connected to the force sensor over a wired or wireless network including, but not limited to, the TCP/IP protocol suite, Ethernet, and other networking formats and protocols. The trigger generation computing device may use the communication port to receive data and/or instructions encoded in electrical signals (e.g., voltage signals) from the force sensor.

The data and/or instructions received from the force sensor may be used by the trigger generation computing device to measure and monitor in real-time the associated force values and to trace the force profile of the assembly process. The trigger generation computing device may monitor the force profile to detect one or more characteristic force features associated with a trigger condition. Upon satisfaction or detection of a trigger condition or upon satisfaction or detection of some other condition, the trigger generation computing device may generate a trigger instruction or signal. The trigger generation computing device may use the communication port to send the trigger instruction or signal to the motion control computing device to control an aspect of the motion of the motion generator. The trigger instruction or signal may be used to accelerate, decelerate, start, stop or otherwise control the motion of the motion generator during the assembly process.

The trigger generation computing device may include one or more output devices, e.g., a display device, a printer, etc., for outputting the specifications for one or more trigger conditions, the detection of a trigger condition, or any other information associated with the assembly process. In an exemplary embodiment, the trigger generation computing device may output raw data associated with the assembly process, e.g., the forces generated and associated insertion distances and times. In an exemplary embodiment, the trigger generation computing device may determine and output data associated with the assembly process, e.g., a display of a force profile graph in real-time during the assembly process, other visualizations of the assembly process, and the like. The trigger generation computing device may output real-time data received from the force sensor during the assembly process or non real-time data that is stored in a storage device.

In an exemplary embodiment, the input device and the output device may be provided in one integral device so that a user may view and alter any parameters associated with a trigger condition on the same device. In another exemplary embodiment, the input device and the output device may be provided as separate devices.

An exemplary trigger generation computing device may include, but is not limited to, the ControlMonitor CoMo View® control monitor manufactured by the Kistler Group.

FIG. 77 illustrates a block diagram of an exemplary computing device 1700 that may be used in exemplary embodiments as the trigger generation computing device and/or as the motion control computing device. In an exemplary embodiment, the motion control computing device and the trigger generation computing device may be provided integrally as the same computing device. In another exemplary embodiment, the motion control computing device and the trigger generation computing device may be provided separately as separate computing devices.

In an exemplary embodiment, the motion control computing device and/or the trigger generation computing device may be provided integrally with an assembly system. In another exemplary embodiment, the motion control computing device and/or the trigger generation computing device may be provided separately from the assembly system.

The computing device 1700 includes one or more computer-readable media for storing one or more computer-executable instructions or software for implementing exemplary embodiments. The computer-readable media may include, but are not limited to, one or more types of hardware memory, non-transitory tangible media, etc. For example, memory 1706 included in the computing device 1700 may store computer-executable instructions or software for implementing exemplary embodiments. The computing device 1700 includes processor 1702 and one or more processor(s) 1702′ for executing computer-executable instructions or software stored in the memory 1706 and other programs for controlling system hardware. Processor 1702 and processor(s) 1702′ may each be a single core processor or multiple core (1704 and 1704′) processor.

Virtualization may be employed in the computing device 1700 so that infrastructure and resources in the computing device may be shared dynamically. A virtual machine 1714 may be provided to handle a process running on multiple processors so that the process appears to be using only one computing resource rather than multiple computing resources. Multiple virtual machines may also be used with one processor.

Memory 1706 may include a computer system memory or random access memory, such as DRAM, SRAM, EDO RAM, etc. Memory 1706 may include other types of memory as well, or combinations thereof.

A user may interact with the computing device 1700 through a visual display device 1718, such as a computer monitor, which may display one or more user interfaces 1720 or any other information on the assembly process. The visual display device 1718 may also display other aspects or elements of exemplary embodiments. The computing device 1700 may include other I/O devices such a keyboard or a multi-point touch interface 1708 and a pointing device 1710, for example a mouse, for receiving input from a user. The keyboard 1708 and the pointing device 1710 may be connected to the visual display device 1718. The computing device 1700 may include other suitable conventional I/O peripherals. The computing device 1700 may also include a storage device 1724, such as a hard-drive, CD-ROM or other computer readable media, for storing data and computer-readable instructions or software that implement exemplary embodiments.

The computing device 1700 may include a network interface 1712 configured to interface via one or more network devices 1722 with one or more networks, e.g., Local Area Network (LAN), Wide Area Network (WAN) or the Internet through a variety of connections including, but not limited to, standard telephone lines, LAN or WAN links (e.g., 802.11, T1, T3, 56 kb, X.25), broadband connections (e.g., ISDN, Frame Relay, ATM), wireless connections, controller area network (CAN), or some combination of any or all of the above. The network interface 1712 may include a built-in network adapter, network interface card, PCMCIA network card, card bus network adapter, wireless network adapter, USB network adapter, modem or any other device suitable for interfacing the computing device 1700 to any type of network capable of communication and performing the operations described herein. Moreover, the computing device 1700 may be any computer system, such as a workstation, desktop computer, server, laptop, handheld computer or other form of computing or telecommunications device that is capable of communication and that has sufficient processor power and memory capacity to perform the operations described herein.

The computing device 1700 may run any suitable operating system 1716, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. The operating system 1716 may be run in native mode or emulated mode.

### VII. EXEMPLIFICATION OF ASSEMBLY OF A SYRINGE WITH A HOUSING OF AN AUTOMATIC INJECTION DEVICE

The following experimental examples are associated with an exemplary system, device and method for assembling a syringe with a housing of an automatic injection device.

### A. First Set of Experiments

### A. (i) Summary

Exemplary syringe insertion systems were tested to determine whether the systems were capable of inserting pre-filled syringes into the housing of automatic injection devices to the proper depth based on a force profile. Three exemplary syringe types (a first type, i.e., Type 1, a second type, i.e., Type 2 and a third type, i.e., Type 3) were inserted to the proper depths in an auto injector using exemplary insertion stations relying on a force profile to control the insertion process.

Test results indicated that exemplary syringe insertion systems repeatedly and reliably inserted all three syringe types to the proper depth in the auto injector. The angular orientation of the rigid needle shield within the proximal cap was determined to not have any observable effect on the insertion of the syringe. A trigger force of about 2 N was used in some exemplary embodiments, which resulted in an optimal insertion depth of about 7.89 mm (±0.09) for the Type 2 syringes, about 8.06 mm (±0.07) for the Type 1 syringes, and about 8.00 mm (±0.03) for the Type 3 syringes. Exemplary syringes that had lengths beyond (+2 mm, −5 mm) of the syringe length specification of about 81.8 mm (±1.3) were also inserted to the proper depth using exemplary insertion stations, demonstrating that the robustness of the exemplary syringe insertion process can account for syringe variability, manufacturing and process variability as well as material variability.

### A. (ii) Experimental Methodology and Results

Tables 1 and 2 summarize exemplary equipment and device components, respectively, used in testing the insertion of exemplary syringes into the housing of exemplary automatic injection devices.

TABLE 1
Exemplary equipment used in testing the insertion of exemplary syringes
into the housing of exemplary automatic injection devices

| Description | Manufacturer |
|---|---|
| Sortimat assembly prototype | Sortimat Technology GmbH & Co. |
| Zwick force tester | Zwick |
| Calipers | VWR International, LLC |

TABLE 2
Exemplary device components used in testing the insertion
of syringes into the housing automatic injection devices

Item
Syringes of a first type, i.e., Type 1
Syringes of a second type, i.e., Type 2
Syringes of a third type, i.e., Type 3
HUMIRA ® autoinjector subassemblies

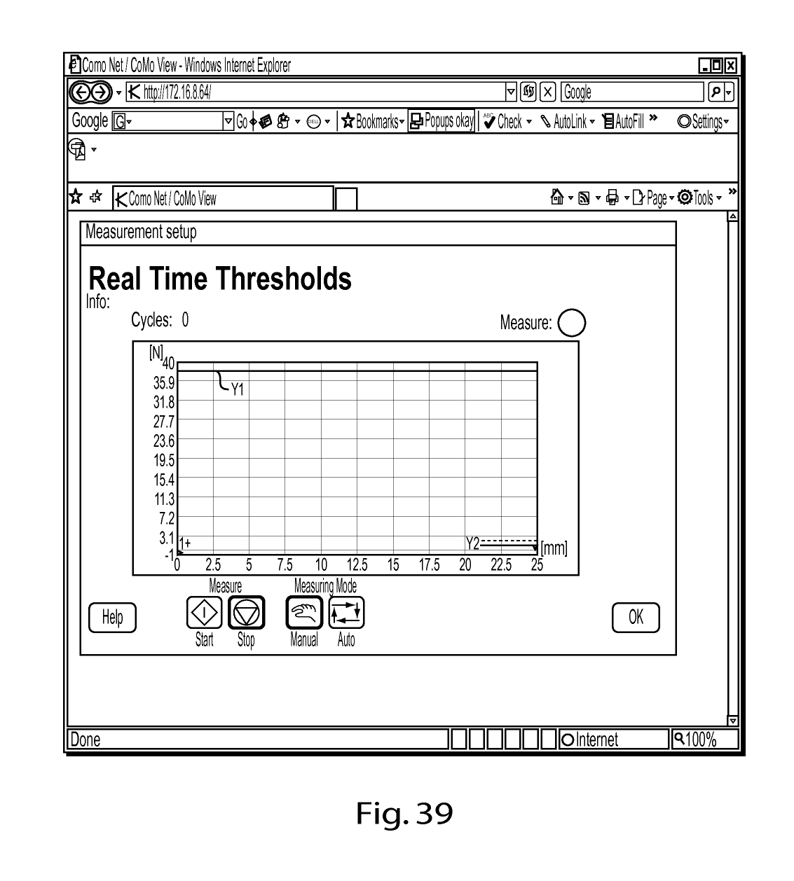

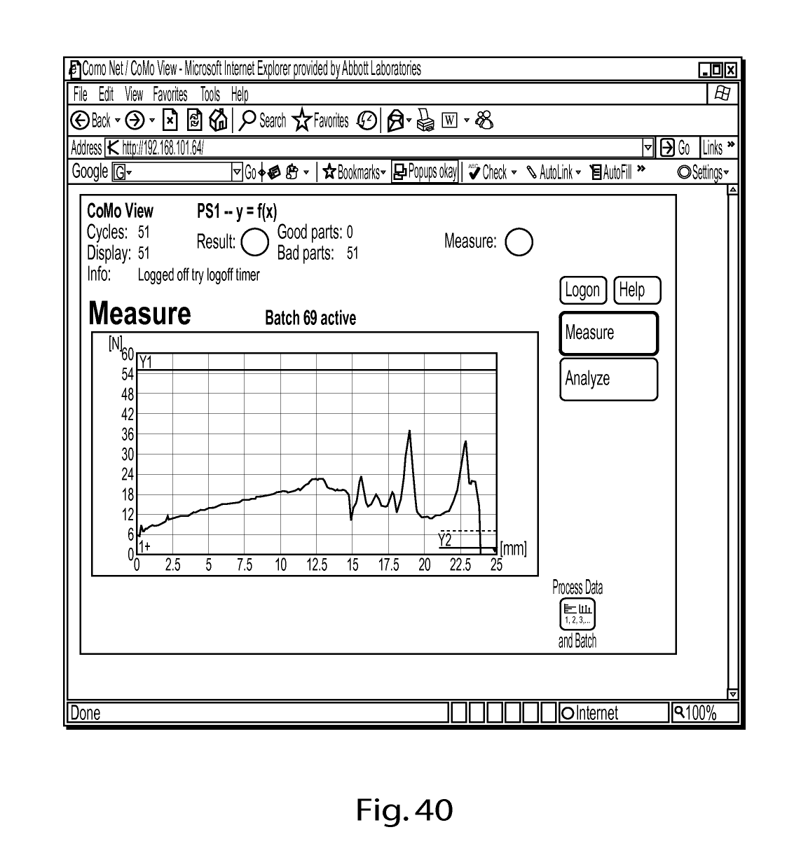

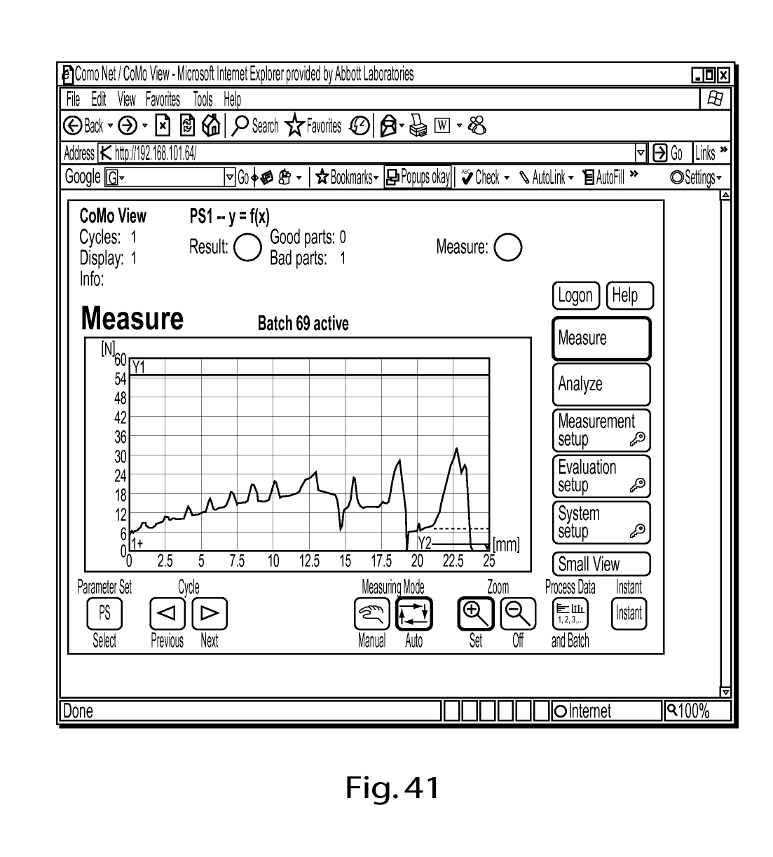

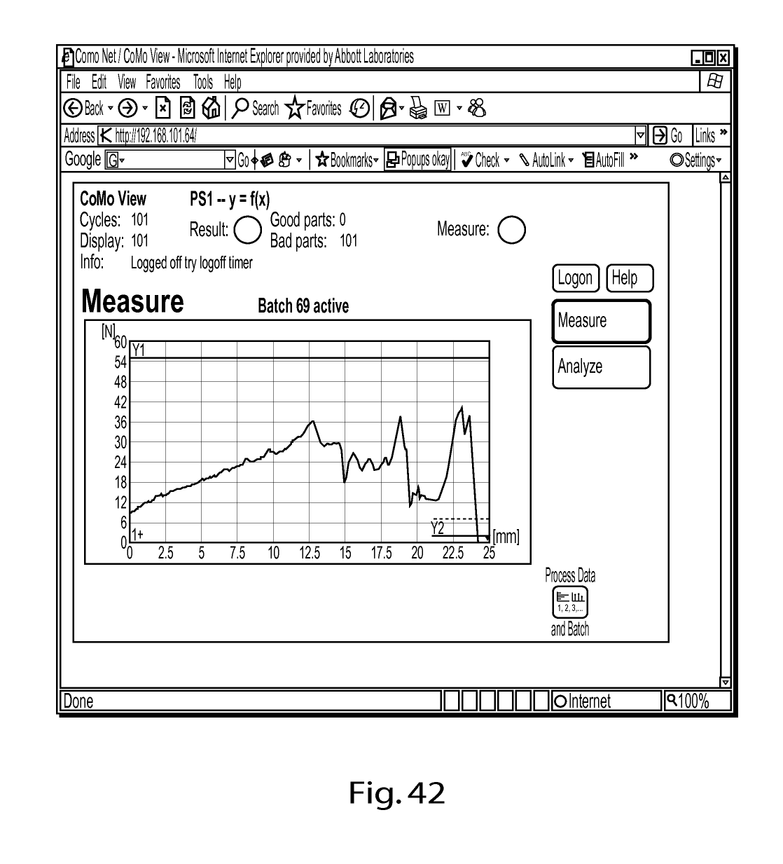

The insertion process of each syringe generated a force profile that was displayed on a user interface provided on a ComoView® control monitor and stored on a computer-readable storage device in a suitable computer file, e.g., an excel file or in an original .bin file. FIG. 39 illustrates an empty screen print from the user interface provided on the ComoView® control monitor. A force profile may be generated for display on the user interface shown in FIG. 39 during the syringe insertion process, one or more trigger conditions may be specified, and detection of a trigger condition may be indicated. FIGS. 40, 41 and 42 illustrate the force profiles generated during the syringe insertion process of the three tested syringe types: the Type 1 syringes, the Type 2 syringes, and the Type 3 syringes, respectively.

A total of 450 syringes (150 syringes of each type, 50 syringes at each trigger condition) were inserted to an optimal depth using the three exemplary trigger conditions, as summarized in Table 3. Each trigger condition included satisfaction of a trigger force (Y₂) and satisfaction of an earlier force that is indirectly indicated by a trigger hysteresis (Y_{Hysteresis}).

TABLE 3
Summary of three exemplary trigger conditions
used in testing insertion of exemplary syringes

| Trigger Force (Y₂) | Trigger Hysteresis (Y_{Hysteresis}) |
|---|---|
| 0.5 N | 2 N |
| 2 N | 5 N |
| 5 N | 5 N |

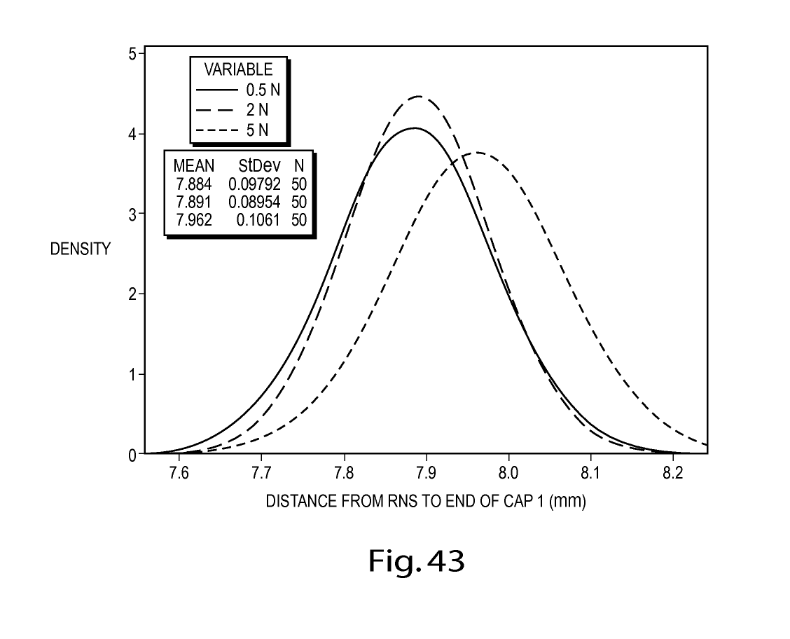

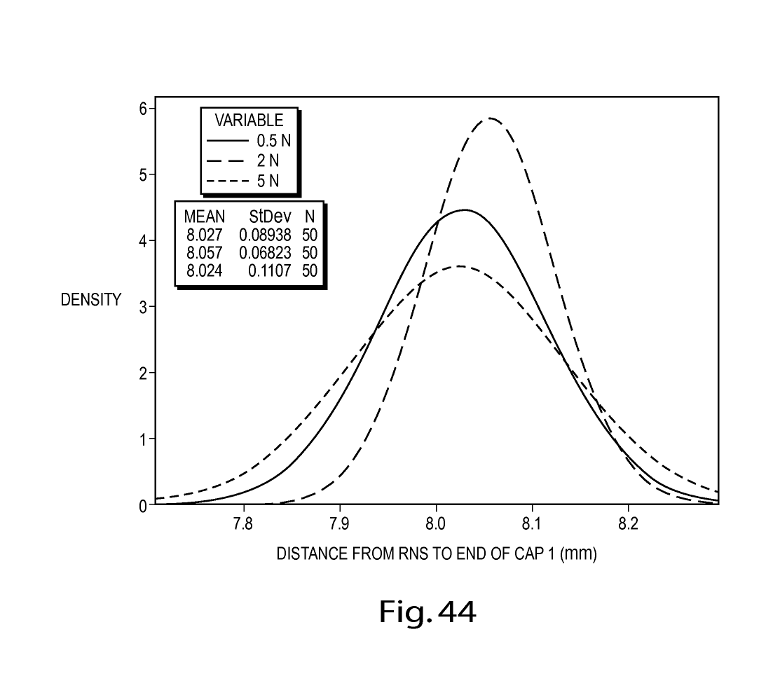

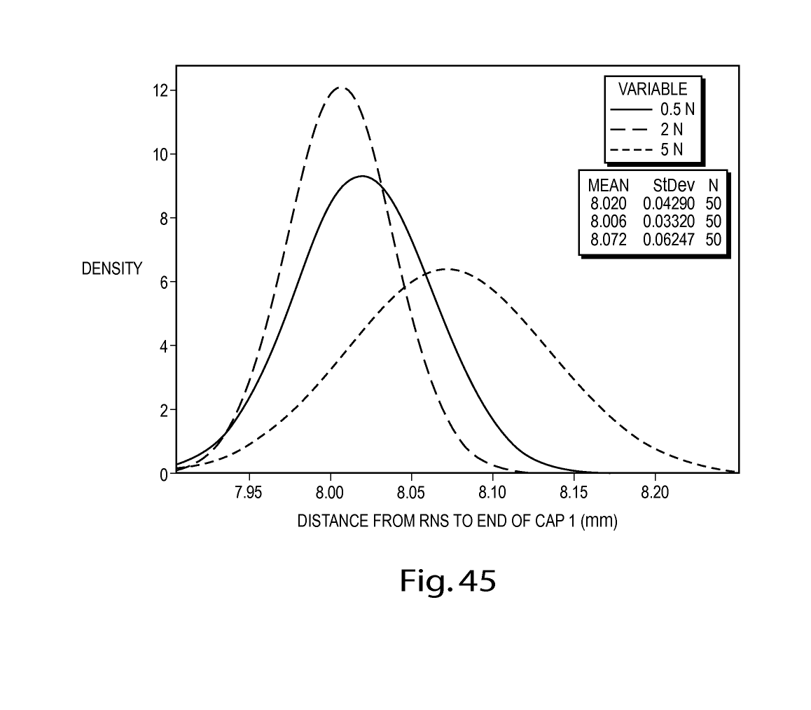

In each case, after completion of the syringe insertion process, the insertion depth of the proximal end of the rigid needle shield was measured from the proximal end of the proximal proximal cap 24 (“Cap 1”). FIG. 43 illustrates a histogram of the number of tested Type 2 syringes (y-axis) that achieved different exemplary insertion depths in mm (x-axis), for the three exemplary trigger conditions summarized in Table 3. FIG. 44 illustrates a histogram of the number of tested Type 1 syringes (y-axis) that achieved different exemplary insertion depths in mm (x-axis), for the three exemplary trigger conditions summarized in Table 3. FIG. 45 illustrates a histogram of the number of tested Type 3 syringes (y-axis) that achieved different exemplary insertion depths in mm (x-axis), for the three exemplary trigger conditions summarized in Table 3.

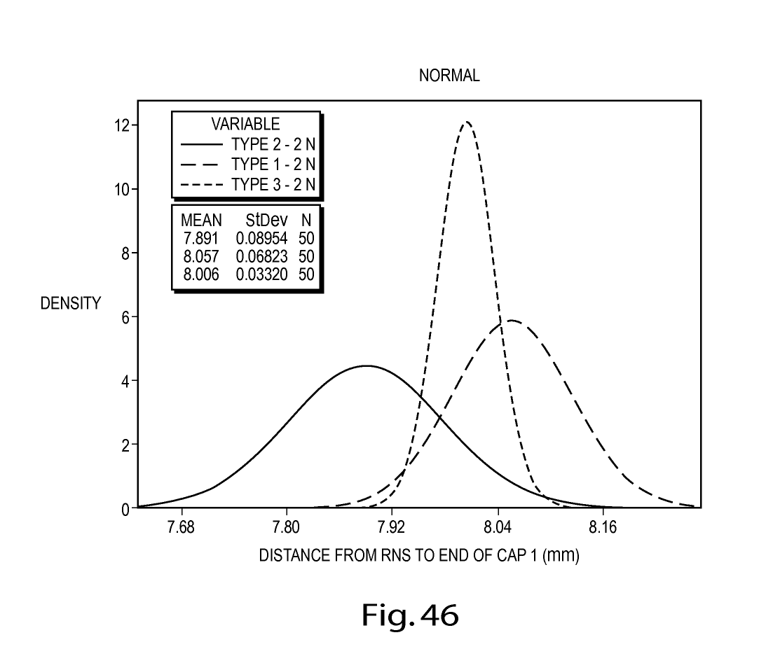

The experimental results indicated that the trigger condition with a trigger force of about 2 N and a trigger hysteresis of about 5 N had the best repeatability in achieving consistent rigid needle shield insertion depths. The experimental results also indicated some differences in the insertion depth for the different syringe types. FIG. 46 illustrates a histogram of the number of tested Type 1, Type 2 and Type 3 syringes (y-axis) that achieved different exemplary insertion depths in mm (x-axis) for the above-mentioned trigger condition (trigger force of 2N and trigger hysteresis of 5 N).

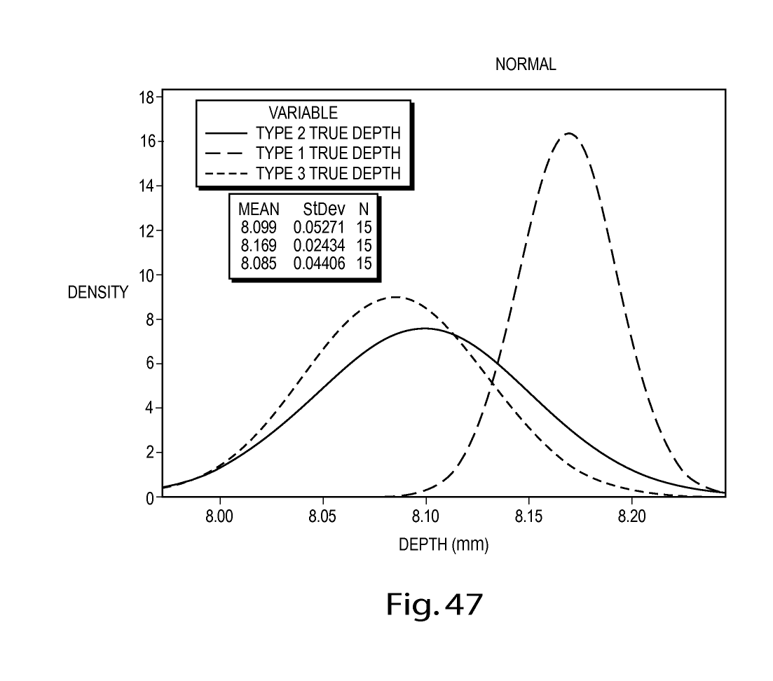

After the tested syringe insertion, the rigid needle shield for each syringe type was pushed back ever so slightly so that the distal end of the rigid needle shield came to rest at or near the friction point in the proximal cap (“Cap 1”). The minor adjustment involved pushing the rigid needle shield over a distance of about 0.1 mm. This indicated that the syringe insertion process was pushing the distal end of the rigid needle shield to a minimal distance past the friction point in the proximal cap, whereas the desired resting point of the distal end of the rigid needle shield was substantially at the friction point. The insertion depths were then re-measured. FIG. 47 illustrates a histogram of the number of tested Type 1, Type 2 and Type 3 syringes (y-axis) that achieved different exemplary insertion depths in mm (x-axis) after this minor adjustment.

A. (iii) Evaluation of Different Angular Orientations of the Rigid Needle Shield in the Proximal Cap

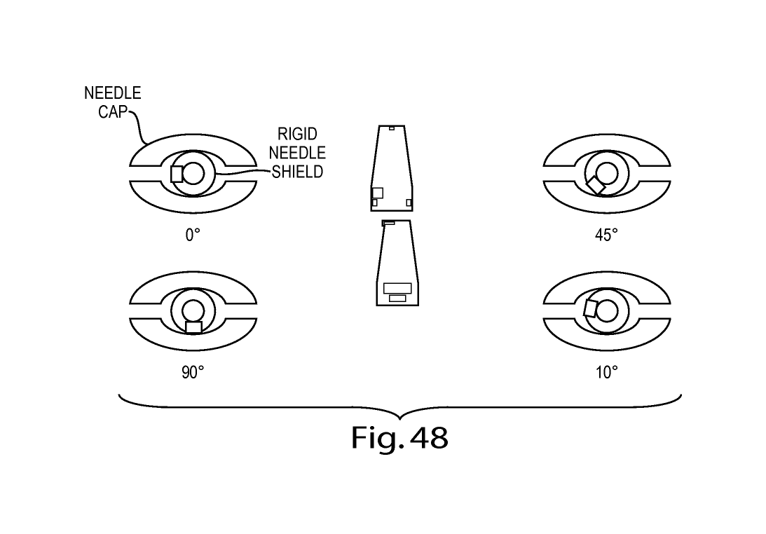

A total of 300 exemplary syringes (100 syringes from each syringe type, 25 syringes at each orientation) were tested in which the rigid needle shields were at different exemplary angular orientations (about 0°, 45°, 90°, 10°) with respect to the proximal cap. FIG. 48 illustrates the different angular orientations tested.

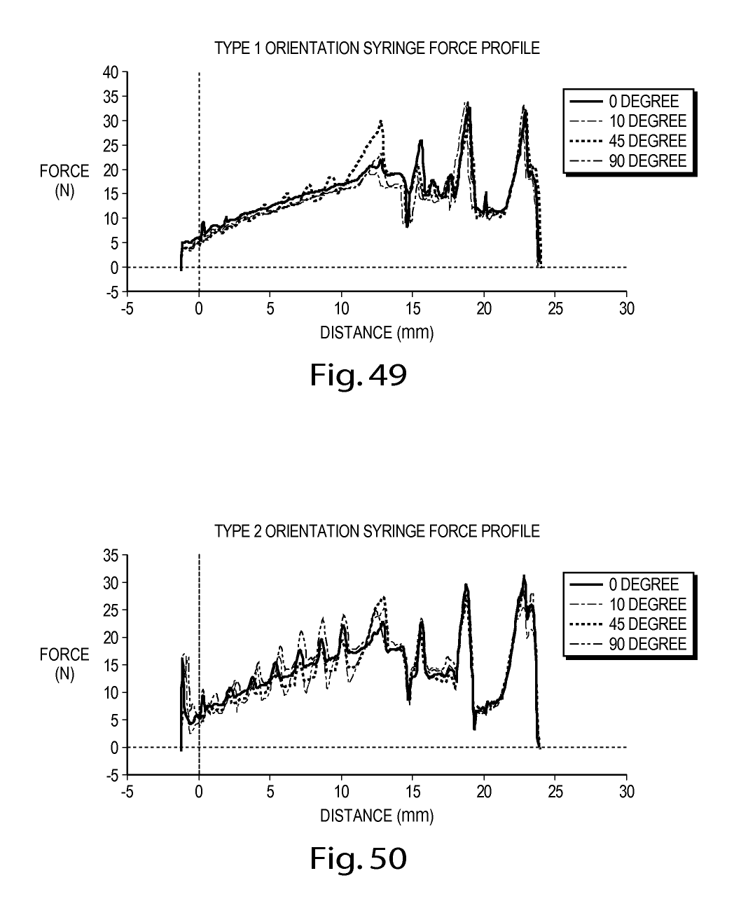

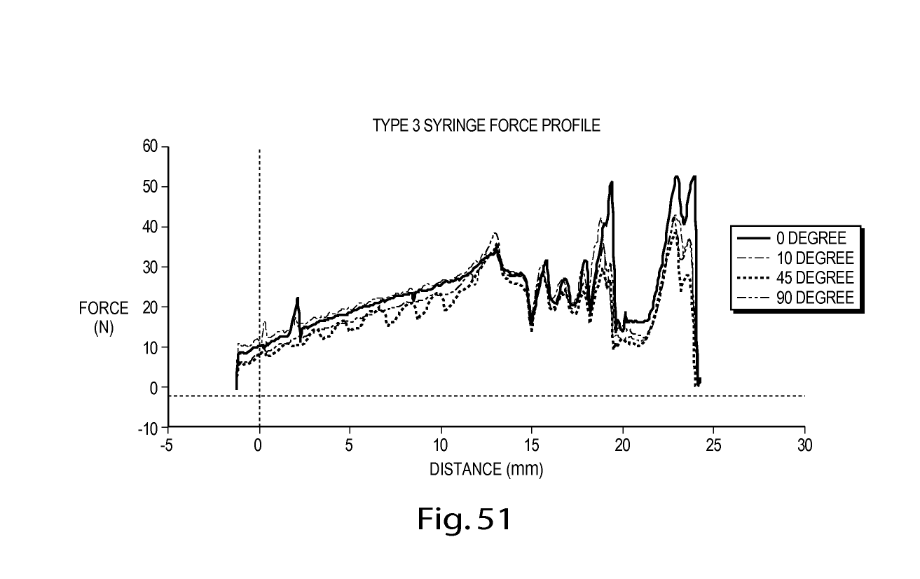

Exemplary syringe insertion systems inserted all 300 syringes past the friction point in the proximal cap with no significant differences observed in the force profiles for the different angular orientations tested. However, the 45° orientation resulted in a saw-tooth insertion force profile for the Type 1 and Type 3 syringes, due to the ribs on the conical part of the rigid needle shield that engaged with the friction point in the proximal cap. Insertion of the Type 2 syringes resulted in saw-tooth force profile in all four orientations. FIGS. 49-51 illustrate exemplary force profiles generated at the four exemplary orientations for the Type 1, Type 2 and Type 3 syringes, respectively.

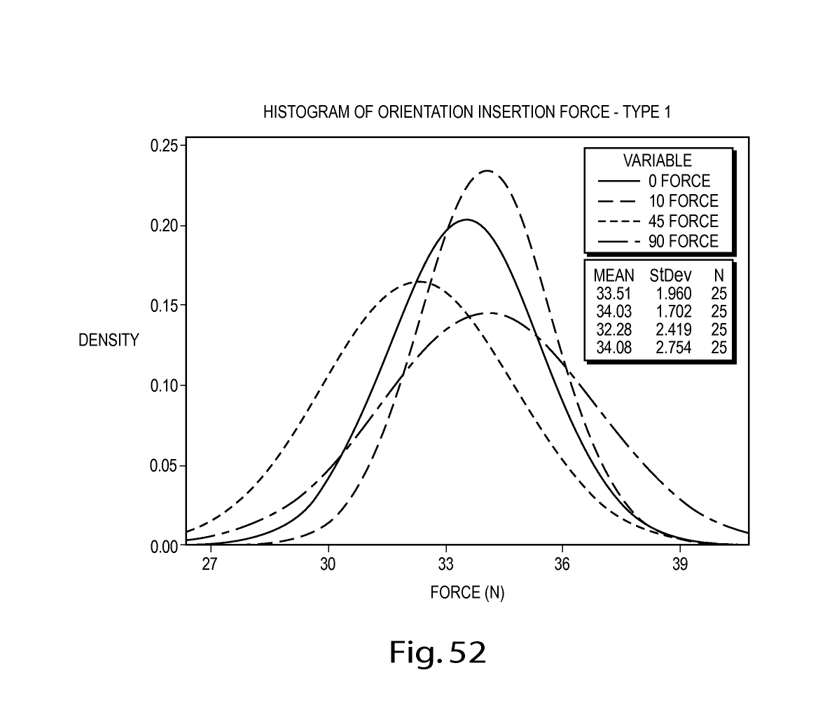

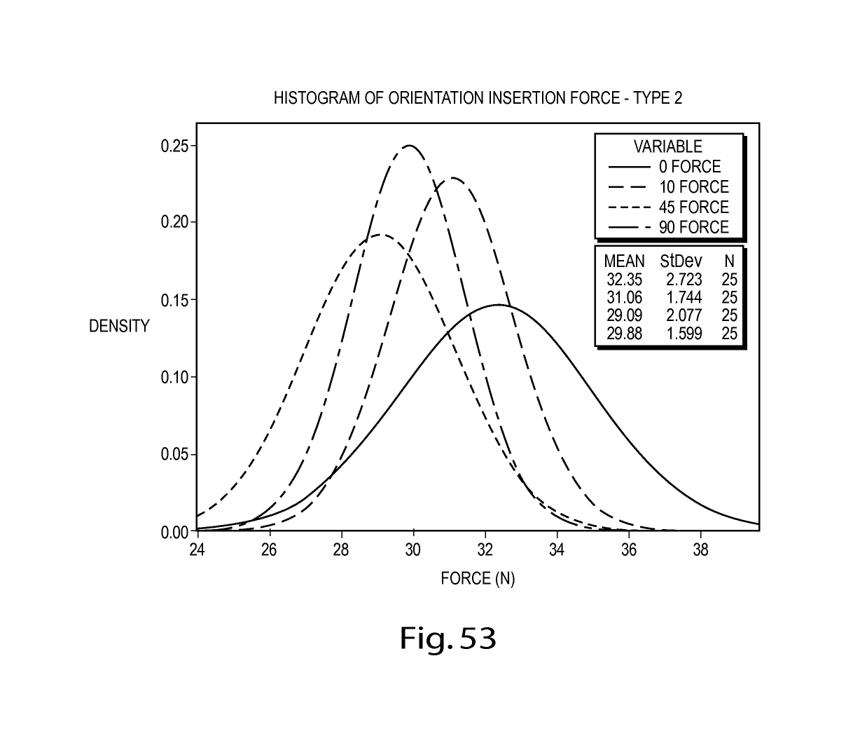

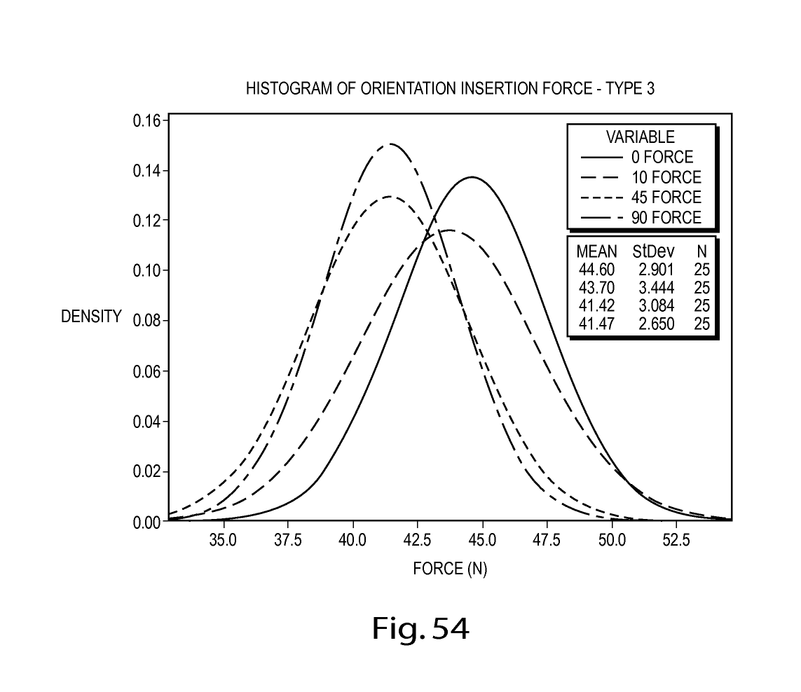

FIGS. 52-54 illustrate a histogram of the number of tested Type 1, Type 2 and Type 3, respectively, syringes (y-axis) that showed different exemplary maximum force values (x-axis) at the four exemplary orientations. The Type 3 syringes experienced the highest maximum forces during insertion with a maximum force of about 44.60 N (±2.9), the Type 1 syringes experienced intermediate maximum forces with a maximum force of about 34.08 N (±2.8), and the Type 2 syringes experienced the lowest maximum forces with a maximum force of about 32.35 N (±2.7). No substantial force differences were observed for the four different orientations tested. The maximum forces experienced by the Type 2 and Type 3 syringes were at the 0° orientation, while the maximum forces experienced by the Type 1 syringes were at the 90° orientation.

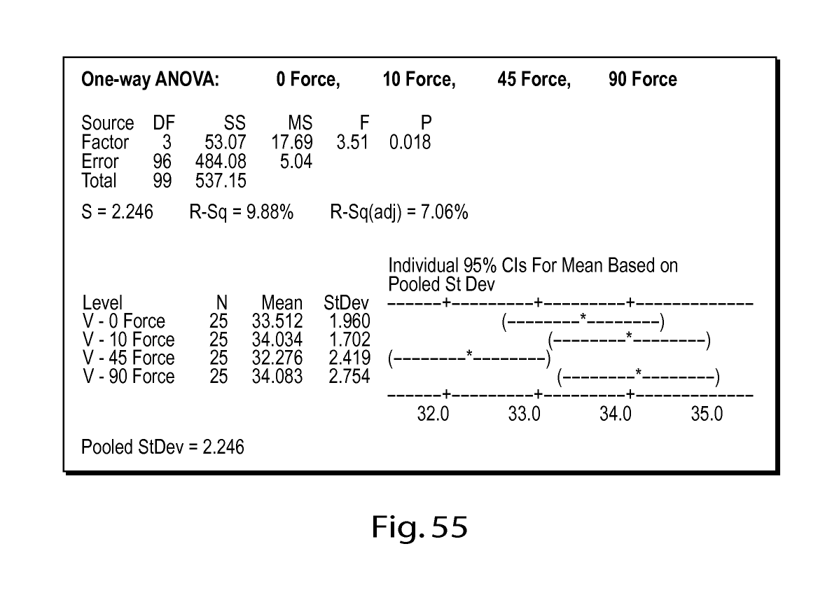

For the tested Type 1 syringes, the 90° orientation resulted in the highest insertion force of about 34.08 N (±2.8), with the 10° orientation resulting in a comparable force of about 34.03 N (±1.7). A 0.5 N reduction was observed at the 0° orientation and a 2 N reduction was observed at the 45° orientation. ANOVA analysis for the tested Type 1 syringes indicated that the different orientations produced significant differences between the 45° orientation and the 10°/45°/90° orientations, as shown in FIG. 55.

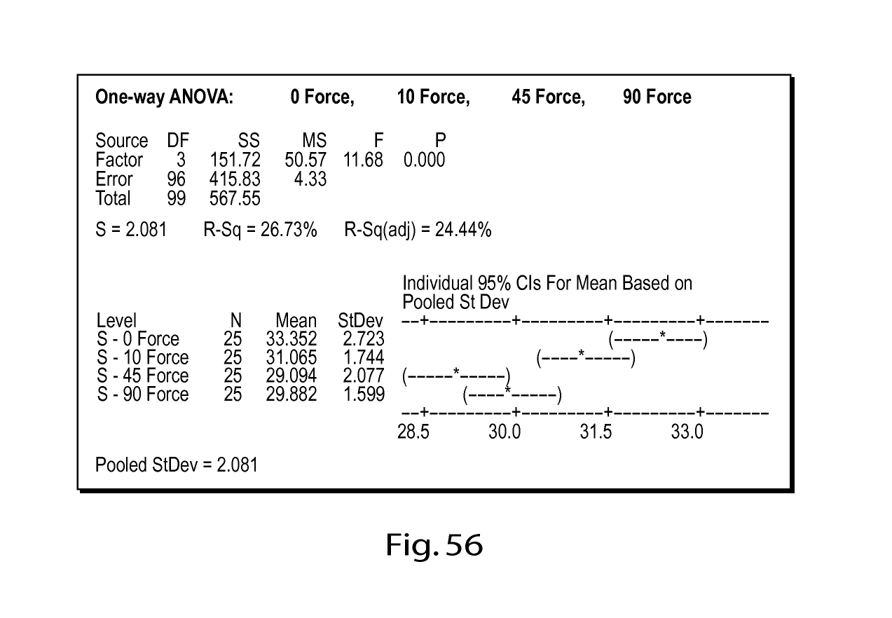

For the tested Type 2 syringes, the 0° orientation resulted in the highest insertion force of about 32.35 N (±2.7). A 2 N reduction was observed at the 45° and 90° orientations and a 1 N reduction was observed at the 10° orientation for the tested Type 2 syringes. ANOVA (analysis of variance) analysis for the tested Type 2 syringes indicated that the different orientations produced significant differences between the 0° orientation and the 10°/45°/90° orientations, as shown in FIG. 56.

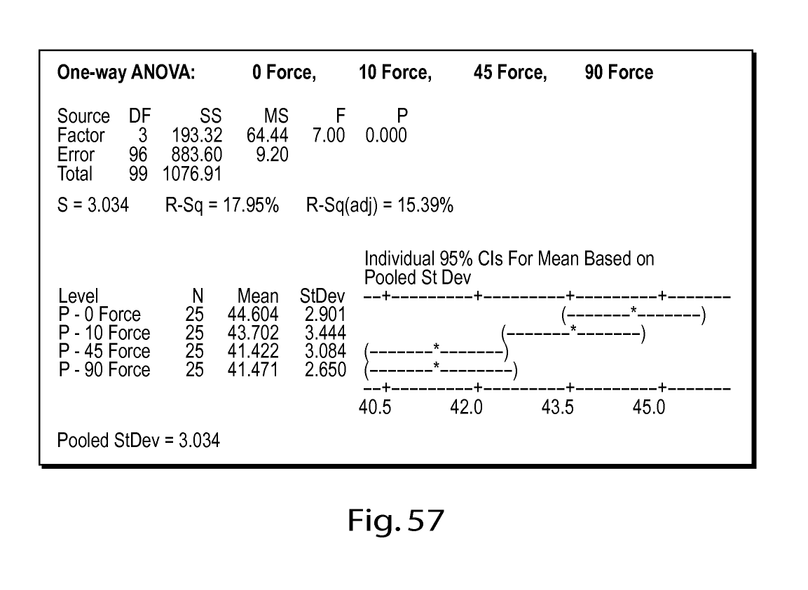

For the tested Type 3 syringes, the 0° orientation resulted in the highest insertion force of about 44.60 N (±2.9). A 3 N reduction was observed at the 45° and 90° orientations and a 1 N reduction was observed at the 10° orientation. ANOVA analysis for the tested Type 3 syringes indicated that the different orientations produced significant differences between the 0°/10° orientations and the 45°/90° orientations, as shown in FIG. 57.

As no significant visual difference was observed among the orientations, all four orientations were evaluated to determine exemplary forces required to remove the proximal cap from the housing of the automatic injection device. Twenty-five autoinjector subassemblies were tested for proximal cap removal force for each syringe type, and all four orientations were represented in the testing.

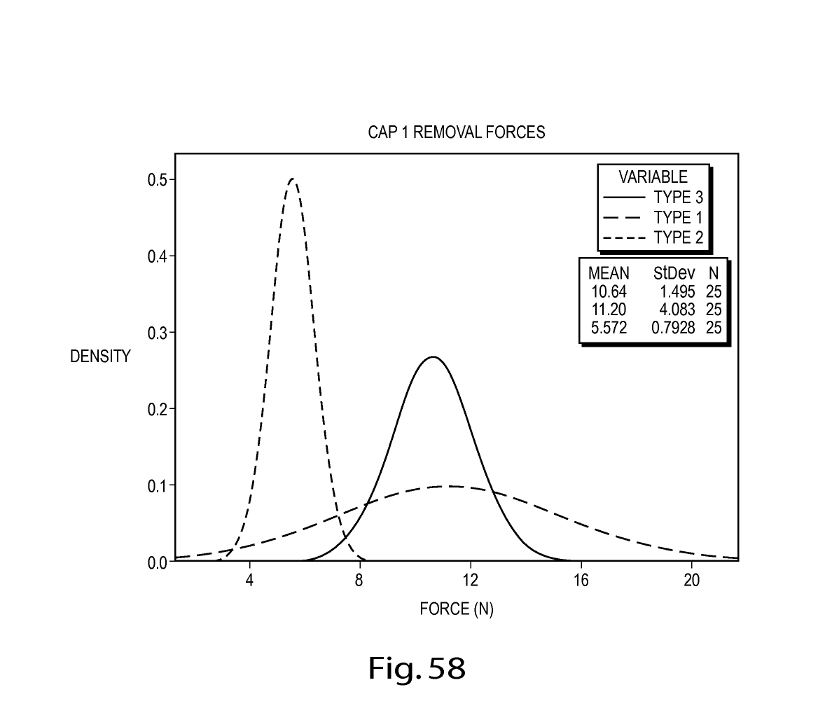

FIG. 58 illustrates a histogram of the number of tested syringes (y-axis) against different proximal cap removal forces in N (x-axis) for the three tested syringes types. The Type 1 syringes showed the highest overall removal forces with an average force of about 11.2 N (±4.08), the Type 3 syringes showed intermediate removal forces with an average force of about 10.6 N (±1.50), and the Type 2 syringes showed the lowest removal forces with an average force of about 5.6 N (±0.79).

### A. (iv) Testing Insertion of Syringes of Different Lengths

To ensure that exemplary syringes insertion systems are able to accurately insert a rigid needle shield and syringe to a specified depth regardless of the syringe length, syringes of different exemplary lengths were tested.

In one method, to increase the length of some test syringes for testing purposes, a grommet (rubber O-ring) was attached at the distal end of the syringe. Exemplary rubber grommets had widths of about 1.75, 2.60 and 3.63 mm. In another method, to increase the length of the test syringes for testing purposes, a steel ring was placed at the proximal end of the syringe below the rigid needle shield. Exemplary steel rings had widths of about 1.57, 2.67 and 3.65 mm. Exemplary rubber grommets and steel rings used in elongating exemplary syringes are summarized in Table 4.

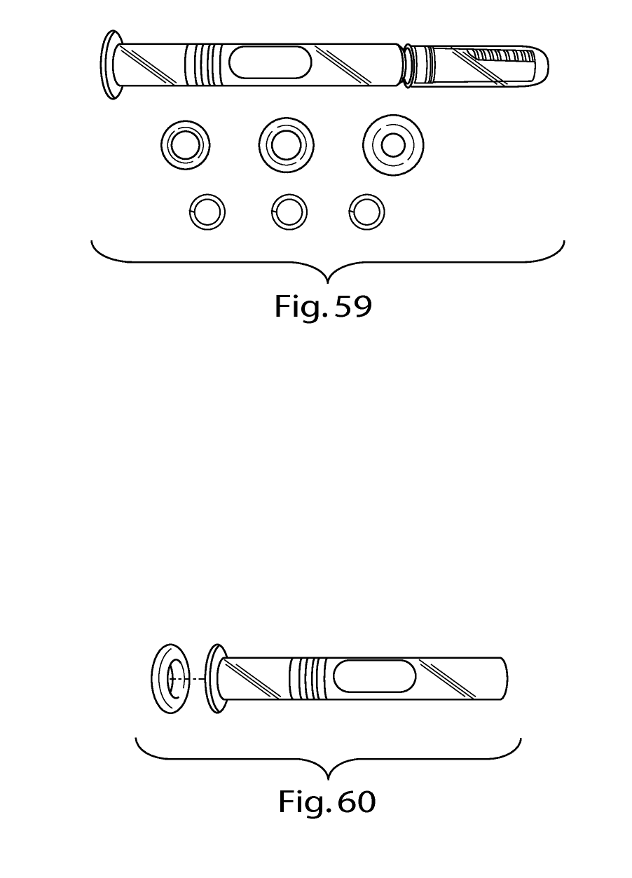

FIG. 59 illustrates an exemplary syringe fitted with a rigid needle shield, exemplary rubber grommets and exemplary steel rings used in testing syringe insertion.

FIG. 60 illustrates an exemplary rubber grommet placed at a distal end of an exemplary syringe to increase the length of the syringe.

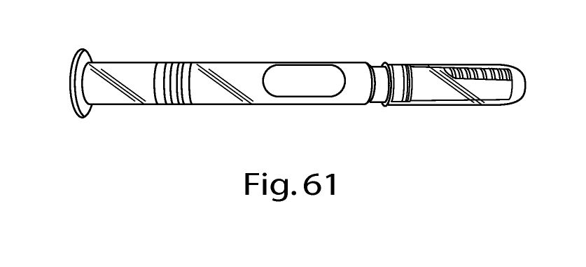

FIG. 61 illustrates a steel ring placed at a proximal end of an exemplary syringe below the rigid needle shield to increase the length of the syringe.

TABLE 4
Summary of exemplary items used
in elongating exemplary syringes

| Type of Elongation Item | Width (mm) |
|---|---|
| Rubber grommet | 1.75 |
| Rubber grommet | 2.60 |
| Rubber grommet | 3.63 |
| Steel ring | 1.57 |
| Steel ring | 2.67 |
| Steel ring | 3.65 |

In another method, to decrease the length of some test syringes for testing purposes, the rigid needle shield was cut at the proximal end to reduce the overall syringe insertion distance. Table 5 summarizes different items used in lengthening exemplary syringes and cuts applied to the rigid needle shield. Table 5 includes the length of the syringes before and after elongation/shortening, as well as the insertion depths of the rigid needle shield in the proximal cap as achieved by an exemplary syringe insertion process.

TABLE 5
Summary of exemplary syringe lengths
used in testing syringe insertion

| | | | | Rigid Needle |
|---|---|---|---|---|
| | | | | Shield (RNS) |
| | Original | | | Insertion Depth |
| Syringe | Length | Type of Adjustment to | Modified | in Proximal cap |
| Type | (mm) | Syringe Length | Length (mm) | (mm) |
| Type 2 | 81.58 | 1.75 mm grommet | 83.33 | 7.89 |
| Type 3 | 81.71 | 1.75 mm grommet | 83.46 | 8.03 |
| Type 1 | 81.33 | 1.75 mm grommet | 83.08 | 7.88 |
| Type 2 | 81.56 | 2.60 mm grommet | 84.16 | 7.94 |
| Type 3 | 81.58 | 2.60 mm grommet | 84.18 | 7.91 |
| Type 1 | 81.52 | 2.60 mm grommet | 84.12 | 7.96 |
| Type 2 | 81.48 | 3.63 mm grommet | 85.11 | 5.69 |
| Type 3 | 81.61 | 3.63 mm grommet | 85.24 | 5.3 |
| Type 1 | 81.27 | 3.63 mm grommet | 84.9 | 5.48 |
| Type 2 | 81.41 | 1.57 m steel ring | 84.12 | 7.96 |
| Type 3 | 81.65 | 1.57 m steel ring | 82.72 | 8 |
| Type 1 | 81.14 | 1.57 m steel ring | 83.8 | 8.01 |
| Type 2 | 81.5 | 2.67 mm steel ring | 83.84 | 7.94 |
| Type 3 | 81.66 | 2.67 mm steel ring | 84.27 | 3.67 |
| Type 1 | 80.92 | 2.67 mm steel ring | 84.52 | 2.64 |
| Type 2 | 81.12 | 3.65 mm steel ring | 85.27 | 3.05 |
| Type 3 | 81.64 | 3.65 mm steel ring | 84.78 | 3.3 |
| Type 1 | 81.29 | 3.65 mm steel ring | 84.11 | 2.94 |
| Type 2 | 81.31 | Cut RNS | 80.54 | 8.56 |
| Type 3 | 81.58 | Cut RNS | 80.69 | 8.67 |
| Type 1 | 81.31 | Cut RNS | 80.48 | 8.47 |
| Type 2 | 81.33 | Cut RNS | 79.79 | 8.28 |
| Type 3 | 81.6 | Cut RNS | 80.15 | 8.74 |
| Type 1 | 81.04 | Cut RNS | 79.74 | 8.73 |
| Type 2 | 81.28 | Cut RNS | 77.07 | 13.71 |
| Type 3 | 81.56 | Cut RNS | 78.75 | 11.19 |
| Type 1 | 81.2 | Cut RNS | 78.21 | 10.82 |

Experimental results indicated that all of the tested syringes were inserted to the specified insertion depth, except for the syringes with lengths greater than about 84 mm. At these high lengths, insertion was initiated prior to the normal starting point, which caused the trigger condition to be detected after the rigid needle shield passed the friction point in the proximal cap. As a result, the system did not register the trigger condition and proceeded to insert the syringe farther into the housing. The overall test results indicated that variability in the syringes, variability in manufacturing and processes as well as variability in materials can be accommodated in exemplary syringe insertion systems when the syringes are within the supplier specifications.

Syringes with broken flanges were tested using exemplary syringe insertion systems and compared to control syringes that did not have broken flanges. The syringes with broken flanges were inserted to optimal insertion depths by exemplary insertion systems, and the force profiles for the broken syringes did not show significant differences compared to the control syringes. The test results indicated that the functioning and performance of exemplary insertion systems were not affected by broken flanges in the syringes.

Cracked syringes held together by the syringe label were tested using exemplary syringe insertion systems and compared to intact control syringes. The cracked syringes were inserted to optimal insertion depths by exemplary insertion systems, and the force profiles for the cracked syringes did not show significant differences compared to the control syringes. The test results indicated that the functioning and performance of exemplary insertion systems were not affected by cracked syringes.

### B. Second Set of Experiments

### B. (i) One-Step Insertion Phase

In a set of experiments, exemplary syringe insertion systems were configured to operate in two phases: an earlier approach phase and a later insertion phase. The speed and acceleration/deceleration settings of the syringe insertion process were set based on the two phases. In some exemplary embodiments, the approach phase had an acceleration/deceleration of about 20,000 mm/s² and a speed of about 2,200 mm/min, and the insertion phase had an acceleration/deceleration of about 80,000 mm/s² and a speed of about 7,500 mm/min. In some exemplary embodiments, the insertion phase had an acceleration/deceleration of about 80,000 mm/s² and a speed of about 3,750 mm/min.

Exemplary trigger conditions were set using a ComoView® control monitor manufactured by the Kistler Group. The trigger conditions each included a trigger force (Y₂) and a trigger hysteresis (Y_{hysteresis}).

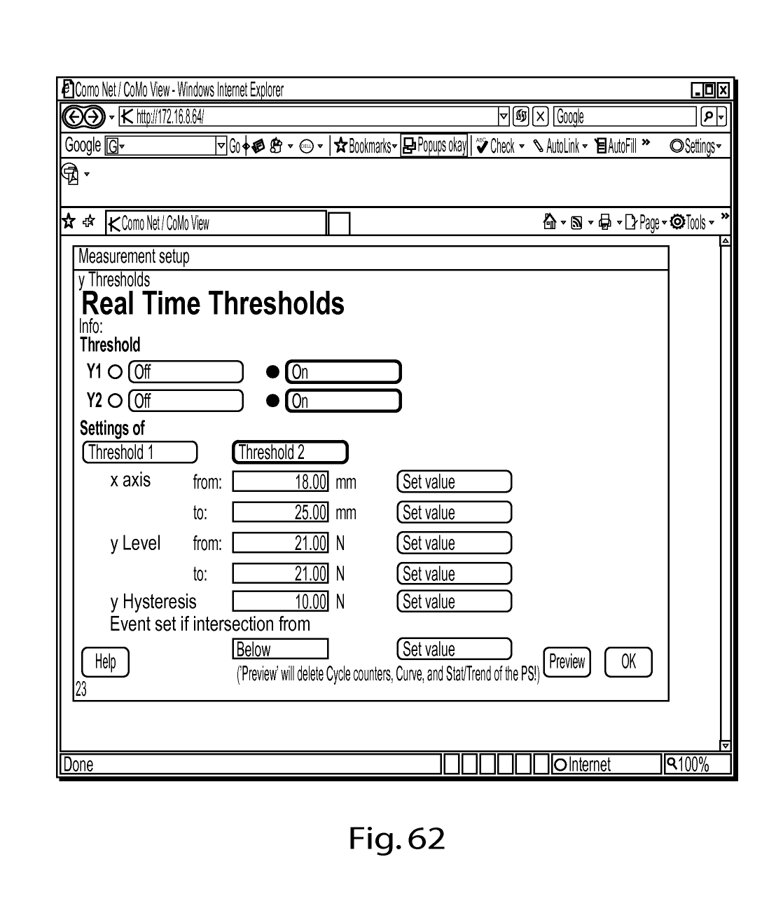

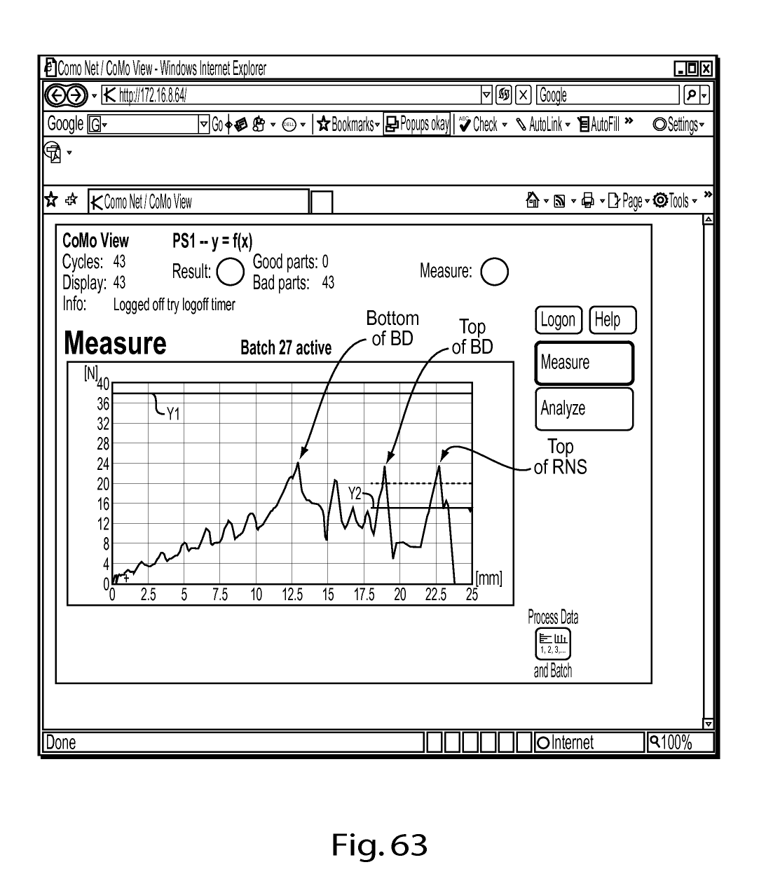

FIG. 62 illustrates a user interface showing a trigger force setting of about 21 N and a trigger hysteresis setting of about 10 N from a “below” approach. Since the insertion speed was fast in this test, the trigger condition was set for the force peak that corresponded to the distal end of the Becton Dickinson (BD) logo on the rigid needle shield, as shown in FIG. 63 (“Top of BD”).

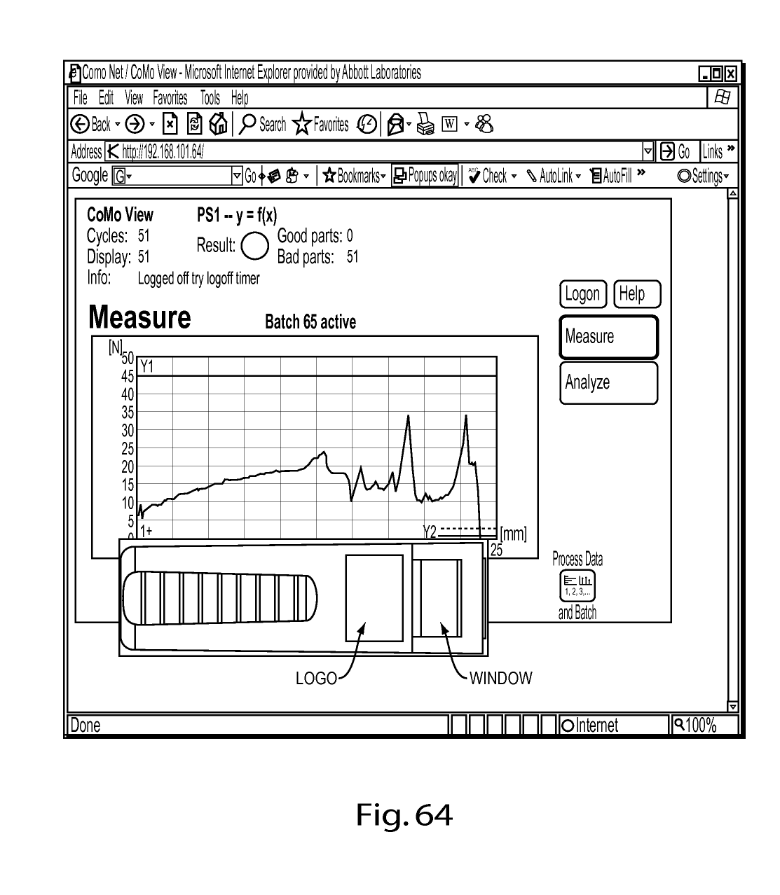

FIG. 64 illustrates an exemplary rigid needle shield and corresponding force profile, in which different features on the shield correspond to different features in the force profile. A trigger plus move was set for about 4.0 mm, i.e. the motion generator was operated to move the syringe an additional 4.0 mm after detection of the trigger condition. This trigger plus move allowed for a more accurate stopping point.

The following steps were followed in testing an exemplary syringe insertion system using the specified parameters. In a first step, the syringe was inserted into the housing of the automatic injection device before placing the assembly into the syringe insertion system. In a second step, the assembly was placed in and coupled to the syringe insertion system. In a third step, an approach phase was performed in which a mechanical member provided with a force sensor approached the distal end of the syringe. In a fourth step, the approach phase was ended when the force sensor reached the distal end of the syringe. In a fifth step, an insertion phase was performed in which the mechanical member with the force sensor was used to drive the syringe into the housing. In a sixth step, the insertion phase was ended when the trigger condition was detected. In a seventh step, the trigger plus move was performed to move the syringe an additional 4.0 mm into the housing after detection of the trigger condition. In an eighth step, the syringe insertion process was subsequently ended, and the insertion depth of the syringe in the housing was measured with calipers.

One hundred syringes were inserted to a specified depth to have the distal end of the rigid needle shield sit at the friction point in the proximal cap. By selecting a peak in the force profile prior to the final characteristic peak and using a trigger plus move to move the syringe a farther distance after the trigger condition, a more accurate insertion depth was achieved. The insertion depth of the rigid needle shield in the proximal cap was measured for each sample and parameters from the force profile were identified.

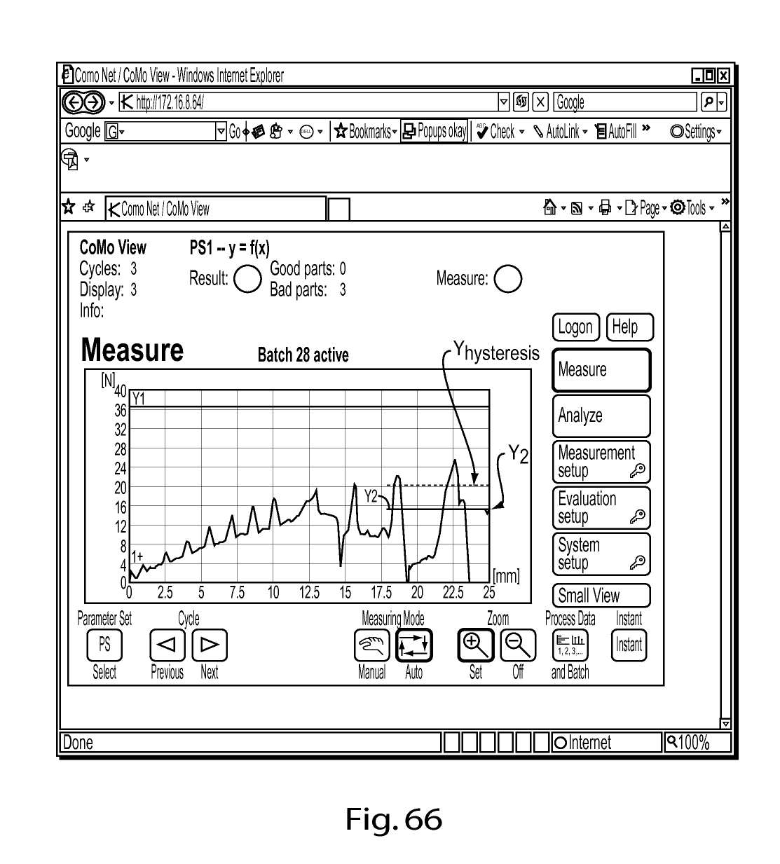

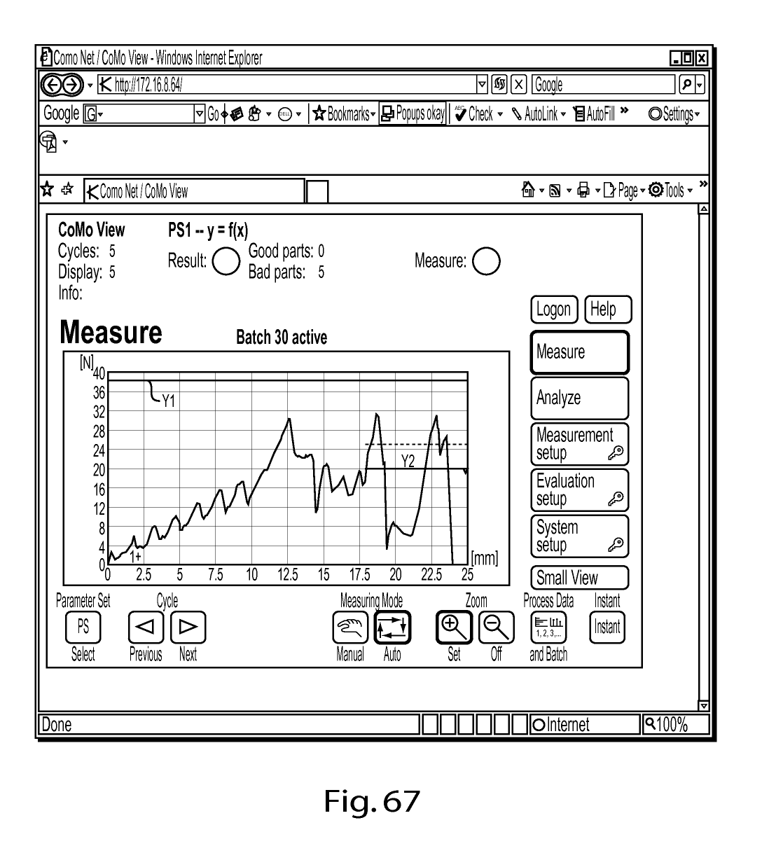

FIG. 63 illustrates an exemplary force profile generated during the syringe insertion process for the Type 1 syringes. FIG. 66 illustrates an exemplary force profile generated during the syringe insertion process for the Type 2 syringes. FIG. 67 illustrates an exemplary force profile generated during the syringe insertion process for the Type 3 syringes.

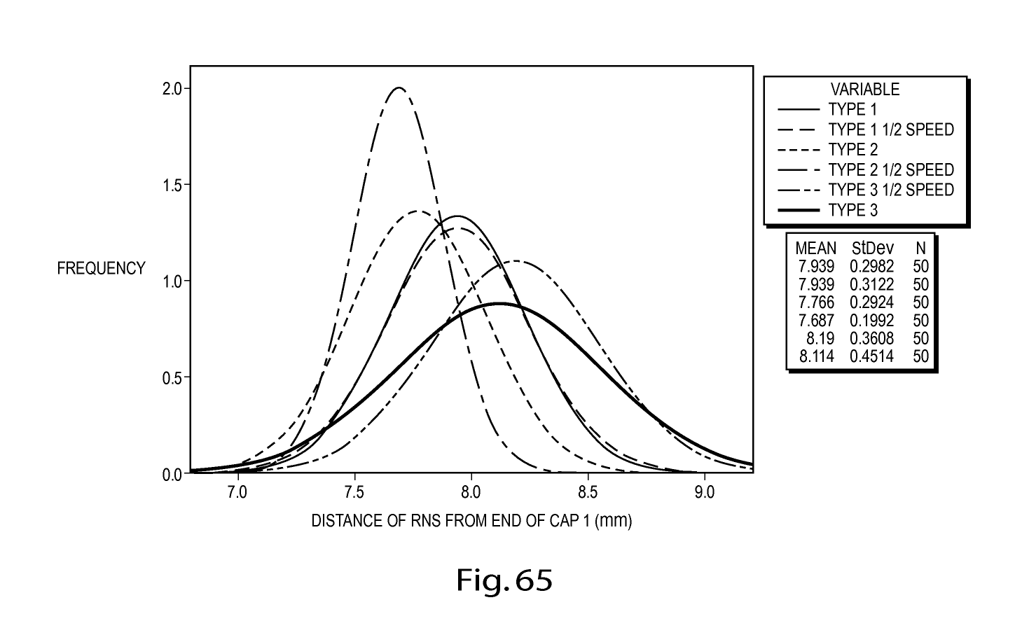

FIG. 65 illustrates a histogram of the number of tested syringes (y-axis) that achieved different exemplary insertion depths in mm (x-axis) based on the above-described syringe insertion settings.

Table 6 summarizes the experimental results.

TABLE 6
Summary of insertion depths (in mm) achieved
by exemplary syringe insertion systems

| | Syringe | Syringe | Syringe | | Syringe | Syringe | Syringe |
|---|---|---|---|---|---|---|---|
| Sample # | Type 1 | Type 2 | Type 3 | Sample # | Type 1 | Type 2 | Type 3 |
| 1 | 7.70 | 7.64 | 8.00 | 51 | 7.92 | 7.92 | 7.99 |
| 2 | 7.77 | 7.49 | 7.82 | 52 | 8.04 | 7.85 | 7.96 |
| 3 | 7.7 | 7.51 | 7.95 | 53 | 7.85 | 7.71 | 7.91 |
| 4 | 7.86 | 7.60 | 7.92 | 54 | 8.70 | 7.85 | 7.95 |
| 5 | 7.78 | 7.77 | 7.99 | 55 | 8.59 | 7.48 | 7.97 |
| 6 | 7.97 | 7.72 | 7.72 | 56 | 7.91 | 7.60 | 7.91 |
| 7 | 7.98 | 8.37 | 7.78 | 57 | 7.77 | 7.58 | 8.55 |
| 8 | 7.92 | 7.66 | 8.51 | 58 | 7.66 | 7.41 | 7.83 |
| 9 | 8.67 | 7.62 | 7.93 | 59 | 7.90 | 7.64 | 8.51 |
| 10 | 7.74 | 7.78 | 7.91 | 60 | 7.74 | 7.70 | 7.93 |
| 11 | 7.87 | 7.50 | 7.01 | 61 | 7.83 | 7.78 | 7.89 |
| 12 | 7.87 | 7.61 | 8.32 | 62 | 7.64 | 7.68 | 7.87 |
| 13 | 7.83 | 7.67 | 7.78 | 63 | 7.78 | 7.68 | 8.70 |
| 14 | 7.55 | 7.82 | 7.19 | 64 | 7.60 | 7.78 | 8.06 |
| 15 | 8.89 | 7.64 | 7.66 | 65 | 7.68 | 7.82 | 8.69 |
| 16 | 7.94 | 7.63 | 7.36 | 66 | 7.58 | 8.00 | 8.64 |
| 17 | 7.83 | 7.62 | 7.92 | 67 | 7.48 | 7.76 | 7.81 |
| 18 | 7.85 | 7.72 | 7.79 | 68 | 7.94 | 7.49 | 8.03 |
| 19 | 7.56 | 7.76 | 8.02 | 69 | 7.79 | 7.93 | 7.87 |
| 20 | 8.50 | 7.77 | 9.05 | 70 | 7.52 | 7.92 | 8.05 |
| 21 | 7.91 | 7.49 | 8.90 | 71 | 7.74 | 7.45 | 7.92 |
| 22 | 7.93 | 8.00 | 8.11 | 72 | 7.93 | 7.62 | 8.62 |
| 23 | 7.81 | 7.61 | 7.99 | 73 | 7.67 | 7.82 | 8.66 |
| 24 | 7.92 | 7.61 | 8.90 | 74 | 7.82 | 7.68 | 7.97 |
| 25 | 7.88 | 7.63 | 8.00 | 75 | 7.85 | 7.83 | 7.87 |
| 26 | 7.72 | 8.41 | 8.74 | 76 | 7.76 | 7.84 | 8.05 |
| 27 | 7.68 | 7.15 | 8.80 | 77 | 8.45 | 7.46 | 7.99 |
| 28 | 8.90 | 7.59 | 8.74 | 78 | 7.72 | 8.35 | 8.41 |
| 29 | 8.67 | 7.73 | 7.97 | 79 | 7.91 | 7.45 | 7.85 |
| 30 | 8.06 | 7.68 | 8.15 | 80 | 7.76 | 7.78 | 7.95 |
| 31 | 7.93 | 7.62 | 8.01 | 81 | 7.82 | 7.50 | 7.97 |
| 32 | 7.86 | 7.63 | 7.91 | 82 | 7.98 | 7.61 | 8.37 |
| 33 | 7.92 | 8.89 | 7.86 | 83 | 8.18 | 7.78 | 8.88 |
| 34 | 7.86 | 8.11 | 8.10 | 84 | 7.91 | 7.73 | 7.84 |
| 35 | 7.83 | 7.92 | 7.88 | 85 | 7.75 | 7.78 | 8.72 |
| 36 | 7.58 | 7.63 | 7.98 | 86 | 7.92 | 7.68 | 8.49 |
| 37 | 7.91 | 7.63 | 8.66 | 87 | 8.87 | 7.63 | 8.67 |
| 38 | 8.03 | 7.87 | 8.61 | 88 | 7.85 | 7.53 | 8.01 |
| 39 | 7.98 | 7.80 | 8.71 | 89 | 8.50 | 7.64 | 7.89 |
| 40 | 8.05 | 7.66 | 8.66 | 90 | 7.79 | 7.76 | 7.92 |
| 41 | 8.09 | 7.81 | 7.91 | 91 | 8.54 | 7.85 | 8.02 |
| 42 | 7.85 | 7.65 | 7.87 | 92 | 7.96 | 7.68 | 7.84 |
| 43 | 7.99 | 7.90 | 8.69 | 93 | 7.82 | 7.80 | 8.92 |
| 44 | 8.07 | 8.64 | 7.85 | 94 | 7.78 | 7.78 | 8.77 |
| 45 | 7.92 | 7.74 | 8.02 | 95 | 8.01 | 7.27 | 8.52 |
| 46 | 7.70 | 7.67 | 7.88 | 96 | 8.06 | 7.80 | 8.75 |
| 47 | 7.94 | 7.78 | 7.86 | 97 | 8.62 | 7.57 | 7.93 |
| 48 | 7.78 | 7.77 | 8.74 | 98 | 8.07 | 7.16 | 7.91 |
| 49 | 7.57 | 8.07 | 7.91 | 99 | 7.88 | 7.43 | 8.76 |
| 50 | 7.78 | 7.72 | 8.65 | 100 | 8.10 | 7.49 | 7.91 |
| Average | 7.94 | 7.77 | 8.11 | Average | 7.94 | 7.69 | 8.19 |
| StDev | 0.30 | 0.29 | 0.45 | StDev | 0.31 | 0.20 | 0.36 |

At the 7,000 mm/min insertion speed, the average insertion depth for the Type 1 syringes was about 7.94 mm (±0.30), for the Type 2 syringes about 7.77 mm (±0.29) and for the Type 3 syringes about 8.11 mm (±0.45). At the 3,750 mm/min insertion speed, the average insertion depth for the Type 1 syringes was about 7.94 mm (±0.31), for the Type 2 syringes about 7.69 mm (±0.20) and for the Type 3 syringes about 8.19 mm (±0.36). All tested rigid needle shields were inserted past the friction point in the proximal cap. The differences in the insertion depths were due to variability in the rigid needle shields and the speeds at which the motion generator stopped.

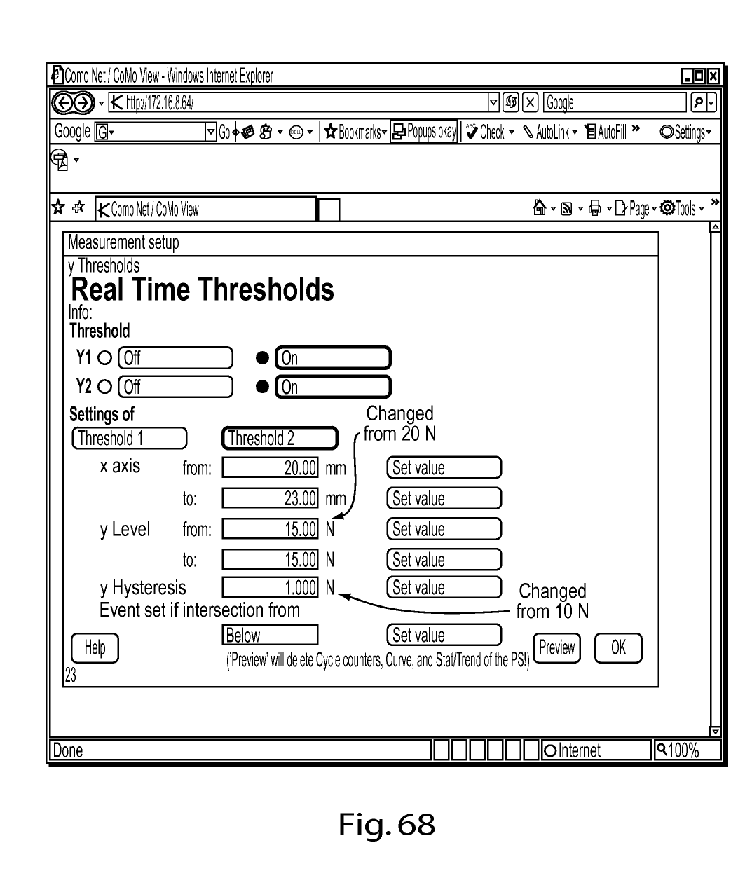

In a different set of tests, the trigger condition settings were changed from 20 N to 15 N for Y₂ and from 10 N to 1 N for Y_{Hysteresis}, as shown in the user interface of FIG. 68. The new settings made it possible to set a start depth of approximately 6.0 mm and adjust the distance moved beyond the trigger condition for each desired insertion depth. For different desired insertion depths, the motion generator settings were changed to move the syringe and rigid needle shield a farther distance after detecting the trigger condition in a trigger plus move, as summarized in Table 7. The desired insertion depth in this example is measured from the proximal end of the proximal cap to the proximal end of the rigid needle shield in the assembled automatic injection device. This farther distance is denoted as “Servo Setting Zone E.”

TABLE 7
Summary of desired insertion depths and
corresponding motion generator settings

| Desired Insertion Depth (mm) | Servo Setting Zone E (mm) |
|---|---|
| 6.0 | 4.0 |
| 6.5 | 3.5 |
| 7.0 | 3.0 |
| 7.5 | 2.5 |
| 8.0 | 2.0 |
| 8.5 | 1.5 |
| 9.0 | 1.0 |
| 9.5 | 0.5 |
| 10.0 | 0.0 |

A desired insertion depth range of about 6.0 mm to about 10.0 mm was used in 0.5 mm increments. This provided four exemplary set points above and four exemplary set points below the nominal depth. Twenty Type 1 syringes were inserted at each 0.5 mm interval. The rigid needle shield of each test syringe was measured, along with the insertion depths of the rigid needle shield in the proximal cap

The test syringes inserted beyond the 7 mm insertion depth showed greater errors as the syringe barrel was inserted past the friction point in the proximal cap. Since the glass of the syringe body was smooth, the syringe could be forced back in the distal direction out of the friction point.

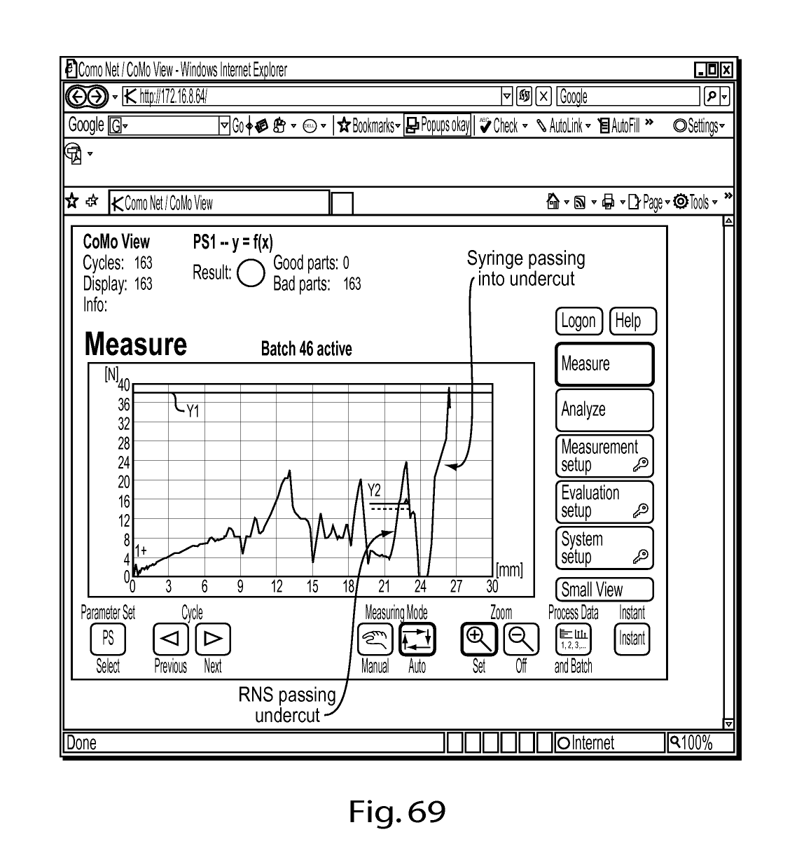

FIG. 69 illustrates the force profile generated by the 6.0 mm desired depth. FIG. 69 shows a high and sharp final peak that corresponds to the syringe barrel beginning to pass by the friction point in the proximal cap. The third peak immediately before the final peak corresponds to the distal end of the rigid needle shield passing the friction point in the proximal cap.

The overall length of the rigid needle shield was not a significant factor into the variability of the insertion depths achieved.

For a desired insertion depth of about 10 mm, the average actual insertion depth of twenty Type 1 syringes was about 9.93 mm (±0.13). For a desired insertion depth of about 9.5 mm, the average actual insertion depth of twenty Type 1 syringes was about 9.65 mm (±0.17). For a desired insertion depth of about 9.0 mm, the average actual insertion depth was about 9.13 mm (±0.18). For a desired insertion depth of about 8.5 mm, the average actual insertion depth was about 8.14 mm (±0.47). For a desired insertion depth of about 8.0 mm, the average actual insertion depth was about 7.78 mm (±0.22). For a desired insertion depth of about 7.5 mm, the average actual insertion depth was about 7.21 mm (±0.31). For a desired insertion depth of about 7.0 mm, the average actual insertion depth was about 7.21 mm (±0.15). For a desired insertion depth of about 6.5 mm, the average actual insertion depth was about 7.31 mm (±0.25). For a desired insertion depth of about 6.0 mm, the average actual insertion depth was about 6.17 mm (±0.63).

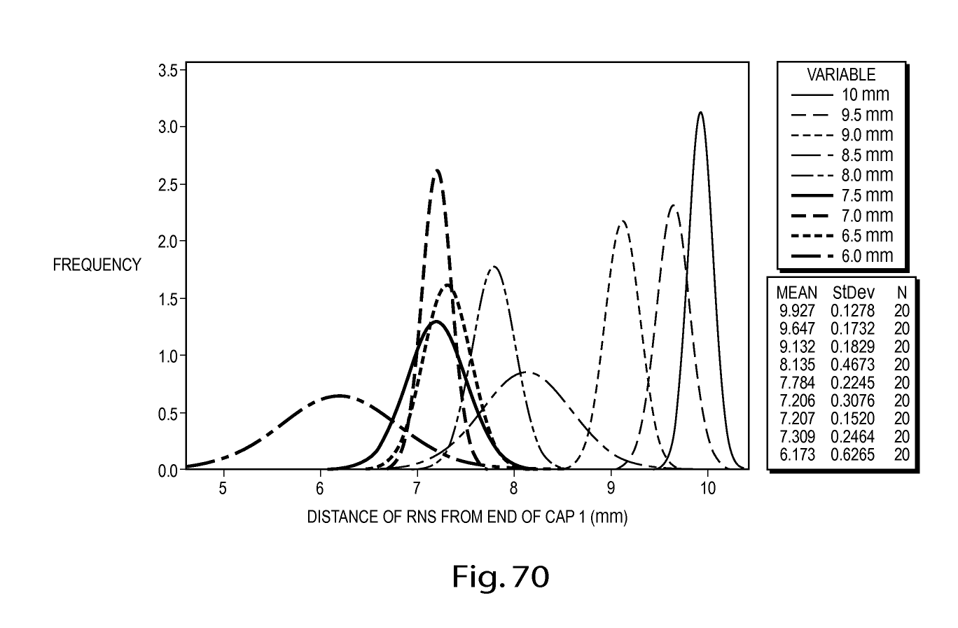

FIG. 70 illustrates a histogram of the number of tested syringes (y-axis) against the actual insertion depths achieved in mm (x-axis) for different desired insertion depths. Table 8 summarizes the experimental results shown in FIG. 70.

TABLE 8
Summary of desired and actual insertion depths

| | Desired Insertion | Rigid Needle Shield | Actual Insertion |
|---|---|---|---|
| Sample # | Depth (mm) | Length (mm) | Depth (mm) |
| 1 | 10 | 26.22 | 9.78 |
| 2 | 10 | 26.45 | 9.82 |
| 3 | 10 | 26.17 | 10.1 |
| 4 | 10 | 26.16 | 9.8 |
| 5 | 10 | 26.2 | 9.99 |
| 6 | 10 | 26.22 | 10.21 |
| 7 | 10 | 26.27 | 9.97 |
| 8 | 10 | 26.29 | 9.94 |
| 9 | 10 | 26.3 | 9.77 |
| 10 | 10 | 26.27 | 9.98 |
| 11 | 10 | 26.29 | 9.88 |
| 12 | 10 | 26.29 | 9.8 |
| 13 | 10 | 26.27 | 9.78 |
| 14 | 10 | 26.25 | 9.76 |
| 15 | 10 | 26.19 | 10.06 |
| 16 | 10 | 26.24 | 9.89 |
| 17 | 10 | 26.21 | 9.98 |
| 18 | 10 | 26.19 | 9.98 |
| 19 | 10 | 26.19 | 9.97 |
| 20 | 10 | 26.3 | 10.08 |

| | |
|---|---|
| Average | 9.927 |
| Max | 10.21 |
| Min | 9.76 |
| StDev | 0.12782 |

| | | | |
|---|---|---|---|
| 21 | 9.5 | 26.35 | 9.49 |
| 22 | 9.5 | 26.34 | 9.64 |
| 23 | 9.5 | 26.29 | 9.34 |
| 24 | 9.5 | 26.29 | 9.45 |
| 25 | 9.5 | 26.27 | 9.51 |
| 26 | 9.5 | 26.33 | 9.9 |
| 27 | 9.5 | 26.32 | 9.68 |
| 28 | 9.5 | 26.31 | 9.44 |
| 29 | 9.5 | 26.28 | 9.6 |
| 30 | 9.5 | 26.21 | 9.43 |
| 31 | 9.5 | 26.28 | 9.82 |
| 32 | 9.5 | 26.26 | 9.66 |
| 33 | 9.5 | 26.19 | 9.65 |
| 34 | 9.5 | 26.21 | 9.79 |
| 35 | 9.5 | 26.23 | 9.87 |
| 36 | 9.5 | 26.21 | 9.8 |
| 37 | 9.5 | 26.25 | 9.92 |
| 38 | 9.5 | 26.23 | 9.55 |
| 39 | 9.5 | 26.24 | 9.6 |
| 40 | 9.5 | 26.24 | 9.81 |

| | |
|---|---|
| Average | 9.6475 |
| Max | 9.92 |
| Min | 9.34 |
| StDev | 0.173232 |

| | | | |
|---|---|---|---|
| 41 | 9 | 26.33 | 9.19 |
| 42 | 9 | 26.31 | 8.92 |
| 43 | 9 | 26.23 | 9.27 |
| 44 | 9 | 26.27 | 9.32 |
| 45 | 9 | 26.24 | 8.93 |
| 46 | 9 | 26.21 | 9 |
| 47 | 9 | 26.21 | 9 |
| 48 | 9 | 26.28 | 8.88 |
| 49 | 9 | 26.27 | 9.07 |
| 50 | 9 | 26.3 | 8.97 |
| 51 | 9 | 26.31 | 9.36 |
| 52 | 9 | 26.21 | 9.25 |
| 53 | 9 | 26.2 | 9.36 |
| 54 | 9 | 26.22 | 9.46 |
| 55 | 9 | 26.27 | 9.35 |
| 56 | 9 | 26.24 | 9.16 |
| 57 | 9 | 26.21 | 9.01 |
| 58 | 9 | 26.21 | 9.19 |
| 59 | 9 | 26.34 | 8.87 |
| 60 | 9 | 26.2 | 9.08 |

| | |
|---|---|
| Average | 9.132 |
| Max | 9.46 |
| Min | 8.87 |
| StDev | 0.18286 |

| | | | |
|---|---|---|---|
| 61 | 8.5 | 26.22 | 8.21 |
| 62 | 8.5 | 26.29 | 8.73 |
| 63 | 8.5 | 26.23 | 8.87 |
| 64 | 8.5 | 26.29 | 7.91 |
| 65 | 8.5 | 26.28 | 8.56 |
| 66 | 8.5 | 26.26 | 8.65 |
| 67 | 8.5 | 26.22 | 8.2 |
| 68 | 8.5 | 26.23 | 7.78 |
| 69 | 8.5 | 26.25 | 8.22 |
| 70 | 8.5 | 26.22 | 7.91 |
| 71 | 8.5 | 26.32 | 7.45 |
| 72 | 8.5 | 26.22 | 7.78 |
| 73 | 8.5 | 26.21 | 7 |
| 74 | 8.5 | 26.22 | 8.04 |
| 75 | 8.5 | 26.23 | 8.08 |
| 76 | 8.5 | 26.23 | 8.11 |
| 77 | 8.5 | 26.22 | 8.04 |
| 78 | 8.5 | 26.27 | 8.94 |
| 79 | 8.5 | 26.25 | 8.18 |
| 80 | 8.5 | 26.22 | 8.04 |

| | |
|---|---|
| Average | 8.135 |
| Max | 8.94 |
| Min | 7 |
| StDev | 0.467282 |

| | | | |
|---|---|---|---|
| 81 | 8 | 26.28 | 7.86 |
| 82 | 8 | 26.25 | 7.22 |
| 83 | 8 | 26.26 | 7.5 |
| 84 | 8 | 26.23 | 7.84 |
| 85 | 8 | 26.23 | 7.78 |
| 86 | 8 | 26.24 | 7.84 |
| 87 | 8 | 26.24 | 8.11 |
| 88 | 8 | 26.21 | 7.79 |
| 89 | 8 | 26.25 | 8.12 |
| 90 | 8 | 26.22 | 7.98 |
| 91 | 8 | 26.36 | 8 |
| 92 | 8 | 26.24 | 7.64 |
| 93 | 8 | 26.23 | 7.86 |
| 94 | 8 | 26.23 | 8.1 |
| 95 | 8 | 26.27 | 7.67 |
| 96 | 8 | 26.24 | 7.66 |
| 97 | 8 | 26.26 | 7.85 |
| 98 | 8 | 26.25 | 7.68 |
| 99 | 8 | 26.3 | 7.57 |
| 100 | 8 | 26.31 | 7.62 |

| | |
|---|---|
| Average | 7.7845 |
| Max | 8.12 |
| Min | 7.22 |
| StDev | 0.224511 |

| | | | |
|---|---|---|---|
| 101 | 7.5 | 26.23 | 7.37 |
| 102 | 7.5 | 26.35 | 7.32 |
| 103 | 7.5 | 26.28 | 6.94 |
| 104 | 7.5 | 26.21 | 6.11 |
| 105 | 7.5 | 26.28 | 7.56 |
| 106 | 7.5 | 26.2 | 6.99 |
| 107 | 7.5 | 26.24 | 7.27 |
| 108 | 7.5 | 26.2 | 7.45 |
| 109 | 7.5 | 26.22 | 7.13 |
| 110 | 7.5 | 26.23 | 7.07 |
| 111 | 7.5 | 26.29 | 7.09 |
| 112 | 7.5 | 26.25 | 7.2 |
| 113 | 7.5 | 26.22 | 7.23 |
| 114 | 7.5 | 26.21 | 7.26 |
| 115 | 7.5 | 26.26 | 7.22 |
| 116 | 7.5 | 26.25 | 7.17 |
| 117 | 7.5 | 26.23 | 7.46 |
| 118 | 7.5 | 26.18 | 7.45 |
| 119 | 7.5 | 26.24 | 7.4 |
| 120 | 7.5 | 26.27 | 7.42 |

| | |
|---|---|
| Average | 7.2055 |
| Max | 7.56 |
| Min | 6.11 |
| StDev | 0.307596 |

| | | | |
|---|---|---|---|
| 121 | 7 | 26.14 | 7.03 |
| 122 | 7 | 26.19 | 7.08 |
| 123 | 7 | 26.16 | 7.09 |
| 124 | 7 | 26.16 | 7.46 |
| 125 | 7 | 26.31 | 7.54 |
| 126 | 7 | 26.21 | 7.23 |
| 127 | 7 | 26.24 | 7.09 |
| 128 | 7 | 26.28 | 7.35 |
| 129 | 7 | 26.29 | 7.09 |
| 130 | 7 | 26.26 | 7.3 |
| 131 | 7 | 26.26 | 7.26 |
| 132 | 7 | 26.23 | 7.17 |
| 133 | 7 | 26.27 | 7.16 |
| 134 | 7 | 26.26 | 7.33 |
| 135 | 7 | 26.28 | 7.26 |
| 136 | 7 | 26.22 | 7.35 |
| 137 | 7 | 26.21 | 7.21 |
| 138 | 7 | 26.24 | 7.07 |
| 139 | 7 | 26.25 | 7.13 |
| 140 | 7 | 26.25 | 6.93 |

| | |
|---|---|
| Average | 7.2065 |
| Max | 7.54 |
| Min | 6.93 |
| StDev | 0.152048 |

| | | | |
|---|---|---|---|
| 141 | 6.5 | 26.21 | 7.3 |
| 142 | 6.5 | 26.25 | 7.13 |
| 143 | 6.5 | 26.23 | 7.56 |
| 144 | 6.5 | 26.22 | 7.2 |
| 145 | 6.5 | 26.22 | 7.38 |
| 146 | 6.5 | 26.23 | 7.56 |
| 147 | 6.5 | 26.22 | 7.21 |
| 148 | 6.5 | 26.22 | 7.64 |
| 149 | 6.5 | 26.35 | 7.15 |
| 150 | 6.5 | 26.27 | 7.41 |
| 151 | 6.5 | 26.22 | 7.23 |
| 152 | 6.5 | 26.24 | 7.35 |
| 153 | 6.5 | 26.21 | 7.53 |
| 154 | 6.5 | 26.24 | 7.31 |
| 155 | 6.5 | 26.29 | 7.3 |
| 156 | 6.5 | 26.24 | 6.95 |
| 157 | 6.5 | 26.23 | 6.81 |
| 158 | 6.5 | 26.25 | 7.73 |
| 159 | 6.5 | 26.25 | 7.52 |
| 160 | 6.5 | 26.23 | 6.9 |

| | |
|---|---|
| Average | 7.3085 |
| Max | 7.73 |
| Min | 6.81 |
| StDev | 0.246412 |

| | | | |
|---|---|---|---|
| 161 | 6 | 26.2 | 7.42 |
| 162 | 6 | 26.25 | 6.41 |
| 163 | 6 | 26.24 | 5.84 |
| 164 | 6 | 26.26 | 5.91 |
| 165 | 6 | 26.25 | 5.74 |
| 166 | 6 | 26.21 | 6.08 |
| 167 | 6 | 26.22 | 6.21 |
| 168 | 6 | 26.24 | 6.3 |
| 169 | 6 | 26.24 | 7.85 |
| 170 | 6 | 26.24 | 6.08 |
| 171 | 6 | 26.2 | 6.02 |
| 172 | 6 | 26.25 | 5.88 |
| 173 | 6 | 26.19 | 5.91 |
| 174 | 6 | 26.23 | 5.73 |
| 175 | 6 | 26.27 | 5.36 |
| 176 | 6 | 26.21 | 5.48 |
| 177 | 6 | 26.21 | 5.92 |
| 178 | 6 | 26.28 | 6.16 |
| 179 | 6 | 26.13 | 7.18 |
| 180 | 6 | 26.3 | 5.98 |

| | | Average | 6.173 |
|---|---|---|---|
| | | Max | 7.85 |
| | | Min | 5.36 |
| | | StDev | 0.626486 |
| Average | 26.24628 | | |
| Max | 26.45 | | |
| Min | 26.13 | | |
| StDev | 0.04366 | | |

### B. (ii) Two-Step Insertion Phase

The speeds used for insertion in the above-described one-step insertion phase demonstrated that a longer time was required to stop the motion generator after a trigger instruction or signal was received from the ComoView® control monitor. In the one-step insertion phase, a peak profile was selected prior to the final peak and a trigger plus move was used to move the syringe a farther distance into the housing after detecting the trigger condition.

Through experimentation, it was discovered that the accuracy, reliability and repeatability the syringe insertion depths could be improved if exemplary syringe insertion systems were configured to operate in three phases. In a set of experiments: an earlier approach phase, an intermediate fast insertion phase and a later slow insertion phase were implemented. The speed and acceleration/deceleration settings of the syringe insertion process were set based on the three phases. The fast insertion phase began after the approach phase and covered approximately 15 mm of the insertion depth at an acceleration/deceleration of about 30,000 mm/s² and a speed of about 7,000 mm/min. The slow insertion phase then proceeded for the remainder of the distance required at an acceleration/deceleration of about 80,000 mm/s² and a speed of about 1,000 mm/min. With a slower insertion speed during the slow insertion phase, the distance required to stop the motion generator was greatly reduced, which resulted in more precise stoppage of the syringe insertion process. The insertion method was reconfigured to have three steps. The slower speed allowed for an approximate 0.2 mm stop after the detection of a trigger condition.

With the improved stoppage precision, a lower force setting near the end of the last force peak can be used as the trigger condition. This trigger condition corresponded to the distal end of the rigid needle shield passing by the friction point in the proximal cap. This improved the accuracy of the depth placement of the rigid needle shield and syringe by using a low force on the downward slope of the last force peak. Previously, in the one-step insertion stage, a trigger condition was set on a peak prior to the final peak, which could introduce inaccurate depth placements based on varying rigid needle shield lengths.

Using the new two-stage insertion settings, a total of sixty samples were inserted either above or below the friction point of the proximal cap. A below depth setting of about 1.0 mm past nominal in the proximal direction, and an above depth setting of about 1.0 mm less than nominal in the distal direction were used. For the above nominal setting, a different trigger condition setting was required for the ComoView® control monitor. Ten syringes were inserted above and ten syringes were inserted below the nominal setting for each syringe type (Type 1, Type 2, Type 3).

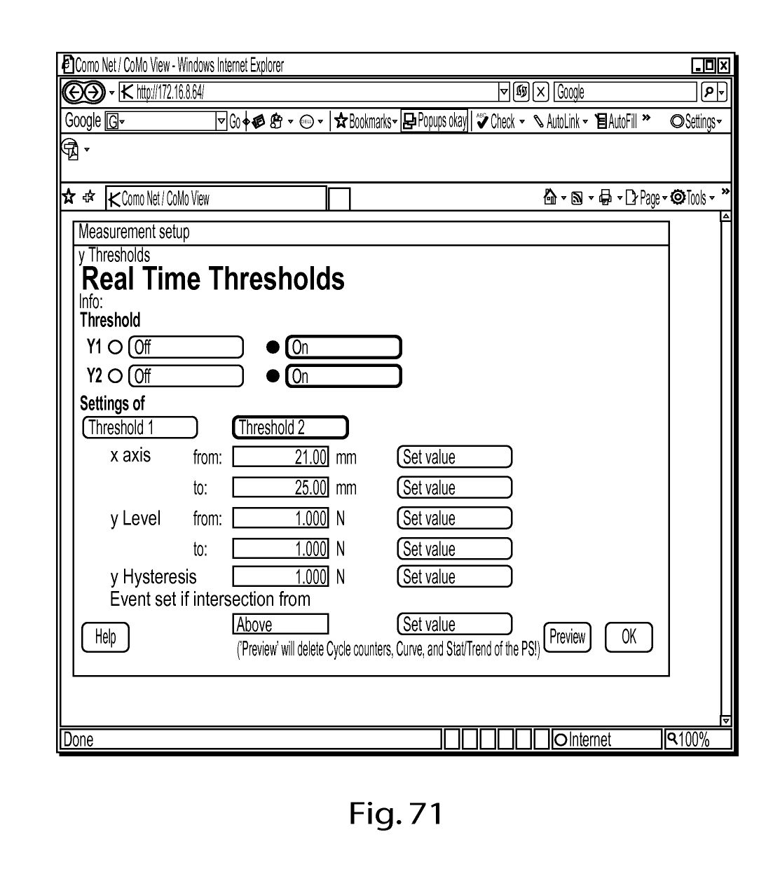

FIG. 71 illustrates a user interface showing an exemplary trigger condition setting in which the trigger force is about 1 N, the trigger hysteresis is about 1 N from above, and the x-axis range is from about 21 mm to about 25 mm.

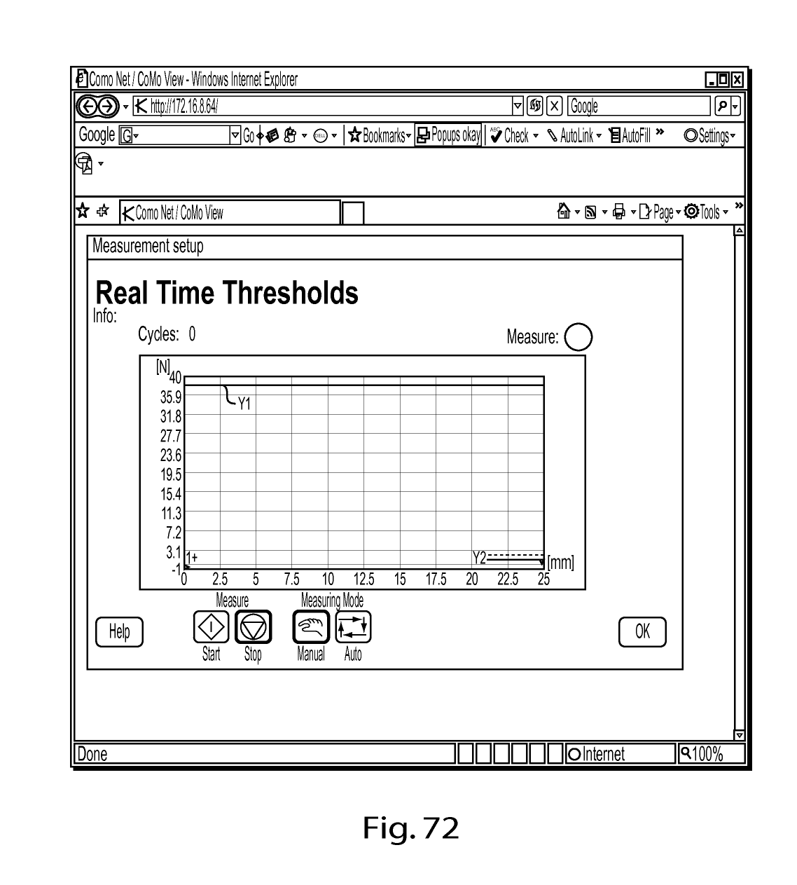

FIG. 72 illustrates an empty user interface and graph used to plot a force profile generated by the syringe insertion process. FIG. 72 shows the trigger condition settings shown in FIG. 71.

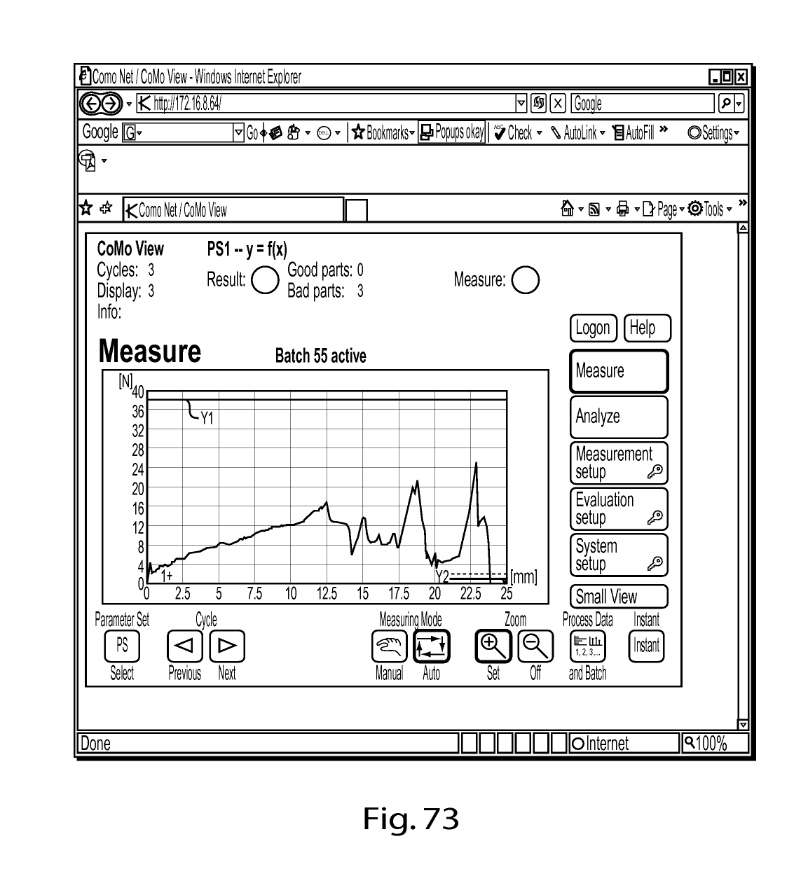

FIG. 73 illustrates a user interface and graph of a force profile generated by an exemplary Type 1 syringe during the syringe insertion process.

Table 9 summarizes the optimal insertion depths achieved by the two-step insertion process based on sixty test syringes (twenty of each syringe type).

TABLE 9
Summary of optimal insertion depths (in mm) achieved
by exemplary two-step insertion process

| Sample # | Syringe Type 1 | Syringe Type 2 | Syringe Type 3 |
|---|---|---|---|
| 1 | 7.80 | 7.83 | 7.94 |
| 2 | 8.04 | 7.79 | 8.11 |
| 3 | 8.15 | 7.73 | 8.00 |
| 4 | 8.06 | 8.10 | 7.95 |
| 5 | 8.13 | 7.88 | 8.04 |
| 6 | 7.96 | 7.77 | 8.04 |
| 7 | 8.05 | 7.83 | 8.08 |
| 8 | 8.12 | 8.00 | 8.02 |
| 9 | 8.09 | 7.94 | 8.00 |
| 10 | 7.82 | 7.75 | 8.03 |
| 11 | 7.96 | 7.86 | 8.02 |
| 12 | 8.14 | 7.71 | 8.15 |
| 13 | 8.09 | 8.16 | 8.09 |
| 14 | 7.98 | 7.93 | 7.99 |
| 15 | 8.13 | 7.97 | 7.93 |
| 16 | 8.19 | 7.90 | 7.96 |
| 17 | 8.01 | 7.80 | 7.98 |
| 18 | 7.79 | 7.91 | 7.94 |
| 19 | 8.14 | 7.81 | 7.97 |
| 20 | 8.00 | 7.72 | 8.03 |
| Average | 8.0325 | 7.8695 | 8.0135 |
| StDev | 0.118981 | 0.122538 | 0.060199 |

The average insertion depth for the Type 1 syringes was about 8.03 mm (±0.12), for the Type 2 syringes about 7.87 mm (±0.12), and for the Type 3 syringes about 8.01 mm (±0.06). The standard deviation of the insertion depths for the two-step insertion process was about two to four fold better than the previously tested one-step insertion process.

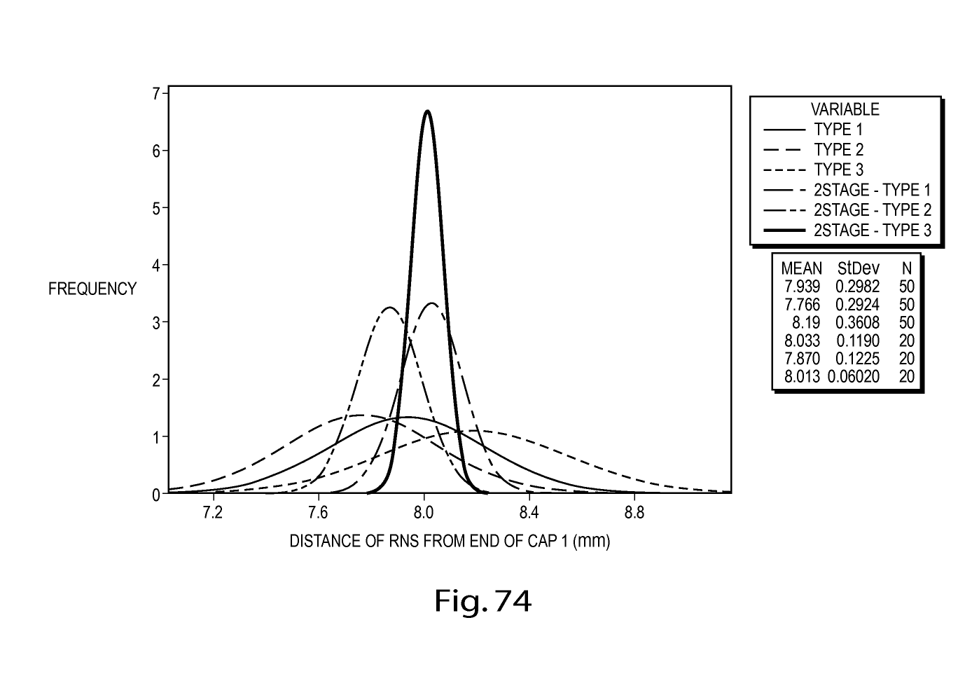

FIG. 74 illustrates a comparative histogram of the number of tested syringes for each syringe type (y-axis) against the achieved insertion depths in mm (x-axis), for both the one-step insertion process and the two-step insertion process.

To confirm the accuracy of the two-step insertion process, a trigger hysteresis 1.0 mm above an exemplary trigger force and a trigger hysteresis 1.0 mm below an exemplary trigger force were evaluated. For the below nominal case, a trigger plus move of 1.0 mm was used.

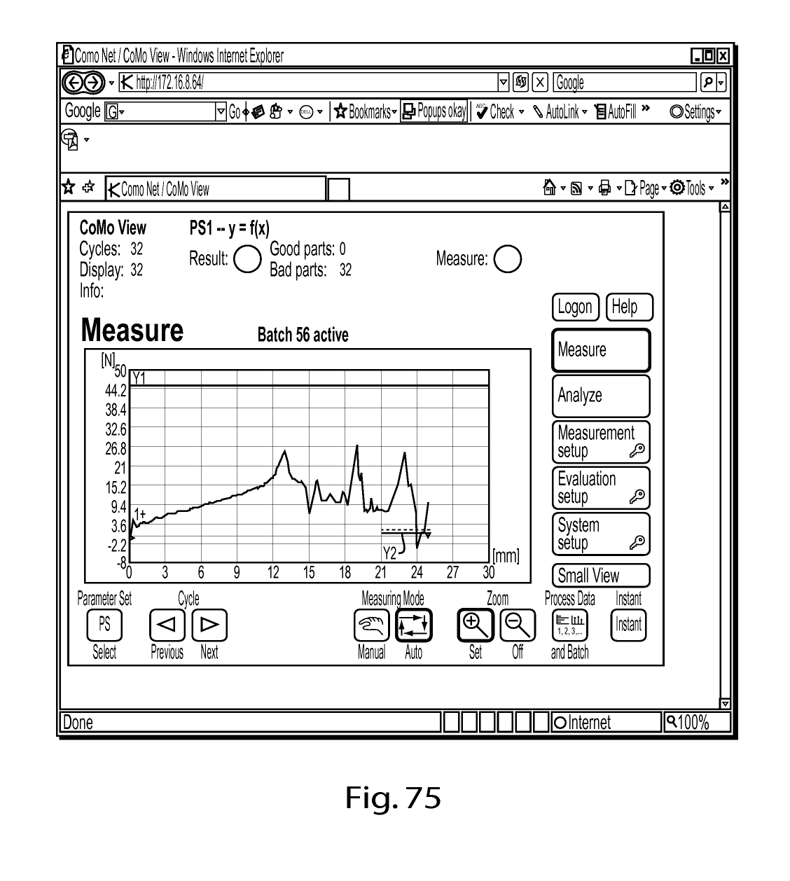

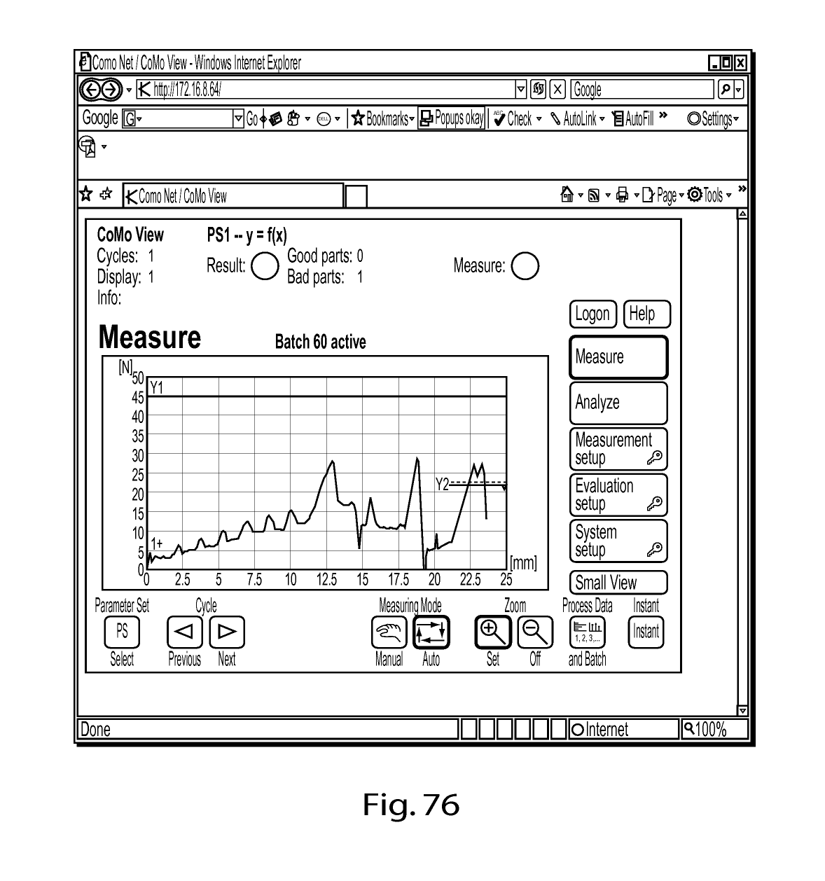

FIG. 75 illustrates a force profile generated by insertion of a Type 1 syringe insertion at 1.0 mm above nominal, and FIG. 76 illustrates a force profile generated by insertion of a Type 2 syringe at 1.0 mm below nominal. Exemplary insertion systems were able to insert the tested syringes to optimal insertion depths.

Table 10 summarizes insertion depths achieved for different exemplary syringes inserted using an exemplary two-step insertion process.

TABLE 10
Summary of insertion depths (in mm) achieved
using an exemplary two-step insertion process

| Sample # | Syringe Type 1 | Syringe Type 2 | Syringe Type 3 |
|---|---|---|---|
| 1 | 9.81 | 8.99 | 9.63 |
| 2 | 9.76 | 9.55 | 9.76 |
| 3 | 9.78 | 9.55 | 8.1 |
| 4 | 9.65 | 9.68 | 9.53 |
| 5 | 9.73 | 8.95 | 9.62 |
| 6 | 9.62 | 9.71 | 9.76 |
| 7 | 9.65 | 9.66 | 8.12 |
| 8 | 9.6 | 7.89 | 9.54 |
| 9 | 9.8 | 9.72 | 9.55 |
| 10 | 9.75 | 9.65 | 10.3 |
| Average | 9.715 | 9.335 | 9.391 |
| StDev | 0.077924 | 0.583138 | 0.711578 |

Table 11 summarizes insertion depths achieved for different exemplary syringes inserted using an exemplary two-step insertion process.

TABLE 11
Summary of insertion depths (in mm) achieved
using an exemplary two-step insertion process

| Sample # | Syringe Type 1 | Syringe Type 2 | Syringe Type 3 |
|---|---|---|---|
| 1 | 7.3 | 7.66 | 7.46 |
| 2 | 7.41 | 7.66 | 7.37 |
| 3 | 7.44 | 7.83 | 7.49 |
| 4 | 7.28 | 7.81 | 7.27 |
| 5 | 7.47 | 7.67 | 7.44 |
| 6 | 7.34 | 7.73 | 7.42 |
| 7 | 7.33 | 7.71 | 7.37 |
| 8 | 7.28 | 7.76 | 7.28 |
| 9 | 7.26 | 7.83 | 7.31 |
| 10 | 7.36 | 7.65 | 7.12 |
| Average | 7.347 | 7.731 | 7.353 |
| StDev | 0.072273 | 0.072641 | 0.111161 |

Improved insertion depths were achieved using the two-step insertion process as compared to the one-step insertion process.

### VIII. INCORPORATION BY REFERENCE

The contents of all references, including patents and patent applications, cited throughout this application are hereby incorporated herein by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

### IX. EQUIVALENTS

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than shown.

What is claimed is:

1. A method for assembling a sub-assembly of components for use in forming an automatic injection device, the method comprising:
cooperatively coupling a first component of the automatic injection device to a second component of the automatic injection device;
detecting one or more forces exerted against the cooperative coupling of the first component to the second component;
generating a trigger instruction upon verifying that one or more of the detected forces satisfy or do not satisfy one or more predefined force values; and
automatically controlling the cooperative coupling of the first component to the second component in response to the trigger instruction.

2. The method of claim 1, wherein the trigger instruction is generated upon matching a first detected force to a first predefined range of forces over a first predefined distance moved by the first component.

3. The method of claim 1, wherein the trigger instruction is generated upon matching a plurality of detected forces to a predefined range of forces over a predefined range of distances moved by the first component.

4. The method of claim 1, wherein the trigger instruction is generated upon matching the one or more detected forces to a predefined feature of a force profile.

5. The method of claim 4, wherein the predefined feature of the force profile is a peak.

6. The method of claim 4, wherein the predefined feature of the force profile is a trough.

7. The method of claim 1, wherein the trigger instruction indicates that the first component has reached a desired position relative to the second component, and wherein controlling the cooperative coupling of the first component to the second component comprises:
terminating the cooperative coupling of the first component to the second component.

8. The method of claim 1, further comprising:
determining that the first component has been driven toward the second component over a predefined distance; and
decelerating a rate of movement of the first component toward the second component.

9. The method of claim 1, wherein the trigger instruction is generated when the one or more detected forces are lower than the one or more predefined force values to indicate that a biasing mechanism positioned between the first and second components is absent, and wherein controlling the cooperative coupling of the first component to the second component comprises:
terminating the cooperative coupling of the first component to the second component; and
discarding the first component and the second component.

10. The method of claim 1, wherein the trigger instruction is generated when the one or more detected forces are higher than the one or more predefined force values to indicate that a biasing mechanism positioned between the first and second components is misaligned with the first and second components, and wherein controlling the cooperative coupling of the first component to the second component comprises:
terminating the cooperative coupling of the first component to the second component; and
discarding the first component, the second component and the biasing mechanism.

11. The method of claim 1, wherein the trigger instruction indicates that the first component is improperly assembled with the second component, and wherein controlling the cooperative coupling of the first component to the second component comprises:
terminating the cooperative coupling of the first component to the second component; and
discarding the first component and the second component.

12. The method of claim 1, wherein the trigger instruction indicates that the first component is properly assembled with the second component, and wherein controlling the cooperative coupling of the first component to the second component comprises:
terminating the cooperative coupling of the first component to the second component; and
providing an indication that the first component and the second component are properly assembled.

13. The method of claim 1, wherein the sub-assembly is a syringe housing sub-assembly, the first component is a shroud deployable to protect an injection needle, and the second component is a syringe carrier for movably holding a syringe within the automatic injection device.

14. The method of claim 13, further comprising:
testing deployment of the shroud by partially deploying the shroud after cooperative coupling of the shroud to the syringe carrier;
detecting one or more forces generated during the partial deployment of the shroud; and
based on the one or more forces detected during the partial deployment of the shroud, automatically determining whether the shroud is successfully deployed.

15. The method of claim 1, wherein the sub-assembly is a firing mechanism sub-assembly, the first component is a plunger and the second component is a firing body configured to actuate the plunger.

16. The method of claim 15, further comprising:
detecting one or more forces generated after cooperative coupling of the plunger to the firing body; and
based on the one or more forces detected after assembly of the plunger with the firing body, testing undesirable decoupling of the plunger from the firing body.

17. The method of claim 1, wherein the first component is a syringe assembly and the second component is a housing assembly, the syringe assembly comprising one or more structural features on an outer surface, the housing assembly comprising a friction point on an inner surface, and wherein the one or more detected forces are generated as the one or more structural features of the syringe assembly are inserted past the friction point of the housing assembly.

18. The method of claim 17, further comprising:
providing the syringe assembly, the syringe assembly comprising:
a syringe body for holding a therapeutic agent, the syringe body having a proximal end and a distal end,
a needle coupled to the proximal end of the syringe body, and
a rigid needle shield provided over the needle and coupled to the proximal end of the syringe body for protectively covering the needle;
wherein the one or more structural features are provided on an outer surface of the rigid needle shield.

19. The method of claim 18, further comprising:
providing the housing assembly of the automatic injection device, the housing assembly comprising:
a housing body extending between a proximal end and a distal end, the housing body including an internal bore for accommodating the syringe body of the syringe assembly, and
a needle cap coupled to the proximal end of the housing body, the needle cap including an internal bore for accommodating the rigid needle shield of the syringe assembly;
wherein the friction point in the housing assembly is provided in the internal bore of the needle cap.

20. A system for assembling a sub-assembly of components for use in forming an automatic injection device, the system comprising:
an assembly station for cooperatively coupling a first component of the automatic injection device to a second component of the automatic injection device;
a force detection mechanism configured to detect one or more forces exerted against the cooperative coupling of the first component to the second component; and
a controller programmed to automatically generate a trigger instruction upon verifying that the one or more of the detected forces satisfy or do not satisfy one or more predefined force values;
wherein the assembly station is configured to automatically control the cooperative coupling of the first component to the second component in response to the trigger instruction.

21. The system of claim 20, wherein the sub-assembly is a syringe housing sub-assembly, the first component is a shroud deployable to protect an injection needle, and the second component is a syringe carrier for movably holding a syringe within the automatic injection device.

22. The system of claim 21, wherein the assembly station is further configured to test deployment of the shroud by partially deploying the shroud after assembly of the shroud with the syringe carrier, wherein the force detection mechanism is further configured to detect one or more forces generated during the partial deployment of the shroud, and wherein the controller is further programmed to determine whether the shroud is successfully deployed based on the one or more forces detected during the partial deployment of the shroud.

23. The system of claim 20, wherein the sub-assembly is a firing mechanism sub-assembly, the first component is a plunger and the second component is a firing body configured to actuate the plunger.

24. The system of claim 23, wherein the force detection mechanism is further configured to detect one or more forces generated after assembly of the plunger with the firing body, and wherein the controller is further programmed to test undesirable decoupling of the plunger from the firing body based on the one or more forces detected after assembly of the plunger with the firing body.

25. The system of claim 20, wherein the first component is a syringe assembly and the second component is a housing assembly, the syringe assembly comprising one or more structural features on an outer surface, the housing assembly comprising a friction point on an inner surface, and wherein the one or more detected forces are generated as the one or more structural features of the syringe assembly are inserted past the friction point of the housing assembly.

26. The system of claim 25, wherein the syringe assembly comprises:
a syringe body for holding a therapeutic agent, the syringe body having a proximal end and a distal end;
a needle coupled to the proximal end of the syringe body; and
a rigid needle shield provided over the needle and coupled to the proximal end of the syringe body for protectively covering the needle;
wherein the one or more structural features are provided on an outer surface of the rigid needle shield.

27. The system of claim 26, wherein the housing assembly comprises:
a housing body extending between a proximal end and a distal end, the housing body including an internal bore for accommodating the syringe body of the syringe assembly; and
a needle cap coupled to the proximal end of the housing body, the needle cap including an internal bore for accommodating the rigid needle shield of the syringe assembly;
wherein the friction point in the housing assembly is provided in the internal bore of the needle cap.

28. The system of claim 20, wherein the trigger instruction indicates that the first component has reached a desired position relative to the second component, and wherein the assembly station is configured to terminate the cooperative coupling of the first component to the second component in response to the trigger instruction.

29. The system of claim 20, wherein the controller is further programmed to determine that the first component has been driven toward the second component over a predefined distance, and wherein the assembly station is further configured to decelerate the rate of movement of the first component toward the second component upon driving the first component over the predefined distance.

30. The system of claim 20, wherein the controller is programmed to generate the trigger instruction when the one or more detected forces are lower than a predefined force value to indicate that a biasing mechanism positioned between the first and second components is absent, and wherein the assembly station is further configured to terminate the cooperative coupling of the first component to the second component and to discard the first component and the second component in response to the trigger instruction.

31. The system of claim 20, wherein the controller is programmed to generate the trigger instruction when the one or more detected forces are higher than a predefined force value to indicate that a biasing mechanism positioned between the first and second components is misaligned with the first and second components, and wherein the assembly station is further configured to terminate the cooperative coupling of the first component to the second component and to discard the first component, the second component and the biasing mechanism in response to the trigger instruction.

32. The system of claim 20, wherein the controller is programmed to generate the trigger instruction to indicate that the first component is improperly assembled with the second component, and wherein the assembly station is further configured to terminate the cooperative coupling of the first component to the second component and to discard the first component and the second component in response to the trigger instruction.

33. The system of claim 20, wherein the controller is programmed to generate the trigger instruction to indicate that the first component is properly assembled with the second component, and wherein the assembly station is further configured to terminate the cooperative coupling of the first component to the second component and to provide an indication that the first component and the second component are properly assembled in response to the trigger instruction.

34. A method for assembling an automatic injection device, the method comprising:
inserting a syringe assembly of the automatic injection device into a housing assembly of the automatic injection device, the syringe assembly comprising a first outer diameter greater than a second outer diameter, an inner surface of the housing assembly having a friction point;
detecting one or more forces generated as the first outer diameter and the second outer diameter of the syringe assembly are inserted past the friction point in the housing assembly;
generating a trigger instruction upon matching one or more of the detected forces to one or more predefined forces values; and
controlling the insertion of the syringe assembly into the housing assembly in response to the trigger instruction.

35. The method of claim 34, further comprising:
providing the syringe assembly, the syringe assembly comprising:
a syringe body for holding a therapeutic agent, the syringe body having a proximal end and a distal end,
a needle coupled to the proximal end of the syringe body, and
a rigid needle shield provided over the needle and coupled to the proximal end of the syringe body for protectively covering the needle.

36. The method of claim 35, wherein the first outer diameter corresponds to a first feature protruding from an outer surface of the rigid needle shield.

37. The method of claim 36, wherein the first feature is provided at a distal end of the rigid needle shield adjacent to the proximal end of the syringe body.

38. The method of claim 35, wherein the first outer diameter corresponds to a first feature protruding from an outer surface of the syringe body.

39. The method of claim 38, wherein the first feature is provided at the proximal end of the syringe body.

40. The method of claim 35, further comprising:
providing the housing assembly of the automatic injection device, the housing assembly comprising:
a housing body extending between a proximal end and a distal end, the housing body including an internal bore for accommodating the syringe body of the syringe assembly, and
a needle cap coupled to the proximal end of the housing body, the needle cap including an internal bore for accommodating the rigid needle shield of the syringe assembly;
wherein the friction point in the housing assembly is provided in the internal bore of the needle cap.

41. The method of claim 34, wherein generating the trigger instruction comprises:
matching a first detected force value generated at a first time to a first predefined force value;
matching a second detected force value generated at a second later time to a second predefined force value; and
determining that the first and second detected force values are detected within a predefined insertion range of the syringe assembly into the housing assembly.
